(19) United States
(12) Patent Application Publication
(10) Pub. No.: US 2008/0175914 A1
(43) Pub. Date: Jul. 24, 2008

(54) Pyrazolo[3,4-b]Pyridine Compounds, and their Use as Phosphodiesterase Inhibitors

(75) Inventors: David George ALLEN, Stevenage (GB); Diane Mary Coe, Stevenage (GB); Caroline Mary Cook, Stevenage (GB); Michael Dennis Dowle, Stevenage (GB); Christopher David Edlin, Stevenage (GB); Julie Nicole Hamblin, Stevenage (GB); Martin Redpath Johnson, Stevenage (GB); Paul Spencer Jones, Stevenage (GB); Richard Graham Knowles, Stevenage (GB); Mika Kristian Lindvall, Emeryville, CA (US); Charlotte Jane Mitchell, Stevenage (GB); Alison Judith Redgrave, Stevenage (GB); Peter Ward, Stevenage (GB)

Correspondence Address: GLAXOSMITHKLINE, Corporate Intellectual Property - UW2220, P.O. Box 1539, King of Prussia, PA, 19406-0939, US

(73) Assignee: Glaxo Group Limited

(21) Appl. No.: 12/013,529

(22) Filed: Jan. 14, 2008

Related U.S. Application Data
(63) Continuation of application No. 10/527,866, filed on Sep. 27, 2005.

(30) Foreign Application Priority Data
Sep. 16, 2002 (GB) 0221455.9
Dec. 23, 2002 (GB) 0230045.7
Mar. 21, 2003 (GB) 0306595.0
Apr. 7, 2003 (GB) 0308017.3
Aug. 21, 2003 (GB) 0319708.4
Sep. 9, 2003 (GB) 0321074.7

(51) Int. Cl. A61K 9/14; C07D 487/04; A61P 29/00; A61P 11/00; A61P 37/08; A61K 31/437

(52) U.S. Cl. 424/489; 546/120; 514/303

(57) ABSTRACT

The invention relates to a compound of formula (I) or a salt thereof:

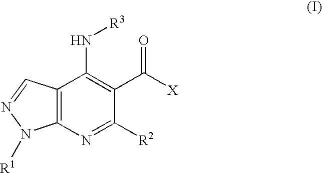

wherein:
R¹ is C₁₋₄alkyl, C₁₋₃fluoroalkyl, —CH₂CH₂OH or —CH₂CH₂CO₂C₁₋₂alkyl;
R² is a hydrogen atom (H), methyl or C₁fluoroalkyl;
R³ is optionally substituted C₃₋₈cycloalkyl or optionally substituted mono-unsaturated-C₅₋₇cycloalkenyl or an optionally substituted heterocyclic group of sub-formula (aa), (bb) or (cc);

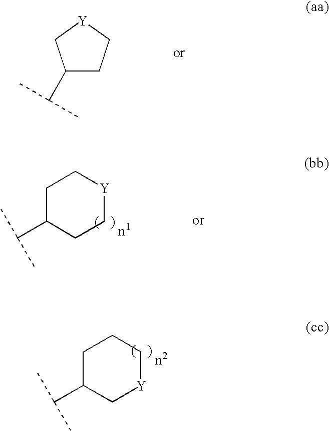

in which n¹ and n² independently are 1 or 2; and in which Y is O, S, SO₂, or NR¹⁰;
or R³ is a bicyclic group (dd) or (ee):
and wherein X is NR⁴R⁵ or OR^{5a}.

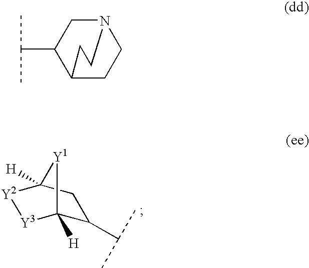

The compounds are phosphodiesterase (PDE) inhibitors, in particular PDE4 inhibitors. Also provided is the use of a compound of formula (I), or a pharmaceutically acceptable salt thereof, in the manufacture of a medicament for the treatment and/or prophylaxis of an inflammatory and/or allergic disease in a mammal such as a human, for example chronic obstructive pulmonary disease (COPD), asthma, or allergic rhinitis.

### CROSS-REFERENCE TO PREVIOUS APPLICATION

This application is continuation application derived from U.S. Ser. No. 10/527,866 filed 27 Sep. 2005 (pending) which is a 371 of Application No. PCT/EP2003/011814 filed 12 Sep. 2003.

### BACKGROUND OF THE INVENTION

The present invention relates to pyrazolopyridine compounds, processes for their preparation, intermediates usable in these processes, and pharmaceutical compositions containing the compounds. The invention also relates to the use of the pyrazolopyridine compounds in therapy, for example as inhibitors of phosphodiesterases and/or for the treatment and/or prophylaxis of inflammatory and/or allergic diseases such as chronic obstructive pulmonary disease (COPD), asthma, rheumatoid arthritis or allergic rhinitis.

U.S. Pat. No. 3,979,399, U.S. Pat. No. 3,840,546, and U.S. Pat. No. 3,966,746 (E.R.Squibb & Sons) disclose 4-amino derivatives of pyrazolo[3,4-b]pyridine-5-carboxamides wherein the 4-amino group NR₃R₄ can be an acyclic amino group wherein R₃ and R₄ may each be hydrogen, lower alkyl (e.g. butyl), phenyl, etc.; NR₃R₄ can alternatively be a 3-6-membered heterocyclic group such as pyrrolidino, piperidino and piperazino. The compounds are disclosed as central nervous system depressants useful as ataractic, analgesic and hypotensive agents.

U.S. Pat. No. 3,925,388, U.S. Pat. No. 3,856,799, U.S. Pat. No. 3,833,594 and U.S. Pat. No. 3,755,340 (E.R.Squibb & Sons) disclose 4-amino derivatives of pyrazolo[3,4-b]pyridine-5-carboxylic acids and esters. The 4-amino group NR₃R₄ can be an acyclic amino group wherein R₃ and R₄ may each be hydrogen, lower alkyl (e.g. butyl), phenyl, etc.; NR₃R₄ can alternatively be a 5-6-membered heterocyclic group in which an additional nitrogen is present such as pyrrolidino, piperidino, pyrazolyl, pyrimidinyl, pyridazinyl or piperazinyl. The compounds are mentioned as being central nervous system depressants useful as ataractic agents or tranquilisers, as having antiinflammatory and analgesic properties. The compounds are mentioned as increasing the intracellular concentration of adenosine-3′,5′-cyclic monophosphate and for alleviating the symptoms of asthma.

H. Hoehn et al., J. Heterocycl. Chem., 1972, 9(2), 235-253 discloses a series of 1H-pyrazolo[3,4-b]pyridine-5-carboxylic acid derivatives with 4-hydroxy, 4-chloro, 4-alkoxy, 4-hydrazino, and 4-amino substituents.

CA 1003419, CH 553 799 and T. Denzel, Archiv der Pharmazie, 1974, 307(3), 177-186 disclose 4,5-disubstituted 1H-pyrazolo[3,4-b]pyridines unsubstituted at the 1-position.

Japanese laid-open patent application JP-2002-20386-A (Ono Yakuhin Kogyo KK) published on 23 Jan. 2002 discloses pyrazolopyridine compounds of the following formula:

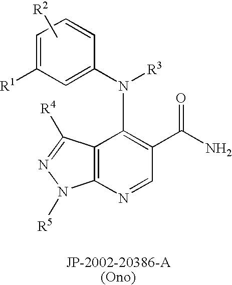

wherein R¹ denotes 1) a group —OR⁶, 2) a group —SR⁷, 3) a C2-8 alkynyl group, 4) a nitro group, 5) a cyano group, 6) a C1-8 alkyl group substituted by a hydroxy group or a C1-8 alkoxy group, 7) a phenyl group, 8) a group —C(O)R⁸, 9) a group —SO₂NR⁹R¹⁰, 10) a group —NR¹¹SO₂R¹², 11) a group —NR¹³C(O)R¹⁴ or 12) a group —CH═NR¹⁵. R⁶ and R⁷ denote i) a hydrogen atom, ii) a C1-8 alkyl group, iii) a C1-8 alkyl group substituted by a C1-8 alkoxy group, iv) a trihalomethyl group, v) a C3-7 cycloalkyl group, vi) a C1-8 alkyl group substituted by a phenyl group or vii) a 3-15 membered mono-, di- or tricyclic hetero ring containing 1-4 nitrogen atoms, 1-3 oxygen atoms and/or 1-3 sulphur atoms. R² denotes 1) a hydrogen atom or 2) a C1-8 alkoxy group. R³ denotes 1) a hydrogen atom or 2) a C1-8 alkyl group. R⁴ denotes 1) a hydrogen atom, 2) a C1-8 alkyl group, 3) a C3-7 cycloalkyl group, 4) a C1-8 alkyl group substituted by a C3-7 cycloalkyl group, 5) a phenyl group which may be substituted by 1-3 halogen atoms or 6) a 3-15 membered mono-, di- or tricyclic hetero ring containing 1-4 nitrogen atoms, 1-3 oxygen atoms and/or 1-3 sulphur atoms. R⁵ denotes 1) a hydrogen atom, 2) a C1-8 alkyl group, 3) a C3-7 cycloalkyl group, 4) a C1-8 alkyl group substituted by a C3-7 cycloalkyl group or 5) a phenyl group which may be substituted by 1-3 substituents. In group R³, a hydrogen atom is preferred. In group R⁴, methyl, ethyl, cyclopropyl, cyclobutyl or cyclopentyl are preferred. The compounds of JP-2002-20386-A are stated as having PDE4 inhibitory activity and as being useful in the prevention and/or treatment of inflammatory diseases and many other diseases.

EP 0 076 035 A1 (ICI Americas) discloses pyrazolo[3,4-b]pyridine derivatives as central nervous system depressants useful as tranquilisers or ataractic agents for the relief of anxiety and tension states.

The compound cartazolate, ethyl 4-(n-butylamino)-1-ethyl-1H-pyrazolo[3,4-b]-pyridine-5-carboxylate, is known. J. W. Daly et al., Med. Chem. Res., 1994, 4, 293-306 and D. Shi et al., Drug Development Research, 1997, 42, 41-56 disclose a series of 4-(amino)substituted 1H-pyrazolo[3,4-b]pyridine-5-carboxylic acid derivatives, including ethyl 4-cyclopentylamino-1-methyl-1H-pyrazolo[3,4-b]pyridine-5-carboxylate, and their affinities and antagonist activities at A₁- and A_{2A}-adenosine receptors, and the latter paper discloses their affinities at various binding sites of the GABA_{A}-receptor channel. S. Schenone et al., Bioorg. Med. Chem. Lett., 2001, 11, 2529-2531 and F. Bondavalli et al., J. Med. Chem., 2002, vol. 45 (Issue 22, 24 Oct. 2002, allegedly published on Web Sep. 24, 2002), pp. 4875-4887 disclose a series of 4-amino-1-(2-chloro-2-phenylethyl)-1H-pyrazolo[3,4-b]pyridine-5-carboxylic acid ethyl esters as A₁-adenosine receptor ligands.

WO 02/060900 A2 appears to disclose, as MCP-1 antagonists for treatment of allergic, inflammatory or autoimmune disorders or diseases, a series of bicyclic heterocyclic compounds with a —C(O)—NR⁴—C(O)—NR⁵R⁶ substituent, including isoxazolo[5,4-b]pyridines and 1H-pyrazolo[3,4-b]pyridines (named as pyrazolo[5,4-b]pyridines) with the —C(O)—NR⁴—C(O)—NR⁵R⁶ group as the 5-substituent and optionally substituted at the 1-, 3-, 4-, and/or 6-positions. Bicyclic heterocyclic compounds with a —C(O)NH₂ substituent instead of the —C(O)—NR⁴—C(O)—NR⁵R⁶ substituent are alleged to be disclosed in WO 02/060900 as intermediates in the synthesis of the —C(O)—NR⁴—C(O)—NR⁵R⁶ substituted compounds.

It is desirable to find new compounds which bind to, and preferably inhibit, phosphodiesterase type IV (PDE4).

### SUMMARY OF THE INVENTION

The present invention provides a compound of formula (I) or a salt thereof (in particular, a pharmaceutically acceptable salt thereof):

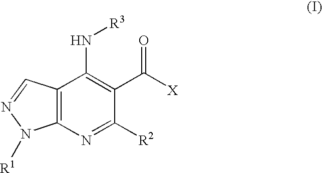

wherein:
R¹ is C₁₋₄alkyl, C₁₋₃fluoroalkyl, —CH₂CH₂OH or —CH₂CH₂CO₂C₁₋₂alkyl;
R² is a hydrogen atom (H), methyl or C₁fluoroalkyl;
R³ is optionally substituted C₃₋₈cycloalkyl or optionally substituted mono-unsaturated-C₅₋₇cycloalkenyl or an optionally substituted heterocyclic group of sub-formula (aa), (bb) or (cc);

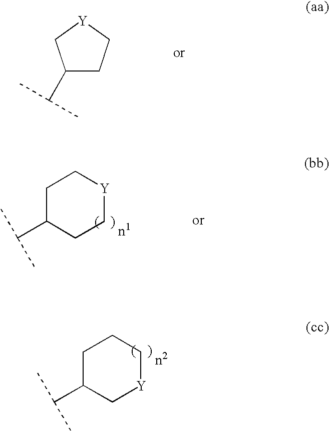

in which n¹ and n² independently are 1 or 2; and in which Y is O, S, SO₂, or NR¹⁰; where R¹⁰ is a hydrogen atom (H), C₁₋₄alkyl (e.g. methyl or ethyl), C₁₋₂fluoroalkyl, CH₂C(O)NH₂, C(O)NH₂, C(O)—C₁₋₂alkyl, C(O)—C₁fluoroalkyl or —C(O)—CH₂O—C₁₋₂alkyl;
and wherein in R³ the C₃₋₈cycloalkyl or the heterocyclic group of sub-formula (aa), (bb) or (cc) is optionally substituted with one or two substituents independently being (e.g. being) oxo (═O); OH; C₁₋₂alkoxy; C₁₋₂fluoroalkoxy (e.g. trifluoromethoxy); NHR²¹ wherein R²¹ is a hydrogen atom (H) or C₁₋₅ straight-chain alkyl (e.g. H or C₁₋₄ straight-chain alkyl); C₁₋₂alkyl; C₁₋₂fluoroalkyl (e.g. C₁fluoroalkyl such as —CH₂F or —CHF₂); —CH₂OH; —CH₂CH₂OH; —CH₂NHR²² wherein R²² is H or C₁₋₂alkyl; —C(O)OR²³ wherein R²³ is H or C₁₋₂alkyl; —C(O)NHR²⁴ wherein R²⁴ is H or C₁₋₂alkyl; —C(O)R²⁵ wherein R²⁵ is C₁₋₂alkyl; fluoro; hydroxyimino (═N—OH); or (C₁₋₄alkoxy)imino (═N—OR²⁶ where R²⁶ is C₁₋₄alkyl); and wherein any OH, alkoxy, fluoroalkoxy or NHR²¹ substituent is not substituted at the R³ ring carbon attached (bonded) to the —NH— group of formula (I) and is not substituted at either R³ ring carbon bonded to the Y group of the heterocyclic group (aa), (bb) or (cc);
and wherein, when R³ is optionally substituted mono-unsaturated-C₅₋₇cycloalkenyl, then the cycloalkenyl is optionally substituted with one or two substituents being fluoro or C₁₋₂alkyl provided that if there are two substituents then they are not both C₂alkyl, and the R³ ring carbon bonded to the —NH— group of formula (I) does not partake in the cycloalkenyl double bond;
or R³ is a bicyclic group of sub-formula (dd):

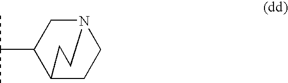

or of sub-formula (ee):

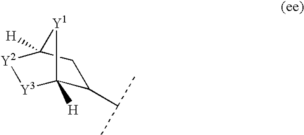

wherein Y¹, Y² and Y³ independently are CH₂ or oxygen (O) provided that no more than one of Y¹, Y² and Y³ is oxygen (O);
and X is NR⁴R⁵ or OR^{5a}, in which:
R⁴ is a hydrogen atom (H); C₁₋₆alkyl; C₁₋₃fluoroalkyl; or C₂₋₆alkyl substituted by one substituent R¹¹; and
R⁵ is a hydrogen atom (H); C₁₋₈alkyl; C₁₋₈ fluoroalkyl; C₃₋₈cycloalkyl optionally substituted by a C₁₋₂alkyl group; or —(CH₂)ₙ⁴—C₃₋₈cycloalkyl optionally substituted, in the —(CH₂)ₙ⁴— moiety or in the C₃₋₈cycloalkyl moiety, by a C₁₋₂alkyl group, wherein n⁴ is 1, 2 or 3;
or R⁵ is C₂₋₆alkyl substituted by one or two independent substituents R¹¹;
wherein each substituent R¹¹, independently of any other R¹¹ substituent present, is: hydroxy (OH); C₁₋₆alkoxy; phenyloxy; benzyloxy; —NR¹²R¹³; —NR¹⁵—C(O)R¹⁶; —NR¹⁵—C(O)—O—R¹⁶; —NR¹⁵—C(O)—NH—R¹⁵; or —NR¹⁵—SO₂R¹⁶; and wherein any R¹¹ substituent which is OH, alkoxy or —NR¹²R¹³ is not substituted at any carbon atom, of any R⁴ or R⁵ substituted alkyl, which is bonded to the nitrogen of NR⁴R⁵;
or R⁵ is —(CH₂)ₙ¹¹—C(O)R¹⁶; —(CH₂)ₙ¹²—C(O)NR¹²R¹³; —CHR¹⁹—C(O)NR¹²R¹³; —(CH₂)ₙ¹²—C(O)OR¹⁶; —(CH₂)ₙ¹²—C(O)OH; —CHR¹⁹—C(O)OR¹⁶; —CHR¹⁹—C(O)OH; —(CH₂)ₙ¹²—SO₂—NR¹²R¹³; —(CH₂)ₙ¹²—SO₂R¹⁶; or —(CH₂)ₙ¹²—CN; wherein n¹¹ is 0, 1, 2, 3 or 4 and n¹² is 1, 2, 3 or 4;
or R⁵ is —(CH₂)ₙ¹³-Het wherein n¹³ is 0, 1, 2, 3 or 4 and Het is a 4-, 5-, 6- or 7-membered saturated or partly-saturated heterocyclic ring containing one or two ring-hetero-atoms independently selected from O, S, and N; wherein any ring-hetero-atoms present are not bound to the —(CH₂)ₙ¹³— moiety when n¹³ is 1 and are not bound to the nitrogen of NR⁴R⁵ when n¹³ is 0; wherein any ring-nitrogens which are present and which are not unsaturated (i.e. which do not partake in a double bond) are present as NR¹⁷ where R¹⁷ is as defined herein; and wherein one or two of the carbon ring-atoms independently are optionally substituted by C₁₋₂alkyl;
or R⁵ is phenyl optionally substituted with, independently, one, two or three of: a halogen atom; C₁₋₆alkyl (e.g. C₁₋₄alkyl or C₁₋₂alkyl); C₁₋₂fluoroalkyl (e.g. trifluoromethyl); C₁₋₄alkoxy (e.g. C₁₋₂alkoxy); C₁₋₂fluoroalkoxy (e.g. trifluoromethoxy); C₃₋₆cycloalkyloxy; —C(O)R^{16a}; —C(O)OR³⁰; —S(O)₂—R^{16a} (e.g. C₁₋₂alkylsulphonyl or C₁₋₂alkyl-SO₂—); R^{16a}—S(O)₂—NR^{15a}— (e.g. C₁₋₂alkyl-SO₂—NH—); R⁷R⁸N—S(O)₂—; C₁₋₂alkyl-C(O)—R^{15a}N—S(O)₂—; C₁₋₄alkyl-S(O)—, Ph-S(O)—, R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-S(O)₂—CH₂—; R⁷R⁸N—S(O)₂—CH₂—; C₁₋₂alkyl-S(O)₂—NR^{15a}—CH₂—; —CH₂—OH; —CH₂CH₂—OH; —CH₂—NR⁷R⁸; —CH₂—CH₂—NR⁷R⁸; —CH₂—C(O)OR³⁰; —CH₂—C(O)—NR⁷R⁸; —CH₂—NR^{15a}—C(O)—C₁₋₃alkyl; —(CH₂)ₙ¹⁴-Het¹ where n¹⁴ is 0 or 1; cyano (CN); Ar^{5a}; or phenyl, pyridinyl or pyrimidinyl wherein the phenyl, pyridinyl or pyrimidinyl independently are optionally substituted by one or two of fluoro, chloro, C₁₋₂alkyl, C₁fluoroalkyl, C₁₋₂alkoxy or C₁fluoroalkoxy; or where two adjacent substituents taken together are —O—(CMe₂)—O— or —O—(CH₂)ₙ¹⁴—O— where n¹⁴ is 1 or 2;
wherein R⁷ and R⁸ are independently a hydrogen atom (H); C₁₋₄alkyl (e.g. C₁₋₂alkyl such as methyl); C₃₋₆cycloalkyl; or phenyl optionally substituted by one or two of: fluoro, chloro, C₁₋₂alkyl, C₁fluoroalkyl, C₁₋₂alkoxy or C₁fluoroalkoxy; or R⁷ and R⁸ together are —(CH₂)ₙ⁶— or —C(O)—(CH₂)ₙ⁷— or —C(O)—(CH₂)ₙ⁷—C(O)— or —(CH₂)ₙ⁸—X⁷—(CH₂)ₙ⁹— or —C(O)—X⁷—(CH₂)ₙ¹⁰— in which: n⁶ is 3, 4, 5 or 6, n⁷ is 2, 3, 4, or 5 (preferably n⁷ is 2, 3 or 4), n⁸ and n⁹ and n¹¹ independently are 2 or 3 (preferably independently 2), and X⁷ is O or NR¹⁴ wherein R¹⁴ is H, C₁₋₂alkyl or C(O)Me (preferably H or C₁₋₂alkyl);
   or R⁵ has the sub-formula (x), (y), (y1) or (z):

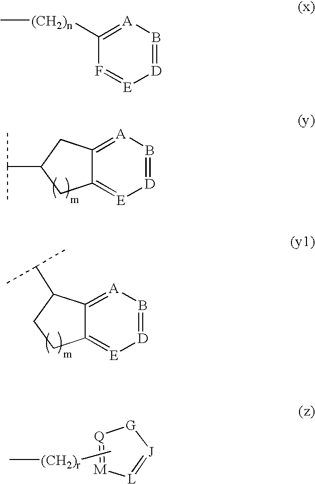

wherein in sub-formula (x), n=0, 1 or 2; in sub-formula (y) and (y1), m=1 or 2; and in sub-formula (z), r=0, 1 or 2;
wherein in sub-formula (x) and (y) and (y1), none, one or two of A, B, D, E and F are independently nitrogen or nitrogen-oxide (N⁺—O⁻) provided that no more than one of A, B, D, E and F is nitrogen-oxide; and the remaining of A, B, D, E and F are independently CH or CR⁶;
provided that when n is 0 in sub-formula (x) then one or two of A, B, D, E and F are independently nitrogen or nitrogen-oxide (N⁺—O⁻) and no more than one of A, B, D, E and F is nitrogen-oxide;
wherein, each R⁶, independently of any other R⁶ present, is: a halogen atom; C₁₋₆alkyl (e.g. C₁₋₄alkyl or C₁₋₂alkyl); C₁₋₄fluoroalkyl (e.g. C₁₋₂fluoroalkyl); C₁₋₄alkoxy (e.g. C₁₋₂alkoxy); C₁₋₂fluoroalkoxy; C₃₋₆cycloalkyloxy; —C(O)R^{16a}; —C(O)OR³⁰; —S(O)₂—R^{16a} (e.g. C₁₋₂alkylsulphonyl, that is C₁₋₂alkyl-SO₂—); R^{16a}—S(O)₂—NR^{15a}— (e.g. C₁₋₂alkyl-SO₂—NH—); R⁷R⁸N—S(O)₂—; C₁₋₂alkyl-C(O)—R^{15a}N—S(O)₂—; C₁₋₄alkyl-S(O)—, Ph-S(O)—, R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-S(O)₂—CH₂—; R⁷R⁸N—S(O)₂—CH₂—; C₁₋₂alkyl-S(O)₂—NR^{15a}—CH₂—; —CH₂—OH; —CH₂CH₂—OH; —CH₂—NR⁷R⁸; —CH₂—CH₂—NR⁷R⁸; —CH₂—C(O)OR³⁰; —CH₂—C(O)—NR⁷R⁸; —CH₂—NR^{15a}—C(O)—C₁₋₃alkyl; —(CH₂)ₙ¹⁴-Het¹ where n¹⁴ is 0 or 1; cyano (CN); Ar^{5b}; or phenyl, pyridinyl or pyrimidinyl wherein the phenyl, pyridinyl or pyrimidinyl independently are optionally substituted by one or two of fluoro, chloro, C₁₋₂alkyl, C₁fluoroalkyl, C₁₋₂alkoxy or C₁fluoroalkoxy; or where two adjacent R⁶ taken together are —O—(CMe₂)—O— or —O—(CH₂)ₙ¹⁴—O— where n¹⁴ is 1 or 2;
wherein R⁷ and R⁸ are as herein defined;
wherein sub-formula (y) and (y1), independently, are optionally substituted by oxo (═O) at a ring carbon adjacent the 6-membered aromatic ring (for example, sub-formula (y) can optionally be

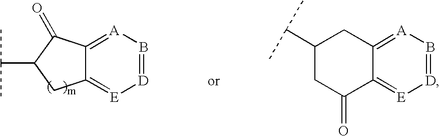

or sub-formula (y1) can optionally be

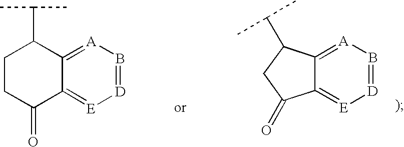

wherein in sub-formula (z), G is O or S or NR⁹ wherein R⁹ is a hydrogen atom (H), C₁₋₄alkyl or C₁₋₄fluoroalkyl; none, one, two or three of J, L, M and Q are nitrogen; and the remaining of J, L, M and Q are independently CH or CR⁶ where R⁶, independently of any other R⁶ present, is as defined herein;
or R⁴ and R⁵ taken together are —(CH₂)ₚ¹— or —C(O)—(CH₂)ₚ²— or —(CH₂)ₚ³—X⁵—(CH₂)ₚ⁴— or —C(O)—X⁵—(CH₂)ₚ⁵—, in which: p¹=3, 4, 5 or 6 (preferably p=4 or 5), p² is 2, 3, 4, or 5 (preferably p² is 2, 3 or 4), and p³ and p⁴ and p⁵ independently are 2 or 3 (independently preferably 2) and X⁵ is O or NR¹⁷;
and wherein, when R⁴ and R⁵ taken together are —(CH₂)ₚ¹— or —C(O)—(CH₂)ₚ²—, the NR⁴R⁵ heterocycle is optionally substituted by one R¹⁸ substituent wherein R¹⁸ is: C₁₋₄alkyl (e.g. C₁₋₂alkyl); C₁₋₂fluoroalkyl; C₃₋₆cycloalkyl; C₁₋₂alkoxy (not substituted at a ring-carbon bonded to the NR⁴R⁵ ring-nitrogen); C₁fluoroalkoxy (not substituted at a ring-carbon bonded to the NR⁴R⁵ ring-nitrogen); OH (not substituted at a ring-carbon bonded to the NR⁴R⁵ ring-nitrogen); —(CH₂)ₚ⁷—C(O)R¹⁶ wherein p⁷ is 0, 1, 2 or 3 (preferably p⁷ is 0 or 1); —(CH₂)ₚ⁷—C(O)OR¹⁶; —(CH₂)ₚ⁷—OC(O)R¹⁶; —(CH₂)ₚ⁷—C(O)NR¹²R¹³; —(CH₂)ₚ⁷—NR¹⁵C(O)R¹⁶; —(CH₂)ₚ⁷—NR¹⁵C(O)NR¹²R¹³; —(CH₂)ₚ⁷—NR¹⁵C(O)OR¹⁶; —(CH₂)ₚ⁷—SO₂R¹⁶; —(CH₂)ₚ⁷—SO₂ NR¹²R¹³; —(CH₂)ₚ⁷—NR¹⁵SO₂R¹⁶; —(CH₂)ₚ⁷—OH; —(CH₂)ₚ⁷—OR¹⁶; or phenyl optionally substituted by one or two of: a halogen atom, C₁₋₂alkyl, C₁fluoroalkyl, C₁₋₂alkoxy or C₁fluoroalkoxy;
   or R⁴ and R⁵ taken together are —(CH₂)ₚ¹— or —C(O)—(CH₂)ₚ²— or —(CH₂)ₚ³—X⁵—(CH₂)ₚ⁴— or —C(O)—X⁵—(CH₂)ₚ⁵— as defined herein, and wherein the NR⁴R⁵ heterocycle is fused to a phenyl ring optionally substituted on the phenyl by one or two of: a halogen atom, C₁₋₂alkyl, C₁fluoroalkyl, C₁₋₂alkoxy or C₁fluoroalkoxy; and
   R^{5a} is C₁₋₈alkyl; C₁₋₈ fluoroalkyl; C₃₋₈cycloalkyl; —(CH₂)ₙ^{4a}—C₃₋₆cycloalkyl wherein n^{4a} is 1 or 2; phenyl optionally substituted with one or two of: a halogen atom, C₁₋₂alkyl, trifluoromethyl, C₁₋₂alkoxy or trifluoromethoxy; or R^{5a} has the sub-formula (x), (y) or (z) as defined herein
   and wherein:
   R¹² and R¹³ independently are H; C₁₋₅alkyl (e.g. C₁₋₃alkyl); C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
   or R¹² and R¹³ together are —(CH₂)ₙ⁶— or —C(O)—(CH₂)ₙ⁷— or —C(O)—(CH₂)ₙ⁷—C(O)— or —(CH₂)ₙ⁸—X¹²—(CH₂)ₙ⁹— or —C(O)—X¹²—(CH₂)ₙ¹⁰— in which: n⁶ is 3, 4, 5 or 6 (preferably n⁶ is 4 or 5), n⁷ is 2, 3, 4, or 5 (preferably n⁷ is 2, 3 or 4), n⁸ and n⁹ and n¹¹ independently are 2 or 3 (independently preferably 2) and X¹² is O or NR^{14a} wherein R^{14a} is H, C₁₋₂alkyl or C(O)Me (preferably H or C₁₋₂alkyl);
   R¹⁵ is a hydrogen atom (H); C₁₋₄alkyl (e.g. ^{t}Bu or C₁₋₂alkyl e.g. methyl); C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
   R^{15a}, independent of other R^{15a}, is a hydrogen atom (H) or C₁₋₄alkyl (e.g. H, ^{t}Bu or C₁₋₂alkyl such as methyl; preferably R^{15a} is H or C₁₋₂alkyl, more preferably H);
   R¹⁶ and R^{16a} independently are:
C₁₋₆alkyl (e.g. C₁₋₄alkyl or C₁₋₂alkyl);
C₃₋₆cycloalkyl (e.g. C₅₋₆cycloalkyl) optionally substituted by one oxo (═O), OH or C₁₋₂alkyl substituent (e.g. optionally substituted at the 3- or 4-position of a C₅₋₆cycloalkyl ring; and/or preferably unsubstituted C₃₋₆cycloalkyl);
C₃₋₆cycloalkyl-CH₂— (e.g. C₅₋₆cycloalkyl-CH₂—);
pyridinyl (e.g. pyridin-2-yl) optionally substituted on a ring carbon atom by one of: a halogen atom, C₁₋₂alkyl, C₁fluoroalkyl, C₁₋₂alkoxy or C₁fluoroalkoxy;
Ar^{5c};
phenyl optionally substituted by one or two of: a halogen atom, C₁₋₂alkyl, C₁fluoroalkyl, C₁₋₂alkoxy or C₁fluoroalkoxy;
benzyl optionally substituted at an aromatic carbon atom by one or two of: a halogen atom, C₁₋₂alkyl, C₁fluoroalkyl, C₁₋₂alkoxy or C₁fluoroalkoxy; or
a 4-, 5-, 6- or 7-membered saturated heterocyclic ring connected at a ring-carbon and containing one or two ring-hetero-atoms independently selected from O, S, and N; wherein any ring-nitrogens which are present are present as NR²⁷ where R²⁷ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen;
   wherein Ar^{5a}, Ar^{5b} and Ar^{5c} independently is/are a 5-membered aromatic heterocyclic ring containing one O, S or NR^{15a} in the 5-membered ring, wherein the 5-membered ring can optionally additionally contain one or two N atoms, and wherein the heterocyclic ring is optionally substituted on a ring carbon atom by one of: a halogen atom, C₁₋₂alkyl, C₁fluoroalkyl, —CH₂OH, —CH₂—OC₁₋₂alkyl, OH (including the keto tautomer thereof) or —CH₂—NR²⁸R²⁹ wherein R²⁸ and R²⁹ independently are H or methyl;
   and R¹⁷ is a hydrogen atom (H); C₁₋₄alkyl (e.g. C₁₋₂alkyl); C₁₋₂fluoroalkyl; C₃₋₆cycloalkyl; —(CH₂)ₚ⁶—C(O)R¹⁶ wherein p⁶ is 0, 1, 2 or 3 (preferably p⁶ is 0); —(CH₂)ₚ⁶—C(O)NR¹²R¹³; —(CH₂)ₚ⁶—C(O)OR¹⁶; —(CH₂)ₚ⁶—C(O)OH; —SO₂R¹⁶; —C(O)—CH₂—NR¹²R¹³; —C(O)—CH₂—NR^{15a}—C(O)—C₁₋₃ alkyl; —C(O)—CH₂—O—C₁₋₃alkyl; or phenyl or benzyl wherein the phenyl or benzyl is optionally substituted at an aromatic carbon atom by one or two of: a halogen atom, C₁₋₂alkyl, C₁fluoroalkyl, C₁₋₂alkoxy or C₁fluoroalkoxy;
   R¹⁹ is C₁₋₄alkyl; —(CH₂)ₙ²⁰—OR²⁰ wherein n²⁰ is 1, 2, 3 or 4 and R²⁰ is a hydrogen atom (H) or C₁₋₄alkyl; —CH(Me)-OH; —CH₂—SH; —CH₂—CH₂—S-Me; benzyl; or (4-hydroxyphenyl)methyl (i.e. 4-hydroxy-benzyl); and
   R³⁰, independent of other R³⁰, is a hydrogen atom (H), C₁₋₄alkyl or C₃₋₆cycloalkyl; and
   Het¹, independent of other Het¹, is a 4-, 5-, 6- or 7-membered saturated heterocyclic ring connected at a ring-carbon and containing one or two ring-hetero-atoms independently selected from O, S, and N; wherein any ring-nitrogens which are present are present as NR³¹ where R³¹ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen;
   provided that:
   when R³ is the heterocyclic group of sub-formula (bb), n¹ is 1, and Y is NR¹⁰, then: either (a) R¹⁰ is not C₁₋₄alkyl, C₁₋₂fluoroalkyl or CH₂C(O)NH₂;
   or (b) R¹⁰ is methyl and the compound is: 1-ethyl-N-(2-ethylbutyl)-4-[(1-methylpiperidin-4-yl)amino]-1H-pyrazolo[3,4-b]pyridine-5-carboxamide or 1-ethyl-N-(4-fluorophenyl)-4-[(1-methylpiperidin-4-yl)amino]-1H-pyrazolo[3,4-b]pyridine-5-carboxamide.

Preferably, where X is OR^{5a}, the compound is other than the compound wherein R¹ is methyl, X is OEt, and R³ is cyclopentyl.

In one optional embodiment of the invention, R¹ is C₁₋₄alkyl or C₁₋₂fluoroalkyl. Alternatively or additionally, in one optional embodiment of the invention, R² is a hydrogen atom (H).

Alternatively or additionally, in one optional embodiment of the invention, R³ is C₃₋₈cycloalkyl or a heterocyclic group being

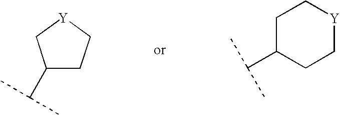

in which Y is O, S, SO₂, or NR¹⁰; where R¹⁰ is hydrogen, C₁₋₄alkyl, C₁₋₂fluoroalkyl, C(O)—C₁₋₂alkyl, or C(O)—CF₃;
and wherein in R³ the C₃₋₈cycloalkyl or heterocyclic group is optionally substituted with one or two substituents being OH, C₁₋₂alkoxy, trimethoxy, or C₁₋₂alkyl; and wherein any OH, alkoxy or trimethoxy substituent is not substituted at the (R³) ring carbon attached (bonded) to the —NH— group of formula (I) and is not substituted at either (R³) ring carbon bonded to the Y group of the heterocyclic group.

Alternatively or additionally, in one optional embodiment of the invention, R⁴ is hydrogen, C₁₋₂alkyl or C₁₋₂fluoroalkyl.

Alternatively or additionally, in one optional embodiment of the invention, R⁵ is hydrogen, C₁₋₈alkyl, C₁₋₈ fluoroalkyl, or C₃₋₈cycloalkyl; or phenyl optionally substituted with one or two of: a halogen atom, C₁₋₂alkyl, trifluoromethyl, C₁₋₂alkoxy or trifluoromethoxy; or R⁵ has the sub-formula (x), (y) or (z):

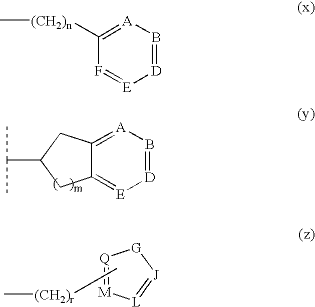

wherein in sub-formula (x), n=1 or 2; in sub-formula (y), m=1 or 2; and in sub-formula (z), r=1 or 2;

wherein in sub-formula (x) and (y), none, one or two of A, B, D, E and F are nitrogen; and the remaining of A, B, D, E and F are CH or CR⁶ where R⁶ is a halogen atom, C₁₋₄alkyl, C₁₋₄fluoroalkyl, C₁₋₂alkoxy, C₁₋₂fluoroalkoxy, C₁₋₂alkylsulphonyl (C₁₋₂alkyl-SO₂—), C₁₋₂alkyl-SO₂—NH—, R⁷R⁸N—SO₂—, R⁷R⁸N—CO—, R⁷R⁸N, OH, C₁₋₄alkoxymethyl, or C₁₋₂alkyl-SO₂—CH₂—, wherein R⁷ and R⁸ are independently hydrogen or C₁₋₂alkyl;

wherein in sub-formula (z), G is O or S or NR⁹ wherein R⁹ is C₁₋₄alkyl or C₁₋₄fluoroalkyl; none, one or two of J, L, M and Q are nitrogen; and the remaining of J, L, M and Q are CH or CR⁶ where R⁶ is as defined herein.

In the alternative to the above R⁴ and/or R⁵ optional embodiments, in one optional embodiment of the invention, R⁴ and R⁵ taken together can be —(CH₂)ₚ¹— where p¹=3, 4 or 5 (preferably p¹=4 or 5).

In one optional embodiment of the invention, R³ is optionally substituted C₃₋₈cycloalkyl or an optionally substituted heterocyclic group of sub-formula (aa), (bb) or (cc);

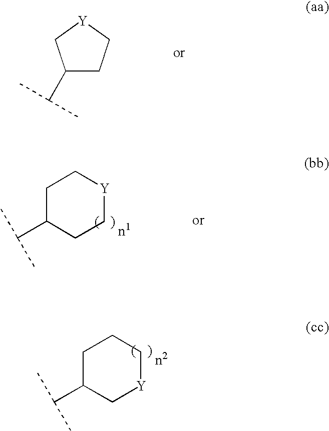

in which n¹ and n² independently are 1 or 2; and in which Y is O, S, SO₂, or NR¹⁰; where R¹⁰ is a hydrogen atom (H), C₁₋₄alkyl (e.g. methyl or ethyl), C₁₋₂fluoroalkyl, CH₂C(O)NH₂, C(O)NH₂, C(O)—C₁₋₂alkyl, or C(O)—C₁fluoroalkyl;

and wherein in R³ the C₃₋₈cycloalkyl or the heterocyclic group of sub-formula (aa), (bb) or (cc) is optionally substituted with one or two substituents being oxo (═O), OH, C₁₋₂alkoxy, C₁₋₂fluoroalkoxy (e.g. trifluoromethoxy), or C₁₋₂alkyl; and wherein any OH, alkoxy or fluoroalkoxy substituent is not substituted at the R³ ring carbon attached (bonded) to the —NH— group of formula (I) and is not substituted at either R³ ring carbon bonded to the Y group of the heterocyclic group (aa), (bb) or (cc).

Alternatively or additionally to the above optional R³ definition, in one optional embodiment of the invention, X is NR⁴R⁵ or OR^{5a}, in which:

R⁴ is a hydrogen atom (H); C₁₋₆alkyl; C₁₋₃fluoroalkyl; or C₂₋₆alkyl substituted by one substituent R¹¹; and
R⁵ is a hydrogen atom (H); C₁₋₈alkyl; C₁₋₈ fluoroalkyl; C₃₋₈cycloalkyl optionally substituted by a C₁₋₂alkyl group; or —(CH₂)ₙ⁴—C₃₋₈cycloalkyl optionally substituted, in the —(CH₂)ₙ⁴— moiety or in the C₃₋₈cycloalkyl moiety, by a C₁₋₂alkyl group, wherein n⁴ is 1, 2 or 3;
or R⁵ is C₂₋₆alkyl substituted by one or two independent substituents R¹¹;
wherein each substituent R¹¹, independently of any other R¹¹ substituent present, is: hydroxy (OH); C₁₋₆alkoxy; phenyloxy; benzyloxy; —NR¹²R¹³; —NR¹⁵—C(O)R¹⁶; —NR¹⁵—C(O)—O—R¹⁶; —NR¹⁵—C(O)—NH—R¹⁵; or —NR¹⁵—SO₂R¹⁶; and wherein any R¹¹ substituent which is OH, alkoxy or —NR¹²R¹³ is not substituted at any carbon atom, of any R⁴ or R⁵ substituted alkyl, which is bonded to the nitrogen of NR⁴R⁵;
or R⁵ is —(CH₂)ₙ¹¹—C(O)R¹⁶; —(CH₂)ₙ¹²—C(O)NR¹²R¹³; —CHR¹⁹—C(O)NR¹²R¹³; —(CH₂)ₙ¹²—C(O)OR¹⁶; —CHR¹⁹—C(O)OR¹⁶; —(CH₂)ₙ¹²—SO₂—NR¹²R¹³; —(CH₂)ₙ¹²—SO₂R¹⁶; or —(CH₂)ₙ¹²—CN; wherein n¹¹ is 0, 1, 2, 3 or 4 and n¹² is 1, 2, 3 or 4;
or R⁵ is —(CH₂)ₙ¹³-Het wherein n¹³ is 0, 1, 2, 3 or 4 and Het is a 4-, 5-, 6- or 7-membered saturated or partly-saturated heterocyclic ring containing one or two ring-hetero-atoms independently selected from O, S, and N; wherein any ring-hetero-atoms present are not bound to the —(CH₂)ₙ¹³— moiety when n¹³ is 1 and are not bound to the nitrogen of NR⁴R⁵ when n¹³ is 0; wherein any ring-nitrogens which are present and which are not unsaturated (i.e. which do not partake in a double bond) are present as NR¹⁷ where R¹⁷ is as defined herein; and wherein one or two of the carbon ring-atoms independently are optionally substituted by C₁₋₂alkyl;
or R⁵ is phenyl optionally substituted with one or two of: a halogen atom; C₁₋₄alkyl (e.g. C₁₋₂alkyl); C₁₋₂fluoroalkyl (e.g. trifluoromethyl); C₁₋₄alkoxy (e.g. C₁₋₂alkoxy); C₁₋₂-fluoroalkoxy (e.g. trifluoromethoxy); C₁₋₂alkylsulphonyl (C₁₋₂alkyl-SO₂—); C₁₋₂alkyl-SO₂—NH—; R⁷R⁸N—SO₂—; R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-SO₂—CH₂—; cyano (CN); or phenyl optionally substituted by one or two of fluoro, chloro, C₁₋₂alkyl, C₁fluoroalkyl, C₁₋₂alkoxy or C₁fluoroalkoxy;
wherein R⁷ and R⁸ are independently a hydrogen atom (H); C₁₋₄alkyl (e.g. C₁₋₂alkyl such as methyl); C₃₋₆cycloalkyl; or phenyl optionally substituted by one or two of: fluoro, chloro, C₁₋₂alkyl, C₁fluoroalkyl, C₁₋₂alkoxy or C₁fluoroalkoxy; or R⁷ and R⁸ together are —(CH₂)ₙ⁶— or —C(O)—(CH₂)ₙ⁷— or —C(O)—(CH₂)ₙ⁷—C(O)— or —(CH₂)ₙ⁸—X⁷—(CH₂)ₙ⁹— or —C(O)—X⁷—(CH₂)ₙ¹⁰— in which: n⁶ is 3, 4, 5 or 6, n⁷ is 2, 3, 4, or 5 (preferably n⁷ is 2, 3 or 4), n⁸ and n⁹ and n¹¹ independently are 2 or 3, and X⁷ is O or NR¹⁴ wherein R¹⁴ is H or C₁₋₂alkyl;
   or R⁵ has the sub-formula (x), (y) or (z):

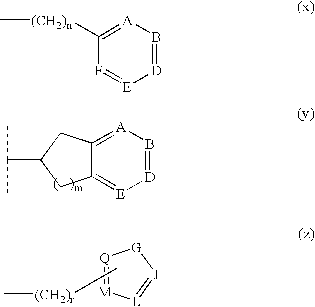

wherein in sub-formula (x), n=1 or 2; in sub-formula (y), m=1 or 2; and in sub-formula (z), r=0, 1 or 2;
wherein in sub-formula (x) and (y), none, one or two of A, B, D, E and F are nitrogen; and the remaining of A, B, D, E and F are independently CH or CR⁶;
where R⁶ is a halogen atom; C₁₋₄alkyl (e.g. C₁₋₂alkyl); C₁₋₄fluoroalkyl (e.g. C₁₋₂fluoroalkyl); C₁₋₄alkoxy (e.g. C₁₋₂alkoxy); C₁₋₂fluoroalkoxy; C₁₋₂alkylsulphonyl (C₁₋₂alkyl-SO₂—); C₁₋₂alkyl-SO₂—NH—; R⁷R⁸N—SO₂—; R⁷R⁸N—CO—; —NR¹⁵—C(O)R¹⁶;
R⁷R⁸N; OH; C₁₋₄alkoxymethyl; C₁₋₄alkoxyethyl; C₁₋₂alkyl-SO₂—CH₂—; cyano (CN); or phenyl optionally substituted by one or two of fluoro, chloro, C₁₋₂alkyl, C₁fluoroalkyl, C₁₋₂alkoxy or C₁fluoroalkoxy; wherein R⁷ and R⁸ are as herein defined;
wherein in sub-formula (z), G is O or S or NR⁹ wherein R⁹ is a hydrogen atom (H), C₁₋₄alkyl or C₁₋₄fluoroalkyl; none, one, two or three of J, L, M and Q are nitrogen; and the remaining of J, L, M and Q are independently CH or CR⁶ where R⁶ is as defined herein;
or R⁴ and R⁵ taken together are —(CH₂)ₚ¹— or —C(O)—(CH₂)ₚ²— or —(CH₂)ₚ³—X⁵—(CH₂)ₚ⁴— or —C(O)—X⁵—(CH₂)ₚ⁵—, in which: p¹=3, 4, 5 or 6 (preferably p=4 or 5), p² is 2, 3, 4, or 5 (preferably p² is 2, 3 or 4), and p³ and p⁴ and p⁵ independently are 2 or 3 (independently preferably 2) and X⁵ is O or NR¹⁷;
wherein R¹⁷ is a hydrogen atom (H); C₁₋₄alkyl (e.g. C₁₋₂alkyl); C₁₋₂fluoroalkyl; C₃₋₆cycloalkyl; —(CH₂)ₚ⁶—C(O)R¹⁶ wherein p⁶ is 0, 1, 2 or 3 (preferably p⁶ is 0); —(CH₂)ₚ⁶—C(O)NR¹²R¹³; —(CH₂)ₚ⁶—C(O)OR¹⁶; —SO₂R¹⁶; or phenyl or benzyl wherein the phenyl or benzyl is optionally substituted at an aromatic carbon atom by one or two of: a halogen atom, C₁₋₂alkyl, C₁fluoroalkyl, C₁₋₂alkoxy or C₁fluoroalkoxy;
and wherein, when R⁴ and R⁵ taken together are —(CH₂)ₚ¹— or —C(O)—(CH₂)ₚ²—, the NR⁴R⁵ heterocycle is optionally substituted by one R¹⁸ substituent wherein R¹⁸ is: C₁₋₄alkyl (e.g. C₁₋₂alkyl); C₁₋₂fluoroalkyl; C₃₋₆cycloalkyl; C₁₋₂alkoxy (not substituted at a ring-carbon bonded to the NR⁴R⁵ ring-nitrogen); C₁fluoroalkoxy (not substituted at a ring-carbon bonded to the NR⁴R⁵ ring-nitrogen); OH (not substituted at a ring-carbon bonded to the NR⁴R⁵ ring-nitrogen); —(CH₂)ₚ⁷—C(O)R¹⁶ wherein p⁷ is 0, 1, 2 or 3 (preferably p⁷ is 0 or 1); —(CH₂)ₚ⁷—C(O)OR¹⁶; —(CH₂)ₚ⁷—OC(O)R¹⁶; —(CH₂)ₚ⁷—C(O)NR¹²R¹³; —(CH₂)ₚ⁷—NR¹⁵C(O)R¹⁶; —(CH₂)ₚ⁷—NR¹⁵C(O)NR¹²R¹³; —(CH₂)ₚ⁷—NR¹⁵C(O)OR¹⁶; —(CH₂)ₚ⁷—SO₂R¹⁶; —(CH₂)ₚ⁷—SO₂ NR¹²R¹³; —(CH₂)ₚ⁷—NR¹⁵SO₂R¹⁶; —(CH₂)ₚ⁷—OH; —(CH₂)ₚ⁷—OR¹⁶; or phenyl optionally substituted by one or two of: a halogen atom, C₁₋₂alkyl, C₁fluoroalkyl, C₁₋₂alkoxy or C₁fluoroalkoxy;
   or R⁴ and R⁵ taken together are —(CH₂)ₚ¹— or —C(O)—(CH₂)ₚ²— or —(CH₂)ₚ³—X⁵—(CH₂)ₚ⁴— or —C(O)—X⁵—(CH₂)ₚ⁵— as defined herein, and wherein the NR⁴R⁵ heterocycle is fused to a phenyl ring optionally substituted on the phenyl by one or two of: a halogen atom, C₁₋₂alkyl, C₁fluoroalkyl, C₁₋₂alkoxy or C₁fluoroalkoxy; and
   R^{5a} is C₁₋₈alkyl; C₁₋₈ fluoroalkyl; C₃₋₈cycloalkyl; phenyl optionally substituted with one or two of: a halogen atom, C₁₋₂alkyl, trifluoromethyl, C₁₋₂alkoxy or trifluoromethoxy; or R^{5a} has the sub-formula (x), (y) or (z) as defined herein
   and wherein:
   R¹² and R¹³ independently are H; C₁₋₅alkyl (e.g. C₁₋₃alkyl); C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
   or R¹² and R¹³ together are —(CH₂)ₙ⁶— or —C(O)—(CH₂)ₙ⁷— or —C(O)—(CH₂)ₙ⁷—C(O)— or —(CH₂)ₙ⁸—X¹²—(CH₂)ₙ⁹— or —C(O)—X¹²—(CH₂)ₙ¹⁰— in which: n⁶ is 3, 4, 5 or 6 (preferably n⁶ is 4 or 5), n⁷ is 2, 3, 4, or 5 (preferably n⁷ is 2, 3 or 4), n⁸ and n⁹ and n¹¹ independently are 2 or 3 (independently preferably 2) and X¹² is O or NR¹⁴ wherein R¹⁴ is H or C₁₋₂ alkyl;
   R¹⁵ is a hydrogen atom (H); C₁₋₄alkyl (e.g. ^{t}Bu or C₁₋₂alkyl e.g. methyl); C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
   R¹⁶ is C₁₋₄alkyl (e.g. C₁₋₂alkyl); C₃₋₆cycloalkyl; pyridinyl (e.g. pyridin-2-yl); or phenyl optionally substituted by one or two of: a halogen atom, C₁₋₂alkyl, C₁fluoroalkyl, C₁₋₂alkoxy or C₁fluoroalkoxy; and
   R¹⁹ is C₁₋₄alkyl; —(CH₂)ₙ²⁰—OR²⁰ wherein n²⁰ is 1, 2, 3 or 4 and R²⁰ is a hydrogen atom (H) or C₁₋₄alkyl; —CH(Me)-OH; —CH₂—SH; —CH₂—CH₂—S-Me; benzyl; or (4-hydroxyphenyl)methyl (i.e. 4-hydroxy-benzyl).

In compounds, for example in the compounds of formula (I) (or formula (IA) or formula (IB), see later), an “alkyl” group or moiety may be straight-chain or branched. Alkyl groups, for example C₁₋₈alkyl or C₁₋₆alkyl or C₁₋₄alkyl or C₁₋₃alkyl or C₁₋₂alkyl, which may be employed include C₁₋₆alkyl or C₁₋₄alkyl or C₁₋₃alkyl or C₁₋₂alkyl such as methyl, ethyl, n-propyl, n-butyl, n-pentyl, or n-hexyl or any branched isomers thereof such as isopropyl, t-butyl, sec-butyl, isobutyl, 3-methylbutan-2-yl, 2-ethylbutan-1-yl, or the like.

A corresponding meaning is intended for “alkoxy”, “alkylene”, and like terms derived from alkyl. For example, “alkoxy” such as C₁₋₆alkoxy or C₁₋₄alkoxy or C₁₋₂alkoxy includes methoxy, ethoxy, propyloxy, and oxy derivatives of the alkyls listed above. “Alkylsulfonyl” such as C₁₋₄alkylsulfonyl includes methylsulfonyl (methanesulfonyl), ethylsulfonyl, and others derived from the alkyls listed above. “Alkylsulfonyloxy” such as C₁₋₄alkylsulfonyloxy includes methanesulfonyloxy (methylsulfonyloxy), ethanesulfonyloxy, et al.

“Cycloalkyl”, for example C₃₋₈cycloalkyl, includes cyclopropyl, cyclobutyl, cyclopentyl, cyclohexyl, cycloheptyl, cyclooctyl, and the like. Preferably, a C₃₋₈cycloalkyl group is C₃₋₆cycloalkyl or C₅₋₆cycloalkyl, that is contains a 3-6 membered or 5-6 membered carbocyclic ring.

“Fluoroalkyl” includes alkyl groups with one, two, three, four, five or more fluorine substituents, for example C₁₋₄fluoroalkyl or C₁₋₃fluoroalkyl or C₁₋₂fluoroalkyl such as monofluoromethyl, difluoromethyl, trifluoromethyl, pentafluoroethyl, 2,2,2-trifluoroethyl (CF₃CH₂—), 2,2-difluoroethyl (CHF₂CH₂—), 2-fluoroethyl (CH₂FCH₂—), etc. “Fluoroalkoxy” includes C₁₋₄fluoroalkoxy or C₁₋₂fluoroalkoxy such as trifluoromethoxy, pentafluoroethoxy, monofluoromethoxy, difluoromethoxy, etc. “Fluoroalkylsulfonyl” such as C₁₋₄fluoroalkylsulfonyl includes trifluoromethanesulfonyl, pentafluoroethylsulfonyl, etc.

A halogen atom (“halo”) present in compounds, for example in the compounds of formula (I), can be a fluorine, chlorine, bromine or iodine atom (“fluoro”, “chloro”, “bromo” or “iodo”).

When the specification states that atom or moiety A is “bonded” or “attached” to atom or moiety B, it means that atom/moiety A is directly bonded to atom/moiety B usually by means of one or more covalent bonds, and excludes A being indirectly attached to B via one or more intermediate atoms/moieties (e.g. excludes A-C-B); unless it is clear from the context that another meaning is intended.

Preferably, R¹ is C₁₋₄alkyl (e.g. methyl, ethyl, n-propyl, isopropyl or n-butyl), C₁₋₃fluoroalkyl or —CH₂CH₂OH; R¹ is more preferably C₁₋₃alkyl (e.g. methyl, ethyl or n-propyl), C₁₋₂fluoroalkyl, or —CH₂CH₂OH; still more preferably C₁₋₃alkyl, C₂fluoroalkyl or —CH₂CH₂OH such as methyl, ethyl, n-propyl or —CH₂CH₂OH. Yet more preferably, R¹ is C₂₋₃alkyl (e.g. ethyl or n-propyl), C₂fluoroalkyl (e.g. C₁fluoroalkyl-CH₂— such as CF₃—CH₂—) or —CH₂CH₂OH; in particular ethyl, n-propyl or —CH₂CH₂OH. R¹ is most preferably ethyl.

Preferably, R² is a hydrogen atom (H) or methyl, more preferably a hydrogen atom (H).

Preferably, in R³ there is one substituent or no substituent.

In one optional embodiment, R³ is the optionally substituted C₃₋₈cycloalkyl or the optionally substituted heterocyclic group of sub-formula (aa), (bb) or (cc). In this embodiment, optionally, in R³, the C₃₋₈cycloalkyl or the heterocyclic group of sub-formula (aa), (bb) or (cc) is optionally substituted with one or two substituents independently being (e.g. being) oxo (═O), OH, C₁₋₂alkoxy, C₁₋₂fluoroalkoxy (e.g. trifluoromethoxy), or C₁₋₂alkyl; and wherein any OH, alkoxy or fluoroalkoxy substituent is not substituted at the R³ ring carbon attached (bonded) to the —NH— group of formula (I) and is not substituted at either R³ ring carbon bonded to the Y group of the heterocyclic group (aa), (bb) or (cc).

In one optional embodiment, where R³ is optionally substituted C₃₋₈cycloalkyl, it is not optionally substituted C₅cycloalkyl, i.e. not optionally substituted cyclopentyl. In this case, more preferably, R³ is optionally substituted C₆₋₈cycloalkyl.

Where R³ is optionally substituted C₃₋₈cycloalkyl, it is more preferably optionally substituted C₆cycloalkyl (i.e. cyclohexyl); for example C₆cycloalkyl optionally substituted with one or two substituents independently being (e.g. being) oxo (═O), OH, C₁₋₂alkoxy, C₁₋₂fluoroalkoxy (e.g. trifluoromethoxy), or C₁₋₂alkyl, and wherein any OH, alkoxy or fluoroalkoxy substituent is not substituted at the R³ ring carbon attached (bonded) to the —NH— group of formula (I).

Where R³ is optionally substituted C₃₋₈cycloalkyl, the one or two optional substituents preferably comprise (e.g. is or independently are (e.g. is or are)) oxo (═O); OH; C₁ alkoxy; C₁fluoroalkoxy (e.g. trifluoromethoxy); NHR²¹ wherein R²¹ is a hydrogen atom (H) or C₁₋₂ straight-chain alkyl; C₁₋₂alkyl such as methyl; C₁fluoroalkyl such as —CH₂F or —CHF₂; —CH₂OH; —CH₂NHR²² wherein R²² is H; —C(O)OR²³ wherein R²³ is H or methyl; —C(O)NHR²⁴ wherein R²⁴ is H or methyl; —C(O)R²⁵ wherein R²⁵ is methyl; fluoro; hydroxyimino (═N—OH); or (C₁₋₂alkoxy)imino (═N—OR²⁶ where R²⁶ is C₁₋₂alkyl); and wherein any OH, alkoxy, fluoroalkoxy or NHR²¹ substituent is not substituted at the R³ ring carbon attached (bonded) to the —NH— group of formula (I) and is not substituted at either R³ ring carbon bonded to the Y group of the heterocyclic group (aa), (bb) or (cc).

More preferably, where R³ is optionally substituted C₃₋₈cycloalkyl, the one or two optional substituents comprise (e.g. is or independently are (e.g. is or are)) oxo (═O); OH; NHR²¹ wherein R²¹ is a hydrogen atom (H); C₁₋₂alkyl such as methyl; C₁fluoroalkyl such as —CH₂F or —CHF₂; —C(O)OR²³ wherein R²³ is H or methyl; —C(O)NHR²⁴ wherein R²⁴ is H or methyl; fluoro; hydroxyimino (═N—OH); or (C₁₋₂alkoxy)imino (═N—OR²⁶ where R²⁶ is C₁₋₂alkyl).

Still more preferably, where R³ is optionally substituted C₃₋₈cycloalkyl, the one or two optional substituents comprise (e.g. is or independently are (e.g. is or are)) oxo (═O); OH; NHR²¹ wherein R²¹ is a hydrogen atom (H); methyl; —CH₂F; —CHF₂; —C(O)OR²³ wherein R²³ is H; fluoro; hydroxyimino (═N—OH); or (C₁₋₂alkoxy)imino (═N—OR²⁶ where R²⁶ is C₁₋₂alkyl). Yet more preferably, where R³ is optionally substituted C₃₋₈cycloalkyl, the one or two optional substituents comprise (e.g. is or independently are (e.g. is or are)) oxo (═O); OH; methyl; fluoro; hydroxyimino (═N—OH); or (C₁₋₂alkoxy)imino (═N—OR²⁶ where R²⁶ is C₁₋₂alkyl).

Most preferably, where R³ is optionally substituted C₃₋₈cycloalkyl, the one or two optional substituents comprise (e.g. is or independently are (e.g. is or are)) OH, oxo (═O) or oximo (═N—OH). For example, the one or two optional substituents can comprise (e.g. is or are) OH and/or oxo (═O).

Optionally, in R³, the C₃₋₈cycloalkyl can be unsubstituted.

Where R³ is optionally substituted C₃₋₈cycloalkyl, e.g. optionally substituted C₅₋₈cycloalkyl such as optionally substituted C₆cycloalkyl (optionally substituted cyclohexyl), the one or two optional substituents if present preferably comprise a substituent (for example is or are substituent(s)) at the 3-, 4- or 5-position(s) of the R³ cycloalkyl ring. (In this connection, the 1-position of the R³ cycloalkyl ring is deemed to be the connection point to the —NH— in formula (I)).

Where R³ is optionally substituted C₃₋₈cycloalkyl, any OH, alkoxy, fluoroalkoxy, —CH₂OH, —CH₂CH₂OH, —CH₂NHR²², —C(O)OR²³, —C(O)NHR²⁴, —C(O)R²⁵ or fluoro substituent (particularly any OH substituent) is more preferably at the 3-, 4- or 5-position, e.g. 3- or 5-position, of the R³ cycloalkyl (e.g. C₆₋₈cycloalkyl) ring. For example, any OH, alkoxy, fluoroalkoxy, —CH₂OH, —CH₂CH₂OH, —CH₂NHR²², —C(O)OR²³, —C(O)NHR²⁴, —C(O)R²⁵ or fluoro substituent (particularly any OH substituent) can be at the 3-position of a R³ C₅cycloalkyl (cyclopentyl) ring or at the 3-, 4- or 5-position, e.g. 3- or 5-position, of a R³ C₆cycloalkyl (cyclohexyl) ring. (In this connection, and also below, the 1-position of the R³ cycloalkyl ring is deemed to be the connection point to the —NH— in formula (I)).

Where R³ is optionally substituted C₃₋₈cycloalkyl, any NHR²¹ substituent is preferably at the 2-, 3-, 4- or 5-position, preferably the 2- or 3-position or more preferably the 3-position, of the R³ cycloalkyl (e.g. C₆₋₈cycloalkyl e.g. cyclohexyl) ring.

Where R³ is optionally substituted C₃₋₈cycloalkyl, any alkyl or fluoroalkyl substituent is preferably at the 1-, 2-, 3-, 4- or 5-position, more preferably the 1-, 2-, 3- or 5-position, still more preferably the 1- or 3-position, of the R³ cycloalkyl (e.g. C₆₋₈cycloalkyl e.g. cyclohexyl) ring.

Where R³ is optionally substituted C₃₋₈cycloalkyl, any oxo (═O), hydroxyimino (═N—OH); or (C₁₋₄alkoxy)imino (═N—OR²⁶) substituent is preferably at the 3- or 4-position, preferably at the 4-position, of the R³ cycloalkyl (e.g. C₆₋₈cycloalkyl e.g. cyclohexyl) ring.

Where R³ is optionally substituted C₃₋₈cycloalkyl, R³ is preferably cyclohexyl (i.e. unsubstituted), or cyclohexyl substituted by one oxo (═O), OH, NHR²¹, C₁₋₂alkyl, C₁₋₂fluoroalkyl, —CH₂OH, —C(O)OR²³, —C(O)NHR²⁴, —C(O)R²⁵, fluoro, hydroxyimino (═N—OH), (C₁₋₄alkoxy)imino (═N—OR²⁶) substituent, or cyclohexyl substituted by two fluoro substituents. More preferably, R³ is cyclohexyl (i.e. unsubstituted), or cyclohexyl substituted by one oxo (═O), OH, NHR²¹, C₁₋₂alkyl, C₁₋₂fluoroalkyl, —C(O)OR²³, fluoro, hydroxyimino (═N—OH) or (C₁₋₄alkoxy)imino (═N—OR²⁶) substituent, or cyclohexyl substituted by two fluoro substituents. Still more preferably R³ is cyclohexyl (i.e. unsubstituted) or cyclohexyl substituted by one oxo (═O), hydroxyimino (═N—OH), C₁₋₂alkyl or OH substituent. The optional substituent can be at the 3- or 4-position, e.g. 3-position, of the R³ cyclohexyl ring; more preferably any OH substituent is preferably at the 3-position of the R³ cyclohexyl ring, and/or any oxo (═O), hydroxyimino (═N—OH) or (C₁₋₄alkoxy)imino (═N—OR²⁶) substituent is preferably at the 4-position of the R³ cyclohexyl ring.

Where R³ is optionally substituted C₆cycloalkyl, R³ can for example be 4-hydroxy-cyclohexyl (i.e. 4-hydroxycyclohexan-1-yl), but R³ is more preferably cyclohexyl (i.e. unsubstituted), 3-hydroxy-cyclohexyl (i.e. 3-hydroxycyclohexan-1-yl), 4-oxo-cyclohexyl (i.e. 4-oxocyclohexan-1-yl), 4-(hydroxyimino)cyclohexyl (i.e. 4-(hydroxyimino)cyclohexan-1-yl), 4-(C₁₋₂alkoxyimino)cyclohexyl, 1-methylcyclohexyl or 3-methylcyclohexyl. Where R³ is optionally substituted C₆cycloalkyl, R³ is most preferably cyclohexyl (i.e. unsubstituted), 4-oxo-cyclohexyl (i.e. 4-oxocyclohexan-1-yl) or 4-(hydroxyimino)cyclohexyl (i.e. 4-(hydroxyimino)cyclohexan-1-yl).

Where R³ is optionally substituted C₅cycloalkyl (optionally substituted cyclopentyl), R³ can for example be cyclopentyl (i.e. unsubstituted) or 3-hydroxy-cyclopentyl.

Where R³ is optionally substituted mono-unsaturated-C₅₋₇cycloalkenyl, preferably it is optionally substituted mono-unsaturated-C₅₋₆cycloalkenyl, more preferably optionally substituted mono-unsaturated-C₆cycloalkenyl (i.e. optionally substituted mono-unsaturated-cyclohexenyl=optionally substituted cyclohexenyl). Still more preferably, the R³ cyclohexenyl is optionally substituted cyclohex-3-en-1-yl.

Where R³ is optionally substituted mono-unsaturated-C₅₋₇cycloalkenyl, preferably the R³ cycloalkenyl is optionally substituted with one or two substituents being fluoro or methyl provided that if there are two substituents then they are not both methyl. Preferably, the R³ cycloalkenyl is optionally substituted with one substituent being fluoro or C₁₋₂alkyl (e.g. methyl); more preferably the R³ cycloalkenyl is substituted with one fluoro substituent or is unsubstituted. For R³ cycloalkenyl, the optional substituent(s) can be at the 1-, 2-, 3-, 4- or 5-position(s) of the cycloalkenyl ring.

Where R³ is the heterocyclic group of sub-formula (aa), (bb) or (cc), then Y is preferably O, S, SO₂, NH or N—C(O)methyl, more preferably O, NH or N—C(O)methyl, still more preferably O or N—C(O)methyl, most preferably O. (When Y is NH or N—C(O)methyl, then R¹⁰ is H or C(O)methyl).

Preferably, R¹⁰ is a hydrogen atom (H), methyl, ethyl, C(O)NH₂, C(O)methyl or C(O)—CF₃—Optionally, R¹⁰ can be a hydrogen atom (H), methyl, ethyl, C(O)methyl or C(O)—CF₃, more preferably H, C(O)methyl or C(O)—CF₃, still more preferably H or C(O)methyl.

Where R³ is the heterocyclic group of sub-formula (aa), (bb) or (cc), then it is preferable that R³ is the heterocyclic group of sub-formula (aa) or (bb), more preferably of sub-formula (bb).

In sub-formula (bb), n¹ is preferably 1. In sub-formula (cc), n² is preferably 1. That is, six-membered rings are preferred in the R³ heterocyclic group.

Suitably, in R³, the heterocyclic group of sub-formula (aa), (bb) or (cc) is unsubstituted (In this connection, where Y is NR¹⁰, R¹⁰ is not classified as a substituent).

In the R³ heterocyclic group of sub-formula (aa), (bb) or (cc), the one or two optional substituents preferably comprise (e.g. is or independently are ((e.g. is or are)) OH; oxo (═O); C₁₋₂alkyl (e.g. methyl) or C₁₋₂fluoroalkyl (e.g. C₁fluoroalkyl such as —CH₂F or —CHF₂). More preferably, in the R³ heterocyclic group of sub-formula (aa), (bb) or (cc), the one or two optional substituents comprise (e.g. is or independently are ((e.g. is or are)) OH and/or oxo; most preferably the one or two optional substituents comprise (e.g. is or are) oxo (═O). In the R³ heterocyclic group of sub-formula (aa), (bb) or (cc), any oxo (═O) substituents are preferably on a carbon atom bonded (adjacent) to X, and/or can be at the 2-, 3-, 4- or 5-position(s) of the R³ heterocyclic ring. (In this connection, the 1-position of the R³ heterocyclic ring is deemed to be the connection point to the —NH— in formula (I)). Preferably, only C₁₋₂alkyl, C₁₋₂fluoroalkyl, fluoro or oxo (═O) substitution or no substitution is allowed at each of the 2- and 6-positions of the R³ heterocyclic ring.

When R³ is the heterocyclic group of sub-formula (aa) and Y is NR¹⁰, then preferably R¹⁰ is not C(O)-Me. More preferably, when R³ is the heterocyclic group of sub-formula (aa) and Y is NR¹⁰, then R¹⁰ is preferably not C(O)R, i.e. or e.g. R¹⁰ is preferably not C(O)NH₂, C(O)—C₁₋₂alkyl or C(O)—C₁fluoroalkyl. In one embodiment, Y is O, S, SO₂ or NH when R³ is the heterocyclic group of sub-formula (aa).

Optionally, according to one embodiment of the invention, NHR³ is not

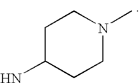

More preferably, when R³ is the heterocyclic group of sub-formula (bb) and Y is NR¹⁰, and optionally when n¹ is 1, then preferably R¹⁰ is not methyl. More preferably, when R³ is the heterocyclic group of sub-formula (bb) and Y is NR¹⁰, and optionally when n¹ is 1, then R¹⁰ is preferably not alkyl or substituted alkyl, i.e. or e.g. R¹⁰ is preferably not C₁₋₄alkyl (e.g. methyl or ethyl), C₁₋₂fluoroalkyl or CH₂C(O)NH₂. In one embodiment, when R³ is the heterocyclic group of sub-formula (bb), Y is preferably O, S, SO₂ or NR¹⁰, wherein R¹⁰ is H, C(O)NH₂, C(O)—C₁₋₂alkyl or C(O)—C₁fluoroalkyl, or more preferably Y is H or C(O)Me. More preferably, for sub-formula (bb), Y is O or NR¹⁰.

Where R³ is a bicyclic group of sub-formula (dd) or (ee), preferably it is of sub-formula (ee). In sub-formula (ee), preferably Y¹, Y² and Y³ are all CH₂.

Preferably, NHR³ is of sub-formula (a), (a1), (b), (c), (c 1), (c 2), (c 3), (c 4), (c 5), (c 6), (c 7), (d), (e), (f), (g), (g1), (g2), (g3), (g4), (h), (i), (j), (k), (k1), (L), (m), (m1), (m2), (m3), (m4), (m5), (n), (o), (o1), (o2), (o3), (o4), (o5), (p), (p1), (p2), (p3), (p4), (p5), (p6), (p7), (p8) or (q):

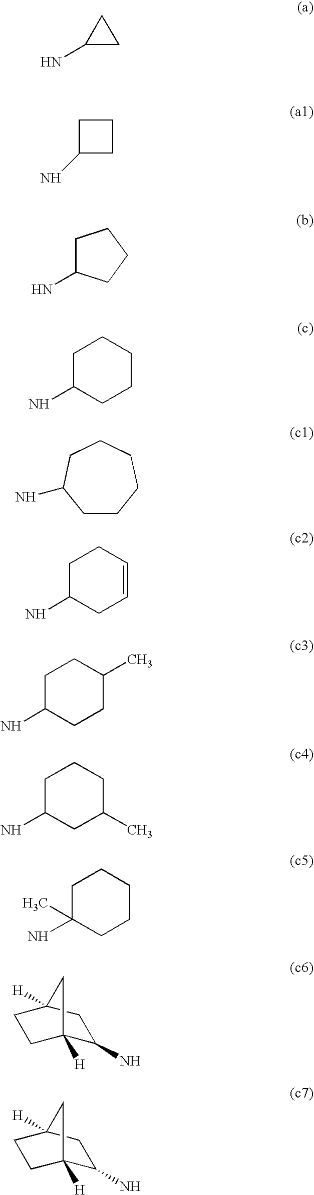

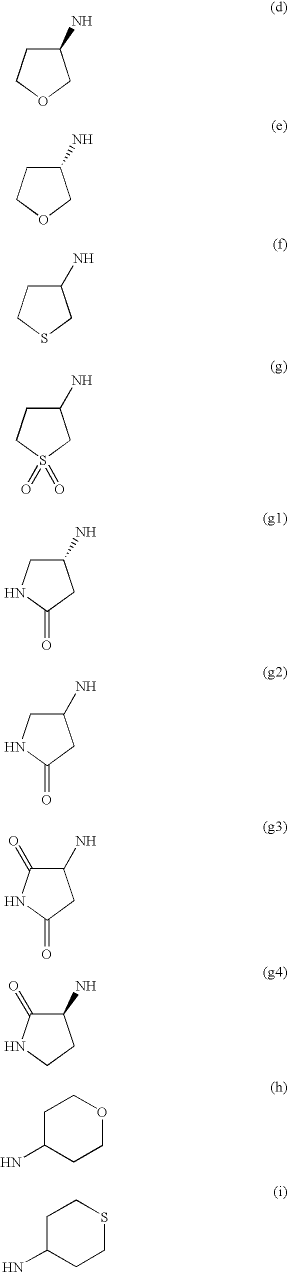

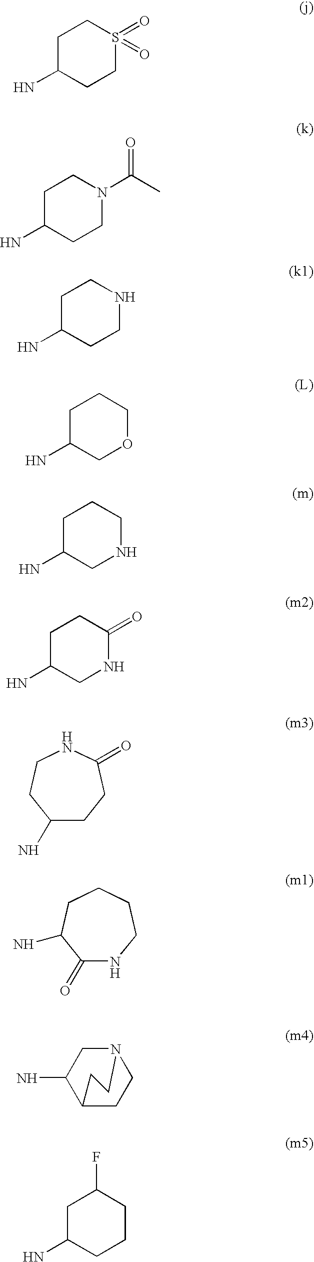

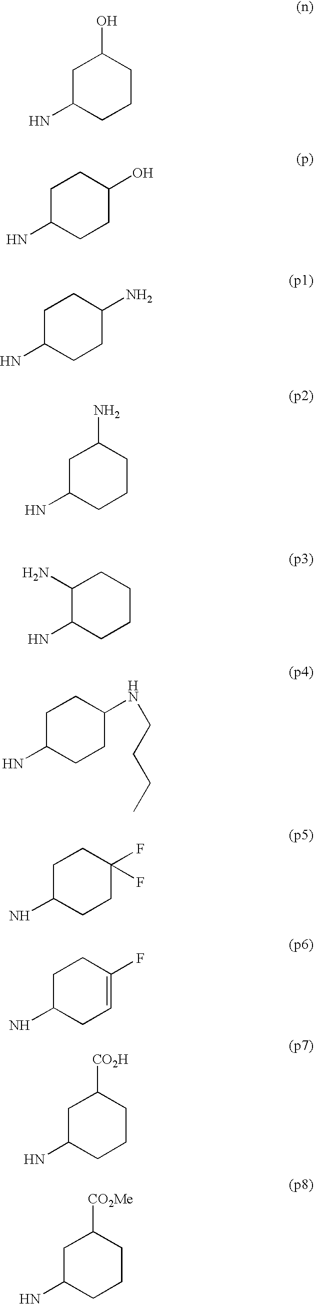

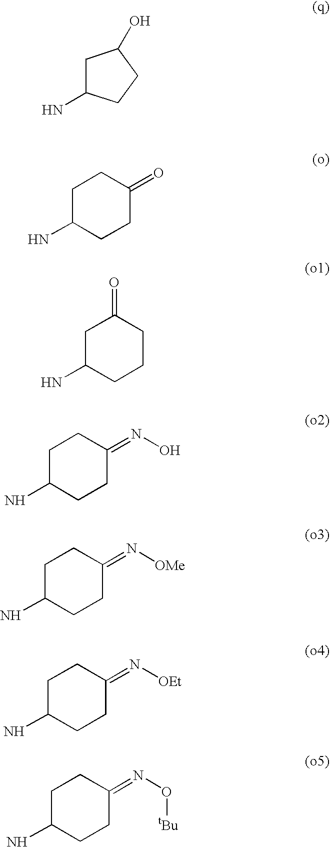

In the sub-formulae (a) to (q) etc above, the —NH— connection point of the NHR³ group to the 4-position of the pyrazolopyridine of formula (I) is underlined.

Preferably, NHR³ is of sub-formula (c), (c1), (c 2), (c 3), (c 4), (c 5), (c 6), (c 7), (d), (e), (f), (g1), (g4), (h), (i), (j), (k), (k1), (L), (m), (m1), (m2), (m3), (m5), (n), (o), (o1), (o2), (o3), (o4), (o5), (p), (p2), (p3), (p5), (p6), (p7) or (q). More preferably, NHR³ is of sub-formula (c), (c1), (c 4), (c 5), (h), (i), (j), (k), (m1), (m2), (n), (o), (o2), (o3), (p2), (p5), (p6) or (q). Still more preferably, NHR³ is of sub-formula (c), (h), (k), (n), (o) or (o2); for example (c), (h), (o) or (o2). Most preferably, R³ is tetrahydro-2H-pyran-4-yl; that is NHR³ is most preferably of sub-formula (h), as shown above.

According to one embodiment, NHR³ is of sub-formula (a), (b), (c), (d), (e), (f), (g), (g1), (g2), (g3), (h), (i), (j), (k), (L), (m), (m1), (n), (o), (o1), (p) or (q). In this embodiment, preferably, NHR³ is of sub-formula (c), (d), (e), (f), (g1), (h), (i), (j), (k), (m), (m1), (n), (O), (o1), (p), or (q); and more preferably in this embodiment, NHR³ is of sub-formula (c), (h), (i), (j), (k), (m1), (n), (o) or (q). Still more preferably in this embodiment, NHR³ is of sub-formula (c), (h), (k), (n) or (o). Most preferably, R³ is tetrahydro-2H-pyran-4-yl; that is NHR³ is most preferably of sub-formula (h), as shown above.

According to another embodiment, NHR³ is of sub-formula (a), (b), (c), (d), (e), (f), (g), (h), (i), (j) or (k). In this embodiment, preferably, NHR³ is of sub-formula (c), (d), (e), (f), (h), (i), (j) or (k); and more preferably in this embodiment, NHR³ is of sub-formula (c), (h), (i), (j) or (k). Most preferably, R³ is tetrahydro-2H-pyran-4-yl; that is NHR³ is most preferably of sub-formula (h), as shown above.

When NHR³ is of sub-formula (n), then preferably it is a cis-(3-hydroxycyclohex-1-yl)amino group, eg in any enantiomeric form or mixture of forms but preferably racemic.

Preferably, X is NR⁴R⁵.

Where R⁴ is C₁₋₁₆alkyl, then preferably it is C₁₋₄alkyl or C₁₋₂alkyl. Where R⁴ is C₁₋₃fluoroalkyl then preferably it is C₁₋₂fluoroalkyl.

Most preferably, R⁴ is a hydrogen atom (H).

Where R⁴ is C₂₋₆alkyl substituted by one substituent R¹¹, then preferably R⁴ is C₂₋₄alkyl (e.g. C₂₋₃alkyl) substituted by one substituent R¹¹. More preferably, R⁴ is —(CH₂)ₙ³—R¹¹ wherein n³ is 2, 3 or 4. Still more preferably, n³ is 2 and/or R⁴ is —(CH₂)ₙ³—OH.

When R⁵ is C₂₋₆alkyl substituted by one or two independent substituents R¹¹, it is preferable that R⁵ is C₂₋₄alkyl (e.g. C₂₋₃alkyl) substituted by one or two independent substituents R¹¹. When R⁵ is C₂₋₆alkyl (e.g. C₂₋₄alkyl or C₂₋₃alkyl) substituted by one or two independent substituents R¹¹, it is preferable that R⁵ is C₂₋₆alkyl (e.g. C₂₋₄alkyl or C₂₋₃alkyl) substituted by one substituent R¹¹. It is more preferable that R⁵ is —(CH₂)ₙ⁵—R¹¹ wherein n⁵ is 2, 3 or 4. Preferably n⁵ is 2 or 3, more preferably 2.

Preferably, each substituent R¹¹, independently of any other R¹¹ substituent present, is: hydroxy (OH); C₁₋₆alkoxy (e.g. C₁₋₄alkoxy such as t-butyloxy, ethoxy or methoxy); phenyloxy; benzyloxy; —NR¹²R¹³; —NR¹⁵—C(O)R¹⁶; —NR¹⁵—C(O)—NH—R¹⁵; or —NR¹⁵—SO₂R¹⁶ (more preferably C₁₋₆alkoxy, —NR¹⁵—C(O)—NH—R¹⁵, or —NR¹⁵—SO₂R¹⁶; most preferably —NR¹⁵—SO₂R¹⁶). In all cases, any R¹¹ substituent which is OH, alkoxy or —NR¹²R¹³ is not substituted at any carbon atom, of any R⁴ or R⁵ substituted alkyl, which is bonded to the nitrogen of NR⁴R⁵.

Where R⁵ is C₁₋₈alkyl, then preferably it is C₁₋₅alkyl or C₁₋₃alkyl. Where R⁵ is C₁₋₈fluoroalkyl then preferably it is C₁₋₃fluoroalkyl or C₁₋₂fluoroalkyl. Where R⁵ is C₃₋₈cycloalkyl optionally substituted by a C₁₋₂alkyl group, then preferably the C₃₋₈cycloalkyl is not substituted at the ring-carbon bonded to the nitrogen of NR⁴R⁵. Where R⁵ is optionally substituted C₃₋₈cycloalkyl, then more preferably it is C₃₋₈cycloalkyl (i.e. unsubstituted).

When R⁵ is optionally substituted —(CH₂)ₙ⁴—C₃₋₈cycloalkyl wherein n⁴ is 1, 2 or 3, then n⁴ is preferably 1 or 2 or more preferably 1, and/or preferably R⁵ is optionally substituted —(CH₂)ₙ⁴—C₅₋₆cycloalkyl or optionally substituted —(CH₂)ₙ⁴—C₆cycloalkyl. When R⁵ is optionally substituted —(CH₂)ₙ⁴—C3-cycloalkyl, preferably it is not substituted. Most preferably R⁵ is (cyclohexyl)methyl-, that is —CH₂-cyclohexyl.

When R¹⁹ is C₁₋₄alkyl, then preferably it is isobutyl, sec-butyl, or C₁₋₃alkyl such as methyl or isopropyl. When R¹⁹ is —(CH₂)ₙ²⁰—OR²⁰, then preferably n²⁰ is 1 and/or preferably R²⁰ is a hydrogen atom (H).

When R⁵ is —(CH₂)ₙ¹¹—C(O)R¹⁶; —(CH₂)ₙ¹²—C(O)NR¹²R¹³; —CHR¹⁹—C(O)NR¹²R¹³; —(CH₂)ₙ¹²—C(O)OR¹⁶; —CHR¹⁹—C(O)OR¹⁶; —(CH₂)ₙ¹²—SO₂—NR¹²R¹³; —(CH₂)ₙ¹²—SO₂R¹⁶; or —(CH₂)ₙ¹²—CN; then in one embodiment of the invention R⁵ can be: —(CH₂)ₙ¹¹—C(O)R¹⁶; —(CH₂)ₙ¹²—C(O)NR¹²R¹³; —(CH₂)ₙ¹²—C(O)OR¹⁶; —(CH₂)ₙ¹²—SO₂—NR¹²R¹³; —(CH₂)ₙ¹²—SO₂R¹⁶; or —(CH₂)ₙ¹²—CN.

When R⁵ is —(CH₂)ₙ¹¹—C(O)R¹⁶; —(CH₂)ₙ¹²—C(O)NR¹²R¹³; —(CH₂)ₙ¹²—C(O)OR¹⁶; —(CH₂)ₙ¹²—SO₂—NR¹²R¹³; —(CH₂)ₙ¹²—SO₂R¹⁶; or —(CH₂)ₙ¹²—CN; then R⁵ can for example be —(CH₂)ₙ¹¹—C(O)R¹⁶; —(CH₂)ₙ¹²—C(O)NR¹²R¹³; or —(CH₂)ₙ¹²—CN; preferably —(CH₂)ₙ¹¹—C(O)R¹⁶.

Preferably, n¹¹ is 1, 2, 3 or 4; more preferably n¹¹ is 1 or 2. Advantageously, n¹² is 1 or 2.

When R⁵ is —(CH₂)ₙ¹³-Het, it is preferable that n¹³ is 0, 1 or 2, more preferably 0 or 1.

Preferably, Het is a 5- or 6-membered saturated or partly-saturated heterocyclic ring and/or preferably is a 4-, 5-, 6- or 7-membered saturated heterocyclic ring. Preferably, the heterocyclic ring Het contains one ring-hetero-atom selected from O, S and N. Preferably, the carbon ring-atoms in Het are not substituted. Het is most preferably one of:

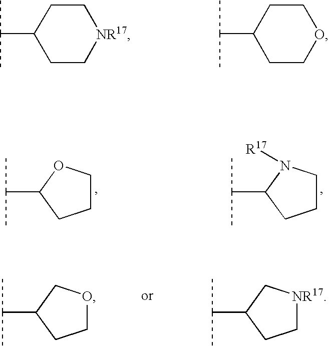

When R⁵ is optionally substituted phenyl, then preferably it is phenyl optionally substituted with one or two of the substituents defined herein.

When R⁵ is optionally substituted phenyl, then preferably R⁵ is phenyl optionally substituted with, independently, one, two or three (preferably one or two; or one) of: a halogen atom (preferably fluoro and/or chloro); C₁₋₂alkyl; C₁₋₂fluoroalkyl (e.g. trifluoromethyl); C₁₋₂alkoxy (e.g. methoxy); trifluoromethoxy; C₁₋₂alkylsulphonyl (C₁₋₂alkyl-SO₂—); C₁₋₂alkyl-SO₂—NH—; R⁷R⁸N—SO₂—; R⁷R⁸N—CO—; —NR¹⁵—C(O)R¹⁶; R⁷R⁸N; OH; C₁₋₂alkoxymethyl; C₁₋₂alkyl-SO₂—CH₂—; cyano (CN); or phenyl optionally substituted by one of fluoro, C₁₋₂alkyl, C₁fluoroalkyl, C₁₋₂alkoxy or C₁fluoroalkoxy. More preferably R⁵ is phenyl optionally substituted with one or two (preferably one) of: a halogen atom, C₁₋₂alkyl, trifluoromethyl, C₁₋₂alkoxy, trifluoromethoxy, R⁷R⁸N—SO₂—, R⁷R⁸N—CO—, or C₁₋₂alkyl-SO₂—CH₂—. When R⁵ is optionally substituted phenyl, then preferably one or all of the one or two optional substituents are substituted at the meta- (3- and/or 5-) and/or para- (4-) position(s) of the phenyl ring with respect to the phenyl ring-carbon bonded to the nitrogen of NR⁴R⁵.

Preferably, R⁷ and/or R⁸ are independently a hydrogen atom (H); C₁₋₂alkyl such as methyl; C₃₋₆cycloalkyl; or phenyl optionally substituted by one of: fluoro, chloro, C₁₋₂alkyl, C₁fluoroalkyl, C₁₋₂alkoxy or C₁fluoroalkoxy; or R⁷ and R⁸ together are —(CH₂)ₙ⁶— or —(CH₂)ₙ⁸—X⁷—(CH₂)ₙ⁹— wherein X⁷ is NR¹⁴ or preferably O.

When R⁷ is cycloalkyl or optionally substituted phenyl, then preferably R⁸ is neither cycloalkyl nor optionally substituted phenyl.

Most preferably, R⁷ and/or R⁸ independently are a hydrogen atom (H) or C₁₋₂alkyl. It is preferable that R⁷ is a hydrogen atom (H).

Preferably n⁶ is 4 or 5. Preferably n⁷ is 2, 3 or 4. Preferably, n⁸, n⁹ and/or n¹⁰ is/are independently 2.

In general, it is preferable that R⁵ has the sub-formula (x) or (y) or (y1) or (z).

When R⁵ has the sub-formula (x) or (y) or (y1) or (z), then preferably R⁵ has the sub-formula (x) or (y) or (y1) or has the sub-formula (x) or (y) or (z). More preferably R⁵ has the sub-formula (x) or (y), most preferably (x). In one embodiment, R⁵ has the sub-formula (z).

Preferably, n is 1 or 2. More preferably, n=1. Preferably, m=1. Preferably, r=1 or 2, more preferably 1.

In sub-formula (x), (y) and/or (y1), it is preferred that none, one or two of A, B, D, E and F are nitrogen; none, one, two or three of A, B, D, E and F are CR⁶; and the remaining of A, B, D, E and F are CH. More preferably, none, one or two of A, B, D, E and F are nitrogen; none, one or two of A, B, D, E and F are CR⁶; and the remaining of A, B, D, E and F are CH.

In sub-formula (x), (y) and/or (y1), preferably, none or one of A, B, D, E and F are nitrogen, and/or preferably none, one or two of A, B, D, E and F are CR⁶.

Preferably, sub-formula (x) is: benzyl; phenethyl (Ph-C₂H₄—); benzyl substituted on the phenyl ring with one or two R⁶ substituents; phenethyl (Ph-C₂H₄—) substituted on the phenyl ring with one or two R⁶ substituents; or one of the following:

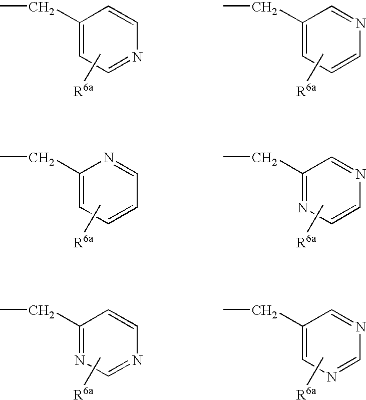

, wherein R^{6a} is either R⁶ as defined herein or (preferably) hydrogen.

Most preferably, sub-formula (x) is benzyl or pyridinylmethyl

[e.g. pyridin-4-ylmethyl (i.e.

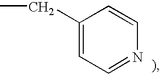

pyridin-3-ylmethyl, or preferably pyridin-2-ylmethyl (i.e.

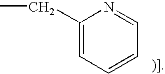

Preferably, sub-formula (y) is:

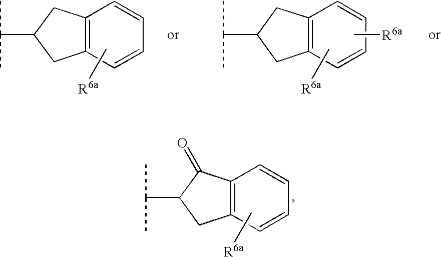

wherein R^{6a} is or independently are either R⁶ as defined herein or preferably hydrogen. Preferably, sub-formula (y) is not substituted by oxo (═O) at the carbon between the 6-membered aromatic ring and the carbon bonded to the nitrogen of NR⁴R⁵.

Preferably, sub-formula (y1) is:

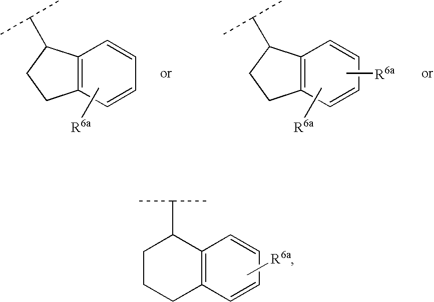

wherein R^{6a} is or independently are either R⁶ as defined herein or preferably hydrogen.

Preferably, in sub-formula (z), none, one or two of J, L, M and Q are nitrogen.

In sub-formula (x), (y) and/or (z), preferably, each R⁶, independently of any other R⁶ present, is a fluorine, chlorine, bromine or iodine atom, methyl, ethyl, n-propyl, isopropyl, C₄alkyl, trifluoromethyl, —CH₂OH, methoxy, ethoxy, C₁fluoroalkoxy (e.g. trifluoromethoxy or difluoromethoxy), OH, C₁₋₃alkylS(O)₂— (such as methylsulphonyl which is MeS(O)₂—), C₁₋₃alkylS(O)₂—NH— such as methyl-SO₂—NH—, Me₂N—S(O)₂—, H₂N—S(O)₂—, —CONH₂, —CONHMe, —CO₂H, cyano (CN), NMe₂, t-butoxymethyl, or C₁₋₃alkylS(O)₂—CH₂— such as methyl-SO₂—CH₂—. More preferably, each R⁶, independently of any other R⁶ present, is a fluorine, chlorine, bromine or iodine atom, methyl, ethyl, n-propyl, isopropyl, isobutyl, trifluoromethyl, —CH₂OH, methoxy, ethoxy, C₁fluoroalkoxy (e.g. trifluoromethoxy or difluoromethoxy), C₁₋₃alkylS(O)₂— such as methylsulphonyl, C₁₋₃alkylS(O)₂—NH— such as methyl-SO₂—NH—, Me₂N—S(O)₂—, H₂N—S(O)₂—, —CONH₂, or C₁₋₃alkylS(O)₂—CH₂— such as methyl-SO₂—CH₂. Still more preferably, each R⁶, independently of any other R⁶ present, is a fluorine, chlorine or bromine atom, methyl, ethyl, n-propyl, isopropyl, trifluoromethyl, —CH₂OH, methoxy, difluoromethoxy, methylsulphonyl, methyl-SO₂—NH— or methyl-SO₂—CH₂—.

The above preferred R⁶ substituents are also, independently, the preferred phenyl optional and independent substituents for where R⁵ is optionally substituted phenyl.

In sub-formula (x) and/or (y), preferably, one, two or three R⁶ substituents are present in B, D and/or E; so that for example in sub-formula (x), one, two or three R⁶ substituents are present in the meta- (3- and/or 5-) and/or para- (4-) positions with respect to the —(CH₂)ₙ— side-chain.

Preferably, R⁵ has the sub-formula (x), n is 1 and none of A, B, D, E and F are nitrogen or nitrogen-oxide (N⁺—O⁻); and all of A, B, D, E and F are independently CH or CR⁶; that is R⁵ has the sub-formula (x) and is optionally substituted benzyl. In this embodiment, preferably, a R⁶ substituent is present at the 4-position with respect to the —(CH₂)ₙ— side-chain (that is D is CR⁶: i.e. a R⁶ substituent is present in D); and/or preferably a R⁶ substituent is present at the 3- and/or 5-position with respect to the —(CH₂)ₙ— side-chain (that is B and/or E is CR⁶: i.e. one or two R⁶ substituents are present in B and/or E). For monosubstitution, i.e. where one of A, B, D, E and F is CR⁶, then the one R⁶ substituent is preferably present at the 4-position with respect to the —(CH₂)ₙ— side-chain (i.e. D is CR⁶). Where there is disubstitution, that is where two of A, B, D, E and F are independently CR⁶, then 3,4-disubstitution (B+D or D+E are independently CR⁶), 2,4-disubstitution (A+D or D+F are independently CR⁶) or 2,3-disubstitution (A+B or E+F are independently CR⁶) is preferred.

In sub-formula (x) and/or (y), any optional R⁶ substituent can optionally be present only in B, D and/or E, so that in sub-formula (x) any optional R⁶ substituent is present only in the meta- (3- and/or 5-) and/or para- (4-) positions with respect to the —(CH₂)ₙ— side-chain. Alternatively, in sub-formula (x), any optional R⁶ substituent can be present in the ortho- (2- and/or 6-) position with respect to the —(CH₂)ₙ— side-chain, either alone or in combination with one or more other optional R⁶ substituents.

Overall for R⁵, it is preferable that R⁵ is a hydrogen atom (H); C₁₋₆alkyl (e.g. C_{1, 2 or 3}alkyl or C₃₋₆alkyl); C₁₋₄fluoroalkyl, C₃₋₆cycloalkyl (e.g. C₅₋₆cycloalkyl), (C₅₋₆cycloalkyl)methyl-, phenyl optionally substituted with one or two of: a fluorine or chlorine atom, methyl, trifluoromethyl, methoxy or trifluoromethoxy; or R⁵ has the sub-formula (x), (y) or (z), for example as described above.

Still more preferably, R⁵ is a hydrogen atom (H), methyl, ethyl, n-propyl, iso-propyl, 2-ethylbutan-1-yl, cyclopentyl, cyclohexyl, (cyclohexyl)methyl-, optionally substituted phenyl e.g. fluorophenyl e.g. 4-fluorophenyl, optionally substituted benzyl, or optionally substituted pyridinylmethyl, or R⁵ has the sub-formula (z).

Optionally, R⁵ can be benzyl, pyridinylmethyl (e.g. pyridin-4-ylmethyl, pyridin-3-ylmethyl, or preferably pyridin-2-ylmethyl), or 4-fluorophenyl.

In one preferable embodiment, R⁵ has the sub-formula (x) and is: benzyl, (monoalkyl-phenyl)methyl, [mono(fluoroalkyl)-phenyl]methyl, (monohalo-phenyl)methyl, (monoalkoxy-phenyl)methyl, [mono(fluoroalkoxy)-phenyl]methyl, [mono(N,N-dimethylamino)-phenyl]methyl, [mono(methyl-SO₂—NH—)-phenyl]methyl, [mono(methyl-SO₂—)-phenyl]methyl, (dialkyl-phenyl)methyl, (monoalkyl-monohalo-phenyl)methyl, [mono(fluoroalkyl)-monohalo-phenyl]methyl, (dihalo-phenyl)methyl, (dihalo-monoalkyl-phenyl)methyl, [dihalo-mono(hydroxymethyl)-phenyl]methyl, or (dialkoxy-phenyl)methyl such as (3,4-dimethoxy-phenyl)methyl. The substituents can preferably be further defined, as defined in preferable embodiments herein.

In one preferable embodiment, R⁵ is of sub-formula (x) and is: (monoalkyl-phenyl)methyl, [mono(fluoroalkyl)-phenyl]methyl, (monohalo-phenyl)methyl, (monoalkoxy-phenyl)methyl, [mono(fluoroalkoxy)-phenyl]methyl, [mono(N,N-dimethylamino)-phenyl]methyl, (dialkyl-phenyl)methyl, (monoalkyl-monohalo-phenyl)methyl, (dihalo-phenyl)methyl or (dihalo-monoalkyl-phenyl)methyl or [dihalo-mono(hydroxymethyl)-phenyl]methyl. More preferably, in this embodiment, R⁵ is:
(monoC₁₋₃alkyl-phenyl)methyl such as (4-C₁₋₃alkyl-phenyl)methyl;
(mono C₁fluoroalkyl-phenyl)methyl such as (4-C₁fluoroalkyl-phenyl)methyl;
(monoC₁₋₂alkoxy-phenyl)methyl such as (4-C₁₋₂alkoxy-phenyl)methyl;
[mono(C₁fluoroalkoxy)-phenyl]methyl such as (4-C₁fluoroalkoxy-phenyl)methyl;
(diC₁₋₂alkyl-phenyl)methyl or (dimethyl-phenyl)methyl such as (3,4-dimethyl-phenyl)methyl, (2,4-dimethyl-phenyl)methyl, (3,5-dimethyl-phenyl)methyl, (2,3-dimethyl-phenyl)methyl or (2,5-dimethyl-phenyl)methyl; more preferably (3,4-dimethyl-phenyl)methyl or (2,4-dimethyl-phenyl)methyl;
(monoC₁₋₂alkyl-monohalo-phenyl)methyl or (monoC₁₋₂alkyl-monochloro-phenyl)methyl such as (4-methyl-3-chloro-phenyl)methyl, (3-methyl-4-chloro-phenyl)methyl, (2-methyl-4-chloro-phenyl)methyl;
(dihalo-phenyl)methyl such as (2-chloro-4-fluorophenyl)methyl or (2,4-difluoro-phenyl)methyl or (4-bromo-2-fluorophenyl)methyl or preferably (4-chloro-2-fluorophenyl)methyl; for example (dichloro-phenyl)methyl such as (3,4-dichloro-phenyl)methyl or (2,4-dichloro-phenyl)methyl or (2,6-dichloro-phenyl)methyl or preferably (2,3-dichloro-phenyl)methyl;
(dihalo-monoC₁₋₂alkyl-phenyl)methyl e.g. (2,4-dichloro-6-methyl-phenyl)methyl; or
[dihalo-mono(hydroxymethyl)-phenyl]methyl such as [2,3-dichloro-6-(hydroxymethyl)-phenyl]methyl.

In an alternative preferable embodiment, R⁵ has the sub-formula (z), and one or preferably none of J, L, M or Q is CR⁶, and/or R⁹ is a hydrogen atom (H) or methyl. Preferably r is 1. Preferably, for (z), R⁶ is independently OH (including any keto tautomer thereof), or more preferably C₁₋₂alkyl (e.g. methyl) or C₁fluoroalkyl.

Preferably NR⁴R⁵ is not NH₂. R⁵ is preferably not a hydrogen atom (H).

When R⁴ and R⁵ taken together are optionally substituted —(CH₂)ₚ¹— or optionally substituted —C(O)—(CH₂)ₚ²— or —(CH₂)ₚ³—X⁵—(CH₂)ₚ⁴— or —C(O)—X⁵—(CH₂)ₚ⁵— or a partially unsaturated derivative of any of the foregoing, preferably R⁴ and R⁵ taken together are optionally substituted —(CH₂)ₚ¹— or optionally substituted —C(O)—(CH₂)ₚ²— or —(CH₂)ₚ³—X⁵—(CH₂)ₚ⁴— or —C(O)—X⁵—(CH₂)ₚ⁵— (i.e. not a partially unsaturated derivative of any of these).

When R⁴ and R⁵ taken together are —(CH₂)ₚ¹— optionally substituted by R¹⁸, or —C(O)—(CH₂)ₚ²— optionally substituted by R¹⁸, or —(CH₂)ₚ³—X⁵—(CH₂)ₚ⁴—, NR⁴R⁵ can for example be

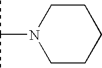

optionally substituted by R¹⁸, or

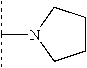

optionally substituted by R¹⁸, or

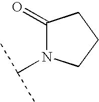

optionally substituted by R¹⁸, or

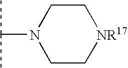

(i.e. R⁴ and R⁵ taken together are —(CH₂)₂—N(R¹⁷)—(CH₂)₂—), or

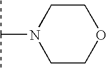

(i.e. R⁴ and R⁵ taken together are —(CH₂)₂—O—(CH₂)₂—).

Preferably, R¹⁷ is a hydrogen atom (H); C₁₋₄alkyl (e.g. C₁₋₂alkyl); C₃₋₆cycloalkyl; —(CH₂)ₚ⁶—C(O)R¹⁶, or the optionally substituted phenyl or benzyl. More preferably, R¹⁷ is H; C₁₋₂alkyl; —(CH₂)ₚ⁶—C(O)R¹⁶ or the optionally substituted phenyl.

When R⁴ and R⁵ taken together are —(CH₂)ₚ¹— or —C(O)—(CH₂)ₚ²—, the NR⁴R⁵ heterocycle is preferably not substituted by R¹⁸.

When R⁴ and R⁵ taken together are —(CH₂)ₚ¹— or —C(O)—(CH₂)ₚ²—, and if the NR⁴R⁵ heterocycle is substituted by R¹⁸, then optionally R¹⁸ is not substituted at a ring-carbon bonded to the NR⁴R⁵ ring-nitrogen.

When R⁴ and R⁵ taken together are —(CH₂)ₚ¹— or —C(O)—(CH₂)ₚ²— or —(CH₂)ₚ³—X⁵—(CH₂)ₚ⁴— or —C(O)—X⁵—(CH₂)ₚ⁵— or a partially unsaturated derivative of any of these, and wherein the NR⁴R⁵ heterocycle is fused to a phenyl ring optionally substituted on the phenyl by one or two of: a halogen atom, C₁₋₂alkyl, C₁fluoroalkyl, C₁₋₂alkoxy or C₁fluoroalkoxy; then in one embodiment of the invention NR⁴R⁵ is

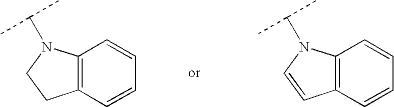

wherein the phenyl is optionally substituted by one or two of: a halogen atom, C₁₋₂alkyl, C₁fluoroalkyl, C₁₋₂alkoxy or C₁fluoroalkoxy.

In one embodiment of the invention, NR⁷R⁸ and/or NR¹²R¹³ can for example independently be

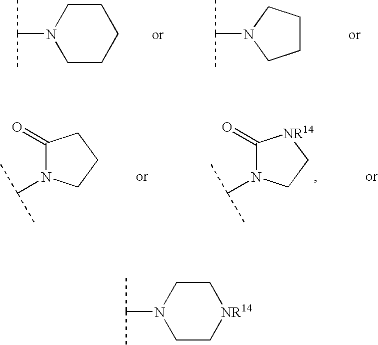

(i.e. R¹² and R¹³ together or R⁷ and R⁸ together are —(CH₂)₂—N(R¹⁴)—(CH₂)₂—), or

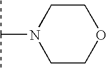

(i.e. R¹² and R¹³ together or R⁷ and R⁸ together are —(CH₂)₂—O—(CH₂)₂—), or NMe₂.

Preferably, R¹⁵ is a hydrogen atom (H) or C₁₋₄alkyl (e.g. ^{t}Bu or C₁₋₂alkyl e.g. methyl); more preferably, R¹⁵ is a hydrogen atom (H).

Preferably, however, R⁴ and R⁵ are not taken together, i.e. are not taken together to form the NR⁴R⁵ ring systems described herein.

(Similar preferences apply for R^{5a} as for R⁵, except that R^{5a} cannot be a hydrogen atom. Most preferably, R^{5a} is ethyl.)

In an especially preferable embodiment, NR⁴R⁵ is the NR⁴R⁵ group as defined in any one of: Examples 21-98, 100-182, 187-188, 191-200, 201-203, 210-353, 355-651, 653-658, 660-664 and 665-686.

It is particularly preferred that the compound of formula (I) or the salt thereof is:
Ethyl 4-(cyclopentylamino)-1-ethyl-1H-pyrazolo[3,4-b]pyridine-5-carboxylate,
Ethyl 4-(cyclohexylamino)-1-ethyl-1H-pyrazolo[3,4-b]pyridine-5-carboxylate,
Ethyl 1-ethyl-4-(tetrahydro-2H-pyran-4-ylamino)-1H-pyrazolo[3,4-b]pyridine-5-carboxylate,
Ethyl 4-[(1-acetylpiperidin-4-yl)amino]-1-ethyl-1H-pyrazolo[3,4-b]pyridine-5-carboxylate,
Ethyl 4-(cyclopentylamino)-1-methyl-1H-pyrazolo[3,4-b]pyridine-5-carboxylate (not this compound per se, and for the use or method of treatment preferably not this compound),
Ethyl 1-methyl-4-(tetrahydro-2H-pyran-4-ylamino)-1H-pyrazolo[3,4-b]pyridine-5-carboxylate,
Ethyl 1-ethyl-4-[(3S)-tetrahydrofuran-3-ylamino]-1H-pyrazolo[3,4-b]pyridine-5-carboxylate,
Ethyl 1-ethyl-4-[(3R)-tetrahydrofuran-3-ylamino]-1H-pyrazolo[3,4-b]pyridine-5-carboxylate,
Ethyl 1-ethyl-4-(tetrahydro-2H-thiopyran-4-ylamino)-1H-pyrazolo[3,4-b]pyridine-5-carboxylate,
Ethyl 1-ethyl-4-(tetrahydrothien-3-ylamino)-1H-pyrazolo[3,4-b]pyridine-5-carboxylate,
Ethyl 4-(cyclopropylamino)-1-ethyl-1H-pyrazolo[3,4-b]pyridine-5-carboxylate,
Ethyl 4-[(1,1-dioxidotetrahydrothien-3-yl)amino]-1-ethyl-1H-pyrazolo[3,4-b]pyridine-5-carboxylate,
Ethyl 4-[(1,1-dioxidotetrahydro-2H-thiopyran-4-yl)amino]-1-ethyl-1H-pyrazolo[3,4-b]pyridine-5-carboxylate,
N-Benzyl-1-ethyl-4-(tetrahydro-2H-pyran-4-ylamino)-1H-pyrazolo[3,4-b]pyridine-5-carboxamide,
1-Ethyl-N-(4-fluorophenyl)-4-(tetrahydro-2H-pyran-4-ylamino)-1H-pyrazolo[3,4-b]pyridine-5-carboxamide,
N-Cyclopentyl-4-(cyclopentylamino)-1-ethyl-1H-pyrazolo[3,4-b]pyridine-5-carboxamide,
4-(Cyclohexylamino)-N-cyclopentyl-1-ethyl-1H-pyrazolo[3,4-b]pyridine-5-carboxamide,
N-Cyclopentyl-1-ethyl-4-(tetrahydro-2H-pyran-4-ylamino)-1H-pyrazolo[3,4-b]pyridine-5-carboxamide,
4-[(1-Acetylpiperidin-4-yl)amino]-N-cyclopentyl-1-ethyl-1H-pyrazolo[3,4-b]pyridine-5-carboxamide,
N-Cyclopentyl-1-ethyl-5-(pyrrolidin-1-ylcarbonyl)-1H-pyrazolo[3,4-b]pyridin-4-amine,
N-Cyclohexyl-1-ethyl-5-(pyrrolidin-1-ylcarbonyl)-1H-pyrazolo[3,4-b]pyridin-4-amine,
1-Ethyl-5-(pyrrolidin-1-ylcarbonyl)-N-tetrahydro-2H-pyran-4-yl-1H-pyrazolo[3,4-b]pyridin-4-amine,
4-(Cyclopentylamino)-1-ethyl-N-(pyridin-4-ylmethyl)-1H-pyrazolo[3,4-b]pyridine-5-carboxamide,
4-(Cyclohexylamino)-1-ethyl-N-(pyridin-4-ylmethyl)-1H-pyrazolo[3,4-b]pyridine-5-carboxamide,
1-Ethyl-N-(pyridin-4-ylmethyl)-4-(tetrahydro-2H-pyran-4-ylamino)-1H-pyrazolo[3,4-b]pyridine-5-carboxamide,
4-(Cyclopentylamino)-1-ethyl-1H-pyrazolo[3,4-b]pyridine-5-carboxamide,
4-(Cyclohexylamino)-1-ethyl-1H-pyrazolo[3,4-b]pyridine-5-carboxamide,
1-Ethyl-4-(tetrahydro-2H-pyran-4-ylamino)-1H-pyrazolo[3,4-b]pyridine-5-carboxamide,
N-Benzyl-4-(cyclopentylamino)-1-ethyl-1H-pyrazolo[3,4-b]pyridine-5-carboxamide,
N-Benzyl-4-(cyclohexylamino)-1-ethyl-1H-pyrazolo[3,4-b]pyridine-5-carboxamide,
4-[(1-Acetylpiperidin-4-yl)amino]-N-benzyl-1-ethyl-1H-pyrazolo[3,4-b]pyridine-5-carboxamide,
4-(Cyclopentylamino)-1-ethyl-N-(2-ethylbutyl)-1H-pyrazolo[3,4-b]pyridine-5-carboxamide,
4-(Cyclohexylamino)-1-ethyl-N-(2-ethylbutyl)-1H-pyrazolo[3,4-b]pyridine-5-carboxamide,
1-Ethyl-N-(2-ethylbutyl)-4-(tetrahydro-2H-pyran-4-ylamino)-1H-pyrazolo[3,4-b]pyridine-5-carboxamide,
1-Ethyl-N-(2-ethylbutyl)-4-[(1-methylpiperidin-4-yl)amino]-1H-pyrazolo[3,4-b]pyridine-5-carboxamide,
4-[(1-Acetylpiperidin-4-yl)amino]-1-ethyl-N-(2-ethylbutyl)-1H-pyrazolo[3,4-b]pyridine-5-carboxamide,
4-(Cyclopentylamino)-1-ethyl-N-(4-fluorophenyl)-1H-pyrazolo[3,4-b]pyridine-5-carboxamide,
4-(Cyclohexylamino)-1-ethyl-N-(4-fluorophenyl)-1H-pyrazolo[3,4-b]pyridine-5-carboxamide,
1-Ethyl-N-(4-fluorophenyl)-4-[(1-methylpiperidin-4-yl)amino]-1H-pyrazolo[3,4-b]pyridine-5-carboxamide,
4-[(1-Acetylpiperidin-4-yl)amino]-1-ethyl-N-(4-fluorophenyl)-1H-pyrazolo[3,4-b]pyridine-5-carboxamide,
4-(Cyclopentylamino)-1-ethyl-N-n-propyl-1H-pyrazolo[3,4-b]pyridine-5-carboxamide,
4-(Cyclohexylamino)-1-ethyl-N-n-propyl-1H-pyrazolo[3,4-b]pyridine-5-carboxamide,
1-Ethyl-N-n-propyl-4-(tetrahydro-2H-pyran-4-ylamino)-1H-pyrazolo[3,4-b]pyridine-5-carboxamide,
4-[(1-Acetylpiperidin-4-yl)amino]-1-ethyl-N-n-propyl-1H-pyrazolo[3,4-b]pyridine-5-carboxamide,
4-[(1-Acetylpiperidin-4-yl)amino]-1-ethyl-N-(pyridin-4-ylmethyl)-1H-pyrazolo[3,4-b]pyridine-5-carboxamide,
N-Benzyl-4-(cyclopentylamino)-1-methyl-1H-pyrazolo[3,4-b]pyridine-5-carboxamide,
N-Benzyl-4-(cyclohexylamino)-1-methyl-1H-pyrazolo[3,4-b]pyridine-5-carboxamide,
N-Benzyl-1-methyl-4-(tetrahydro-2H-pyran-4-ylamino)-1H-pyrazolo[3,4-b]pyridine-5-carboxamide,
4-(Cyclopentylamino)-N-(2-ethylbutyl)-1-methyl-1H-pyrazolo[3,4-b]pyridine-5-carboxamide,
4-(Cyclohexylamino)-N-(2-ethylbutyl)-1-methyl-1H-pyrazolo[3,4-b]pyridine-5-carboxamide,
N-(2-Ethylbutyl)-1-methyl-4-(tetrahydro-2H-pyran-4-ylamino)-1H-pyrazolo[3,4-b]pyridine-5-carboxamide,
4-(Cyclopentylamino)-N-(4-fluorophenyl)-1-methyl-1H-pyrazolo[3,4-b]pyridine-5-carboxamide,
4-(Cyclohexylamino)-N-(4-fluorophenyl)-1-methyl-1H-pyrazolo[3,4-b]pyridine-5-carboxamide,
N-(4-Fluorophenyl)-1-methyl-4-(tetrahydro-2H-pyran-4-ylamino)-1H-pyrazolo[3,4-b]pyridine-5-carboxamide,
4-(Cyclopentylamino)-1-methyl-1H-pyrazolo[3,4-b]pyridine-5-carboxamide,
4-(Cyclohexylamino)-1-methyl-1H-pyrazolo[3,4-b]pyridine-5-carboxamide,
4-[(1-Acetylpiperidin-4-yl)amino]-N-benzyl-1-methyl-1H-pyrazolo[3,4-b]pyridine-5-carboxamide,
1-Ethyl-N-methyl-4-(tetrahydro-2H-pyran-4-ylamino)-1H-pyrazolo[3,4-b]pyridine-5-carboxamide,
1-Ethyl-N,N-dimethyl-4-(tetrahydro-2H-pyran-4-ylamino)-1H-pyrazolo[3,4-b]pyridine-5-carboxamide,
1-Ethyl-N-ethyl-4-(tetrahydro-2H-pyran-4-ylamino)-1H-pyrazolo[3,4-b]pyridine-5-carboxamide,
1-Ethyl-N-isopropyl-4-(tetrahydro-2H-pyran-4-ylamino)-1H-pyrazolo[3,4-b]pyridine-5-carboxamide,
N-Benzyl-1-ethyl-4-[(3S)-tetrahydrofuran-3-ylamino]-1H-pyrazolo[3,4-b]pyridine-5-carboxamide,
N-Benzyl-1-ethyl-4-[(3R)-tetrahydrofuran-3-ylamino]-1H-pyrazolo[3,4-b]pyridine-5-carboxamide,
N-Benzyl-1-ethyl-4-(tetrahydrothien-3-ylamino)-1H-pyrazolo[3,4-b]pyridine-5-carboxamide,
N-Benzyl-4-(cyclopropylamino)-1-ethyl-1H-pyrazolo[3,4-b]pyridine-5-carboxamide,
N-Benzyl-4-[(1,1-dioxidotetrahydrothien-3-yl)amino]-1-ethyl-1H-pyrazolo[3,4-b]pyridine-5-carboxamide,
N-Benzyl-4-[(1,1-dioxidotetrahydro-2H-thiopyran-4-yl)amino]-1-ethyl-1H-pyrazolo[3,4-b]pyridine-5-carboxamide,
N-Benzyl-1-ethyl-4-(tetrahydro-2H-thiopyran-4-ylamino)-1H-pyrazolo[3,4-b]pyridine-5-carboxamide,
1-Ethyl-N-(4-fluorophenyl)-4-[(3S)-tetrahydrofuran-3-ylamino]-1H-pyrazolo[3,4-b]pyridine-5-carboxamide,
1-Ethyl-N-(4-fluorophenyl)-4-[(3R)-tetrahydrofuran-3-ylamino]-1H-pyrazolo[3,4-b]pyridine-5-carboxamide,
1-Ethyl-N-(4-fluorophenyl)-4-(tetrahydro-2H-thiopyran-4-ylamino)-1H-pyrazolo[3,4-b]pyridine-5-carboxamide,
1-Ethyl-N-(4-fluorophenyl)-4-(tetrahydrothien-3-ylamino)-1H-pyrazolo[3,4-b]pyridine-5-carboxamide,
4-(Cyclopropylamino)-1-ethyl-N-(4-fluorophenyl)-1H-pyrazolo[3,4-b]pyridine-5-carboxamide,
4-[(1,1-Dioxidotetrahydrothien-3-yl)amino]-1-ethyl-N-(4-fluorophenyl)-1H-pyrazolo[3,4-b]pyridine-5-carboxamide, or
4-[(1,1-Dioxidotetrahydro-2H-thiopyran-4-yl)amino]-1-ethyl-N-(4-fluorophenyl)-1H-pyrazolo[3,4-b]pyridine-5-carboxamide;
   or a salt thereof, e.g. a pharmaceutically acceptable salt thereof.

The structures of these specific compounds are given in Examples 1-98 hereinafter.

Alternatively, it is particularly preferred that the compound of formula (I) or the salt thereof is:
1-Ethyl-N-[4-(methylsulfonyl)benzyl]-4-(tetrahydro-2H-pyran-4-ylamino)-1H-pyrazolo[3,4-b]pyridine-5-carboxamide,
1-Ethyl-N-[3-(methylsulfonyl)benzyl]-4-(tetrahydro-2H-pyran-4-ylamino)-1H-pyrazolo[3,4-b]pyridine-5-carboxamide,
1-Ethyl-5-{[5-methoxy-6-(trifluoromethyl)-2,3-dihydro-1H-indol-1-yl]carbonyl}-N-tetrahydro-2H-pyran-4-yl-1H-pyrazolo[3,4-b]pyridin-4-amine,
N-[(5-Chloropyridin-2-yl)methyl]-1-ethyl-4-(tetrahydro-2H-pyran-4-ylamino)-1H-pyrazolo[3,4-b]pyridine-5-carboxamide,
N-(4-Chlorobenzyl)-1-ethyl-N-isopropyl-4-(tetrahydro-2H-pyran-4-ylamino)-1H-pyrazolo[3,4-b]pyridine-5-carboxamide,
N-(3-Chlorobenzyl)-1-ethyl-N-(2-hydroxyethyl)-4-(tetrahydro-2H-pyran-4-ylamino)-1H-pyrazolo[3,4-b]pyridine-5-carboxamide,
1-Ethyl-N-[(5-methyl-3-phenylisoxazol-4-yl)methyl]-4-(tetrahydro-2H-pyran-4-ylamino)-1H-pyrazolo[3,4-b]pyridine-5-carboxamide,
N-(2-tert-Butoxyethyl)-1-ethyl-4-(tetrahydro-2H-pyran-4-ylamino)-1H-pyrazolo[3,4-b]pyridine-5-carboxamide,
1-Ethyl-4-(tetrahydro-2H-pyran-4-ylamino)-N-(1,3-thiazol-2-ylmethyl)-1H-pyrazolo[3,4-b]pyridine-5-carboxamide,
1-Ethyl-N-(pyrimidin-4-ylmethyl)-4-(tetrahydro-2H-pyran-4-ylamino)-1H-pyrazolo[3,4-b]pyridine-5-carboxamide,
1-Ethyl-N-[(2-methyl-1,3-thiazol-4-yl)methyl]-4-(tetrahydro-2H-pyran-4-ylamino)-1H-pyrazolo[3,4-b]pyridine-5-carboxamide,
N-[3-(tert-Butoxymethyl)benzyl]-1-ethyl-4-(tetrahydro-2H-pyran-4-ylamino)-1H-pyrazolo[3,4-b]pyridine-5-carboxamide,
1-Ethyl-N-{2-[methyl(methylsulfonyl)amino]ethyl}-4-(tetrahydro-2H-pyran-4-ylamino)-1H-pyrazolo[3,4-b]pyridine-5-carboxamide,
1-Ethyl-N-(pyrazin-2-ylmethyl)-4-(tetrahydro-2H-pyran-4-ylamino)-1H-pyrazolo[3,4-b]pyridine-5-carboxamide,
1-Ethyl-5-{[4-(pyridin-2-ylcarbonyl)piperazin-1-yl]carbonyl}-N-tetrahydro-2H-pyran-4-yl-1H-pyrazolo[3,4-b]pyridin-4-amine,
N-(2-Chloro-6-fluorobenzyl)-1-ethyl-4-(tetrahydro-2H-pyran-4-ylamino)-1H-pyrazolo[3,4-b]pyridine-5-carboxamide,
1-Ethyl-N-[(6-oxo-1,6-dihydropyridin-3-yl)methyl]-4-(tetrahydro-2H-pyran-4-ylamino)-1H-pyrazolo[3,4-b]pyridine-5-carboxamide,
N-[3-(Aminocarbonyl)benzyl]-1-ethyl-4-(tetrahydro-2H-pyran-4-ylamino)-1H-pyrazolo[3,4-b]pyridine-5-carboxamide,
1-Ethyl-N-{4-[(methylamino)carbonyl]phenyl}-4-(tetrahydro-2H-pyran-4-ylamino)-1H-pyrazolo[3,4-b]pyridine-5-carboxamide,
1-Ethyl-N-[2-(1-methyl-1H-imidazol-4-yl)ethyl]-4-(tetrahydro-2H-pyran-4-ylamino)-1H-pyrazolo[3,4-b]pyridine-5-carboxamide,
N-{2-[(Anilinocarbonyl)amino]ethyl}-1-ethyl-4-(tetrahydro-2H-pyran-4-ylamino)-1H-pyrazolo[3,4-b]pyridine-5-carboxamide,
1-Ethyl-N-(1H-tetrazol-5-ylmethyl)-4-(tetrahydro-2H-pyran-4-ylamino)-1H-pyrazolo[3,4-b]pyridine-5-carboxamide,
1-Ethyl-4-(tetrahydro-2H-pyran-4-ylamino)-N-[2-(1H-1,2,4-triazol-1-yl)ethyl]-1H-pyrazolo[3,4-b]pyridine-5-carboxamide,
1-Ethyl-4-(tetrahydro-2H-pyran-4-ylamino)-N-[4-(trifluoromethyl)phenyl]-1H-pyrazolo[3,4-b]pyridine-5-carboxamide,
tert-Butyl 4-({[1-ethyl-4-(tetrahydro-2H-pyran-4-ylamino)-1H-pyrazolo[3,4-b]pyridin-5-yl]carbonyl}amino)piperidine-1-carboxylate,
1-Ethyl-N-{3-[(methylsulfonyl)amino]propyl}-4-(tetrahydro-2H-pyran-4-ylamino)-1H-pyrazolo[3,4-b]pyridine-5-carboxamide,
N-[2-(Dimethylamino)benzyl]-1-ethyl-4-(tetrahydro-2H-pyran-4-ylamino)-1H-pyrazolo[3,4-b]pyridine-5-carboxamide,
1-Ethyl-N-[(1-ethylpyrrolidin-2-yl)methyl]-4-(tetrahydro-2H-pyran-4-ylamino)-1H-pyrazolo[3,4-b]pyridine-5-carboxamide,
1-Ethyl-N-(tetrahydrofuran-2-ylmethyl)-4-(tetrahydro-2H-pyran-4-ylamino)-1H-pyrazolo[3,4-b]pyridine-5-carboxamide,
1-ethyl-N-tetrahydro-2H-pyran-4-yl-4-(tetrahydro-2H-pyran-4-ylamino)-1H-pyrazolo[3,4-b]pyridine-5-carboxamide,
N-{4-[(Dimethylamino)sulfonyl]benzyl}-1-ethyl-4-(tetrahydro-2H-pyran-4-ylamino)-1H-pyrazolo[3,4-b]pyridine-5-carboxamide,
1-Ethyl-N-{3-[(methylsulfonyl)amino]benzyl}-4-(tetrahydro-2H-pyran-4-ylamino)-1H-pyrazolo[3,4-b]pyridine-5-carboxamide,
1-Ethyl-N-(4-methoxyphenyl)-4-(tetrahydro-2H-pyran-4-ylamino)-1H-pyrazolo[3,4-b]pyridine-5-carboxamide,
1-Ethyl-N-[3-(2-oxopyrrolidin-1-yl)propyl]-4-(tetrahydro-2H-pyran-4-ylamino)-1H-pyrazolo[3,4-b]pyridine-5-carboxamide,
1-Ethyl-N-[2-(1-methylpyrrolidin-2-yl)ethyl]-4-(tetrahydro-2H-pyran-4-ylamino)-1H-pyrazolo[3,4-b]pyridine-5-carboxamide,
1-Ethyl-N-(pyridin-3-ylmethyl)-4-(tetrahydro-2H-pyran-4-ylamino)-1H-pyrazolo[3,4-b]pyridine-5-carboxamide,
1-Ethyl-N-(1-methylpiperidin-4-yl)-4-(tetrahydro-2H-pyran-4-ylamino)-1H-pyrazolo[3,4-b]pyridine-5-carboxamide,
1-Ethyl-N-(1-ethylpropyl)-4-(tetrahydro-2H-pyran-4-ylamino)-1H-pyrazolo[3,4-b]pyridine-5-carboxamide,
1-Ethyl-N-(2-piperidin-1-ylethyl)-4-(tetrahydro-2H-pyran-4-ylamino)-1H-pyrazolo[3,4-b]pyridine-5-carboxamide,
1-Ethyl-N-(3-morpholin-4-ylpropyl)-4-(tetrahydro-2H-pyran-4-ylamino)-1H-pyrazolo[3,4-b]pyridine-5-carboxamide,
N-(3-Ethoxypropyl)-1-ethyl-4-(tetrahydro-2H-pyran-4-ylamino)-1H-pyrazolo[3,4-b]pyridine-5-carboxamide,
N-(Cyclohexylmethyl)-1-ethyl-4-(tetrahydro-2H-pyran-4-ylamino)-1H-pyrazolo[3,4-b]pyridine-5-carboxamide,
N-[3-(Dimethylamino)propyl]-1-ethyl-4-(tetrahydro-2H-pyran-4-ylamino)-1H-pyrazolo[3,4-b]pyridine-5-carboxamide,
1-Ethyl-N-neopentyl-4-(tetrahydro-2H-pyran-4-ylamino)-1H-pyrazolo[3,4-b]pyridine-5-carboxamide,
1-ethyl-N-(4-methoxybenzyl)-4-(tetrahydro-2H-pyran-4-ylamino)-1H-pyrazolo[3,4-b]pyridine-5-carboxamide,
1-Ethyl-N-{2-[(phenylsulfonyl)amino]ethyl}-4-(tetrahydro-2H-pyran-4-ylamino)-1H-pyrazolo[3,4-b]pyridine-5-carboxamide,
N-[2-(Acetylamino)ethyl]-1-ethyl-4-(tetrahydro-2H-pyran-4-ylamino)-1H-pyrazolo[3,4-b]pyridine-5-carboxamide,
1-Ethyl-N-{2-[(methylsulfonyl)amino]ethyl}-4-(tetrahydro-2H-pyran-4-ylamino)-1H-pyrazolo[3,4-b]pyridine-5-carboxamide,
1-Ethyl-N-{2-[(2-methoxyphenyl)(methyl)amino]ethyl}-4-(tetrahydro-2H-pyran-4-ylamino)-1H-pyrazolo[3,4-b]pyridine-5-carboxamide,
1-Ethyl-N-(2-oxo-2-phenylethyl)-4-(tetrahydro-2H-pyran-4-ylamino)-1H-pyrazolo[3,4-b]pyridine-5-carboxamide,
N-(2,5-Difluorobenzyl)-1-ethyl-4-(tetrahydro-2H-pyran-4-ylamino)-1H-pyrazolo[3,4-b]pyridine-5-carboxamide,
1-Ethyl-4-(tetrahydro-2H-pyran-4-ylamino)-N-[4-(trifluoromethyl)benzyl]-1H-pyrazolo[3,4-b]pyridine-5-carboxamide,
N,1-Diethyl-N-propyl-4-(tetrahydro-2H-pyran-4-ylamino)-1H-pyrazolo[3,4-b]pyridine-5-carboxamide,
N-Cyclopropyl-1-ethyl-4-(tetrahydro-2H-pyran-4-ylamino)-1H-pyrazolo[3,4-b]pyridine-5-carboxamide,
N-(2-amino-2-oxoethyl)-1-ethyl-4-(tetrahydro-2H-pyran-4-ylamino)-1H-pyrazolo[3,4-b]pyridine-5-carboxamide,
1-Ethyl-N-(3-methoxyphenyl)-4-(tetrahydro-2H-pyran-4-ylamino)-1H-pyrazolo[3,4-b]pyridine-5-carboxamide,
N-(3,4-Difluorobenzyl)-1-ethyl-4-(tetrahydro-2H-pyran-4-ylamino)-1H-pyrazolo[3,4-b]pyridine-5-carboxamide,
Ethyl 3-({[1-ethyl-4-(tetrahydro-2H-pyran-4-ylamino)-1H-pyrazolo[3,4-b]pyridin-5-yl]carbonyl}amino)propanoate,
N-(1-Benzylpiperidin-4-yl)-1-ethyl-4-(tetrahydro-2H-pyran-4-ylamino)-1H-pyrazolo[3,4-b]pyridine-5-carboxamide,
N-Butyl-4-{[1-ethyl-4-(tetrahydro-2H-pyran-4-ylamino)-1H-pyrazolo[3,4-b]pyridin-5-yl]carbonyl}piperazine-1-carboxamide,
1-Ethyl-4-(tetrahydro-2H-pyran-4-ylamino)-N-(1,3,4-thiadiazol-2-yl)-1H-pyrazolo[3,4-b]pyridine-5-carboxamide,
N-(2,3-Dihydro-1H-inden-2-yl)-1-ethyl-4-(tetrahydro-2H-pyran-4-ylamino)-1H-pyrazolo[3,4-b]pyridine-5-carboxamide,
1-Ethyl-N-[2-(2-oxoimidazolidin-1-yl)ethyl]-4-(tetrahydro-2H-pyran-4-ylamino)-1H-pyrazolo[3,4-b]pyridine-5-carboxamide,
N-(3,4-Dimethoxybenzyl)-1-ethyl-4-(tetrahydro-2H-pyran-4-ylamino)-1H-pyrazolo[3,4-b]pyridine-5-carboxamide,
N-(3-Chlorobenzyl)-1-ethyl-4-(tetrahydro-2H-pyran-4-ylamino)-1H-pyrazolo[3,4-b]pyridine-5-carboxamide,
1-Ethyl-5-[(4-methylpiperazin-1-yl)carbonyl]-N-tetrahydro-2H-pyran-4-yl-1H-pyrazolo[3,4-b]pyridin-4-amine,
1-Ethyl-N-(2-hydroxyethyl)-4-(tetrahydro-2H-pyran-4-ylamino)-1H-pyrazolo[3,4-b]pyridine-5-carboxamide,
1-Ethyl-5-{[4-(4-methoxyphenyl)piperazin-1-yl]carbonyl}-N-tetrahydro-2H-pyran-4-yl-1H-pyrazolo[3,4-b]pyridin-4-amine,
1-Ethyl-N-{4-[(methylsulfonyl)methyl]phenyl}-4-(tetrahydro-2H-pyran-4-ylamino)-1H-pyrazolo[3,4-b]pyridine-5-carboxamide,
N-[3-(dimethylamino)-3-oxopropyl]-1-ethyl-4-(tetrahydro-2H-pyran-4-ylamino)-1H-pyrazolo[3,4-b]pyridine-5-carboxamide,
1-Ethyl-N-[(1-methyl-1H-imidazol-5-yl)methyl]-4-(tetrahydro-2H-pyran-4-ylamino)-1H-pyrazolo[3,4-b]pyridine-5-carboxamide,
1-Ethyl-N-{4-[(methylamino)sulfonyl]phenyl}-4-(tetrahydro-2H-pyran-4-ylamino)-1H-pyrazolo[3,4-b]pyridine-5-carboxamide,
N-(2-Cyanoethyl)-1-ethyl-4-(tetrahydro-2H-pyran-4-ylamino)-1H-pyrazolo[3,4-b]pyridine-5-carboxamide,
1-Ethyl-N-[(1-methyl-1H-pyrazol-4-yl)methyl]-4-(tetrahydro-2H-pyran-4-ylamino)-1H-pyrazolo[3,4-b]pyridine-5-carboxamide,
1-Ethyl-N-methyl-N-[(1-methyl-1H-imidazol-2-yl)methyl]-4-(tetrahydro-2H-pyran-4-ylamino)-1H-pyrazolo[3,4-b]pyridine-5-carboxamide,
1-Ethyl-4-(tetrahydro-2H-pyran-4-ylamino)-N-(2-thien-2-ylethyl)-1H-pyrazolo[3,4-b]pyridine-5-carboxamide,
N-[2-(4-Chlorophenyl)ethyl]-1-ethyl-4-(tetrahydro-2H-pyran-4-ylamino)-1H-pyrazolo[3,4-b]pyridine-5-carboxamide,
1-Ethyl-N-[2-(2-methoxyphenyl)ethyl]-4-(tetrahydro-2H-pyran-4-ylamino)-1H-pyrazolo[3,4-b]pyridine-5-carboxamide,
Ethyl 4-(cyclohexylamino)-1-(3-ethoxy-3-oxopropyl)-1H-pyrazolo[3,4-b]pyridine-5-carboxylate,
Ethyl 1-n-propyl-4-(tetrahydro-2H-pyran-4-ylamino)-1H-pyrazolo[3,4-b]pyridine-5-carboxylate,
Ethyl 1-(2-hydroxyethyl)-4-(tetrahydro-2H-pyran-4-ylamino)-1H-pyrazolo[3,4-b]pyridine-5-carboxylate,
N-[4-(Methylsulfonyl)benzyl]-1-n-propyl-4-(tetrahydro-2H-pyran-4-ylamino)-1H-pyrazolo[3,4-b]pyridine-5-carboxamide,
N-(4-Fluorophenyl)-1-n-propyl-4-(tetrahydro-2H-pyran-4-ylamino)-1H-pyrazolo[3,4-b]pyridine-5-carboxamide,
Ethyl 1-ethyl-6-methyl-4-(tetrahydro-2H-pyran-4-ylamino)-1H-pyrazolo[3,4-b]pyridine-5-carboxylate,
Ethyl 4-(cyclohexylamino)-1-ethyl-6-methyl-1H-pyrazolo[3,4-b]pyridine-5-carboxylate,
4-(Cyclohexylamino)-1-ethyl-6-methyl-N-[4-(methylsulfonyl)benzyl]-1H-pyrazolo[3,4-b]pyridine-5-carboxamide,
N-Benzyl-4-(cyclohexylamino)-1-ethyl-6-methyl-1H-pyrazolo[3,4-b]pyridine-5-carboxamide,
4-(Cyclohexylamino)-1-ethyl-N-(4-fluorophenyl)-6-methyl-1H-pyrazolo[3,4-b]pyridine-5-carboxamide,
4-(Cyclohexylamino)-1-ethyl-6-methyl-N-[4-(trifluoromethyl)benzyl]-1H-pyrazolo[3,4-b]pyridine-5-carboxamide,
4-(Cyclohexylamino)-N-(2,3-dihydro-1H-inden-2-yl)-1-ethyl-6-methyl-1H-pyrazolo[3,4-b]pyridine-5-carboxamide,
N-Benzyl-1-ethyl-6-methyl-4-(tetrahydro-2H-pyran-4-ylamino)-1H-pyrazolo[3,4-b]pyridine-5-carboxamide,
N-Benzyl-1-ethyl-4-[(2-oxoazepan-3-yl)amino]-1H-pyrazolo[3,4-b]pyridine-5-carboxamide,
N-Benzyl-1-ethyl-4-[(3-hydroxycyclohexyl)amino]-1H-pyrazolo[3,4-b]pyridine-5-carboxamide,
N-Benzyl-1-ethyl-4-[(4-hydroxycyclohexyl)amino]-1H-pyrazolo[3,4-b]pyridine-5-carboxamide,
N-Benzyl-1-ethyl-4-[(3-hydroxycyclopentyl)amino]-1H-pyrazolo[3,4-b]pyridine-5-carboxamide, or
N-Benzyl-1-ethyl-4-[(4-oxocyclohexyl)amino]-1H-pyrazolo[3,4-b]pyridine-5-carboxamide;
   or a salt thereof, e.g. a pharmaceutically acceptable salt thereof.

The structures of these specific compounds are given in Examples 100-201 hereinafter.

Alternatively, the compound of formula (I) or the salt thereof can be:
1-Ethyl-N-(2-hydroxy-1-methylethyl)-4-(tetrahydro-2H-pyran-4-ylamino)-1H-pyrazolo[3,4-b]pyridine-5-carboxamide, or
Methyl (2S)-2-({[1-ethyl-4-(tetrahydro-2H-pyran-4-ylamino)-1H-pyrazolo[3,4-b]pyridin-5-yl]carbonyl}amino)-3-hydroxypropanoate;
   or a salt thereof, e.g. a pharmaceutically acceptable salt thereof. (See for example Examples 202-203).

Alternatively, it is particularly preferred that the compound of formula (I) or the salt thereof is one of Examples 204 to 664 or one of Examples 665 to 686, as a compound or a salt thereof, e.g. a pharmaceutically acceptable salt thereof. The structures of these specific compounds are given in Examples 204 to 664 and Examples 665 to 686 hereinafter, and their names are given in the Examples section.

In one embodiment, is still further preferred that the compound of formula (I) or the salt thereof is a compound of Example 260, 261, 263, 266, 431, 493, 494, 518, 528, 584, 626, 643, 653, 679, 680, 681, 682, 683, 684, 685 or 686 (more preferably Example 260, 518, 653, 679, 680, 681 or 684), as defined by the structures and/or names described herein, or a salt thereof, e.g. a pharmaceutically acceptable salt thereof. The structures and names of these Examples are described in the Examples section. These Examples are thought to be suitable for inhaled administration.

In another embodiment, is still further preferred that the compound of formula (I) or the salt thereof is a compound of Example 21, 22, 83, 100, 109, 167, 172, 178 or 600, as defined by the structures and/or names described herein, or a salt thereof, e.g. a pharmaceutically acceptable salt thereof. The structures and names of these Examples are described in the Examples section. These Examples are thought to be suitable for oral administration.

A second aspect of the present invention provides a compound of formula (IA) or a salt thereof (in particular, a pharmaceutically acceptable salt thereof):

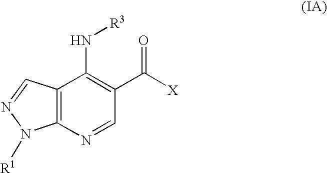

wherein:
X is NR⁴R⁵ or OR^{5a}, in which:
R⁴ is hydrogen, C₁₋₂alkyl or C₁₋₂fluoroalkyl, and
R⁵ is hydrogen, C₁₋₈alkyl, C₁₋₈ fluoroalkyl, or C₃₋₈cycloalkyl, phenyl optionally substituted with one or two of: a halogen atom, C₁₋₂alkyl, trifluoromethyl, C₁₋₂alkoxy or trifluoromethoxy; or R⁵ has the sub-formula (x), (y) or (z):

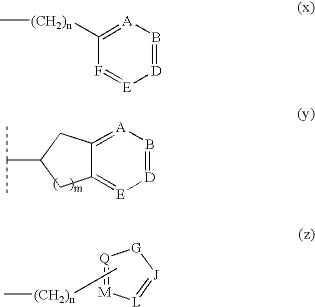

wherein in sub-formula (x) and (z), n=1 or 2; and in sub-formula (y), m=1 or 2;
wherein in sub-formula (x) and (y), none, one or two of A, B, D, E and F are nitrogen; and the remaining of A, B, D, E and F are CH or CR⁶ where R⁶ is a halogen atom, C₁₋₄alkyl, C₁₋₄fluoroalkyl, C₁₋₂alkoxy, C₁₋₂fluoroalkoxy, C₁₋₂alkylsulphonyl (C₁₋₂alkyl-SO₂—), C₁₋₂alkyl-SO₂—NH—, R⁷R⁸N—SO₂—, R⁷R⁸N—CO—, R⁷R⁸N, OH, C₁₋₄alkoxymethyl, or C₁₋₂alkyl-SO₂—CH₂—, wherein R⁷ and R⁸ are independently hydrogen or C₁₋₂alkyl;
wherein in sub-formula (z), G is O or S or NR⁹ wherein R⁹ is C₁₋₄alkyl or C₁₋₄fluoroalkyl; none, one or two of J, L, M and Q are nitrogen; and the remaining of J, L, M and Q are CH or CR⁶ where R⁶ is as defined herein;
   or R⁴ and R⁵ taken together are —(CH₂)ₚ— where p=3, 4 or 5 (preferably p=4);
   R^{5a} is C₁₋₈alkyl; C₁₋₈ fluoroalkyl; C₃₋₈cycloalkyl; phenyl optionally substituted with one or two of: a halogen atom, C₁₋₂alkyl, trifluoromethyl, C₁₋₂alkoxy or trifluoromethoxy; or R^{5a} has the sub-formula (x), (y) or (z) as defined herein;
   R³ is C₃₋₈cycloalkyl or a heterocyclic group being

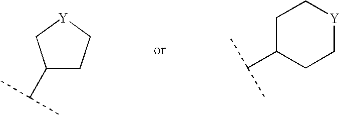

in which Y is O, S, SO₂, or NR¹⁰; where R¹⁰ is hydrogen, C₁₋₄alkyl, C₁₋₂fluoroalkyl, C(O)—C₁₋₂alkyl, or C(O)—CF₃;
and wherein in R³ the C₃₋₈cycloalkyl or heterocyclic group is optionally substituted with one or two substituents being OH, C₁₋₂alkoxy, trimethoxy, or C₁₋₂alkyl group; and wherein any OH, alkoxy or trimethoxy substituent is not substituted at the ring carbon attached to the —NH— group of formula (IA) and is not substituted at either ring carbon bonded to the Y group of the heterocyclic group; and
R¹═C₁₋₄alkyl or C₁₋₂fluoroalkyl.

In formula (IA), preferably, when R³ is the heterocyclic group being

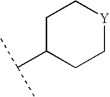

and Y is NR¹⁰, then:
either (a) R¹⁰ is hydrogen, C(O)—C₁₋₂alkyl, or C(O)—CF₃;
or (b) R¹⁰ is methyl and the compound is: 1-ethyl-N-(2-ethylbutyl)-4-[(1-methylpiperidin-4-yl)amino]-1H-pyrazolo[3,4-b]pyridine-5-carboxamide or 1-ethyl-N-(4-fluorophenyl)-4-[(1-methylpiperidin-4-yl)amino]-1H-pyrazolo[3,4-b]pyridine-5-carboxamide.

In formula (IA), preferably, where X is OR^{5a}, the compound is other than the compound wherein R¹ is methyl, X is OEt, and R³ is cyclopentyl.

In formula (IA), in sub-formula (x) and/or (y), it is preferred that none, one or two of A, B, D, E and F are nitrogen; none, one, two or three of A, B, D, E and F are CR⁶; and the remaining of A, B, D, E and F are CH. More preferably, none, one or two of A, B, D, E and F are nitrogen; none or one or two of A, B, D, E and F are CR⁶; and the remaining of A, B, D, E and F are CH. In formula (IA), in sub-formula (x) and/or (y), preferably, none or one of A, B, D, E and F are nitrogen.

In formula (IA), preferably, sub-formula (x) is: benzyl; phenethyl (Ph-C₂H₄—); benzyl or phenethyl being substituted on the phenyl ring with a single R⁶ substituent, or one of the following:

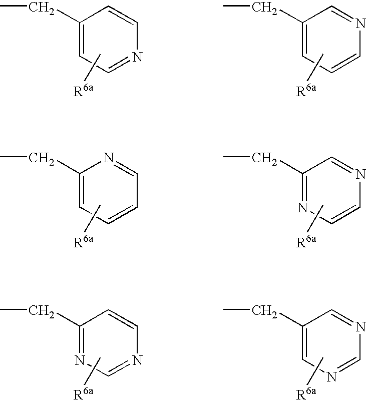

, wherein R^{6a} is either R⁶ as defined herein or (preferably) hydrogen.

In formula (IA), preferably, sub-formula (y) is:

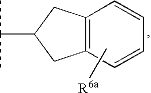

wherein R^{6a} is either R⁶ as defined herein or preferably hydrogen.

Examples 1-99 are examples of compounds or salts of the second aspect of the invention (Formula (IA)).

A third aspect of the present invention provides a compound of formula (IB) or a salt thereof (in particular, a pharmaceutically acceptable salt thereof):

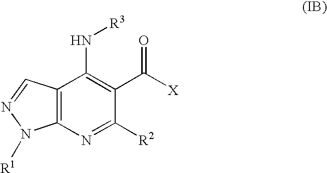

wherein:
R¹ is C₁₋₄alkyl, C₁₋₃fluoroalkyl, —CH₂CH₂OH or —CH₂CH₂CO₂C₁₋₂alkyl;
R² is a hydrogen atom (H), methyl or C₁fluoroalkyl;
R³ is optionally substituted C₃₋₈cycloalkyl or an optionally substituted heterocyclic group of sub-formula (aa), (bb) or (cc);

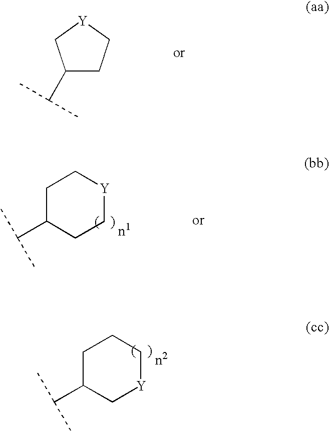

in which n¹ and n² independently are 1 or 2; and in which Y is O, S, SO₂, or NR¹⁰; where R¹⁰ is a hydrogen atom (H), C₁₋₄alkyl (e.g. methyl or ethyl), C₁₋₂fluoroalkyl, CH₂C(O)NH₂, C(O)NH₂, C(O)—C₁₋₂alkyl, or C(O)—C₁fluoroalkyl;
and wherein in R³ the C₃₋₈cycloalkyl or the heterocyclic group of sub-formula (aa), (bb) or (cc) is optionally substituted with one or two substituents being oxo (═O), OH, C₁₋₂alkoxy, C₁₋₂fluoroalkoxy (e.g. trifluoromethoxy), or C₁₋₂alkyl; and wherein any OH, alkoxy or fluoroalkoxy substituent is not substituted at the R³ ring carbon attached (bonded) to the —NH— group of formula (IB) and is not substituted at either R³ ring carbon bonded to the Y group of the heterocyclic group (aa), (bb) or (cc);
and X is NR⁴R⁵ or OR^{5a}, in which:
R⁴ is a hydrogen atom (H); C₁₋₆alkyl; C₁₋₃fluoroalkyl; or C₂₋₆alkyl substituted by one substituent R¹¹; and
R⁵ is a hydrogen atom (H); C₁₋₈alkyl; C₁₋₈ fluoroalkyl; C₃₋₈cycloalkyl optionally substituted by a C₁₋₂alkyl group; or —(CH₂)ₙ⁴—C₃₋₈cycloalkyl optionally substituted, in the —(CH₂)ₙ⁴— moiety or in the C₃₋₈cycloalkyl moiety, by a C₁₋₂alkyl group, wherein n⁴ is 1, 2 or 3;
or R⁵ is C₂₋₆alkyl substituted by one or two independent substituents R¹¹;
wherein each substituent R¹¹, independently of any other R¹¹ substituent present, is: hydroxy (OH); C₁₋₆alkoxy; phenyloxy; benzyloxy; —NR¹²R¹³; —NR¹⁵—C(O)R¹⁶; —NR¹⁵—C(O)—O—R¹⁶; —NR¹⁵—C(O)—NH—R¹⁵; or —NR¹⁵—SO₂R¹⁶; and wherein any R¹¹ substituent which is OH, alkoxy or —NR¹²R¹³ is not substituted at any carbon atom, of any R⁴ or R⁵ substituted alkyl, which is bonded to the nitrogen of NR⁴R⁵;
or R⁵ is —(CH₂)ₙ¹¹—C(O)R¹⁶; —(CH₂)ₙ¹¹—C(O)NR¹²R¹³; —CHR¹⁹—C(O)NR¹²R¹³; —(CH₂)ₙ¹²—C(O)OR¹⁶; —CHR¹⁹—C(O)OR¹⁶; —(CH₂)ₙ¹²—SO₂—NR¹²R¹³; —(CH₂)ₙ¹²—SO₂R¹⁶; or —(CH₂)ₙ¹²—CN; wherein n¹¹ is 0, 1, 2, 3 or 4 and n¹² is 1, 2, 3 or 4;
or R⁵ is —(CH₂)ₙ¹³-Het wherein n¹³ is 0, 1, 2, 3 or 4 and Het is a 4-, 5-, 6- or 7-membered saturated or partly-saturated heterocyclic ring containing one or two ring-hetero-atoms independently selected from O, S, and N; wherein any ring-hetero-atoms present are not bound to the —(CH₂)ₙ¹³— moiety when n¹³ is 1 and are not bound to the nitrogen of NR⁴R⁵ when n¹³ is 0; wherein any ring-nitrogens which are present and which are not unsaturated (i.e. which do not partake in a double bond) are present as NR¹⁷ where R¹⁷ is as defined herein; and wherein one or two of the carbon ring-atoms independently are optionally substituted by C₁₋₂alkyl;
or R⁵ is phenyl optionally substituted with one or two of: a halogen atom; C₁₋₄alkyl (e.g. C₁₋₂alkyl); C₁₋₂fluoroalkyl (e.g. trifluoromethyl); C₁₋₄alkoxy (e.g. C₁₋₂alkoxy); C₁₋₂fluoroalkoxy (e.g. trifluoromethoxy); C₁₋₂alkylsulphonyl (C₁₋₂alkyl-SO₂—); C₁₋₂alkyl-SO₂—NH—; R⁷R⁸N—SO₂—; R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-SO₂—CH₂—; cyano (CN); or phenyl optionally substituted by one or two of fluoro, chloro, C₁₋₂alkyl, C₁fluoroalkyl, C₁₋₂alkoxy or C₁fluoroalkoxy;
wherein R⁷ and R⁸ are independently a hydrogen atom (H); C₁₋₄alkyl (e.g. C₁₋₂alkyl such as methyl); C₃₋₆cycloalkyl; or phenyl optionally substituted by one or two of: fluoro, chloro, C₁₋₂alkyl, C₁fluoroalkyl, C₁₋₂alkoxy or C₁fluoroalkoxy; or R⁷ and R⁸ together are —(CH₂)ₙ⁶— or —C(O)—(CH₂)ₙ⁷— or —C(O)—(CH₂)ₙ⁷—C(O)— or —(CH₂)ₙ⁸—X⁷—(CH₂)ₙ⁹— or —C(O)—X⁷—(CH₂)ₙ¹⁰— in which: n⁶ is 3, 4, 5 or 6, n⁷ is 2, 3, 4, or 5 (preferably n⁷ is 2, 3 or 4), n⁸ and n⁹ and n¹¹ independently are 2 or 3, and X⁷ is O or NR¹⁴ wherein R¹⁴ is H or C₁₋₂alkyl;
   or R⁵ has the sub-formula (x), (y) or (z):

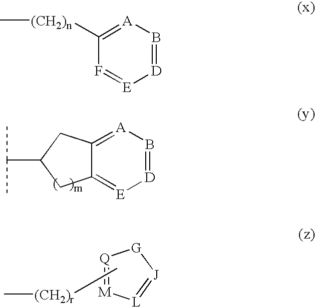

wherein in sub-formula (x), n=1 or 2; in sub-formula (y), m=1 or 2; and in sub-formula (z), r=0, 1 or 2;
wherein in sub-formula (x) and (y), none, one or two of A, B, D, E and F are nitrogen; and the remaining of A, B, D, E and F are independently CH or CR⁶;
where R⁶ is a halogen atom; C₁₋₄alkyl (e.g. C₁₋₂alkyl); C₁₋₄fluoroalkyl (e.g. C₁₋₂fluoroalkyl); C₁₋₄alkoxy (e.g. C₁₋₂alkoxy); C₁₋₂fluoroalkoxy; C₁₋₂alkylsulphonyl (C₁₋₂alkyl-SO₂—); C₁₋₂alkyl-SO₂—NH—; R⁷R⁸N—SO₂—; R⁷R⁸N—CO—; —NR¹⁵—C(O)R¹⁶;
R⁷R⁸N; OH; C₁₋₄alkoxymethyl; C₁₋₄alkoxyethyl; C₁₋₂alkyl-SO₂—CH₂—; cyano (CN); or phenyl optionally substituted by one or two of fluoro, chloro, C₁₋₂alkyl, C₁fluoroalkyl, C₁₋₂alkoxy or C₁fluoroalkoxy; wherein R⁷ and R⁸ are as herein defined;
wherein in sub-formula (z), G is O or S or NR⁹ wherein R⁹ is a hydrogen atom (H), C₁₋₄alkyl or C₁₋₄fluoroalkyl; none, one, two or three of J, L, M and Q are nitrogen; and the remaining of J, L, M and Q are independently CH or CR⁶ where R⁶ is as defined herein;
or R⁴ and R⁵ taken together are —(CH₂)ₚ¹— or —C(O)—(CH₂)ₚ²— or —(CH₂)ₚ³—X⁵—(CH₂)ₚ⁴— or —C(O)—X⁵—(CH₂)ₚ⁵—, in which: p¹=3, 4, 5 or 6 (preferably p=4 or 5), p² is 2, 3, 4, or 5 (preferably p² is 2, 3 or 4), and p³ and p⁴ and p⁵ independently are 2 or 3 (independently preferably 2) and X⁵ is O or NR¹⁷;
wherein R¹⁷ is a hydrogen atom (H); C₁₋₄alkyl (e.g. C₁₋₂alkyl); C₁₋₂fluoroalkyl; C₃₋₆cycloalkyl; —(CH₂)ₚ⁶—C(O)R¹⁶ wherein p6 is 0, 1, 2 or 3 (preferably p⁶ is 0); —(CH₂)ₚ⁶—C(O)NR¹²R¹³; —(CH₂)ₚ⁶—C(O)OR¹⁶; —SO₂R¹⁶; or phenyl or benzyl wherein the phenyl or benzyl is optionally substituted at an aromatic carbon atom by one or two of: a halogen atom, C₁₋₂alkyl, C₁fluoroalkyl, C₁₋₂alkoxy or C₁fluoroalkoxy;
and wherein, when R⁴ and R⁵ taken together are —(CH₂)ₚ¹— or —C(O)—(CH₂)ₚ²—, the NR⁴R⁵ heterocycle is optionally substituted by one R¹⁸ substituent wherein R¹⁸ is: C₁₋₄alkyl (e.g. C₁₋₂alkyl); C₁₋₂fluoroalkyl; C₃₋₆cycloalkyl; C₁₋₂alkoxy (not substituted at a ring-carbon bonded to the NR⁴R⁵ ring-nitrogen); C₁fluoroalkoxy (not substituted at a ring-carbon bonded to the NR⁴R⁵ ring-nitrogen); OH (not substituted at a ring-carbon bonded to the NR⁴R⁵ ring-nitrogen); —(CH₂)ₚ⁷—C(O)R¹⁶ wherein p7 is 0, 1, 2 or 3 (preferably p⁷ is 0 or 1); —(CH₂)ₚ⁷—C(O)OR¹⁶; —(CH₂)ₚ⁷—OC(O)R¹⁶; —(CH₂)ₚ⁷—C(O)NR¹²R¹³; —(CH₂)ₚ⁷—NR¹⁵C(O)R¹⁶; —(CH₂)ₚ⁷—NR¹⁵C(O)NR¹²R¹³; —(CH₂)ₚ⁷—NR¹⁵C(O)OR¹⁶; —(CH₂)ₚ⁷—SO₂R¹⁶; —(CH₂)ₚ⁷—SO₂ NR¹²R¹³; —(CH₂)ₚ⁷—NR¹⁵SO₂R¹⁶; —(CH₂)ₚ⁷—OH; —(CH₂)ₚ⁷—OR¹⁶; or phenyl optionally substituted by one or two of: a halogen atom, C₁₋₂alkyl, C₁fluoroalkyl, C₁₋₂alkoxy or C₁fluoroalkoxy;
   or R⁴ and R⁵ taken together are —(CH₂)ₚ¹— or —C(O)—(CH₂)ₚ²— or —(CH₂)ₚ³—X⁵—(CH₂)ₚ⁴— or —C(O)—X⁵—(CH₂)ₚ⁵— as defined herein, and wherein the NR⁴R⁵ heterocycle is fused to a phenyl ring optionally substituted on the phenyl by one or two of: a halogen atom, C₁₋₂alkyl, C₁fluoroalkyl, C₁₋₂alkoxy or C₁fluoroalkoxy; and
   R^{5a} is C₁₋₈alkyl; C₁₋₈ fluoroalkyl; C₃₋₈cycloalkyl; phenyl optionally substituted with one or two of: a halogen atom, C₁₋₂alkyl, trifluoromethyl, C₁₋₂alkoxy or trifluoromethoxy; or R^{5a} has the sub-formula (x), (y) or (z) as defined herein
   and wherein:
   R¹² and R¹³ independently are H; C₁₋₅alkyl (e.g. C₁₋₃alkyl); C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
   or R¹² and R¹³ together are —(CH₂)ₙ⁶— or —C(O)—(CH₂)ₙ⁷— or —C(O)—(CH₂)ₙ⁷—C(O)— or —(CH₂)ₙ⁸—X¹²—(CH₂)ₙ⁹— or —C(O)—X¹²—(CH₂)ₙ¹⁰— in which: n⁶ is 3, 4, 5 or 6 (preferably n⁶ is 4 or 5), n⁷ is 2, 3, 4, or 5 (preferably n⁷ is 2, 3 or 4), n⁸ and n⁹ and n¹¹ independently are 2 or 3 (independently preferably 2) and X¹² is O or NR¹⁴ wherein R¹⁴ is H or C₁₋₂alkyl;
   R¹⁵ is a hydrogen atom (H); C₁₋₄alkyl (e.g. ^{t}Bu or C₁₋₂alkyl e.g. methyl); C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
   R¹⁶ is C₁₋₄alkyl (e.g. C₁₋₂alkyl); C₃₋₆cycloalkyl; pyridinyl (e.g. pyridin-2-yl); or phenyl optionally substituted by one or two of: a halogen atom, C₁₋₂alkyl, C₁fluoroalkyl, C₁₋₂alkoxy or C₁fluoroalkoxy; and
   R¹⁹ is a hydrogen atom (H); C₁₋₄alkyl (e.g. isobutyl, sec-butyl, or C₁₋₃alkyl such as methyl or isopropyl); —(CH₂)ₙ²⁰—OR²⁰ wherein n²⁰ is 1, 2, 3 or 4 (preferably 1) and R²⁰ is a hydrogen atom (H) or C₁₋₄alkyl (preferably R²⁰ is H); —CH(Me)-OH; —CH₂—SH; —CH₂—CH₂—S-Me; benzyl; or (4-hydroxyphenyl)methyl (i.e. 4-hydroxy-benzyl).

In formula (IB), preferably, when R³ is the heterocyclic group of sub-formula (bb), n¹ is 1, and Y is NR¹⁰, then:

either (a) R¹⁰ is not C₁₋₄alkyl, C₁₋₂fluoroalkyl or CH₂C(O)NH₂;
or (b) R¹⁰ is methyl and the compound is: 1-ethyl-N-(2-ethylbutyl)-4-[(1-methylpiperidin-4-yl)amino]-1H-pyrazolo[3,4-b]pyridine-5-carboxamide or 1-ethyl-N-(4-fluorophenyl)-4-[(1-methylpiperidin-4-yl)amino]-1H-pyrazolo[3,4-b]pyridine-5-carboxamide.

In formula (IB), preferably, where X is OR^{5a}, the compound is other than the compound wherein R¹ is methyl, X is OEt, and R³ is cyclopentyl.

In formula (IB), where R³ is optionally substituted C₃₋₈cycloalkyl, the one or two optional substituents preferably comprise (e.g. is or are) OH and/or oxo (═O). In formula (IB), in the R³ heterocyclic group of sub-formula (aa), (bb) or (cc), the one or two optional substituents preferably comprise (e.g. is or are) OH and/or oxo.

Examples 1-203 are examples of compounds or salts of the third aspect of the invention (Formula (IB)).

### DETAILED DESCRIPTION OF THE INVENTION

The preferred or optional features for the compound or salt of formula (IA) and for the compound or salt of formula (IB) are the same as or similar to the preferred or optional features for the compound or salt of formula (I), with all necessary changes (for example to the formula, to the R groups and/or to substituents) having been made. Generally, whenever formula (I) is mentioned herein, then in alternative embodiments the statement mentioning formula (I) applies to formula (IA) or formula (IB), with all necessary changes having been made.

### Salts, Solvates, Isomers, Tautomeric Forms, Molecular Weights, Etc.

Because of their potential use in medicine, the salts of the compounds of formula (I) are preferably pharmaceutically acceptable. Suitable pharmaceutically acceptable salts can include acid or base addition salts. A pharmaceutically acceptable acid addition salt can be formed by reaction of a compound of formula (I) with a suitable inorganic or organic acid (such as hydrobromic, hydrochloric, sulfuric, nitric, phosphoric, succinic, maleic, acetic, fumaric, citric, tartaric, benzoic, p-toluenesulfonic, methanesulfonic or naphthalenesulfonic acid), optionally in a suitable solvent such as an organic solvent, to give the salt which is usually isolated for example by crystallisation and filtration. A pharmaceutically acceptable acid addition salt of a compound of formula (I) can be for example a hydrobromide, hydrochloride, sulfate, nitrate, phosphate, succinate, maleate, acetate, fumarate, citrate, tartrate, benzoate, p-toluenesulfonate, methanesulfonate or naphthalenesulfonate salt. A pharmaceutically acceptable base addition salt can be formed by reaction of a compound of formula (I) with a suitable inorganic or organic base, optionally in a suitable solvent such as an organic solvent, to give the base addition salt which is usually isolated for example by crystallisation and filtration. Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of one or more carboxylic acid moieties that may be present in the compound of formula (I).

Other non-pharmaceutically acceptable salts, eg. oxalates, may be used, for example in the isolation of compounds of the invention, and are included within the scope of this invention.

The invention includes within its scope all possible stoichiometric and non-stoichiometric forms of the salts of the compounds of formula (I).

Also included within the scope of the invention are all solvates, hydrates and complexes of compounds and salts of the invention.

Certain groups, substituents, compounds or salts included in the present invention may be present as isomers. The present invention includes within its scope all such isomers, including racemates, enantiomers and mixtures thereof.

Certain of the groups, e.g. heteroaromatic ring systems, included in compounds of formula (I) or their salts may exist in one or more tautomeric forms. The present invention includes within its scope all such tautomeric forms, including mixtures.

Especially when intended for oral medicinal use, the compound of formula (I) can optionally have a molecular weight of 1000 or less, for example 800 or less, in particular 650 or less or 600 or less. Molecular weight here refers to that of the unsolvated “free base” compound, that is excluding any molecular weight contributed by any addition salts, solvent (e.g. water) molecules, etc.

### Synthetic Process Routes

The following processes can be used to make the compounds of the invention:

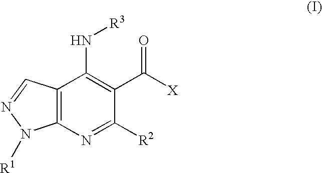

Most of the following synthetic processes following are exemplified for compounds of Formula (I) wherein R² is a hydrogen atom (H). However, some or all of these processes can also be used with appropriate modification, e.g. of starting materials and reagents, for making compounds of Formula (I) wherein R² is other than H.

### Process A

Compounds of formula (I) where X═OR^{5a}, can be prepared according to a method, for example as described by Yu et. al. in J. Med. Chem., 2001, 44, 1025-1027, by reaction of a compound of formula (II) with an amine of formula R³NH₂. The reaction is preferably carried out in the presence of a base such as triethylamine or N,N-diisopropylethylamine, and/or in an organic solvent such as ethanol, dioxane or acetonitrile. The reaction may require heating e.g. to ca. 60-100° C., for example ca. 80-90° C.:

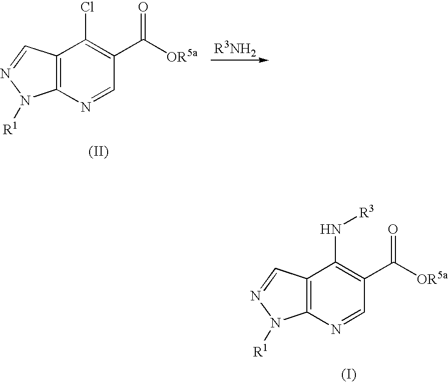

Compounds of formula (II) are also described in the above reference and can be prepared by reaction of a compound of formula (III) with, for example, diethylethoxymethylene malonate (where R^{5a}=Et) with heating, followed by reaction with phosphorous oxychloride, again with heating:

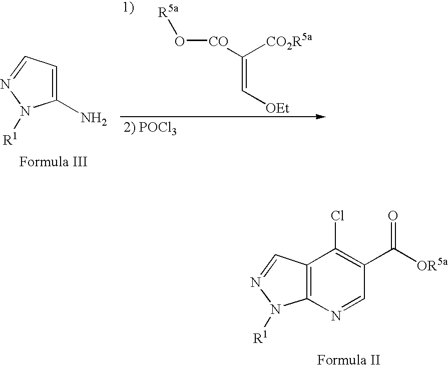

Where the desired amino pyrazole of formula (III) is not commercially available, preparation can be achieved using methods described by Dorgan et. al. in J. Chem. Soc., Perkin Trans. 1, (4), 938-42; 1980, by reaction of cyanoethylhydrazine with a suitable aldehyde of formula R⁴⁰CHO in a solvent such as ethanol, with heating, followed by reduction with, for example sodium in a solvent such as t-butanol. R⁴⁰ should be chosen so as to contain one less carbon atom than R¹, for example R⁴⁰=methyl will afford R¹=ethyl.

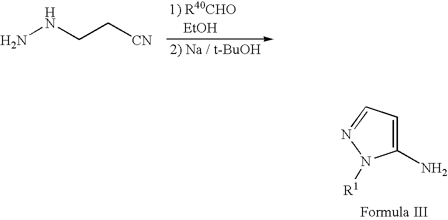

In an alternative embodiment of Process A, the 4-chloro substituent in the compound of formula (II) can be replaced by a halogen atom, such as a bromine atom or preferably a chlorine atom, in a compound of formula (IIA) as defined below. In this embodiment of Process A, the compound of formula (IIA) is reacted with the amine of formula R³NH₂.

### Process B

Compounds of formula (I) where X═NR⁴R⁵, can be prepared by reaction of a compound of formula (IV) with an amine of formula R³NH₂. The reaction is preferably carried out in the presence of a base, such as triethylamine or N,N-diisopropylethylamine, and/or in an organic solvent such as ethanol, THF, dioxane or acetonitrile. The reaction may require heating, e.g. to ca. 60-100° C. or ca. 80-90° C., for example for 8-48 or 12-24 hours:

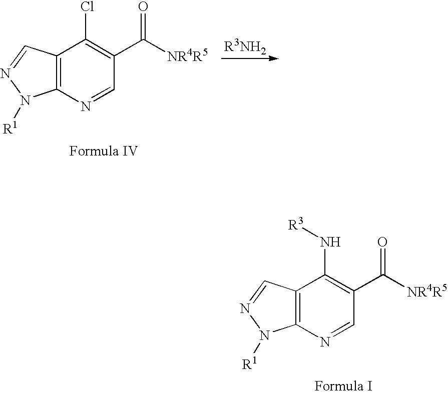

Compounds of formula (IV) can be prepared in a two step procedure as described by Bare et. al. in J. Med. Chem. 1989, 32, 2561-2573. This process involves, first, reaction of a compound of formula (V) with thionyl chloride (or another agent suitable for forming an acid chloride from a carboxylic acid), either in an organic solvent such as chloroform or THF, or as a neat solution. This reaction may require heating and the thus-formed intermediate may or may not be isolated. Step two involves reaction with an amine of formula R⁴R⁵NH, in an organic solvent such as THF or chloroform and may also involve the use of a base such as triethylamine or diisopropylethyl amine:

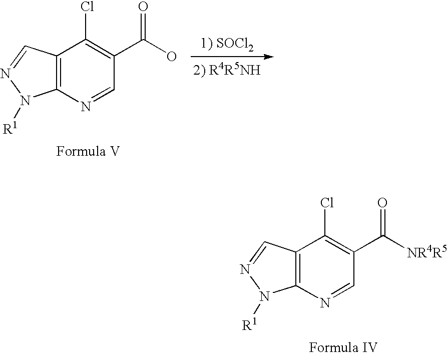

Compounds of formula (V) can be prepared by hydrolysis of an ester of formula (II) according to the method described by Yu et. al. in J. Med. Chem., 2001, 44, 1025-1027. This procedure preferably involves reaction with a base such as sodium hydroxide or potassium hydroxide in a solvent e.g. an aqueous solvent such as aqueous ethanol or aqueous dioxane:

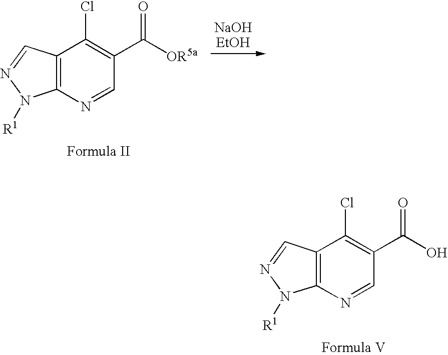

In an alternative embodiment of Process B, the 4-chloro substituent in the compound of formula (IV) can be replaced by a halogen atom, such as a bromine atom or preferably a chlorine atom, in a compound of formula (IVA) as defined below. In this embodiment of Process B, the compound of formula (IVA) is reacted with the amine of formula R³NH₂.

### Process C

Compounds of formula (I) can also be prepared according to a method, for example as described by Bare et. al. in J. Med. Chem. 1989, 32, 2561-2573, which involves reaction of a compound of formula (VI), in which —O—R³⁵ is a leaving group displaceable by an amine, with an amine of formula R³NH₂. The —O—R³⁵ leaving group can be —O—C₁₋₄alkyl (in particular —O-Et) or —O—S(O)₂—R³⁷, wherein R³⁷ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction may be carried out with or without solvent and may require heating:

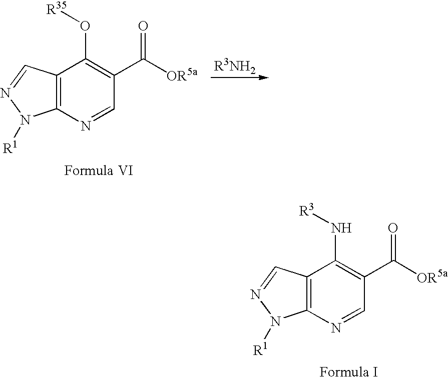

Compounds of formula (VI) (also described in the above reference) can be prepared by reaction of a compound of formula (VII) with a suitable alkylating agent of formula R¹—X, where X is a leaving group such as halogen. The reaction is preferably carried out in the presence of a base such as potassium carbonate, in an anhydrous solvent such as DMF:

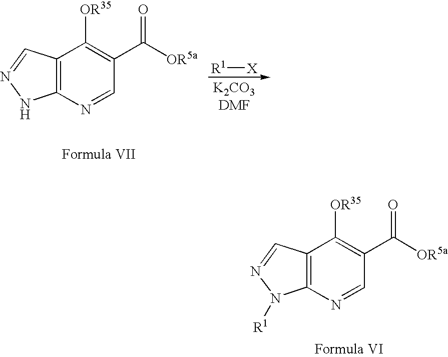

The preparation of compounds of formula VII, e.g. where OR³⁵ is OEt, by oxidative cleavage of compounds of formula VIII is described by Bare et. al. in J. Med. Chem. 1989, 32, 2561-2573 (further referred to Zuleski et. al. in J. Drug. Metab. Dispos., 1985, 13, 139).

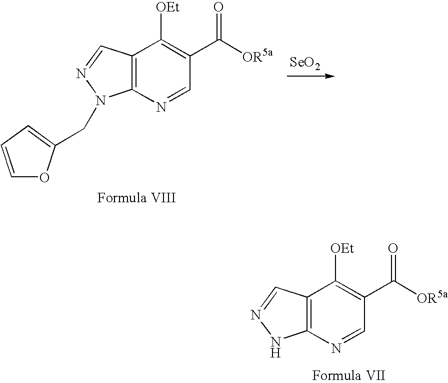

In another embodiment of Process C, the compound of formula (VI) can be replaced by a compound of formula (VIA), wherein X is NR⁴R⁵ or OR^{5a} as defined herein:

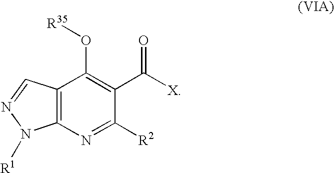

In this embodiment of Process C, the compound of formula (VIA) is reacted with the amine of formula R³NH₂.

### Process D:

To form a compound of formula (I) wherein X═NR⁴R⁵, a compound of formula (I) but wherein X═OH (a carboxylic acid, the compound of formula (IX) as defined below) can be converted into an activated compound of formula (I) but wherein X=a leaving group X¹ substitutable by an amine (a compound of formula (X) as defined below, wherein X¹ is a leaving group substitutable by an amine); and subsequently the activated compound can be reacted with an amine of formula R⁴R⁵NH:

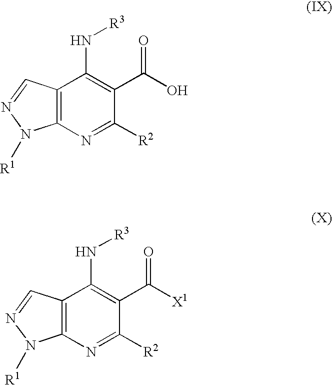

For example, the activated compound (the compound of formula (X)) can be the acid chloride i.e. an activated compound of formula (I) but wherein the leaving group X¹═Cl. This can be formed from the carboxylic acid (X═OH, the compound of formula (IX)) e.g. by reaction with thionyl chloride, either in an organic solvent such as chloroform or without solvent. See for example Examples 81-85. Alternatively, the activated compound (the compound of formula (X)) can be an activated ester wherein the leaving group X¹ is

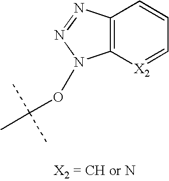

The latter activated compound of formula (X) can be formed from the carboxylic acid (X═OH, the compound of formula (IX)) either:

(a) by reaction of the carboxylic acid with a carbodiimide such as EDC, which is 1-ethyl-3-(3′-dimethylaminopropyl)carbodiimide and is also 1-(3-dimethylaminopropyl)-3-ethylcarbodiimide, or a salt thereof e.g. hydrochloride salt,
preferably followed by reaction of the resulting product with 1-hydroxybenzotriazole (HOBT); reaction (a) usually being carried out in the presence of a solvent (preferably anhydrous) such as dimethyl formamide (DMF) or acetonitrile and/or preferably under anhydrous conditions and/or usually at room temperature (e.g. about 20 to about 25° C.); or
(b) by reaction with 2-(1H-benzotriazole-1-yl)-1,1,3,3-tetramethyluronium tetrafluoroborate (TBTU) or O-(7-Azabenzotriazol-1-yl)-N,N,N′,N′-tetramethyluronium hexafluorophosphate (HATU), in the presence of a base such as diisopropylethylamine (iPr₂NEt=DIPEA), and usually in the presence of a solvent such as dimethyl formamide (DMF) or acetonitrile and/or preferably under anhydrous conditions and/or usually at room temperature (e.g. about 20 to about 25° C.).

The carboxylic acid wherein X═OH (the compound of formula (IX) below) is usually prepared by hydrolysis of the corresponding ester of formula (I) wherein X is OR^{5a}. This ester can itself be prepared by any of Processes A, C, E or F as described herein.

### Process D1

This is the same as Process D, but involves reaction of the activated compound of formula (X), wherein X¹=a leaving group substitutable by an amine (for example a leaving group as defined herein), with an amine of formula R⁴R⁵NH.

### Process E

Compounds of formula (I) can be prepared by reaction of a compound of formula (X¹) with an alkylating agent of formula R¹—X³, where X³ is a leaving group displaceable by the 1-position pyrazolopyridine nitrogen atom of the compound of formula (X¹):

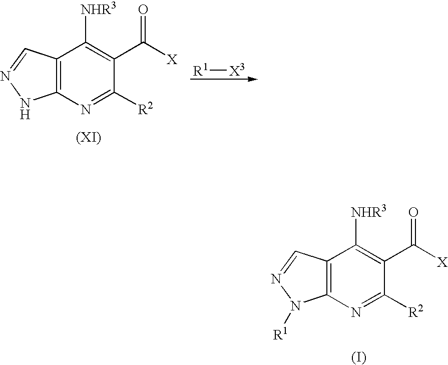

A suitable alkylating agent of formula R¹—X³ can be used. For example, X³ can be a halogen atom such as a chlorine atom or more preferably a bromine or iodine atom, or X³ can be —O—S(O)₂—R³⁶ wherein R³⁶ is C₁₋₄alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction is preferably carried out in the presence of a base; the base can for example comprise or be potassium carbonate, sodium carbonate, sodium hydride, potassium hydride, or a basic resin or polymer such as polymer-bound 2-tert-butylimino-2-diethylamino-1,3-dimethyl-perhydro-1,3,2-diazaphosphorine. The reaction is preferably carried out in the presence of a solvent, e.g. an organic solvent such as DMF; the solvent is preferably anhydrous. Examples of alkylation Process E include Examples 183, 185, 186 and 354.

For preferable methods of making compounds of formula (X¹), see for example (Reference) Examples 19-20, and Intermediates 48 and 54A.

Process F: Conversion of one compound of formula (I) or salt thereof into another compound of formula (I) or salt thereof.

One compound of formula (I) or salt thereof can be converted into another compound of formula (I) or salt thereof. This conversion preferably comprises or is one or more of the following processes F1 to F10:

F1. An oxidation process. For example, the oxidation process can comprise or be oxidation of an alcohol to a ketone (e.g. using Jones reagent, e.g. see Example 205) or oxidation of an alcohol or a ketone to a carboxylic acid. The oxidation process can e.g. comprise or be conversion of a nitrogen-containing compound of formula (I) or salt thereof to the corresponding N-oxide (e.g. using meta-chloroperoxybenzoic acid), for example conversion of a pyridine-containing compound to the corresponding pyridine N-oxide (e.g. Examples 210-212).
F2. A reduction process, for example reduction of a ketone or a carboxylic acid to an alcohol.
F3. Acylation, for example acylation of an amine (e.g. Examples 329-349, Example 353) or hydroxy group.
F4. Alkylation, for example alkylation of an amine or of a hydroxy group.
F5. Hydrolysis, e.g. hydrolysis of an ester to the corresponding carboxylic acid or salt thereof (e.g. Examples 351, 488, 489, 650, 651).
F6. Deprotection, e.g. deprotection (e.g. deacylation or t-butyloxycarbonyl (BOC) removal) of an amine group (e.g. Examples 320, (321), and (352)).
F7. Formation of an ester or amide, for example from the corresponding carboxylic acid.
F8. Conversion of a ketone into the corresponding oxime (e.g. Examples 652, 653, 654 and 680-686).
F9. Sulfonylation, e.g. sulfonamide formation by reaction of an amine with a sulfonyl halide e.g. a sulfonyl chloride (e.g. Examples 322-328).
and/or
F10. Beckmann rearrangement of one compound of formula (I) into another compound of formula (I), preferably using cyanuric chloride (2,4,6-trichloro-1,3,5-triazine) together with a formamide such as DMF, e.g. at room temperature (see L. D. Luca, J. Org. Chem., 2002, 67, 6272-6274). The Beckmann rearrangement can for example comprise conversion of a compound of formula (I) wherein NHR³ is of sub-formula (o2)

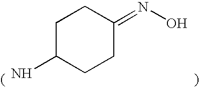

into a compound of formula (I) wherein NHR³ is of sub-formula (m3)

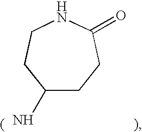

e.g. as illustrated in Examples 658 and 659.

The present invention therefore also provides a method of preparing a compound of formula (I) or a salt thereof:

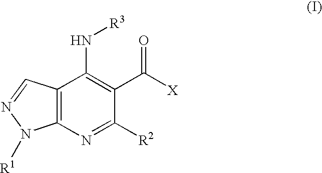

wherein R¹, R² and R³ are as defined herein and X is NR⁴R⁵ or OR^{5a} as defined herein, the method comprising:
(a) for a compound of formula (I) wherein X═OR^{5a}, reaction of a compound of formula (IIA):

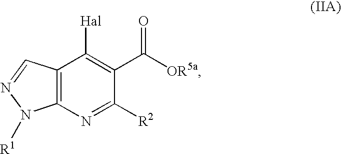

wherein Hal is a halogen atom (such as a bromine atom or preferably a chlorine atom), with an amine of formula R³NH₂, or
(b) for a compound of formula (I) wherein X═NR⁴R⁵, reaction of a compound of formula (IVA):

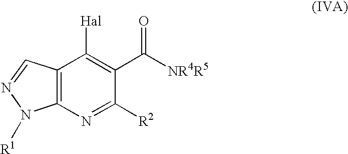

wherein Hal is a halogen atom (such as a bromine atom or preferably a chlorine atom), with an amine of formula R³NH₂, or
(c) reaction of a compound of formula (VIA):

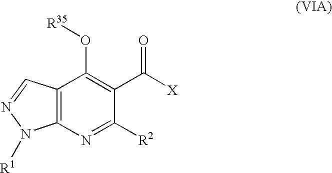

, in which —O—R³⁵ is a leaving group displaceable by an amine (such as —O—C₁₋₄alkyl or —O—S(O)₂—R³⁷),
with an amine of formula R³NH₂; or
(d) to form a compound of formula (I) wherein X═NR⁴R⁵, conversion of a compound of formula (IX) into an activated compound of formula (X) wherein X¹=a leaving group substitutable by an amine:

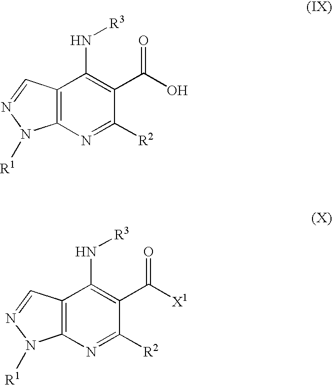

, and subsequent reaction of the activated compound of formula (X) with an amine of formula R⁴R⁵NH; or
(d1) to form a compound of formula (I) wherein X═NR⁴R⁵, reaction of an activated compound of formula (X) as defined above with an amine of formula R⁴R⁵NH; or
(e) reaction of a compound of formula (X¹):

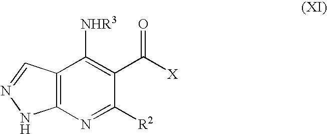

with an alkylating agent of formula R¹—X², where X² is a leaving group displaceable by the 1-position pyrazolopyridine nitrogen atom of the compound of formula (X¹); or
(f) conversion of one compound of formula (I) or salt thereof into another compound of formula (I) or salt thereof,
and optionally converting the compound of formula (I) into a salt thereof e.g. a pharmaceutically acceptable salt thereof.

In methods (d) and/or (d1), the activated compound of formula (X) wherein X¹=a leaving group substitutable by an amine can be the acid chloride i.e. an activated compound of formula (X) wherein X¹═Cl. Alternatively, the activated compound of formula (X) can be an activated ester wherein the leaving group X¹ is

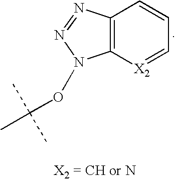

Preferred features of methods (a), (b), (c), (d), (d1) and (e), independently of each other, are as described above for Processes A, B, C, D, DI and E, with all necessary changes being made.

The present invention also provides: (g) a method of preparing a pharmaceutically acceptable salt of a compound of formula (I) comprising conversion of the compound of formula (I) or a salt thereof into the desired pharmaceutically acceptable salt thereof. (See for example Examples 490, 491, 518A, 593).

The present invention also provides a compound of formula (I) or a salt thereof, prepared by a method as defined herein.

### Medical Uses

The present invention also provides a compound of formula (I) or a pharmaceutically acceptable salt thereof for use as an active therapeutic substance in a mammal such as a human. The compound or salt can be for use in the treatment and/or prophylaxis of any of the diseases/conditions described herein (e.g. for use in the treatment and/or prophylaxis of an inflammatory and/or allergic disease in a mammal) and/or for use as a phosphodiesterase inhibitor e.g. for use as a phosphodiesterase 4 (PDE4) inhibitor. “Therapy” may include treatment and/or prophylaxis.

Also provided is the use of a compound of formula (I) or a pharmaceutically acceptable salt thereof in the manufacture of a medicament (e.g. pharmaceutical composition) for the treatment and/or prophylaxis of any of the diseases/conditions described herein in a mammal such as a human, e.g. for the treatment and/or prophylaxis of an inflammatory and/or allergic disease in a mammal such as a human.

Also provided is a method of treatment and/or prophylaxis of any of the diseases/conditions described herein in a mammal (e.g. human) in need thereof, e.g. a method of treatment and/or prophylaxis of an inflammatory and/or allergic disease in a mammal (e.g. human) in need thereof, which method comprises administering to the mammal (e.g. human) a therapeutically effective amount of a compound of formula (I) as herein defined or a pharmaceutically acceptable salt thereof.

Phosphodiesterase 4 inhibitors are thought to be useful in the treatment and/or prophylaxis of a variety of diseases/conditions, especially inflammatory and/or allergic diseases, in mammals such as humans, for example: asthma, chronic obstructive pulmonary disease (COPD) (e.g. chronic bronchitis and/or emphysema), atopic dermatitis, urticaria, allergic rhinitis, allergic conjunctivitis, vernal conjunctivitis, eosinophilic granuloma, psoriasis, rheumatoid arthritis, septic shock, ulcerative colitis, Crohn's disease, reperfusion injury of the myocardium and brain, chronic glomerulonephritis, endotoxic shock, adult respiratory distress syndrome, multiple sclerosis, cognitive impairment (e.g. in a neurological disorder such as Alzheimer's disease), depression, or pain. Ulcerative colitis and/or Crohn's disease are collectively often referred to as inflammatory bowel disease.

In the treatment and/or prophylaxis, the inflammatory and/or allergic disease is preferably chronic obstructive pulmonary disease (COPD), asthma, rheumatoid arthritis or allergic rhinitis in a mammal (e.g. human). More preferably, the treatment and/or prophylaxis is of COPD or asthma in a mammal (e.g. human).

PDE4 inhibitors are thought to be effective in the treatment of asthma (e.g. see M. A. Giembycz, Drugs, February 2000, 59(2), 193-212; Z. Huang et al., Current Opinion in Chemical Biology, 2001, 5: 432-438; H. J. Dyke et al., Expert Opinion on Investigational Drugs, January 2002, 11(1), 1-13; C. Burnouf et al., Current Pharmaceutical Design, 2002, 8(14), 1255-1296; A. M. Doherty, Current Opinion Chem. Biol., 1999, 3(4), 466-473; and refs cited therein).

PDE4 inhibitors are thought to be effective in the treatment of COPD (e.g. see S. L. Wolda, Emerging Drugs, 2000, 5(3), 309-319; Z. Huang et al., Current Opinion in Chemical Biology, 2001, 5: 432-438; H. J. Dyke et al., Expert Opinion on Investigational Drugs, January 2002, 11(1), 1-13; C. Burnouf et al., Current Pharmaceutical Design, 2002, 8(14), 1255-1296; A. M. Doherty, Current Opinion Chem. Biol., 1999, 3(4), 466-473; and refs cited therein). COPD is often characterised by the presence of airflow obstruction due to chronic bronchitis and/or emphysema (S. L. Wolda, Emerging Drugs, 2000, 5(3), 309-319).

PDE4 inhibitors are thought to be effective in the treatment of allergic rhinitis (e.g. see B. M. Schmidt et al., J. Allergy & Clinical Immunology, 108(4), 2001, 530-536).

PDE4 inhibitors are thought to be effective in the treatment of rheumatoid arthritis and multiple sclerosis (e.g. see H. J. Dyke et al., Expert Opinion on Investigational Drugs, January 2002, 11(1), 1-13; C. Burnouf et al., Current Pharmaceutical Design, 2002, 8(14), 1255-1296; and A. M. Doherty, Current Opinion Chem. Biol., 1999, 3(4), 466-473; and refs cited therein). See e.g. A. M. Doherty, Current Opinion Chem. Biol., 1999, 3(4), 466-473 and refs cited therein for atopic dermatitis use.

PDE4 inhibitors have been suggested as having analgesic properties and thus being effective in the treatment of pain (A. Kumar et al., Indian J. Exp. Biol., 2000, 38(1), 26-30).

In the invention, the treatment and/or prophylaxis can be of cognitive impairment e.g. cognitive impairment in a neurological disorder such as Alzheimer's disease. For example, the treatment and/or prophylaxis can comprise cognitive enhancement e.g. in a neurological disorder. See for example: H. T. Zhang et al. in: Psychopharmacology, June 2000, 150(3), 311-316 and Neuropsychopharmacology, 2000, 23(2), 198-204; and T. Egawa et al., Japanese J. Pharmacol., 1997, 75(3), 275-81.

PDE4 inhibitors such as rolipram have been suggested as having antidepressant properties (e.g. J. Zhu et al., CNS Drug Reviews, 2001, 7(4), 387-398; O'Donnell, Expert Opinion on Investigational Drugs, 2000, 9(3), 621-625; and H. T. Zhang et al., Neuropsychopharmacology, October 2002, 27(4), 587-595).

### Pharmaceutical Compositions and Dosing

For use in medicine, the compounds of the present invention are usually administered as a pharmaceutical composition.

The present invention therefore provides in a further aspect a pharmaceutical composition comprising a compound of formula (I) or a pharmaceutically acceptable salt thereof and one or more pharmaceutically acceptable carriers and/or excipients.

The pharmaceutical composition can be for use in the treatment and/or prophylaxis of any of the conditions described herein.

The invention also provides a method of preparing a pharmaceutical composition comprising a compound of formula (I), as herein defined, or a pharmaceutically acceptable salt thereof, and one or more pharmaceutically acceptable carriers and/or excipients,

the method comprising mixing the compound or salt with the one or more pharmaceutically acceptable carriers and/or excipients.

The invention also provides a pharmaceutical composition prepared by said method.

The compounds of formula (I) and/or the pharmaceutical composition may be administered, for example, by oral, parenteral (e.g. intravenous, subcutaneous, or intramuscular), inhaled or nasal administration. Accordingly, the pharmaceutical composition is preferably suitable for oral, parenteral (e.g. intravenous, subcutaneous, or intramuscular), inhaled or nasal administration. More preferably, the pharmaceutical composition is suitable for inhaled or oral administration, e.g. to a mammal such as a human. Inhaled administration involves topical administration to the lung e.g. by aerosol or dry powder composition. Oral administration to a human is most preferred.

A pharmaceutical composition suitable for oral administration can be liquid or solid; for example it can be a syrup, suspension or emulsion, a tablet, a capsule or a lozenge.

A liquid formulation will generally consist of a suspension or solution of the compound or pharmaceutically acceptable salt in a suitable pharmaceutically acceptable liquid carrier(s), for example an aqueous solvent such as water, ethanol or glycerine, or a non-aqueous solvent, such as polyethylene glycol or an oil. The formulation may also contain a suspending agent, preservative, flavouring and/or colouring agent.

A pharmaceutical composition suitable for oral administration being a tablet can comprise one or more pharmaceutically acceptable carriers and/or excipients suitable for preparing tablet formulations. Examples of such carriers include lactose and cellulose. The tablet can also or instead contain one or more pharmaceutically acceptable excipients, for example binding agents, lubricants such as magnesium stearate, and/or tablet disintegrants.

A pharmaceutical composition suitable for oral administration being a capsule can be prepared using encapsulation procedures. For example, pellets containing the active ingredient can be prepared using a suitable pharmaceutically acceptable carrier and then filled into a hard gelatin capsule. Alternatively, a dispersion or suspension can be prepared using any suitable pharmaceutically acceptable carrier, for example an aqueous gum or an oil and the dispersion or suspension then filled into a soft gelatin capsule.

Preferably the composition is in unit dose form such as a tablet or capsule for oral administration, e.g. for oral administration to a human.

A parenteral composition can comprise a solution or suspension of the compound or pharmaceutically acceptable salt in a sterile aqueous carrier or parenterally acceptable oil. Alternatively, the solution can be lyophilised; the lyophilised parenteral pharmaceutical composition can be reconstituted with a suitable solvent just prior to administration.

Compositions for nasal or inhaled administration may conveniently be formulated as aerosols, drops, gels or dry powders.

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide, or an organic propellant such as a chlorofluorocarbon (CFC) or hydrofluorocarbon (HFC). Suitable CFC propellants include dichlorodifluoromethane, trichlorofluoromethane and dichlorotetrafluoroethane. Suitable HFC propellants include 1,1,1,2,3,3,3-heptafluoropropane and 1,1,1,2-tetrafluoroethane. The aerosol dosage forms can also take the form of a pump-atomiser.

For pharmaceutical compositions suitable and/or adapted for inhaled administration, it is preferred that the compound or salt of formula (I) is in a particle-size-reduced form, and more preferably the size-reduced form is obtained or obtainable by micronisation. Micronisation usually involves subjecting the compound/salt to collisional and abrasional forces in a fast-flowing circular or spiral/vortex-shaped airstream often including a cyclone component. The preferable particle size (e.g. D50 value) of the size-reduced (e.g. micronised) compound or salt is about 0.5 to about 10 microns, e.g. about 1 to about 5 microns (e.g. as measured using laser diffraction). For example, it is preferable for the compound or salt of formula (I) to have a particle size defined by: a D10 of about 0.3 to about 3 microns (e.g. about 1 micron), and/or a D50 of about 1 to about 5 microns (e.g. about 2-5 or about 2-3 microns), and/or a D90 of about 2 to about 20 microns or about 3 to about 10 microns (e.g. about 5-8 or about 5-6 microns); for example as measured using laser diffraction. The laser diffraction measurement can use a dry method (suspension of compound/salt in airflow crosses laser beam) or a wet method [suspension of compound/salt in liquid dispersing medium, such as isooctane or (e.g. if compound soluble in isooctane) 0.1% Tween 80 in water, crosses laser beam]. With laser diffraction, particle size is preferably calculated using the Fraunhofer calculation; and/or preferably a Malvern Mastersizer or Sympatec apparatus is used for measurement.

An illustrative non-limiting example of a small-scale micronisation process is now given:

### Micronisation Example: Micronisation of Example 518 or 518A

Purpose: To micronize approximately 600-1000 mg of Example 518 or 518A (described hereinafter) using a Jetpharma MC1 micronizer.
The parent (unmicronised) and micronised materials are analyzed for particle size by laser diffraction and crystallinity by PXRD.

### Equipment and Material

| Equipment/material | Description and specification |
|---|---|
| Jetpharma MC1 Micronizer | Nitrogen supply: Air tank with 275 psi |
| | rate tubing |
| Analytical balance | Sartorius Analytical |
| Top loader balance | Mettler PM400 |
| Digital Caliper | VWR Electronic caliper |
| Vibrational spatula | Auto-spat Dispenser |
| Materials to be micronised | Example 518 or 518A |

The Jetpharma MC1 Micronizer comprises a horizontal disc-shaped milling housing having: a tubular compound inlet (e.g. angled at ca. 30 degrees to the horizontal) for entry of a suspension of unmicronised compound of formula (I) or salt in an gasflow, a separate gas inlet for entry of gases, a gas outlet for exit of gases, and a collection vessel for collecting micronised material. The milling housing has two chambers: an outer annular chamber in gaseous connection with the gas inlet the chamber being for receiving pressurised gas (e.g. air or nitrogen), an disc-shaped inner milling chamber within and coaxial with the outer chamber for micronising the input compound/salt, the two chambers being separated by an annular wall. The annular wall (ring R) has a plurality of narrow-bored holes connecting the inner and outer chambers and circumferentially-spaced-apart around the annular wall. The holes open into the inner chamber directed at an angle (directed part-way between radially and tangentially), and in use act as nozzles directing pressurised gas at high velocity from the outer chamber into the inner chamber and in an inwardly-spiral path (vortex) around the inner chamber (cyclone). The compound inlet is gaseous communication with the inner chamber via a nozzle directed tangentially to the inner chamber, within and near to the annular wall. Upper and lower broad-diameter exit vents in the central axis of the inner milling chamber connect to (a) (lower exit) the collection vessel which has no air outlet, and (b) (upper exit) the gas outlet which leads to a collection bag, filter and a gas exhaust. Inside the tubular compound inlet and longitudinally-movable within it is positioned a venturi inlet (V) for entry of gases. The compound inlet also has a bifurcation connecting to an upwardly-directed material inlet port for inputting material.

In use, the narrow head of the venturi inlet (V) is preferably positioned below and slightly forward of the material inlet port so that when the venturi delivers pressurised gas (eg air or nitrogen) the feed material is sucked into the gasstream thorough the compound inlet and accelerates it into the inner milling chamber tangentially at a subsonic speed. Inside the milling chamber the material is further accelerated to a supersonic speed by the hole/nozzle system around the ring (R) (annular wall) of the milling chamber. The nozzles are slightly angled so that the acceleration pattern of the material is in the form of an inwardly-directed vortex or cyclone. The material inside the milling chamber circulates rapidly and particle collisions occur during the process, causing larger particles to fracture into smaller ones. “Centrifugal” acceleration in the vortex causes the larger particles to remain at the periphery of the inner chamber while progressively smaller particles move closer to the center until they exit the milling chamber, generally through the lower exit, at low pressure and low velocity. The particles that exit the milling chamber are heavier than air and settle downward through the lower exit into the collection vessel, while the exhaust gas rises (together with a minority of small particles of micronised material) and escapes into the atmosphere at low pressure and low velocity.

### Procedure:

The micronizer is assembled. The venturi protrusion distance from input port is adjusted to 1.0 cm respectively (e.g. so that the narrow head of the venturi inlet is positioned below and slightly forward of the material inlet port) and is measured with a micro-caliper to make sure that it is inserted correctly. The ring (R) and venturi (V) pressures are adjusted according to the values specified in the experimental design (refer to experimental section below) by adjusting the valves on the pressure gauges on the micronizer. The setup is checked for leakage by observing if there is any fluctuation in the reading of the pressure gauges.

Note that the venturi (V) pressure is kept at least 2 bars greater than the ring (R) pressure to prevent regurgitation of material, e.g. outwardly from the material inlet port.

Balance performance is checked with calibration weights. Specified amount of the parent material (see section on experimental run) is weighed into a plastic weigh boat. The material is then fed into the micronizer using a vibrational spatula (e.g. V-shaped in cross-section) at a specified feed rate. The material feeding time and equipment pressures are monitored during the micronization process.

Upon completion of the micronising run, the nitrogen supply is shut off and the collection bag is tapped to allow particles to settle into the recovery/collection vessel at the bottom of the micronizer. The collection bag is removed and set aside. The micronised powder in the recovery vessel (collection vessel) and the cyclone (above the recovery vessel) are collected separately into different weighed+labelled collection vials. The weight of the micronised material is recorded. The micronizer is disassembled and residual PDE4 compound on the micronizer inner surface is rinsed with 70/30 isopropyl alcohol/water and collected into a flask. The micronizer is then thoroughly cleaned by rinsing and wiping with suitable solvent and dried before subsequent runs are performed.

### Preferred Experimental Parameters

Parent (unmicronised) material (Procedure 1): Example 518 or 518A
Parent (unmicronised) material (Procedure 2): Example 518
Balance(s) Used: Sartorius analytical
Venturi outlet insertion depth: 10.0 mm

| | | | | Time | |
|---|---|---|---|---|---|
| | | Venturi (V)/ | | needed to | |
| | Material | ring (R) | | feed | |
| Procedure | input | Pressure | Intended | material | Actual feed-rate |
| no. | amount (g) | (bar) | feed-rate | (min + sec) | (g/min) |
| 1 | 0.8795 g | V = 10 bar | 200 mg/min | 4 min 51 sec | 181 mg/min |
| | | R = 6 bar | | | |
| 2 | 0.9075 g | V = 8 bar | 200 mg/min | 4 min 43 sec | 192 mg/min |
| | | R = 5.5 bar | | | |

The above parameters can be varied using the skilled person's knowledge.

Results and/or Observations
% yield=[(Material from vessel+Material from cyclone)/Material input amount]×100

In general, very approximately 50-75% yields are achievable using this method. Procedure 1 has not been completed.

In Procedure 2, a 70.8% yield (0.6427 g) of Example 518 micronised material was obtained, including material from collection vessel and material from inside walls of cyclone.

Particle size analysis of Example 518 micronised material from Procedure 2, using laser diffraction measurement with Malvern Mastersizer longbed version, dispersing medium 0.1% Tween 80 in water, stir rate 1500 rpm, 3 mins sonification prior to final dispersion and analysis, 300 RF (Reverse Fourier) lens, Fraunhofer calculation with Malvern software:
material from collection vessel: D10=0.97 microns, D50=3.86 microns, D90=12.64 microns.
material from inside walls of cyclone: D10=0.95 microns, D50=3.42 microns, D90=9.42 microns.

### ALTERNATIVE EMBODIMENT

Examples of the compounds/salts of the invention other than Examples 518 or 518A can be micronised.

For pharmaceutical compositions suitable and/or adapted for inhaled administration, it is preferred that the pharmaceutical composition is a dry powder inhalable composition. Such a composition can comprise a powder base such as lactose or starch, the compound of formula (I) or salt thereof (preferably in particle-size-reduced form, e.g. in micronised form), and optionally a performance modifier such as L-leucine, mannitol, trehalose and/or magnesium stearate. Preferably, the dry powder inhalable composition comprises a dry powder blend of lactose and the compound of formula (I) or salt thereof. The lactose is preferably lactose hydrate e.g. lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. More preferably, the particle size of the lactose is defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. Most importantly, it is preferable that about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 JD Zwolle, Netherlands).

In the dry powder inhalable composition, preferably, the compound of formula (I) or salt thereof is present in about 0.1% to about 70% (e.g. about 1% to about 50%, e.g. about 5% to about 40%, e.g. about 20 to about 30%) by weight of the composition.

An illustrative non-limiting example of a dry powder inhalable composition follows:

Dry Powder Formulation Example—Dry powder Lactose Blend Preparation

Using a size-reduced e.g. micronised form of the compound of formula (I) or salt thereof (e.g. as prepared in the Micronisation Example above), the dry powder blend is prepared by mixing the required amount of the compound/salt (e.g. 10 mg, 1% w/w) with inhalation-grade lactose containing 10% fines (e.g. 990 mg, 99% w/w) in a Teflon™ (polytetrafluoroethene) pot in a Mikro-dismembrator ball-mill (but without a ball bearing) at ¾ speed (ca. 2000-2500 rpm) for about 4 hours at each blend concentration. The Mikro-dismembrator (available from B. Braun Biotech International, Schwarzenberger Weg 73-79, D-34212 Melsungen, Germany; www.bbraunbiotech.com) comprises a base with an upwardly-projecting and sidewardly-vibratable arm to which is attached the Teflon™ pot. The vibration of the arm achieves blending.

Other blends: 10% w/w compound/salt (50 mg)+90% w/w lactose (450 mg, inhalation-grade lactose containing 10% fines).

Serial dilution of the 1% w/w blend can achieve e.g. 0.1% and 0.3% w/w blends.

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container is rupturable or peel-openable on demand and the dose, e.g. of the dry powder composition, can be administered by inhalation via a device such as the DISKUS™ device, marketed by GlaxoSmithKline. The DISKUS™ inhalation device is usually substantially as described in GB 2,242,134 A. In such device at least one container for the pharmaceutical composition in powder form (the at least one container preferably being a plurality of sealed dose containers mounted longitudinally in a strip or ribbon) is defined between two members peelably secured to one another; the device comprises: means defining an opening station for the said at least one container; means for peeling the members apart at the opening station to open the container; and an outlet, communicating with the opened container, through which a user can inhale the pharmaceutical composition in powder form from the opened container.

In the pharmaceutical composition, a or each dosage unit for oral or parenteral administration preferably contains from 0.01 to 3000 mg, more preferably 0.5 to 1000 mg, of a compound of the formula (I) or a pharmaceutically acceptable salt thereof, calculated as the free base. A or each dosage unit for nasal or inhaled administration preferably contains from 0.001 to 50 mg, more preferably 0.01 to 5 mg, of a compound of the formula (I) or a pharmaceutically acceptable salt thereof, calculated as the free base.

A pharmaceutically acceptable compound or salt of the invention is preferably administered to a mammal (e.g. human) in a daily oral or parenteral dose of 0.001 mg to 50 mg per kg body weight per day (mg/kg/day), for example 0.01 to 20 mg/kg/day or 0.03 to 10 mg/kg/day or 0.1 to 2 mg/kg/day, of the compound of the formula (I) or a pharmaceutically acceptable salt thereof, calculated as the free base.

A pharmaceutically acceptable compound or salt of the invention is preferably administered to a mammal (e.g. human) in a daily nasal or inhaled dose of: 0.0001 to 5 mg/kg/day or 0.0001 to 1 mg/kg/day, e.g. 0.001 to 1 mg/kg/day or 0.001 to 0.3 mg/kg/day or 0.001 to 0.1 mg/kg/day or 0.005 to 0.3 mg/kg/day, of the compound of the formula (I) or a pharmaceutically acceptable salt thereof, calculated as the free base.

The pharmaceutically acceptable compounds or salts of the invention is preferably administered in a daily dose (for an adult patient) of, for example, an oral or parenteral dose of 0.01 mg to 3000 mg per day or 0.5 to 1000 mg per day e.g. 2 to 500 mg per day, or a nasal or inhaled dose of 0.001 to 300 mg per day or 0.001 to 50 mg per day or 0.01 to 30 mg per day or 0.01 to 5 mg per day or 0.02 to 2 mg per day, of the compound of the formula (I) or a pharmaceutically acceptable salt thereof, calculated as the free base.

### Combinations

The compounds, salts and/or pharmaceutical compositions according to the invention may also be used in combination with another therapeutically active agent, for example, a β₂ adrenoreceptor agonist, an anti-histamine, an anti-allergic or an anti-inflammatory agent.

The invention thus provides, in a further aspect, a combination comprising a compound of formula (I) or a pharmaceutically acceptable salt thereof together with another therapeutically active agent, for example, a β₂-adrenoreceptor agonist, an anti-histamine, an anti-allergic, an anti-inflammatory agent or an antiinfective agent.

Preferably, the β₂-adrenoreceptor agonist is salmeterol (e.g. as racemate or a single enantiomer such as the R-enantiomer), salbutamol, formoterol, salmefamol, fenoterol or terbutaline, or a salt thereof (e.g. pharmaceutically acceptable salt thereof), for example the xinafoate salt of salmeterol, the sulphate salt or free base of salbutamol or the fumarate salt of formoterol. Long-acting β₂-adrenoreceptor agonists are preferred, especially those having a therapeutic effect over a 12-24 hour period such as salmeterol or formoterol. Preferably, the β₂-adrenoreceptor agonist is for inhaled administration, e.g. once per day and/or for simultaneous inhaled administration; and more preferably the β₂-adrenoreceptor agonist is in particle-size-reduced form e.g. as defined herein. Preferably, the β₂-adrenoreceptor agonist combination is for treatment and/or prophylaxis of COPD or asthma. Salmeterol or a pharmaceutically acceptable salt thereof, e.g. salmeterol xinofoate, is preferably administered to humans at an inhaled dose of 25 to 50 micrograms twice per day (measured as the free base). The combination with a β₂-adrenoreceptor agonist can be as described in WO 00/12078.

Preferred long acting β₂-adrenoreceptor agonists include those described in WO 02/066422A, WO 03/024439, WO 02/070490 and WO 02/076933.

Especially preferred long-acting β₂-adrenoreceptor agonists include compounds of formula (XX) (described in WO 02/066422):

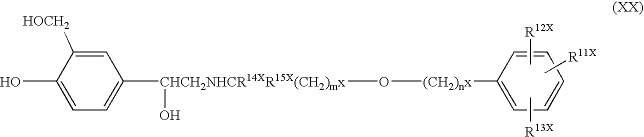

or a salt or solvate thereof, wherein in formula (XX):
m^{X} is an integer of from 2 to 8;
n^{X} is an integer of from 3 to 11,
with the proviso that m_{X}+n^{X} is 5 to 19,
R^{11X} is —XSO₂NR^{16X}R^{17X} wherein X is —(CH₂)ₚ_{ₓ}— or C₂₋₆ alkenylene;
R^{16X} and R^{17X} are independently selected from hydrogen, C₁₋₆alkyl, C₃₋₇cycloalkyl, C(O)NR^{18X}R^{19X}, phenyl, and phenyl (C₁₋₄alkyl)-,
or R^{16X} and R^{17X}, together with the nitrogen to which they are bonded, form a 5-, 6-, or 7-membered nitrogen containing ring, and R^{16X} and R^{17X} are each optionally substituted by one or two groups selected from halo, C₁₋₆alkyl, C₁₋₆haloalkyl, C₁₋₆alkoxy, hydroxy-substituted C₁₋₆alkoxy, —CO₂R^{18X}, —SO₂NR^{18X}R^{19X}, CONR^{18X}R^{19X}, —NR^{18X}C(O)R^{19X}, or a 5-, 6- or 7-membered heterocylic ring;
R^{18X} and R^{19X} are independently selected from hydrogen, C₁₋₆alkyl, C₃₋₆cycloalkyl, phenyl, and phenyl (C₁₋₄alkyl)-; and
p^{X} is an integer of from 0 to 6, preferably from 0 to 4;
R^{12X} and R^{13X} are independently selected from hydrogen, C₁₋₆alkyl, C₁₋₆alkoxy, halo, phenyl, and C₁₋₆haloalkyl; and
R^{14X} and R^{15X} are independently selected from hydrogen and C₁₋₄alkyl with the proviso that the total number of carbon atoms in R^{14X} and R^{15X} is not more than 4.

Preferred β₂-adrenoreceptor agonists disclosed in WO 02/066422 include:
3-(4-{[6-({(2R)-2-hydroxy-2-[4-hydroxy-3-(hydroxymethyl)-phenyl]ethyl}amino)hexyl]oxy}butyl)benzenesulfonamide and
3-(3-{[7-({(2R)-2-hydroxy-2-[4-hydroxy-3-hydroxymethyl)phenyl]ethyl}-amino)heptyl]oxy}propyl)benzenesulfonamide.

A preferred β₂-adrenoreceptor agonist disclosed in WO 03/024439 is: 4-{(1R)-2-[(6-{2-[(2,6-dichlorobenzyl)oxy]ethoxy}hexyl)amino]-1-hydroxyethyl}-2-(hydroxymethyl)phenol.

A combination of a compound of formula (I) or salt together with an anti-histamine is preferably for oral administration (e.g. as a combined composition such as a combined tablet), and can be for treatment and/or prophylaxis of allergic rhinitis. Examples of anti-histamines include methapyrilene, or H₁ antagonists such as cetirizine, loratadine (e.g. Clarityn™), desloratadine (e.g. Clarinex™) or fexofenadine (e.g. Allegra™).

The invention also provides, in a further aspect, a combination comprising a compound of formula (I) or a pharmaceutically acceptable salt thereof together with an anticholinergic compound, e.g. a muscarinic (M) receptor antagonist in particular an M₁, M₂, M₁/M₂, or M₃ receptor antagonist, more preferably a M₃ receptor antagonist, still more preferably a M₃ receptor antagonist which selectively antagonises (e.g. antagonises 10 times or more strongly) the M₃ receptor over the M₁ and/or M₂ receptor. For combinations of anticholinergic compounds/muscarinic (M) receptor antagonist with PDE4 inhibitors, see for example WO 03/011274 A2 and WO 02/069945 A2/US 2002/0193393 A1 and US 2002/052312 A1, and some or all of these publications give examples of anticholinergic compounds/muscarinic (M) receptor antagonists which may be used with the compounds of formula (I) or salts, and/or suitable pharmaceutical compositions. For example, the muscarinic receptor antagonist can comprise or be an ipratropium salt (e.g. ipratropium bromide), an oxitropium salt (e.g. oxitropium bromide), or more preferably a tiotropium salt (e.g. tiotropium bromide); see e.g. EP 418 716 A1 for tiotropium.

The anticholinergic compound or muscarinic (M) receptor antagonist, e.g. M₃ receptor antagonist, is preferably for inhaled administration, more preferably in particle-size-reduced form e.g. as defined herein. More preferably, both the muscarinic (M) receptor antagonist and the compound of formula (I) or the pharmaceutically acceptable salt thereof are for inhaled administration. Preferably, the anticholinergic compound or muscarinic receptor antagonist and the compound of formula (I) or salt are for simultaneous administration. The muscarinic receptor antagonist combination is preferably for treatment and/or prophylaxis of COPD.

Other suitable combinations include, for example, a combination comprising a compound of formula (I) or a pharmaceutically acceptable salt thereof together with another anti-inflammatory agent such as an anti-inflammatory corticosteroid; or a non-steroidal anti-inflammatory drug (NSAID) such as a leukotriene antagonist (e.g. montelukast), an iNOS inhibitor, a tryptase inhibitor, a elastase inhibitor, a beta-2 integrin antagonist, a adenosine 2a agonist, a CCR3 antagonist, or a 5-lipoxogenase inhibitor); or an antiinfective agent (e.g. an antibiotic or antiviral). An iNOS inhibitor is preferably for oral administration. Suitable iNOS inhibitors (inducible nitric oxide synthase inhibitors) include those disclosed in WO 93/13055, WO 98/30537, WO 02/50021, WO 95/34534 and WO 99/62875. Suitable CCR3 inhibitors include those disclosed in WO 02/26722.

In a combination comprising a compound of formula (I) or a pharmaceutically acceptable salt thereof together with an anti-inflammatory corticosteroid (which is preferably for treatment and/or prophylaxis of asthma, COPD or allergic rhinitis), then preferably the anti-inflammatory corticosteroid is fluticasone, fluticasone propionate (e.g. see U.S. Pat. No. 4,335,121), beclomethasone, beclomethasone 17-propionate ester, beclomethasone 17,21-dipropionate ester, dexamethasone or an ester thereof, mometasone or an ester thereof, ciclesonide, budesonide, flunisolide, or a compound as described in WO 02/12266 A1 (e.g. as claimed in any of claims 1 to 22 therein), or a pharmaceutically acceptable salt of any of the above. If the anti-inflammatory corticosteroid is a compound as described in WO 02/12266 A1, then preferably it is Example 1 therein {which is 6α,9α-difluoro-17α-[(2-furanylcarbonyl)oxy]-11β-hydroxy-16α-methyl-3-oxo-androsta-1,4-diene-17β-carbothioic acid S-fluoromethyl ester} or Example 41 therein {which is 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester}, or a pharmaceutically acceptable salt thereof. The anti-inflammatory corticosteroid is preferably for intranasal or inhaled administration. Fluticasone propionate is preferred and is preferably for inhaled administration to a human either (a) at a dose of 250 micrograms once per day or (b) at a dose of 50 to 250 micrograms twice per day.

Also provided is a combination comprising a compound of formula (I) or a pharmaceutically acceptable salt thereof together with β₂-adrenoreceptor agonist and an anti-inflammatory corticosteroid, for example as described in WO 03/030939 A1. Preferably this combination is for treatment and/or prophylaxis of asthma, COPD or allergic rhinitis. The β₂-adrenoreceptor agonist and/or the anti-inflammatory corticosteroid can be as described above and/or as described in WO 03/030939 A1. Most preferably, in this “triple” combination, the β₂-adrenoreceptor agonist is salmeterol or a pharmaceutically acceptable salt thereof (e.g. salmeterol xinafoate) and the anti-inflammatory corticosteroid is fluticasone propionate.

The combinations referred to above may conveniently be presented for use in the form of a pharmaceutical composition and thus a pharmaceutical composition comprising a combination as defined above together with one or more pharmaceutically acceptable carriers and/or excipients represent a further aspect of the invention.

The individual compounds of such combinations may be administered either sequentially or simultaneously in separate or combined pharmaceutical composition.

In one embodiment, the combination as defined herein can be for simultaneous inhaled administration and is disposed in a combination inhalation device. Such a combination inhalation device is another aspect of the invention. Such a combination inhalation device can comprise a combined pharmaceutical composition for simultaneous inhaled administration (e.g. dry powder composition), the composition comprising all the individual compounds of the combination, and the composition being incorporated into a plurality of sealed dose containers mounted longitudinally in a strip or ribbon inside the inhalation device, the containers being rupturable or peel-openable on demand; for example such inhalation device can be substantially as described in GB 2,242,134 A (DISKUS™) and/or as described above. Alternatively, the combination inhalation device can be such that the individual compounds of the combination are administrable simultaneously but are stored separately (or wholly or partly stored separately for triple combinations), e.g. in separate pharmaceutical compositions, for example as described in PCT/EP03/00598 filed on 22 Jan. 2003 (e.g. as described in the claims thereof e.g. claim 1).

The invention also provides a method of preparing a combination as defined herein,
the method comprising either
(a) preparing a separate pharmaceutical composition for administration of the individual compounds of the combination either sequentially or simultaneously, or
(b) preparing a combined pharmaceutical composition for administration of the individual compounds of the combination simultaneously,
wherein the pharmaceutical composition comprises the combination together with one or more pharmaceutically acceptable carriers and/or excipients.

The invention also provides a combination as defined herein, prepared by a method as defined herein.

### Biological Test Methods

### PDE 3, PDE 4B, PDE 4D, PDE 5, PDE 6 Primary Assay Methods

The activity of the compounds can be measured in the assay methods shown below. Preferred compounds of the invention are selective PDE4 inhibitors, i.e. they inhibit PDE4 (e.g. PDE4B and/or PDE4D, preferably PDE4B) more strongly than they inhibit PDE3 and/or more strongly than they inhibit PDE5 and/or more strongly than they inhibit PDE6.

### PDE Enzyme Sources and Literature References

Human recombinant PDE4B, in particular the 2B splice variant thereof (HSPDE4B2B), is disclosed in WO 94/20079 and also M. M. McLaughlin et al., “A low Km, rolipram-sensitive, cAMP-specific phosphodiesterase from human brain: cloning and expression of cDNA, biochemical characterisation of recombinant protein, and tissue distribution of mRNA”, J. Biol. Chem., 1993, 268, 6470-6476. For example, in Example 1 of WO 94/20079, human recombinant PDE4B is described as being expressed in the PDE-deficient yeast Saccharomyces cerevisiae strain GL62, e.g. after induction by addition of 150 uM CuSO₄, and 100,000×g supernatant fractions of yeast cell lysates are described for use in the harvesting of PDE4B enzyme.

Human recombinant PDE4D (HSPDE4D3A) is disclosed in P. A. Baecker et al., “Isolation of a cDNA encoding a human rolipram-sensitive cyclic AMP phoshodiesterase (PDE IVD)”, Gene, 1994, 138, 253-256.

Human recombinant PDE5 is disclosed in K. Loughney et al., “Isolation and characterisation of cDNAs encoding PDE5A, a human cGMP-binding, cGMP-specific 3′,5′-cyclic nucleotide phosphodiesterase”, Gene, 1998, 216, 139-147.

PDE3 was purified from bovine aorta as described by H. Coste and P. Grondin, “Characterisation of a novel potent and specific inhibitor of type V phosphodiesterase”, Biochem. Pharmacol., 1995, 50, 1577-1585.

PDE6 was purified from bovine retina as described by: P. Catty and P. Deterre, “Activation and solubilization of the retinal cGMP-specific phosphodiesterase by limited proteolysis”, Eur. J. Biochem., 1991, 199, 263-269; A. Tar et al. “Purification of bovine retinal cGMP phosphodiesterase”, Methods in Enzymology, 1994, 238, 3-12; and/or D. Srivastava et al. “Effects of magnesium on cyclic GMP hydrolysis by the bovine retinal rod cyclic GMP phosphodiesterase”, Biochem. J., 1995, 308, 653-658.

### Inhibition of PDE 3, PDE 4B, PDE 4D, PDE 5 or PDE 6 Activity: Radioactive Scintillation Proximity Assay (SPA)

The ability of compounds to inhibit catalytic activity at PDE4B or 4D (human recombinant), PDE3 (from bovine aorta), PDE5 (human recombinant) or PDE6 (from bovine retina) was determined by Scintillation Proximity Assay (SPA) in 96-well format. Test compounds (preferably as a solution in DMSO, e.g. 0.5 to 1 microlitre (ul) volume) were preincubated at ambient temperature (room temperature, e.g. 19-23° C.) in Wallac Isoplates (code 1450-514) with PDE enzyme in 50 mM Tris-HCl buffer pH 7.5, 8.3 mM MgCl₂, 1.7 mM EGTA, 0.05% (w/v) bovine serum albumin for 10-30 minutes (usually 30 minutes). The enzyme concentration was adjusted so that no more than 20% hydrolysis of the substrate defined below occurred in control wells without compound, during the incubation. For the PDE3, PDE4B and PDE4D assays, [5′,8-³H]Adenosine 3′,5′-cyclic phosphate (Amersham Pharmacia Biotech, code TRK.559; or Amersham Biosciences UK Ltd, Pollards Wood, Chalfont St Giles, Buckinghamshire HP8 4SP, UK) was added to give 0.05 uCi per well and ˜10 nM final concentration. For the PDE5 and PDE6 assays, [8-³H]Guanosine 3′,5′-cyclic phosphate (Amersham Pharmacia Biotech, code TRK.392) was added to give 0.05 uCi per well and 36 nM final concentration. Plates, e.g. containing approx. 100 ul volume of assay mixture, were mixed on an orbital shaker for 5 minutes and incubated at ambient temperature for 1 hour. Phosphodiesterase SPA beads (Amersham Pharmacia Biotech, code RPNQ 0150) were added (˜1 mg per well) to terminate the assay. Plates were sealed and shaken and allowed to stand at ambient temperature for 35 minutes to 1 hour (preferably 35 minutes) to allow the beads to settle. Bound radioactive product was measured using a WALLAC TRILUX 1450 Microbeta scintillation counter. For inhibition curves, 10 concentrations (1.5 nM-30 uM) of each compound were assayed. Curves were analysed using ActivityBase and XLfit (ID Business Solutions Limited, 2 Ocean Court, Surrey Research Park, Guildford, Surrey GU2 7QB, United Kingdom) Results were expressed as pIC₅₀ values.

In an alternative to the above radioactive SPA assay, PDE4B or PDE4D inhibition can be measured in the following Fluorescence Polarisation (FP) assay:

### Inhibition of PDE4B or PDE4D Activity: Fluorescence Polarisation (FP) Assay

The ability of compounds to inhibit catalytic activity at PDE4B (human recombinant) or PDE4D (human recombinant) was determined by IMAP Fluorescence Polarisation (FP) assay (IMAP Explorer kit, available from Molecular Devices Corporation, Sunnydale, Calif., USA; Molecular Devices code: R8062) in 384-well format. The IMAP FP assay is able to measure PDE activity in an homogenous, non-radioactive assay format. The FP assay uses the ability of immobilised trivalent metal cations, coated onto nanoparticles (tiny beads), to bind the phosphate group of F1-AMP that is produced on the hydrolysis of fluorescein-labelled (F1) cyclic adenosine mono-phosphate (F1-cAMP) to the non-cyclic F1-AMP form. F1-cAMP does not bind. Binding of F1-AMP product to the beads (coated with the immobilised trivalent cations) slows the rotation of the bound F1-AMP and leads to an increase in the fluorescence polarisation ratio of parallel to perpendicular light. Inhibition of the PDE reduces/inhibits this signal increase.

Test compounds (small volume, e.g. 0.5 to 1 ul, of solution in DMSO) were preincubated at ambient temperature (room temperature, e.g. 19-23° C.) in black 384-well microtitre plates (supplier: NUNC, code 262260) with PDE enzyme in 10 mM Tris-HCl buffer pH 7.2, 1 mM MgCl₂, 0.1% (w/v) bovine serum albumin, and 0.05% NaN₃ for 10-30 minutes. The enzyme level was set by experimentation so that reaction was linear throughout the incubation. Fluorescein adenosine 3′,5′-cyclic phosphate (from Molecular Devices Corporation, Molecular Devices code: R7091) was added to give about 40 nM final concentration (final assay volume usually ca. 25-40 ul). Plates were mixed on an orbital shaker for 10 seconds and incubated at ambient temperature for 40 minutes. IMAP binding reagent (as described above, from Molecular Devices Corporation, Molecular Devices code: R7207) was added (60 ul of a 1 in 400 dilution in binding buffer of the kit stock solution) to terminate the assay. Plates were allowed to stand at ambient temperature for 1 hour. The Fluorescence Polarisation (FP) ratio of parallel to perpendicular light was measured using an Analyst™ plate reader (from Molecular Devices Corporation). For inhibition curves, 10 concentrations (1.5 nM-30 uM) of each compound were assayed. Curves were analysed using ActivityBase and XLfit (ID Business Solutions Limited, 2 Ocean Court, Surrey Research Park, Guildford, Surrey GU2 7QB, United Kingdom). Results were expressed as pIC₅₀ values.

In the FP assay, all reagents were dispensed using Multidrop™ (available from Thermo Labsystems Oy, Ratastie 2, PO Box 100, Vantaa 01620, Finland).

For a given PDE4 inhibitor, the PDE4B (or PDE4D) inhibition values measured using the SPA and FP assays can differ slightly. However, in a regression analysis of 100 test compounds, the pIC₅₀ inhibition values measured using SPA and FP assays have been found generally to agree within 0.5 log units, for PDE4B and PDE4D (linear regression coefficient 0.966 for PDE4B and 0.971 for PDE4D; David R. Mobbs et al., “Comparison of the IMAP Fluorescence Polarisation Assay with the Scintillation Proximity Assay for Phosphodiesterase Activity”, poster to be presented at 2003 Molecular Devices UK & Europe User Meeting, 2 Oct. 2003, Down Hall, Harlow, Essex, United Kingdom).

Biological Data obtained for some of the Examples (PDE4B inhibitory activity, either as one reading or as an average of ca. 2-6 readings) are as follows, based on current measurements only. In each of the SPA and FP assays, absolute accuracy of measurement is not possible, and the readings given are accurate only up to about ±0.5 of a log unit, depending on the number of readings made and averaged:

| | PDE4B pIC₅₀ |
|---|---|
| Example number | (±about 0.5) |
| 2 | 8.0 |
| 3 | 7.8 |
| 6 | 6.6 |
| 11 | 7.4 |
| 21 | 8.5 |
| 22 | 7.9 |
| 32 | 7.7 |
| 40 | 8.3 |
| 63 | 6.9 |
| 1, 36, 39, 41, 42, 43, 44, 47, | 7.0 to 7.9 |
| 48, 63, 83, 109, 178, 187 and 600 | |
| 100, 155, 165, 167, 201, | 8.2 to 10.0 |
| 260, 261, 263, 265, 266, | |
| 267, 271, 431, 493, 494, | |
| 495, 498, 518, 518A, 528, | |
| 551, 575, 581, 584, 619, | |
| 622, 624-626, 628, 630, 636, | |
| 638, 643-645, 653, and | |
| 677 to 686 | |
| 196 | 7.9 |
| 198 | 8.5 |

Examples 1-201 were generally tested for PDE4B inhibition using the radioactive SPA assay. Of Examples 207-665, and 677-686, all or almost all (except perhaps for Examples 451, 631-632, 635, 652) were tested for PDE4B inhibition; and of these some were tested by the radioactive SPA assay, some were tested by the FP assay. Examples 1-201, 207-450, 452-630, 633-634, 636-651, 653-665, and 677-686, but excluding reference examples 19-20, have PDE4B inhibitory activities in the range of pIC₅₀=about 6 (±about 0.5) to about 10.0 (±about 0.5). Examples 666-676 are predicted to have PDE4B inhibitory activities in the range of pIC₅₀=about 6 (±about 0.5) to about 1010 (±about 0.5).

The Examples wherein R³=cyclohexyl (NHR³=sub-formula (c)), tetrahydro-2H-pyran-4-yl (NHR³=group (h)), 4-oxocyclohexyl (NHR³=sub-formula (o)), or certain other types of substituted cyclohexyl or certain heterocycles, usually or often (especially with R¹=ethyl) have a higher level of selectivity for PDE4B over PDE5, as measured in the above enzyme inhibition assays, compared to the selectivities of comparable Examples wherein R³=cyclopropyl (NHR³=sub-formula (b)). For example, based on current measurements only, and subject to cumulative assay inaccuracies:
Examples 21, 40, 90, 198 and 201 (wherein NHR³=sub-formula (h), (c), (j), (n) and (o) respectively, R¹=ethyl) have selectivities for PDE4B over PDE5 in the range of about 3 to 20 or more times greater than the selectivity achieved for the equivalent Example 39 wherein R³=cyclopropyl (NHR³=sub-formula (b));
Examples 43 and 44 (wherein NHR³=sub-formula (c) and (h) respectively) have selectivities for PDE4B over PDE5 in the range of about 4 to 8 or more times greater than the selectivity achieved for the equivalent R³=cyclopropyl Example 42;
Examples 22 and 48 (wherein NHR³=sub-formula (h) and (c) respectively) have selectivities for PDE4B over PDE5 in the range of about 2.5 to 10 or more times greater than the selectivity achieved for the equivalent R³=cyclopropyl Example 47; and
Examples 2 and 3 (wherein NHR³=sub-formula (c) and (h) respectively) have selectivities for PDE4B over PDE5 in the range of about 2 to 5 or more times greater than the selectivity achieved for the equivalent R³=cyclopropyl Example 1.
   Emesis: Some known PDE4 inhibitors can cause emesis and/or nausea to greater or lesser extents (e.g. see Z. Huang et al., Current Opinion in Chemical Biology, 2001, 5: 432-438, see especially pages 433-434 and refs cited therein). Therefore, it would be preferable, but not essential, if a PDE4 inhibitory compound or salt of the invention were to cause only limited or manageable emetic side-effects. Emetic side-effects can for example be measured by the emetogenic potential of the compound or salt when administered to ferrets; for example one can measure the time to onset, extent, frequency and/or duration of vomiting, retching and/or writhing in ferrets after oral or parenteral administration of the compound or salt. See for example 1n vivo Assay 4 hereinafter for a measurement method for anti-inflammatory effect, emetic side-effects and therapeutic index (TI) in the ferret. See also for example A. Robichaud et al., “Emesis induced by inhibitors of [PDE IV] in the ferret”, Neuropharmacology, 1999, 38, 289-297, erratum Neuropharmacology, 2001, 40, 465-465. However, optionally, emetic side-effects and therapeutic index (TI) in rats can be conveniently measured by monitoring the pica feeding behaviour of rats after administration of the compound or salt of the invention (see In Vivo Assay 2 below).
   Other side effects: Some known PDE4 inhibitors can cause other side effects such as headache and other central nervous sytem (CNS-) mediated side effects; and/or gastrointestinal (GI) tract disturbances. Therefore, it would be preferable but not essential if a particular PDE4 inhibitory compound or salt of the invention were to cause only limited or manageable side-effects in one or more of these side-effect categories.

### In Vivo Biological Assays

The in vitro enzymatic PDE4B inhibition assay described above should be regarded as being the primary test of biological activity. However, additional in vivo biological tests, which are optional and which are not an essential measure of efficacy or side-effects, are described below.

### In Vivo Assay 1. LPS-Induced Pulmonary Neutrophilia in Rats: Effect of Orally Administered PDE4 Inhibitors

Pulmonary neutrophil influx has been shown to be a significant component to the family of pulmonary diseases like chronic obstructive pulmonary disease (COPD) which can involve chronic bronchitis and/or emphysema (G. F. Filley, Chest. 2000; 117(5); 251s-260s). The purpose of this neutrophilia model is to study the potentially anti-inflammatory effects in vivo of orally administered PDE4 inhibitors on neutrophilia induced by inhalation of aerosolized lipopolysaccharide (LPS), modelling the neutrophil inflammatory component(s) of COPD. See the literature section below for scientific background.

Male Lewis rats (Charles River, Raleigh, N.C., USA) weighing approximately 300-400 grams are pretreated with either (a) test compound suspended in 0.5% methylcellulose (obtainable from Sigma-Aldrich, St Louis, Mo., USA) in water or (b) vehicle only, delivered orally in a dose volume of 10 ml/kg. Generally, dose response curves are generated using the following doses of PDE4 inhibitors: 10.0, 2.0, 0.4, 0.08 and 0.016 mg/kg. Thirty minutes following pretreatment, the rats are exposed to aerosolized LPS (Serotype E. Coli 026:B6 prepared by trichloroacetic acid extraction, obtainable from Sigma-Aldrich, St Louis, Mo., USA), generated from a nebulizer containing a 100 μg/ml LPS solution. Rats are exposed to the LPS aerosol at a rate of 4 L/min for 20 minutes. LPS exposure is carried out in a closed chamber with internal dimensions of 45 cm length×24 cm width×20 cm height. The nebulizer and exposure chamber are contained in a certified fume hood. At 4 hours-post LPS exposure the rats are euthanized by overdose with pentobarbital at 90 mg/kg, administered intraperitoneally. Bronchoalveolar lavage (BAL) is preformed through a 14 gauge blunt needle into the exposed trachea. Five, 5 ml washes are performed to collect a total of 25 ml of BAL fluid. Total cell counts and leukocyte differentials are performed on BAL fluid in order to calculate neutrophil influx into the lung. Percent neutrophil inhibition at each dose (cf. vehicle) is calculated and a variable slope, sigmoidal dose-response curve is generated, usually using Prism Graph-Pad. The dose-response curve is used to calculate an ED50 value (in mg per kg of body weight) for inhibition by the PDE4 inhibitor of the LPS-induced neutrophilia.

Results: Based on current measurements, the compounds of Examples 22, 83 and 155, administered orally in the above procedure, exhibited neutrophilia-inhibition ED50 values in the range of about 0.5 mg/kg to about 2 mg/kg.

Alternative method and results: In an alternative embodiment of the procedure, single oral doses of 10 mg/kg or 1.0 mg/kg of the PDE4 inhibitor (or vehicle) is administered to the rats, and percent neutrophil inhibition is calculated and reported for that specific dose. In this embodiment, based on current measurements, the compounds of Examples 21, 100, 109, 167, 172 and 600, administered orally in the above procedure at a single dose of 1.0 mg/kg, exhibited percent neutrophilia-inhibition in the range of about 19% to about 69% (or in the range of about 32% to about 69% for Examples 21, 100, 109, 167 and 600).

### LITERATURE

Filley G. F. Comparison of the structural and inflammatory features of COPD and asthma. Chest. 2000; 117(5) 251s-260s.
Howell R E, Jenkins L P, Fielding L E, and Grimes D. Inhibition of antigen-induced pulmonary eosinophilia and neutrophilia by selective inhibitors of phosphodiesterase types 3 and 4 in brown Norway rats. Pulmonary Pharmacology. 1995; 8: 83-89.
Spond J, Chapman R, Fine J, Jones H, Kreutner W, Kung T T, Minnicozzi M. Comparison of PDE 4 inhibitors, Rolipram and S B 207499 (Ariflo™), in a rat model of pulmonary neutrophilia. Pulmonary Pharmacology and Therapeutics. 2001; 14: 157-164.
Underwood D C, Osborn R R, Bochnowicz S, Webb E F, Rieman D J, Lee J C, Romanic A M, Adams J L, Hay D W P, and Griswold D E. S B 239063, a p38 MAPK inhibitor, reduces neutrophilia, inflammatory cytokines, MMP-9, and fibrosis in lung. Am J Physiol Lung Cell Mol Physiol. 2000; 279: L895-L902.

### In Vivo Assay 2. Rat Pica Model of Emesis

Background: Selective PDE4 inhibitors have been shown to inhibit inflammation in various in vitro and in vivo models by increasing intracellular levels of cAMP of many immune cells (e.g. lymphocytes, monocytes). However, a side effect of some PDE4 inhibitors in many species is emesis. Because many rat models of inflammation are well characterized, they have been used in procedures (see e.g. In Vivo Assay 1 above) to show beneficial anti-inflammatory effects of PDE 4 inhibitors. However rats have no emetic response (they have no vomit reflex), so that the relationship between beneficial anti-inflammatory effects of PDE 4 inhibitors and emesis is difficult to study directly in rats.

However, in 1991, Takeda et al. (see Literature section below) demonstrated that the pica feeding response is analogous to emesis in rats. Pica feeding is a behavioural response to illness in rats wherein rats eat non-nutritive substances such as earth or in particular clay (e.g. kaolin) which may help to absorb toxins. Pica feeding can be induced by motion and chemicals (especially chemicals which are emetic in humans), and can be inhibited pharmacologically with drugs that inhibit emesis in humans. The Rat Pica Model, In Vivo Assay 2, can determine the level of pica response of rats to PDE 4 inhibition at pharmacologically relevant doses in parallel to in vivo anti-inflammatory Assays in (a separate set of) rats (e.g. In Vivo Assay 1 above). Anti-inflammatory and pica assays in the same species together can provide data on the “therapeutic index” (TI) in the rat of the compounds/salts of the invention. The Rat TI can for example be calculated as the ratio of a) the potentially-emetic Pica Response ED50 dose from Assay 2 to b) the rat anti-inflammatory ED50 dose (e.g. measured by rat neutrophilia-inhibition in eg In Vivo Assay 1), with larger TI ratios possibly indicating lower emesis at many anti-inflammatory doses. This might allow a choice of a non-emetic or minimal-emetic pharmaceutical dose of the compounds or salts of the invention which has an anti-inflammatory effect. It is recognised however that achieving a low-emetic PDE4 inhibitory compound is not essential.

Procedure: On the first day of the experiment, the rats are housed individually in cages without bedding or “enrichment”. The rats are kept off of the cage floor by a wire screen. Pre-weighed food cups containing standard rat chow and clay pellets are placed in the cage. The clay pellets, obtainable from Languna Clay Co, City of Industry, Calif., USA, are the same size and shape as the food pellets. The rats are acclimated to the clay for 72 hours, during which time the cups and food and clay debris from the cage are weighed daily on an electronic balance capable of measuring to the nearest 0.1 grams. By the end of the 72 hour acclimation period the rats generally show no interest in the clay pellets.

At the end of 72 hours the rats are placed in clean cages and the food cups weighed. Rats that are still consuming clay regularly are removed from the study. Immediately prior to the dark cycle (the time when the animals are active and should be eating) the animals are split into treatment groups and dosed orally with a dose of the compound/salt of the invention (different doses for different treatment groups) or with vehicle alone, at a dose volume of 2 ml/kg. In this oral dosing, the compound/salt is in the form of a suspension in 0.5% methylcellulose (obtainable Sigma-Aldrich, St. Louis, Mo., USA) in water. The food and clay cups and cage debris are weighed the following day and the total clay and food consumed that night by each individual animal is calculated.

A dose response is calculated by first converting the data into quantal response, where animals are either positive or negative for the pica response. A rat is “pica positive” if it consumes greater than or equal to 0.3 grams of clay over the mean of is usually calculated using logistic regression performed by the Statistica software statistical package. A Pica Response ED50 value in mg per kg of body weight can then be calculated.

Results: Using the above procedure, and according to current measurements, the compounds of Examples 22, 83 and 155 exhibited a Pica Response ED50 in the range of about 4.8 mg/kg to greater than or equal to about 40 mg/kg. It can be seen that these Pica Response ED50 doses are higher than the neutrophilia-inhibition ED50 values for these three Examples (see In Vivo Assay 1 above), so that a Therapeutic Index (TI) in rats of >2, as measured by Assays 1+2 and according to current measurements, appears at first sight to have been achieved for these three Examples.

The Therapeutic Index (TI) calculated this way is often significantly different to, and often higher than, the TI calculated in the ferret (see In vivo Assay 4 below).

### LITERATURE

Beavo J A, Contini, M., Heaslip, R. J. Multiple cyclic nucleotide phosphodiesterases. Mol. Pharmacol. 1994; 46:399-405.
Spond J, Chapman R, Fine J, Jones H, Kreutner W, Kung T T, Minnicozzi M. Comparison of PDE 4 inhibitors, Rolipram and SB 207499 (Ariflo™), in a rat model of pulmonary neutrophilia. Pulmonary Pharmacology and Therapeudtics. 2001; 14:157-164.
Takeda N, Hasegawa S, Morita M, and Matsunaga T. Pica in rats is analogous to emesis: an animal model in emesis research. Pharmacology, Biochemistry and Behavior. 1991; 45:817-821.
Takeda N, Hasegawa S, Morita M, Horii A, Uno A, Yamatodani A and Matsunaga T. Neuropharmacological mechanisms of emesis. I. Effects of antiemetic drugs on motion- and apomorphine-induced pica in rats. Meth Find Exp Clin Pharmacol. 1995; 17(9) 589-596.
Takeda N, Hasegawa S, Morita M, Horii A, Uno A, Yamatodani A and Matsunaga T. Neuropharmacological mechanisms of emesis. II. Effects of antiemetic drugs on cisplatin-induced pica in rats. Meth Find Exp Clin Pharmacol. 1995; 17(9) 647-652.

In Vivo Assay 3. LPS Induced Pulmonary Neutrophilia in Rats: Effect of Intratracheally Administered PDE4 Inhibitors

This assay is an animal model of inflammation in the lung—specifically neutrophilia induced by lipopolysaccharide (LPS)—and allows the study of putative inhibition of such neutrophilia (anti-inflammatory effect) by intratracheally (i.t.) administered PDE4 inhibitors. The PDE4 inhibitors are preferably in dry powder or wet suspension form. I.t. administration is one model of inhaled administration, allowing topical delivery to the lung.

Animals: Male CD (Sprague Dawley Derived) rats supplied by Charles River, Raleigh, N.C., USA were housed in groups of 5 rats per cage, acclimatised after delivery for at least 7 days with bedding/nesting material regularly changed, fed on SDS diet R1 pelleted food given ad lib, and supplied with daily-changed pasteurised animal grade drinking water.

Device for dry powder administration: Disposable 3-way tap between dosing needle and syringe. A 3-way sterile tap (Vycon Ref 876.00) was weighed, the drug blend or inhalation grade lactose (vehicle control) was then added to the tap, the tap closed to prevent loss of drug, and the tap was re-weighed to determine the weight of drug in the tap. After dosing, the tap was weighed again to determine the weight of drug that had left the tap. The needle, a Sigma Z21934-7 syringe needle 19-gauge 152 mm (6 inches) long with luer hub, was cut by engineering to approximately 132 mm (5.2 inches), a blunt end was made to prevent them damaging the rat's trachea, and the needle weighed prior to and after drug delivery to confirm that no drug was retained in the needles after dosing.

Device for wet suspension administration: This is the similar to the above but a blunt dosing needle, whose forward end was slightly angled to the needle axis, was used, with a flexible plastic portex canula inserted into the needle.

Drugs and Materials: Lipopolysaccharide (LPS) (Serotype:0127:B8) (L3129 Lot 61K4075) was dissolved in phosphate-buffered saline (PBS). PDE4 inhibitors are used in size-reduced (e.g. micronised) form, for example according to the Micronisation Example given above. For dry powder administration of the drug, the Dry Powder Formulation Example given above, comprising drug and inhalation-grade lactose, can be used. The inhalation-grade lactose usually used (Lot E98L4675 Batch 845120) has 10% fines (10% of material under 15 um particle size measured by Malvern particle size). Wet suspensions of the drug can be prepared by adding the required volume of vehicle to the drug; the vehicle used being a mixture of saline/tween (0.2% tween 80). The wet suspension was sonicated for 10 minutes prior to use.

Preparation, and dosing with PDE 4 inhibitor: Rats were anaesthetised by placing the animals in a sealed Perspex chamber and exposing them to a gaseous mixture of isoflourane (4.5%), nitrous oxide (3 litres.minute⁻¹) and oxygen (1 litre.minute⁻¹). Once anaesthetised, the animals were placed onto a stainless steel i.t. dosing support table. They were positioned on their back at approximately a 35° angle. A light was angled against the outside of the throat to highlight the trachea. The mouth was opened and the opening of the upper airway visualised. The procedure varies for wet suspension and dry powder administration of PDE4 inhibitors as follows:

Dosing with a Wet suspension: A portex cannula was introduced via a blunt metal dosing needle that had been carefully inserted into the rat trachea. The animals were intratracheally dosed with vehicle or PDE4 inhibitor via the dosing needle with a new internal canula used for each different drug group. The formulation was slowly (10 seconds) dosed into the trachea using a syringe attached to the dosing needle.

Dosing with a Dry Powder: The three-way tap device and needle were inserted into the rat trachea up to a pre-determined point established to be located approximately 1 cm above the primary bifurcation. Another operator holds the needle at the specified position whilst 2×4 ml of air is delivered through the three-way tap by depressing the syringes (ideally coinciding with the animal inspiring), aiming to expel the entire drug quantity from the tap. After dosing, the needle and tap are removed from the airway and the tap closed off to prevent any retained drug leaving the tap.

After dosing with either wet suspension or dry powder, the animals are then removed from the table and observed constantly until they have recovered from the effects of anaesthesia. The animals are returned to the holding cages and given free access to food and water; they are observed and any unusual behavioural changes noted.

Exposure to LPS: About 2 hours after i.t. dosing with vehicle control or the PDE4 inhibitor, the rats were placed into sealed Perspex containers and exposed to an aerosol of LPS (nebuliser concentration 150 μg.ml⁻¹) for 15 minutes. Aerosols of LPS were generated by a nebuliser (DeVilbiss, USA) and this was directed into the Perspex exposure chamber. Following the 15-minute LPS-exposure period, the animals were returned to the holding cages and allowed free access to both food and water.

[In an alternative embodiment, the rats can exposed to LPS less than 2 hours after i.t. dosing. In another alternative embodiment, the rats can exposed to LPS more than 2 hours (e.g. ca. 4 or ca. 6 hours) after i.t. dosing by vehicle or PDE4 inhibitor, to test whether or not the PDE4 inhibitor has a long duration of action (which is not essential).]

Bronchoalveolar ravage: 4 hours after LPS exposure the animals were killed by overdose of sodium pentobarbitone (i.p.). The trachea was cannulated with polypropylene tubing and the lungs lavaged (washed out) with 3×5 mls of heparinised (25 units.ml⁻¹) phosphate buffered saline (PBS).

Neutrophil cell counts: The Bronchoalveolar lavage (BAL) samples were centrifuged at 1300 rpm for 7 minutes. The supernatant was removed and the resulting cell pellet resuspended in 1 ml PBS. A cell slide of the resuspension fluid was prepared by placing 100 μl of resuspended BAL fluid into cytospin holders and then spun at 5000 rpm for 5 minutes. The slides were allowed to air dry and then stained with Leishmans stain (20 minutes) to allow differential cell counting. The total cells were also counted from the resuspension. From these two counts, the total numbers of neutrophils in the BAL were determined. For a measure of PDE4-inhibitor-induced inhibition of neutrophilia, a comparison of the neutrophil count in rats treated with vehicle and rats treated with PDE4 inhibitors is conducted.

By varying the dose of the PDE4 inhibitor used in the dosing step (e.g. 0.2 or 0.1 mg of PDE4 inhibitor per kg of body weight, down to e.g. 0.01 mg/kg), a dose-response curve can be generated.

### In Vivo Assay 4. Evaluation of Therapeutic Index of PDE 4 Inhibitors in the Conscious Ferret

### 1.1 Materials

The following materials are used for these studies:

PDE4 inhibitors are prepared for oral (p.o.) administration by dissolving in a fixed volume (1 ml) of acetone and then adding cremophor to 20% of the final volume.
Acetone is evaporated by directing a flow of nitrogen gas onto the solution. Once the acetone is removed, the solution is made up to final volume with distilled water. LPS is dissolved in phosphate buffered saline.

### 1.2 Animals

Male ferrets (Mustela Pulorius Furo, weighing 1-2 kg) are transported and allowed to acclimatise for not less than 7 days. The diet comprises SDS diet C pelleted food given ad lib with Whiskers™ cat food given 3 times per week. The animals are supplied with pasteurised animal grade drinking water changed daily.

### 1.3 Experimental Protocol(s)

1.3.1 Dosing with PDE4 Inhibitors

PDE4 inhibitors are administered orally (p.o.), using a dose volume of 1 ml/kg. Ferrets are fasted overnight but allowed free access to water. The animals are orally dosed with vehicle or PDE 4 inhibitor using a 15 cm dosing needle that is passed down the back of the throat into the oesophagus. After dosing, the animals are returned to holding cages fitted with perspex doors to allow observation, and given free access to water. The animals are constantly observed and any emetic episodes (retching and vomiting) or behavioural changes are recorded. The animals are allowed access to food 60-90 minutes after p.o. dosing.

### 1.3.2 Exposure to LPS

Thirty minutes after oral dosing with compound or vehicle control, the ferrets are placed into sealed perspex containers and exposed to an aerosol of LPS (30 μg/ml) for 10 minutes. Aerosols of LPS are generated by a nebuliser (DeVilbiss, USA) and this is directed into the perspex exposure chamber. Following a 10-minute exposure period, the animals are returned to the holding cages and allowed free access to water, and at a later stage, food. General observation of the animals continues for a period of at least 2.5 hours post oral dosing. All emetic episodes and behavioural changes are recorded.

### 1.3.3 Bronchoalveolar Lavage and Cell Counts

Six hours after LPS exposure the animals are killed by overdose of sodium pentobarbitone administered intraperitoneally. The trachea is then cannulated with polypropylene tubing and the lungs lavaged twice with 20 ml heparinised (10 units/ml) phosphate buffered saline (PBS). The bronchoalveolar lavage (BAL) samples are centrifuged at 1300 rpm for 7 minutes. The supernatant is removed and the resulting cell pellet re-suspended in 1 ml PBS. A cell smear of re-suspended fluid is prepared and stained with Leishmans stain to allow differential cell counting. A total cell count is made using the remaining re-suspended sample. From this, the total number of neutrophils in the BAL sample is determined.

### 1.3.4 Pharmacodynamic Readouts

The following parameters are recorded:

a) % inhibition of LPS-induced pulmonary neutrophilia to determine the dose of PDE4 inhibitor which gives 50% inhibition (D50).
b) Emetic episodes—the number of vomits and retches are counted to determine the dose of PDE4 inhibitor that gives a 20% incidence of emesis (D20).
c) A therapeutic index (TI), using this assay, is then calculated for each PDE4 inhibitor using the following equation:

$$Therapeutic index ( TI ) = \frac{D 20 incidence of emesis}{D 50 inhibition of neutrophilia}$$

It is noted that the Therapeutic index (TI) calculated using this in vivo Assay 4 is often significantly different to, and often lower than, that calculated using the rat oral inflammation and pica feeding Assays 1+2.

The calculation of TI using the PDE4 inhibitor roflumilast in this Assay 4 is:

D20 for emesis=0.5 mg/kg p.o., D50 for neutroplilia=0.49 mg/kg p.o., TI=1.02

All publications, including but not limited to patents and patent applications, cited in this specification are herein incorporated by reference as if each individual publication were specifically and individually indicated to be incorporated by reference herein as though fully set forth.

### EXAMPLES

The various aspects of the invention will now be described by reference to the following examples. These examples are merely illustrative and are not to be construed as a limitation of the scope of the present invention. In this section, “Intermediates” represent syntheses of intermediate compounds intended for use in the synthesis of the “Examples”.

### Abbreviations Used Herein:

DMSO dimethyl sulfoxide
DCM dichloromethane
EtOAc ethyl acetate
Et₂O diethyl ether
DMF dimethyl formamide
MeOH methanol
HPLC high pressure liquid chromatography
SPE solid phase extraction
NMR nuclear magnetic resonance (in which: s=singlet, d=doublet, t=triplet, q=quartet, dd=doublet of doublets, m=multiplet, H=no. of protons)
LCMS liquid chromatography/mass spectroscopy
TLC thin layer chromatography
BEMP 2-t-butylimino-2-diethylamino-1,3-dimethylperhydro-1,3,2-diazaphosphazine
EDC 1-(3-Dimethylaminopropyl)-3-ethylcarbodiimide hydrochloride
HATU O-(7-Azabenzotriazol-1-yl)-N,N,N′,N′-tetramethyluronium hexafluorophosphate
HBTU O-(Benzotriazol-1-yl)-N,N,N′,N′-tetramethyluronium hexafluorophosphate
HOBT hydroxybenzotriazole=1-hydroxybenzotriazole
h hours
DIPEA diisopropylethyl amine (iPr₂NEt)
T_{RET} retention time
THF Tetrahydrofuran
Lawesson's reagent 2,4-bis(4-methoxyphenyl)-1,3-dithia-2,4-diphosphetane-2,4-disulphide
Room temperature this is usually in the range of about 20 to about 25° C.

### Machine Methods Used Herein:

### LCMS (Liquid Chromatography/Mass Spectroscopy)

Waters ZQ mass spectrometer operating in positive ion electrospray mode, mass range 100-1000 amu.
UV wavelength: 215-330 nM
Column: 3.3 cm×4.6 mm ID, 3 μm ABZ+PLUS
Flow Rate: 3 ml/min

### Injection Volume: 5 μl

Solvent A: 95% acetonitrile+0.05% formic acid
Solvent B: 0.1% formic acid+10 mMolar ammonium acetate
Gradient: 0% A/0.7 min, 0-100% A/3.5 min, 100% A/1.1 min, 100-0% A/0.2 min

### Mass Directed Autoprep HPLC

The prep column used was a Supelcosil ABZplus (10 cm×2.12 cm)
(usually 10 cm×2.12 cm×5 μm).
UV wavelength: 200-320 nM
Flow: 20 ml/min
Injection Volume: 1 ml; or more preferably 0.5 ml
Solvent A: 0.1% formic acid
Solvent B: 95% acetonitrile+5% formic acid; or more usually 99.95% acetonitrile+0.05% formic acid
Gradient: 100% A/1 min, 100-80% A/9 min, 80-1% A/3.5 min, 1% A/1.4 min, 1-100% A/0.1 min

### Intermediates and Examples

All reagents not detailed in the text below are commercially available from established suppliers such as Sigma-Aldrich. The addresses of the suppliers for some of the starting materials mentioned in the Intermediates and Examples below or the Assays above are as follows:
ABCR GmbH & CO. KG, P.O. Box 21 01 35, 76151 Karlsruhe, Germany
Aceto Color Intermediates (catalogue name), Aceto Corporation, One Hollow Lane, Lake Success, N.Y., 11042-1215, USA
Acros Organics, A Division of Fisher Scientific Company, 500 American Road, Morris Plains, N.J. 07950, USA
Apin Chemicals Ltd., 82 C Milton Park, Abingdon, Oxon OX14 4RY, United Kingdom
Apollo Scientific Ltd., Unit 1A, Bingswood Industrial Estate, Whaley Bridge, Derbyshire SK23 7LY, United Kingdom
Aldrich (catalogue name), Sigma-Aldrich Company Ltd., Dorset, United Kingdom, telephone: +44 1202 733114; Fax: +44 1202 715460; ukcustsv@eumotes.sial.com; or
Aldrich (catalogue name), Sigma-Aldrich Corp., P.O. Box 14508, St. Louis, Mo. 63178-9916, USA; telephone: 314-771-5765; fax: 314-771-5757; custserv@sial.com; or
Aldrich (catalogue name), Sigma-Aldrich Chemie Gmbh, Munich, Germany; telephone: +49 89 6513 0; Fax: +49 89 6513 1169; deorders eumotes.sial.com.
Alfa Aesar, A Johnson Matthey Company, 30 Bond Street, Ward Hill, Mass. 01835-8099, USA
Amersham Biosciences UK Ltd, Pollards Wood, Chalfont St Giles, Buckinghamshire HP8 4SP, United Kingdom
Array Biopharma Inc., 1885 33rd Street, Boulder, Colo. 80301, USA
AstaTech, Inc., 8301 Torresdale Ave., 19C, Philadelphia, Pa. 19136, USA
Austin Chemical Company, Inc., 1565 Barclay Blvd., Buffalo Grove, Ill. 60089, USA
Avocado Research, Shore Road, Port of Heysham Industrial Park, Heysham Lancashire LA3 2XY, United Kingdom
Bayer AG, Business Group Basic and Fine Chemicals, D-51368 Leverkusen, Germany
Berk Univar plc, Berk House, P.O.Box 56, Basing View, Basingstoke, Hants RG21 2E6, United Kingdom
Butt Park Ltd., Braysdown Works, Peasedown St. John, Bath BA2 8LL, United Kingdom
Chemical Building Blocks (catalogue name), Ambinter, 46 quai Louis Bleriot, Paris, F-75016, France
ChemBridge Europe, 4 Clark's Hill Rise, Hampton Wood, Evesham, Worcestershire WR11 6FW, United Kingdom
ChemService Inc., P.O.Box 3108, West Chester, Pa. 19381, USA
Combi-Blocks Inc., 7949 Silverton Avenue, Suite 915, San Diego, Calif. 92126, USA
Dynamit Nobel GmbH, Germany; also available from: Saville Whittle Ltd (UK agents of Dynamit Nobel), Vickers Street, Manchester M40 8EF, United Kingdom
E. Merck, Germany; or E. Merck (Merck Ltd), Hunter Boulevard, Magna Park, Lutterworth, Leicestershire LE17 4XN, United Kingdom
Esprit Chemical Company, Esprit Plaza, 7680 Matoaka Road, Sarasota, Fla. 34243, USA
Exploratory Library (catalogue name), Ambinter, 46 quai Louis Bleriot, Paris, F-75016, France
Fluka Chemie AG, Industriestrasse 25, P.O. Box 260, CH-9471 Buchs, Switzerland
Fluorochem Ltd., Wesley Street, Old Glossop, Derbyshire SK13 7RY, United Kingdom
ICN Biomedicals, Inc., 3300 Hyland Avenue, Costa Mesa, Calif. 92626, USA
Interchim Intermediates (catalogue name), Interchim, 213 Avenue Kennedy, BP 1140, Montlucon, Cedex, 03103, France
Key Organics Ltd., 3, Highfield Industrial Estate, Camelford, Cornwall PL32 9QZ, United Kingdom
Lancaster Synthesis Ltd., Newgate, White Lund, Morecambe, Lancashire LA3 3DY, United Kingdom
Manchester Organics Ltd., Unit 2, Ashville Industrial Estate, Sutton Weaver, Runcorn, Cheshire WA7 3 PF, United Kingdom
Matrix Scientific, P.O. Box 25067, Columbia, S.C. 29224-5067, USA
Maybridge Chemical Company Ltd., Trevillett, Tintagel, Cornwall PL34 0HW, United Kingdom
Maybridge Reactive Intermediates (catalogue name), Maybridge Chemical Company Ltd., Trevillett, Tintagel, Cornwall PL34 0HW, United Kingdom
MicroChemistry Building Blocks (catalogue name), MicroChemistry-RadaPharma, Shosse Entusiastov 56, Moscow, 111 123, Russia
Miteni S.p.A., Via Mecenate 90, Milano, 20138, Italy
Molecular Devices Corporation, Sunnydale, Calif., USA
N.D. Zelinsky Institute, Organic Chemistry, Leninsky prospect 47, 117913 Moscow B-334, Russia
Optimer Building Block (catalogue name), Array BioPharma, 3200 Walnut Street, Boulder, Colo. 80301, USA
Peakdale Molecular Ltd., Peakdale Science Park, Sheffield Road, Chapel-en-le-Frith, High Peak SK23 OPG, United Kingdom
Pfaltz & Bauer, Inc., 172 East Aurora Street, Waterbury, Conn. 06708, USA
Rare Chemicals (catalogue name), Rare Chemicals GmbH, Schulstrasse 6, 24214 Gettorf, Germany
SALOR (catalogue name) (Sigma Aldrich Library of Rare Chemicals), Aldrich Chemical Company Inc, 1001 West Saint Paul Avenue, Milwaukee, Wis. 53233, USA
Sigma (catalogue name), Sigma-Aldrich Corp., P.O. Box 14508, St. Louis, Mo. 63178-9916, USA; see “Aldrich” above for other non-US addresses and other contact details
SIGMA-RBI, One Strathmore Road, Natick, Mass. 01760-1312, USA
Synchem OHG Heinrich-Plett-Strasse 40, Kassel, D-34132, Germany
Syngene International Pvt Ltd, Hebbagodi, Hosur Road, Bangalore, India.
TCI America, 9211 North Harborgate Street, Portland, Oreg. 97203, USA
TimTec Building Blocks A, TimTec, Inc., P O Box 8941, Newark, Del. 19714-8941, USA
Trans World Chemicals, Inc., 14674 Southlawn Lane, Rockville, Md. 20850, USA
Ubichem PLC, Mayflower Close, Chandlers Ford Industrial Estate, Eastleigh, Hampshire SO53 4AR, United Kingdom
Ultrafine (UFC Ltd.), Synergy House, Guildhall Close, Manchester Science Park, Manchester M15 6SY, United Kingdom

Table of Intermediates

| Intermediate | |
|---|---|
| Number | Name |
| 1 | Ethyl 4-chloro-1-ethyl-1H-pyrazolo[3,4-b]pyridine-5-carboxylate |
| 2 | Ethyl 4-ethoxy-1H-pyrazolo[3,4-b]pyridine-5-carboxylate |
| 3 | Ethyl 1-methyl-4-ethoxy-1H-pyrazolo[3,4-b]pyridine-5-carboxylate |
| 4 | Ethyl 1-benzyl-4-ethoxy-1H-pyrazolo[3,4-b]pyridine-5-carboxylate |
| 5 | Ethyl 4-chloro-1-phenyl-1H-pyrazolo[3,4-b]pyridine-5-carboxylate |
| 6 | 1-Acetyl-4-aminopiperidine |
| 7 | 1-Methyl-4-aminopiperidine |
| 8 | 4-Aminotetrahydropyran |
| 8A | Tetrahydro-2H-pyran-4-amine hydrochloride = 4- |
| | Aminotetrahydropyran hydrochloride |
| 9 | (R)-(+)-3-Amino tetrahydrofuran 4-toluene sulphonate |
| 10 | (S)-(−)-3-Amino tetrahydrofuran 4-toluene sulphonate |
| 11 | Tetrahydro-2H-thiopyran-4-amine |
| 12 | Tetrahydro-3-thiopheneamine |
| 13 | Tetrahydro-3-thiopheneamine 1,1-dioxide hydrochloride |
| 14 | Tetrahydro-2H-thiopyran-4-amine-1,1-dioxide hydrochloride |
| 15 | 4-Chloro-1-ethyl-1H-pyrazolo[3,4-b]pyridine-5-carboxylic acid |
| 16 | 4-Chloro-1-ethyl-1H-pyrazolo[3,4-b]pyridine-5-carbonyl chloride |
| 17 | N-Benzyl-4-chloro-1-ethyl-1H-pyrazolo[3,4-b]pyridine-5-carboxamide |
| 18 | 4-Chloro-1-ethyl-N-(2-ethylbutyl)-1H-pyrazolo[3,4-b]pyridine-5-carboxamide |
| 19 | 4-Chloro-1-ethyl-N-(4-fluorophenyl)-1H-pyrazolo[3,4-b]pyridine-5-carboxamide |
| 20 | 4-Chloro-N-cyclopentyl-1-ethyl-1H-pyrazolo[3,4-b]pyridine-5-carboxamide |
| 21 | 4-Chloro-1-ethyl-5-(pyrrolidin-1-ylcarbonyl)-1H-pyrazolo[3,4-b]pyridine |
| 22 | 4-Chloro-1-ethyl-N-(pyridin-4-ylmethyl)-1H-pyrazolo[3,4-b]pyridine-5- |
| | carboxamide |
| 23 | 4-Chloro-1-ethyl-N-propyl-1H-pyrazolo[3,4-b]pyridine-5-carboxamide |
| 24 | 4-Chloro-1-ethyl-1H-pyrazolo[3,4-b]pyridine-5-carboxamide |
| 25 | Ethyl 4-chloro-1-methyl-1H-pyrazolo[3,4-b]pyridine-5-carboxylate |
| 26 | 4-Chloro-1-methyl-1H-pyrazolo[3,4-b]pyridine-5-carboxylic acid |
| 27 | 4-Chloro-1-methyl-1H-pyrazolo[3,4-b]pyridine-5-carbonyl chloride |
| 28 | N-Benzyl-4-chloro-1-methyl-1H-pyrazolo[3,4-b]pyridine-5-carboxamide |
| 29 | 4-Chloro-1-methyl-N-(4-fluorophenyl)-1H-pyrazolo[3,4-b]pyridine-5-carboxamide |
| 30 | 4-Chloro-1-methyl-N-(2-ethylbutyl)-1H-pyrazolo[3,4-b]pyridine-5-carboxamide |
| 31 | 4-Chloro-1-methyl-1H-pyrazolo[3,4-b]pyridine-5-carboxamide |
| 32 | Ethyl 1-ethyl-4-(tetrahydro-2H-pyran-4-ylamino)-1H-pyrazolo[3,4-b]pyridine-5- |
| | carboxylate |
| 33 | 1-Ethyl-4-(tetrahydro-2H-pyran-4-ylamino)-1H-pyrazolo[3,4-b]pyridine-5- |
| | carboxylic acid |
| 34 | Ethyl 1-ethyl-4-[(3S)-tetrahydrofuran-3-ylamino]-1H-pyrazolo[3,4-b]pyridine-5- |
| | carboxylate |
| 35 | Ethyl 1-ethyl-4-[(3R)-tetrahydrofuran-3-ylamino]-1H-pyrazolo[3,4-b]pyridine-5- |
| | carboxylate |
| 36 | Ethyl 1-ethyl-4-(tetrahydro-2H-thiopyran-4-ylamino)-1H-pyrazolo[3,4-b]pyridine- |
| | 5-carboxylate |
| 37 | Ethyl 1-ethyl-4-(tetrahydrothien-3-ylamino)-1H-pyrazolo[3,4-b]pyridine-5- |
| | carboxylate |
| 38 | Ethyl 4-(cyclopropylamino)-1-ethyl-1H-pyrazolo[3,4-b]pyridine-5-carboxylate |
| 39 | Ethyl 4-[(1,1-dioxidotetrahydrothien-3-yl)amino]-1-ethyl-1H-pyrazolo[3,4- |
| | b]pyridine-5-carboxylate |
| 40 | Ethyl 4-[(1,1-dioxidotetrahydro-2H-thiopyran-4-yl)amino]-1-ethyl-1H- |
| | pyrazolo[3,4-b]pyridine-5-carboxylate |
| 41 | 1-Ethyl-4-[(3S)-tetrahydrofuran-3-ylamino]-1H-pyrazolo[3,4-b]pyridine-5- |
| | carboxylic acid |
| 42 | Ethyl 1-ethyl-4-[(3R)-tetrahydrofuran-3-ylamino]-1H-pyrazolo[3,4-b]pyridine-5- |
| | carboxylic acid |
| 43 | 1-Ethyl-4-(tetrahydro-2H-thiopyran-4-ylamino)-1H-pyrazolo[3,4-b]pyridine-5- |
| | carboxylic acid |
| 44 | 1-Ethyl-4-(tetrahydrothien-3-ylamino)-1H-pyrazolo[3,4-b]pyridine-5-carboxylic |
| | acid |
| 45 | 4-(Cyclopropylamino)-1-ethyl-1H-pyrazolo[3,4-b]pyridine-5-carboxylic acid |
| 46 | 4-[(1,1-Dioxidotetrahydrothien-3-yl)amino]-1-ethyl-1H-pyrazolo[3,4-b]pyridine-5- |
| | carboxylic acid |
| 47 | 4-[(1,1-Dioxidotetrahydro-2H-thiopyran-4-yl)amino]-1-ethyl-1H-pyrazolo[3,4- |
| | b]pyridine-5-carboxylic acid |
| 48 | Ethyl 4-(cyclohexylamino)-1H-pyrazolo[3,4-b]pyridine-5-carboxylate |
| 49 | 4-(Cyclohexylamino)-1H-pyrazolo[3,4-b]pyridine-5-carboxylic acid |
| 50 | 1-n-Propyl-4-(tetrahydro-2H-pyran-4-ylamino)-1H-pyrazolo[3,4-b]pyridine-5- |
| | carboxylic acid |
| 51 | Ethyl 4-chloro-1-ethyl-6-methyl-1H-pyrazolo[3,4-b]pyridine-5-carboxylate |
| 52 | 4-(Cyclohexylamino)-1-ethyl-6-methyl-1H-pyrazolo[3,4-b]pyridine-5-carboxylic |
| | acid |
| 53 | 1-Ethyl-6-methyl-4-(tetrahydro-2H-pyran-4-ylamino)-1H-pyrazolo[3,4-b]pyridine- |
| | 5-carboxylic acid |
| 54 | 4-Aminocyclohexanone hydrochloride |
| 76 | 1-Ethyl-4-{[(1SR,3RS)-3-hydroxycyclohexyl]amino}-1H-pyrazolo[3,4- |
| | b]pyridine-5-carboxylic acid |

### Intermediate 1: Ethyl 4-chloro-1-ethyl-1H-pyrazolo[3,4-b]pyridine-5-carboxylate

Prepared from commercially available 5-amino-1-ethyl pyrazole as described by G. Yu et. al. in J. Med. Chem., 2001, 44, 1025-1027:

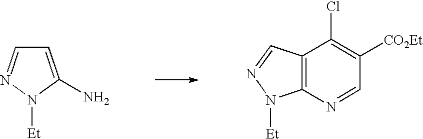

### Intermediate 2: Ethyl 4-ethoxy-1H-pyrazolo[3,4-b]pyridine-5-carboxylate

Can be prepared by oxidative cleavage (SeO₂) of 1-furanylmethyl derivative, as described by T. M. Bare et. al. In J. Med. Chem., 1989, 32, 2561-2573, (further referenced to Zuleski, F. R., Kirkland, K. R., Melgar, M. D.; Malbica, J. Drug. Metab. Dispos., 1985, 13, 139)

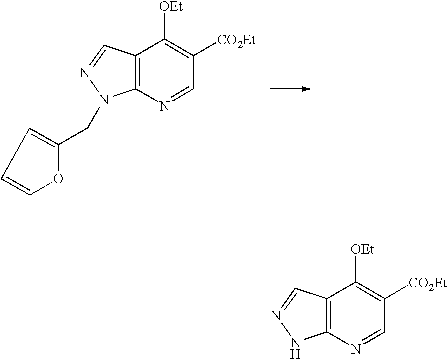

### Intermediate 3: Ethyl 1-methyl-4-ethoxy-1H-pyrazolo[3,4-b]pyridine-5-carboxylate

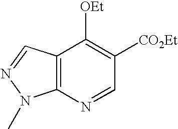

A mixture of Intermediate 2 (0.47 g) and anhydrous potassium carbonate (0.83 g) (previously dried by heating at 100° C.) in anhydrous dimethylformamide (DMF) (4 ml) was treated with iodomethane (0.26 ml) and stirred vigorously for 3 h. The mixture was then filtered and the filtrate concentrated in vacuo to afford a residual oil, which was partitioned between dichloromethane (DCM) (25 ml) and water (25 ml). The layers were separated and the aqueous phase was extracted with further DCM (2×25 ml). The combined organic extracts were dried over anhydrous sodium sulphate and evaporated to an orange solid which was applied to an SPE cartridge (silica, 20 g). The cartridge was eluted sequentially with EtOAc:petrol (1:4, 1:2 and 1:1), then chloroform:methanol (49:1, 19:1 and 9:1). Fractions containing desired material were combined and concentrated in vacuo to afford Intermediate 3 (0.165 g). LCMS showed MH⁺=250; T_{RET}=2.59 min.

### Intermediate 4: Ethyl 1-benzyl-4-ethoxy-1H-pyrazolo[3,4-b]pyridine-5-carboxylate

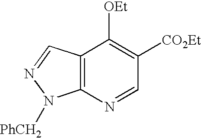

A mixture of Intermediate 2 (0.47 g) and anhydrous potassium carbonate (0.83 g) (previously dried by heating at 100° C.) in anhydrous DMF (4 ml) was treated with benzyl bromide (0.72 g) then stirred vigorously and heated at 55° C. for 4.5 h. The mixture was allowed to cool, then filtered and the filtrate concentrated in vacuo to afford a residual oil, which was partitioned between DCM (25 ml) and water (25 ml). The layers were separated and the aqueous phase was extracted with further DCM (2×25 ml). The combined organic extracts were dried over anhydrous sodium sulphate and evaporated to a yellow oily solid which was dissolved in DCM and applied to an SPE cartridge (silica, 20 g). The cartridge was eluted with a gradient of EtOAc:petrol (1:4, 1:2 and 1:1) then chloroform:methanol (49:1, 19:1 and 9:1). Fractions containing desired material were combined and concentrated in vacuo to afford Intermediate 4 (0.33 g). LCMS showed MH⁺=326; T_{RET}=3.24 min.

### Intermediate 5: Ethyl 4-chloro-1-phenyl-1H-pyrazolo[3,4-b]pyridine-5-carboxylate

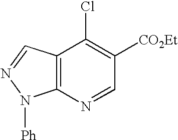

A mixture of 5-amino-1-phenyl pyrazole (2.0 g) and diethylethoxymethylene malonate (2.54 ml) was heated under Dean Stark conditions at 120° C. for 16 h. The solution was cooled, phosphorus oxychloride (16 ml) was then added and the mixture heated under reflux for a further 20 h. Excess phosphorus oxychloride was removed in vacuo and the residue partitioned between diethyl ether and water, proceeding with extreme caution on addition of water. The ethereal layer was washed with further water, then dried over magnesium sulphate and concentrated in vacuo to afford ethyl 4-chloro-1-phenyl-1H-pyrazolo[3,4-b]pyridine-5-carboxylate (2.09 g). LCMS showed MH⁺=302; T_{RET}=3.80 min.

### Intermediate 6: 1-Acetyl-4-aminopiperidine

Prepared from commercially available N1-benzyl-4-aminopiperidine as described by Yamada et. al. In WO 00/42011:

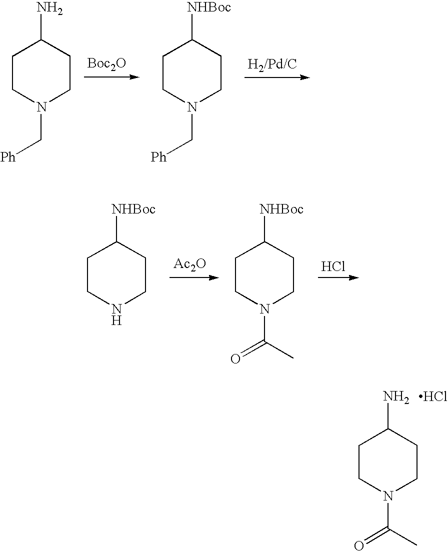

### Intermediate 7: 1-Methyl-4-aminopiperidine

Prepared from commercially available N-methyl-4-piperidone as described by C. M. Andersson et. al. in WO01/66521:

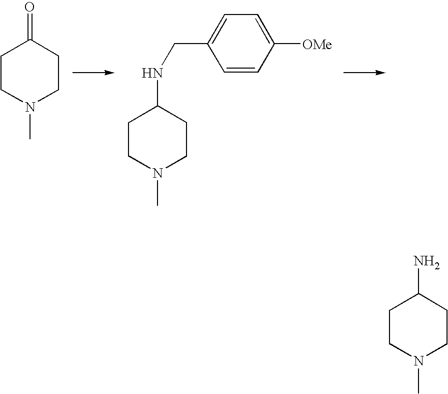

### Intermediate 8: 4-Aminotetrahydropyran

Commercially available from Combi-Blocks Inc., 7949 Silverton Avenue, Suite 915, San Diego, Calif. 92126, USA (CAS 38041-19-9)

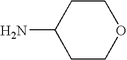

### Intermediate 8A: Tetrahydro-2H-pyran-4-amine hydrochloride-4-Aminotetrahydropyran hydrochloride

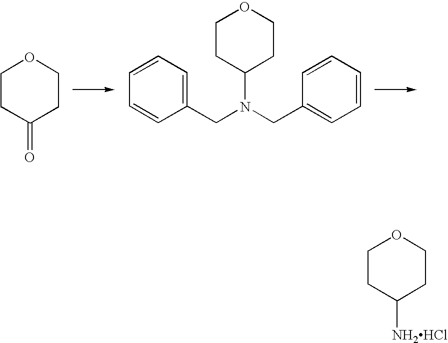

### Step 1: N,N-dibenzyltetrahydro-2H-pyran-4-amine

Dibenzylamine (34.5 g) and acetic acid (6.7 ml) were added to a stirred solution of tetrahydro-4H-pyran-4-one (16.4 g, commercially available from e.g. Aldrich) in dichloromethane (260 ml) at 0° C. to 5° C. After 2.5 h at 0° C. to 5° C., sodium triacetoxyborohydride (38.9 g) was added portionwise, and the mixture was allowed to warm to room temperature. After stirring at room temperature overnight, the reaction mixture was washed successively with 2M-sodium hydroxide (200 ml and 50 ml), water (2×50 ml) and brine (50 ml), then dried and evaporated to give a yellow oil (45 g). This oil was stirred with methanol (50 ml) at 4° C. for 30 min to give the product as a white solid (21.5 g). LCMS showed MH⁺=282; T_{RET}=1.98 min.

### Step 2: Tetrahydro-2H-pyran-4-amine hydrochloride

N,N-dibenzyltetrahydro-2H-pyran-4-amine (20.5 g) was dissolved in ethanol (210 ml) and hydrogenated over 10% palladium on carbon catalyst (4 g) at 100 psi for 72 h at room temperature. The reaction mixture was filtered and the filtrate was adjusted to pH 1 with 2M-hydrogen chloride in diethyl ether. Evaporation of solvents gave a solid which was triturated with diethyl ether to give the product as a white solid (9.23 g). ¹H NMR (400 MHz in d₆-DMSO, 27° C., δppm) 8.24 (br. s, 3H), 3.86 (dd, 12, 4 Hz, 2H), 3.31 (dt, 2, 12 Hz, 2H), 3.20 (m, 1H), 1.84 (m, 2H), 1.55 (dq, 4, 12 Hz, 2H).

### Intermediate 9: (R)-(+)-3-Amino tetrahydrofuran 4-toluenesulphonate

Commercially available from Fluka Chemie AG, Germany (CAS 111769-27-8)

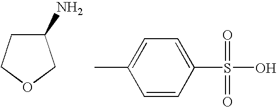

### Intermediate 10: (S)-(−)-3-Amino tetrahydrofuran 4-toluenesulphonate

Commercially available from E. Merck, Germany; or from E. Merck (Merck Ltd), Hunter Boulevard, Magna Park, Lutterworth, Leicestershire LE17 4XN, United Kingdom (CAS 104530-80-5)

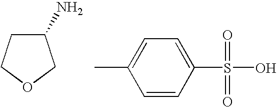

### Intermediate 11: Tetrahydro-2H-thiopyran-4-amine

Prepared from commercially available tetrahydrothiopyran-4-one as described by Subramanian et. al., J. Org. Chem., 1981, 46, 4376-4383. Subsequent preparation of the hydrochloride salt can be achieved by conventional means.

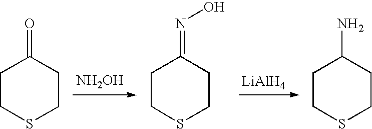

### Intermediate 12: Tetrahydro-3-thiopheneamine

Prepared in an analogous manner to Intermediate 11 from commercially available tetrahydrothiophene-4-one. The oxime formation is described by Grigg et.al., Tetrahedron, 1991, 47, 4477-4494 and the oxime reduction by Unterhalt et. al., Arch. Pharm., 1990, 317-318.

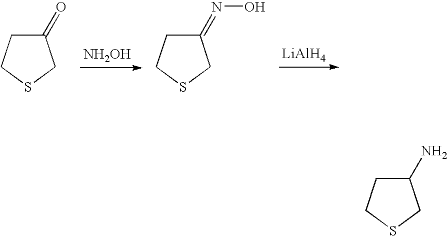

### Intermediate 13: Tetrahydro-3-thiopheneamine 1,1-dioxide hydrochloride

Commercially available from Sigma Aldrich Library of Rare Chemicals (SALOR) (CAS-6338-70-1). Preparation of the hydrochloride salt of the amine can be achieved by conventional means.

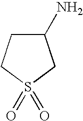

### Intermediate 14: Tetrahydro-2H-thiopyran-4-amine-1,1-dioxide hydrochloride

Prepared in an analogous manner to Intermediate 11 from commercially available tetrahydrothiophene-4-one. Oxidation to 1,1-dioxo-tetrahydro-1λ⁶-thiopyran-4-one is described by Rule et. al., in J. Org. Chem., 1995, 60, 1665-1673. Oxime formation is described by Truce et.al., in J. Org. Chem., 1957, 617, 620 and oxime reduction by Barkenbus et. al., J. Am. Chem. Soc., 1955, 77, 3866. Subsequent preparation of the hydrochloride salt of the amine can be achieved by conventional means.

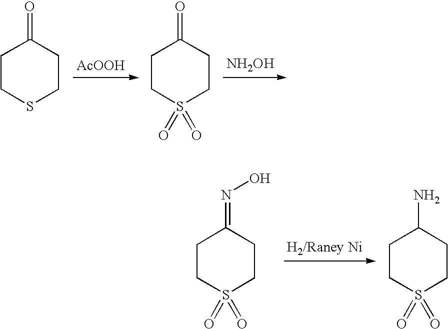

### Intermediate 15: 4-Chloro-1-ethyl-1H-pyrazolo[3,4-b]pyridine-5-carboxylic acid

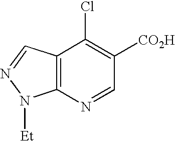

A solution of Intermediate 1 (3.5 g) in dioxane (28 ml) was treated with potassium hydroxide (6.3 g) as a solution in water (20 ml). The mixture was stirred for 2 h, then concentrated in vacuo, acidified to pH 3 with 2M aqueous hydrochloric acid and extracted with ethyl acetate. The layers were separated, the organic layer dried over sodium sulphate, then concentrated in vacuo to afford Intermediate 15 as a white solid (2.4 g). LCMS showed MH⁺=226; T_{RET}=2.62 min.

### Intermediate 17: N-Benzyl-4-chloro-1-ethyl-1H-pyrazolo[3,4-b]pyridine-5-carboxamide

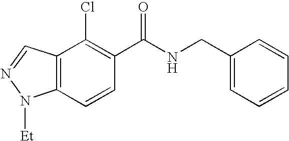 [0628] That is, Intermediate 17 is:

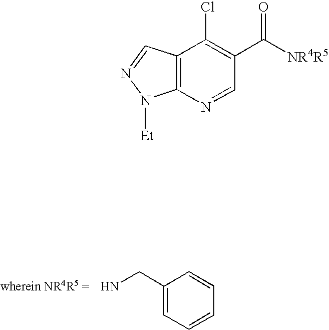

Intermediate 15 (3.5 g) was dried over phosphorus pentoxide for 1 h, then treated with thionyl chloride (47 g). The mixture was stirred and heated at 75° C. for 1.3 h. Excess thionyl chloride was removed in vacuo and the residual oil azeotroped with dichloromethane (DCM) to afford Intermediate 16, presumed to be the acid chloride derivative of Intermediate 15, as a white solid (3.3 g).

Intermediate 16 (0.473 g) was dissolved in tetrahydrofuran (THF) (4 ml) and treated with N,N-diisopropylethylamine (DIPEA) (0.509 ml), then with benzylamine (0.209 g) and the mixture stirred under nitrogen for 0.5 h. The mixture was concentrated in vacuo, then partitioned between dichloromethane and water. The layers were separated and the organics concentrated in vacuo to afford Intermediate 17 (0.574 g). LCMS showed MH⁺=315; T_{RET}=2.90 min.

Similarly prepared were the following:

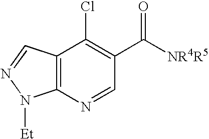

| | | | MH⁺ | T_{RET} |
|---|---|---|---|---|
| | NR⁴R⁵ | Amine reagent | ion | (min) |
| Intermediate 18 | 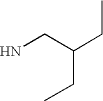 | 2-Ethyl-N-butylamine | 309 | 3.07 |
| Intermediate 19 | 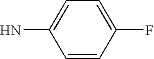 | 4-Fluoroaniline | 319 | 3.08 |
| Intermediate 20 | 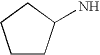 | Cyclopentylamine | 293 | 2.76 |
| Intermediate 21 | 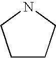 | Pyrrolidine | 279 | 2.46 |

### Intermediate 22: 4-Chloro-1-ethyl-N-(pyridin-4-ylmethyl)-1H-pyrazolo[3,4-b]pyridine-5-carboxamide

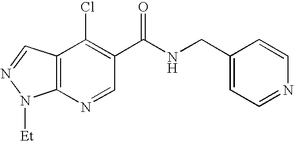

Acid chloride Intermediate 16 was synthesised from Intermediate 15 using the method shown above for Intermediate 17. Intermediate 16 (0.473 g) was dissolved in THF (4 ml) and treated with diisopropylethylamine (DIPEA) (0.509 ml), then with 4-(aminomethyl)pyridine (0.211 g) and the mixture stirred under nitrogen for 0.5 h. The mixture was concentrated in vacuo, then partitioned between DCM and water. The layers were separated and the organics concentrated in vacuo, then applied to an SPE cartridge (silica, 10 g) which was eluted with a gradient of cyclohexane:EtOAc (2:1 increasing stepwise up to 0:1), followed by MeOH:EtOAc (5:95, then 10:90). Fractions containing desired material were combined and concentrated in vacuo to afford Intermediate 22 (0.086 g). LCMS showed MH⁺=316; T_{RET}=1.84 min.

### Intermediate 23: 4-Chloro-1-ethyl-N-n-propyl-1H-pyrazolo[3,4-b]pyridine-5-carboxamide

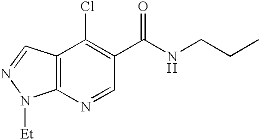

Acid chloride Intermediate 16 was synthesised from Intermediate 15 using the method shown above for Intermediate 17. Intermediate 16 (0.473 g) was dissolved in THF (4 ml) and treated with DIPEA (0.509 ml), then with n-propyl amine (0.115 g) and the mixture stirred under nitrogen for 0.5 h. A further portion of n-propyl amine (0.023 g) was then added and stirring continued for 18 h. The mixture was concentrated in vacuo, then partitioned between DCM and water. The layers were separated and the organics concentrated in vacuo to afford Intermediate 23 (0.405 g). LCMS showed MH⁺=267; T_{RET}=2.54 min.

### Intermediate 24: 4-Chloro-1-ethyl-1H-pyrazolo[3,4-b]pyridine-5-carboxamide

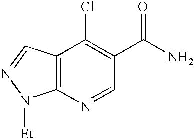

Acid chloride Intermediate 16 was synthesised from Intermediate 15 using the method shown above for Intermediate 17. Intermediate 16 (0.30 g) was dissolved in THF (3 ml) and treated with a 0.5M solution of ammonia in dioxane (4.92 ml). The mixture was stirred under nitrogen for 18 h. A further portion of 0.5M ammonia in dioxane (4.92 ml) was added and stirring continued for 72 h. The mixture was concentrated in vacuo and the residue partitioned between DCM and 2M sodium hydroxide solution. The layers were separated and the organics concentrated to afford Intermediate 24 (0.278 g). LCMS showed MH⁺=225; T_{RET}=2.10 min.

### Intermediate 25: Ethyl 4-chloro-1-methyl-1H-pyrazolo[3,4-b]pyridine-5-carboxylate

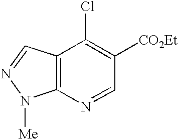

A mixture of 5-amino-1-methylpyrazole (4.0 g) and diethylethoxymethylene malonate (9.16 ml) was heated at 150° C. under Dean Stark conditions for 5 h. Phosphorous oxychloride (55 ml) was carefully added to the mixture and the resulting solution heated at 130° C. under reflux for 18 h. The mixture was concentrated in vacuo, then the residual oil cooled in an ice bath and treated carefully with water (100 ml)(caution: exotherm). The resulting mixture was extracted with DCM (3×100 ml) and the combined organic extracts were dried over anhydrous sodium sulphate and concentrated in vacuo. The residual solid was purified by Biotage chromatography (silica, 90 g), eluting with Et₂0:petrol (1:3). Fractions containing desired material were combined and concentrated in vacuo to afford Intermediate 25 (4.82 g). LCMS showed MH⁺=240; T_{RET}=2.98 min

### Intermediate 26: 4-Chloro-1-methyl-1H-pyrazolo[3,4-b]pyridine-5-carboxylic acid

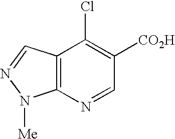

A solution of Intermediate 25 (4.0 g) in dioxane (30 ml) was treated with potassium hydroxide (7.54 g) as a solution in water (20 ml). The mixture was stirred for 16 h, then diluted with water (150 ml) and acidified to pH 3 with 5M aqueous hydrochloric acid. The mixture was stirred in an ice bath for 15 min, then collected by filtration, washed with ice-cold water and dried in vacuo over phosphorous pentoxide to afford Intermediate 26 as a white solid (2.83 g). LCMS showed MH⁺=212; T_{RET}=2.26 min.

### Intermediate 28: N-Benzyl-4-chloro-1-methyl-1H-pyrazolo[3,4-b]pyridine-5-carboxamide

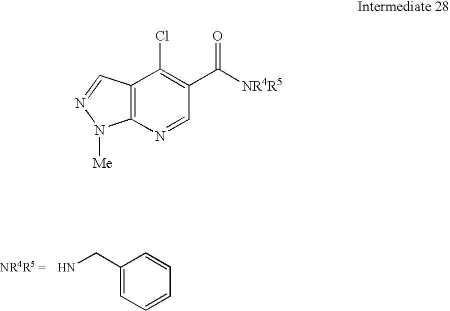

Intermediate 26 (2.5 g) (previously dried over phosphorus pentoxide) was treated with thionyl chloride (25 ml) and the mixture heated under reflux for 1 h. Excess thionyl chloride was removed in vacuo to afford Intermediate 27, presumed to be the acid chloride derivative of Intermediate 26, as a white solid (2.7 g).

Intermediate 27 (0.68 g) was dissolved in THF (10 ml) and treated with DIPEA (0.77 ml), then with benzyl amine (0.339 g) and the mixture stirred under nitrogen for 3 h. The mixture was concentrated in vacuo, then partitioned between DCM (20 ml) and water (10 ml). The layers were separated and the organics concentrated in vacuo to afford Intermediate 28 (0.90 g). LCMS showed MH⁺=301; T_{RET}=2.72 min.

Similarly prepared were the following:

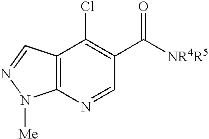

| | | | MH⁺ | T_{RET} |
|---|---|---|---|---|
| | NR⁴R⁵ | Amine reagent | ion | (min) |
| Intermediate 29 | 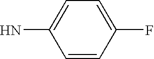 | 4-Fluoroaniline | 305 | 2.91 |
| Intermediate 30 | 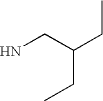 | 2-Ethyl-N-butylamine | 295 | 2.97 |

### Intermediate 31: 4-Chloro-1-methyl-1H-pyrazolo[3,4-b]pyridine-5-carboxamide

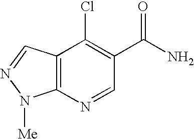

Acid chloride Intermediate 27 was synthesised from Intermediate 26 using the method shown above for Intermediate 28. Intermediate 27 (0.68 g) was then treated with a 0.5M solution of ammonia in dioxane (17.7 ml). Diisopropylethylamine (0.51 ml) was then added and the mixture stirred for 21 h. The mixture was then partitioned between DCM (100 ml) and water (30 ml). An insoluble solid was removed by filtration, washed with water (20 ml) and dried in vacuo over phosphorous pentoxide to afford Intermediate 31 (0.544 g). LCMS showed MH⁺=211; T_{RET}=1.84 min.

### Intermediate 32 (=Example 3): Ethyl 1-ethyl-4-(tetrahydro-2H-pyran-4-ylamino)-1H-pyrazolo[3,4-b]pyridine-5-carboxylate

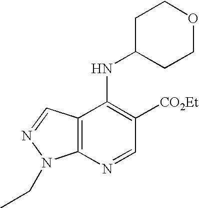

Intermediate 1 (0.20 g) and triethylamine (0.55 ml) were suspended in ethanol (8 ml) and 4-aminotetrahydropyran (0.088 g) was added. The mixture was stirred under nitrogen, heated at 80° C. for 16 h, then concentrated in vacuo. The residue was partitioned between DCM and water. The layers were separated and the organic layer was loaded directly onto an SPE cartridge (silica, 5 g) which was eluted sequentially with; (i) DCM, (ii) DCM:Et₂O (2:1), (iii) DCM:Et₂O (1:1), (iv) Et₂O and (v) EtOAc. Fractions containing desired material were combined and concentrated in vacuo to afford Intermediate 32 (0.21 g). LCMS showed MH⁺=319; T_{RET}=2.93 min.

In an alternative embodiment, Intermediate 32 (=Example 3) can be made as described below under “Example 3”, in particular according to “Example 3, Method B” below.

### Intermediate 33: 1-Ethyl-4-(tetrahydro-2H-pyran-4-ylamino)-1H-pyrazolo[3,4-b]pyridine-5-carboxylic acid

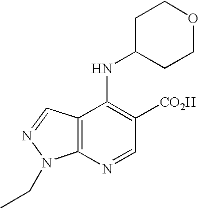

A solution of Intermediate 32 (Example 3) (0.21 g) in ethanol:water (95:5, 10 ml) was treated with sodium hydroxide (0.12 g). The mixture was heated at 50° C. for 8 h, then concentrated in vacuo, dissolved in water and acidified to pH 4 with acetic acid. The resultant white solid was removed by filtration and dried under vacuum to afford Intermediate 33 as an off-white solid (0.156 g). LCMS showed MH⁺=291; T_{RET}=2.11 min.

An alternative preparation of Intermediate 33 is as follows:

A solution of Intermediate 32 (Example 3) (37.8 g) in ethanol:water (4:1, 375 ml) was treated with sodium hydroxide (18.9 g). The mixture was heated at 50° C. for 5 hours, then concentrated in vacuo, dissolved in water and acidified to pH 2 with aqueous hydrochloric acid (2M). The resultant white solid was removed by filtration and dried under vacuum to afford Intermediate 33 as an off-white solid (29.65 g). LCMS showed MH⁺=291; T_{RET}=2.17 min.

### Intermediate 34 (=Example 8): Ethyl 1-ethyl-4-[(3S)-tetrahydrofuran-3-ylamino]-1H-pyrazolo[3,4-b]pyridine-5-carboxylate

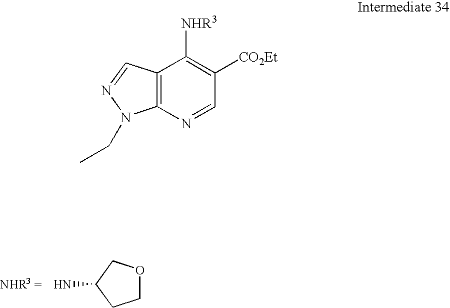

Intermediate 1 (0.05 g) and (S)-(−)-3-aminotetrahydrofuran 4-toluenesulphonate (0.052 g) were suspended in ethanol (1 ml) and triethylamine (0.14 ml) was added. The mixture was stirred under nitrogen and heated at 80° C. for 24 h. After cooling to room temperature, ethanol was removed by evaporation under a stream of nitrogen and the residue partitioned between DCM (2 ml) and water (1.5 ml). The layers were separated and the organic layer concentrated to dryness. Purification was carried out using an SPE cartridge (silica, 5 g), eluting with a gradient of EtOAc:cyclohexane; (1:16 then, 1:8, 1:4, 1:2, 1:1 and 1:0). Fractions containing desired material were combined and concentrated in vacuo to afford Intermediate 34 (=Example 8) (0.052 g). LCMS showed MH⁺=305; T_{RET}=2.70 min.

Similarly prepared were the following:

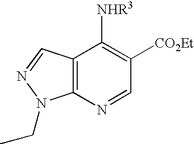

| | | | MH⁺ | T_{RET} |
|---|---|---|---|---|
| | NHR³ | Amine Reagent | ion | (min) |
| Intermediate35 (= Example 9) | 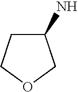 | (R)-(+)-3-Aminotetrahydrofuran4-toluenesulphonate | 305 | 2.73 |
| Intermediate36 (= Example 10) | 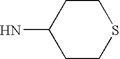 | Intermediate 11 | 335 | 3.21 |
| Intermediate37 (= Example 11) | 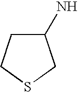 | Intermediate 12 | 321 | 3.10 |
| Intermediate38 (= Example 12) | 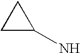 | Cyclopropylamine | 275 | 2.98 |

### Intermediate 39 (=Example 13): Ethyl 4-[(1,1-dioxidotetrahydrothien-3-yl)amino]-1-ethyl-1H-pyrazolo[3,4-b]pyridine-5-carboxylate

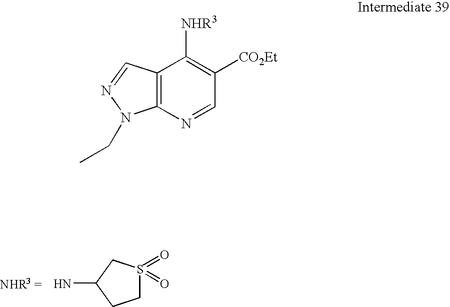

Intermediate 1 (0.05 g) and Intermediate 13 (0.027 g) were suspended in ethanol (1 ml) and triethylamine (0.14 ml) was added. The mixture was stirred under nitrogen and heated at 80° C. for 24 h. After cooling to room temperature, ethanol was removed by evaporation under a stream of nitrogen and the residue partitioned between DCM (2 ml) and water (1.5 ml). The layers were separated and the organic layer concentrated to dryness. Purification was carried out using an SPE cartridge (silica, 5 g), eluting with a gradient of EtOAc:cyclohexane; (1:8 then 1:4, 1:2, 1:1 and 1:0). Fractions containing desired material were combined and concentrated in vacuo to afford Intermediate 39 (=Example 13) (0.045 g) as a mixture of enantiomers. LCMS showed MH⁺=353; T_{RET}=2.60 min.

Similarly prepared was the following:

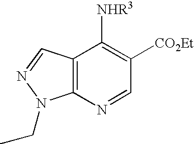

| | | | MH⁺ | T_{RET} |
|---|---|---|---|---|
| | NHR³ | Amine Reagent | ion | (min) |
| Intermediate40(= Example14) | 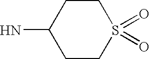 | Intermediate 14 | 367 | 2.64 |

### Intermediate 41: 1-Ethyl-4-[(3S)-tetrahydrofuran-3-ylamino]-1H-pyrazolo[3,4-b]pyridine-5-carboxylic acid

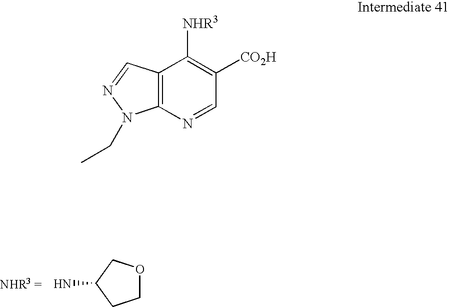

A solution of Intermediate 34 (0.037 g) in ethanol:water (95:5, 3 ml) was treated with sodium hydroxide (0.019 g). The mixture was heated at 50° C. for 16 h, then concentrated in vacuo. The residue was dissolved in water (1.5 ml) and acidified to pH 4 with acetic acid. The resultant white solid precipitate was removed by filtration and dried under vacuum. The filtrate was extracted with ethyl acetate and the organic layer collected and concentrated in vacuo to afford a further portion of white solid. The two solids were combined to afford Intermediate 41 (0.033 g). LCMS showed MH⁺=277; T_{RET}=2.05 min.

Similarly prepared were the following:

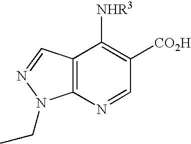

| | | Starting | MH⁺ | T_{RET} |
|---|---|---|---|---|
| | NHR³ | material | ion | (min) |
| Intermediate42 | 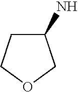 | Intermediate35 | 277 | 2.05 |
| Intermediate43 | 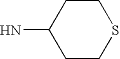 | Intermediate36 | 307 | 2.40 |
| Intermediate44 | 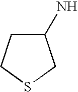 | Intermediate37 | 293 | 2.59 |
| Intermediate45 | 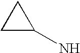 | Intermediate38 | 247 | 2.24 |
| Intermediate46 | 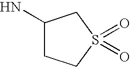 | Intermediate39 | 325 | 2.05 |
| Intermediate47 | 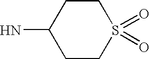 | Intermediate40 | 339 | 2.05 |

### Intermediate 48: Ethyl 4-(cyclohexylamino)-1H-pyrazolo[3,4-b]pyridine-5-carboxylate

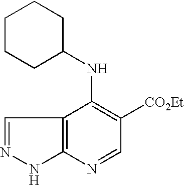

Intermediate 2 (0.69 g) was suspended in cyclohexylamine (1.01 ml), and the mixture was heated at 90° C. for 3 h. The residual mixture was allowed to cool to room temperature and partitioned between chloroform (25 ml) and water (25 ml). The phases were separated and the organic phase was evaporated to dryness. The residue was triturated with Et₂O (25 ml) and the insoluble solid was collected and dried to afford Intermediate 48 as a beige solid (0.58 g). LCMS showed MH⁺=289; T_{RET}=2.91 min.

### Intermediate 49: 4-(Cyclohexylamino)-1H-pyrazolo[3,4-b]pyridine-5-carboxylic acid

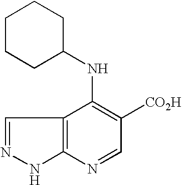

2M-Sodium hydroxide solution (0.5 ml) was added to a stirred suspension of Intermediate 48 (0.2 g) in dioxan (4 ml) and water (0.5 ml). After stirring overnight at room temperature, the reaction mixture was heated at 40° C. for 8 h. A further quantity of 2M-sodium hydroxide solution (1.5 ml) was added, and the reaction mixture was heated at 40° C. for 48 h. The reaction solution was concentrated, diluted with water (10 ml) and acidified with glacial acetic acid. The resulting precipitate was collected by filtration, washed with water and dried to give Intermediate 49 (0.18 g). LCMS showed MH⁺=261; T_{RET}=2.09 min.

### Intermediate 50:1-n-Propyl-4-(tetrahydro-2H-pyran-4-ylamino)-1H-pyrazolo[3,4-b]pyridine-5-carboxylic acid

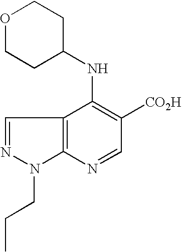

2M-Sodium hydroxide solution (0.7 ml) was added to a stirred suspension of Example 185 (0.23 g, described hereinafter) in ethanol (5 ml) and water (1.5 ml). After stirring overnight at room temperature, a further quantity of 2M-sodium hydroxide solution (0.7 ml) was added, and the reaction mixture was heated at 43° C. for 2.5 h. The reaction solution was concentrated, diluted with water (5 ml) and acidified with 2M-hydrochloric acid. The resulting precipitate was collected by filtration, washed with water and dried to give Intermediate 50 as a white solid (0.14 g). LCMS showed MH⁺=305; T_{RET}=2.42 min.

### Intermediate 51: Ethyl 4-chloro-1-ethyl-6-methyl-1H-pyrazolo[3,4-b]pyridine-5-carboxylate

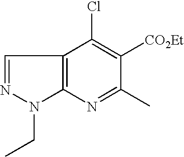

A mixture of 5-amino-1-ethylpyrazole (1.614 g, 14.5 mmol) and diethyl 2-(1-ethoxyethylidene)malonate (3.68 g, 16.0 mmol, as described by P. P. T. Sah, J. Amer. Chem. Soc., 1931, 53, 1836) was heated at 150° C. under Dean Stark conditions for 5 hours. Phosphorous oxychloride (25 ml) was carefully added to the mixture and the resulting solution was heated at 130° C. under reflux for 18 hours. The mixture was concentrated in vacuo, then the residual oil was carefully added, with cooling, to water (100 ml). The resulting mixture was extracted with DCM (3×100 ml) and the combined organic extracts were dried over anhydrous sodium sulphate and concentrated in vacuo. The residual oil was purified by Biotage chromatography (silica, 90 g) eluting with ethyl acetate-petrol (1:19). Fractions containing the desired product were combined and concentrated in vacuo to afford Intermediate 51 (1.15 g). LCMS showed MH⁺=268; T_{RET}=3.18 min.

### Intermediate 52: 4-(Cyclohexylamino)-1-ethyl-6-methyl-1H-pyrazolo[3,4-b]pyridine-5-carboxylic acid

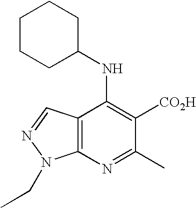

2M-Sodium hydroxide solution (0.39 ml, 0.78 mmol) was added to Example 190 (0.128 g, 0.39 mmol, described hereinafter) in ethanol (1.5 ml), and the mixture was heated at 50° C. for 16 hours. The reaction mixture was concentrated, and the resulting aqueous solution was neutralised with 2M-hydrochloric acid to precipitate a solid which was collected by filtration. The filtrate was applied to an OASIS® hydrophilic-lipophilic balance (HLB) Extraction cartridge * (1 g) which was eluted with water followed by methanol. Evaporation of the methanol fraction gave a solid which was combined with the initial precipitated solid to afford Intermediate 52 (0.083 g) as a white solid, presumed to be the carboxylic acid. * OASIS® HLB Extraction cartridges are available from Waters Corporation, 34 Maple Street, Milford, Mass. 01757, USA. The cartridges include a column containing a copolymer sorbent having a HLB such that when an aqueous solution is eluted through the column, the solute is absorbed or adsorbed into or onto the sorbent, and such that when organic solvent (e.g. methanol) is eluted the solute is released as an organic (e.g. methanol) solution. This is a way to separate the solute from aqueous solvent.

### Intermediate 53: 1-Ethyl-6-methyl-4-(tetrahydro-2H-pyran-4-ylamino)-1H-pyrazolo[3,4-b]pyridine-5-carboxylic acid

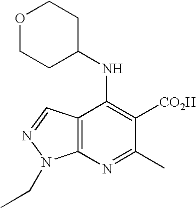

2M-Sodium hydroxide solution (0.75 ml, 1.5 mmol) was added to Example 189 (0.248 g, 0.75 mmol, described hereinafter) in ethanol (2 ml), and the mixture was heated at reflux for 16 hours. The reaction mixture was concentrated, diluted with water (1 ml) and acidified with 2M-hydrochloric acid (0.75 ml) to precipitate a solid which was collected by filtration to afford Intermediate 53 (0.168 g). LCMS showed MH⁺=305; T_{RET}=1.86 min.

### Intermediate 54: 4-Aminocyclohexanone hydrochloride

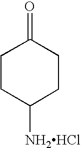

A solution of hydrogen chloride in dioxan (0.5 ml, 2.0 mmol, 4M) was added to a stirred solution of tert-butyl 4-oxocyclohexylcarbamate (0.043 g, 0.20 mmol, commercially available from Astatech Inc., Philadelphia, USA) in dioxan (0.5 ml) and the mixture was stirred at room temperature. After 1 h, the reaction mixture was evaporated to give Intermediate 5 as a cream solid (34 mg). ¹H NMR (400 MHz in d₆-DMSO, 27° C., δppm) 8.09 (br. s, 3H), 3.51 (tt, 11, 3.5 Hz, 1H), 2.45 (m, 2H, partially obscured), 2.29 (m, 2H), 2.16 (m, 2H), 1.76 (m, 2H).

### Intermediate 54A: N-Benzyl-4-(cyclohexylamino)-1H-pyrazolo[3,4-b]pyridine-5-carboxamide

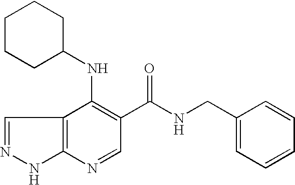

Benzylamine (0.16 ml) was added to a stirred mixture of Intermediate 49 (0.13 g), DIPEA (0.26 ml) and HATU (0.285 g) in DMF (3 ml). The resultant mixture was heated with stirring at 85° C. for 16 hours. Further portions of HATU (0.14 g), DIPEA (0.13 ml) and benzylamine (0.082 ml) were added and the mixture heated for 16 hours at 88° C. The resultant solution was concentrated, diluted with dichloromethane (20 ml) and washed with saturated sodium bicarbonate solution (20 ml), separated by hydrophobic frit and the organic layer concentrated. The residue was purified on a SPE cartridge (silica, 20 g) eluting with 60-80% ethyl acetate in cyclohexane. The residue was purified further on a SPE cartridge (Isolute SCX sulphonic acid cartridge, 5 g ×2), eluting with methanol (2×20 ml) and 10% ammonia in methanol (4×20 ml); the basic fractions were combined and concentrated to give Intermediate 54A as a white solid (0.07 g). LCMS showed MH⁺=350; T_{RET}=2.99 min.

### Intermediate 55: 4-Chloro-1-ethyl-N-{[4-(methyloxy)phenyl]methyl}-1H-pyrazolo[3,4-b]pyridine-5-carboxamide

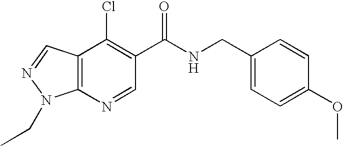

That is, intermediate 55 is:

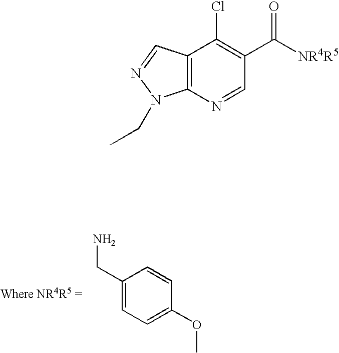

Intermediate 15 (1.04 g) was treated with thionyl chloride (13.22 g). The mixture was stirred and heated at 75° C. for 2 h. Excess thionyl chloride was removed in vacuo and the residual oil azeotroped with toluene to afford Intermediate 16, presumed to be the acid chloride derivative of intermediate 15, as a cream solid (1.12 g).

Intermediate 16 (0.997 g) was dissolved in tetrahydrofuran (THF) (25 ml) and treated with N,N-diisopropylethylamine (1.07 ml) then with 1-[4-(methyloxy)phenyl]methanamine=4-methoxybenzylamine (0.54 ml) (obtainable from e.g. Aldrich, Acros, or Tetrahedron Lett., 2002, 43(48), 8735; or Meindl et al., J. Med. Chem., 1984, 27(9), 1111; or Organic Letters, 2002, 4(12), 2055) and the mixture was stirred for 3 h. The solution was concentrated in vacuo, then partitioned between DCM and water. The layers were separated and the organics concentrated in vacuo. The solid was then triturated in 1:1 ethyl acetate: cyclohexane to give Intermediate 55 (1.27 g). LCMS showed MH⁺=345, T_{RET}=2.86 min.

Similarly prepared were the following:

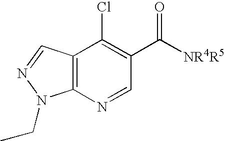

| | | Source of | MH⁺ | T_{RET} |
|---|---|---|---|---|
| | NR⁴R⁵ | HNR⁴R⁵ | ion | (min) |
| Intermediate 56 | 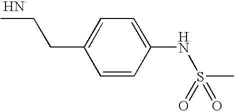 | Lis et al., J. Med.Chem., 1990,33(10), 2883, seeScheme III andref. 24 | 408 | 2.60 |
| Intermediate 57 | 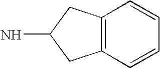 | Maybridge-Int;or Aldrich; orTCI-America | 341 | 3.08 |

### Intermediate 58: 1-Ethyl-4-[(4-oxocyclohexyl)amino]-1H-pyrazolo[3,4-b]pyridine-5-carboxylic acid

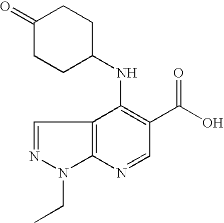

A solution of sodium hydroxide (0.053 g, 1.32 mmol) in water (0.41 ml) was added to a stirred solution of Example 205 (0.1 g, 0.303 mmol) in ethanol (1 ml), and the resulting mixture was heated at 50° C. After 1 h, the cooled reaction mixture was adjusted to pH3 with 2M hydrochloric acid, and extracted with EtOAc (2×6 ml). The combined organic extracts were dried (Na₂SO₄) and evaporated to give Intermediate 58 (0.072 g) as a white solid. LCMS showed MH⁺=303; T_{RET}=2.13 min.

An alternative preparation of Intermediate 58 is as follows:

A solution of sodium hydroxide (0.792 g, 19.8 mmol) in water (6 ml) was added to a stirred solution of Example 205 (1.487 g, 4.5 mmol) in ethanol (15 ml), and the resulting mixture was heated at 50° C. After 1 hour, the cooled reaction mixture was adjusted to pH4 with 2M hydrochloric acid, and extracted with EtOAc (3×30 ml). The combined organic extracts were dried (Na₂SO₄) and evaporated to give Intermediate 58 (1.188 g) as a white solid. LCMS showed MH⁺=303; T_{RET}=2.12 min.

### Intermediate 58A: Ethyl 1-ethyl-4-(tetrahydro-2H-pyran-3-ylamino)-1H-pyrazolo[3,4-b]pyridine-5-carboxylate

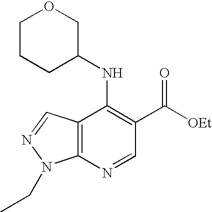

Intermediate 1 (0.76 g, 3.0 mmol)) was dissolved in acetonitrile (10 ml). Tetrahydro-2H-pyran-3-amine hydrochloride (0.5 g, 3.6 mmol, Anales De Quimica, 1988, 84, 148) and N,N-diisopropylethylamine (3.14 ml, 18.0 mmol) were added and the mixture was stirred at 85° C. for 24 h. After 24 h a further portion of tetrahydro-2H-pyran-3-amine hydrochloride (0.14 g, 1.02 mmol) was added and stirring was continued at 85° C. After a further 8 h, the mixture was concentrated in vacuo. The residue was partitioned between DCM (20 ml) and water (12 ml). The layers were separated and the aqueous layer was extracted with further DCM (12 ml). The combined organic extracts were dried (Na₂SO₄), and concentrated in vacuo to give a brown solid which was purified on a SPE cartridge (silica, 20 g) eluting with a gradient of ethyl acetate:cyclohexane (1:16, 1:8, 1:4, 1:2, 1:1, 1:0). Fractions containing the desired material were combined and evaporated to afford Intermediate 58A (0.89 g). LCMS showed MH⁺=319; T_{RET}=2.92 min.

### Intermediate 59: 1-Ethyl-4-(tetrahydro-2H-pyran-3-ylamino)-1H-pyrazolo[3,4-b]pyridine-5-carboxylic acid

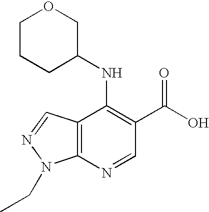

A solution of Intermediate 58A (0.89 g, 2.79 mmol) in ethanol (16.7 ml) was treated with sodium hydroxide (0.47 g, 11.7 mmol) as a solution in water (3.1 ml). The mixture was stirred at 50° C. After 12 h, the reaction mixture was concentrated in vacuo to give a residual oil which was dissolved in water (16 ml), then cooled and acidified to pH 3 with 2M hydrochloric acid. After stirring at 0° C. for 30 min, the resulting precipitate was collected by filtration, washed with cooled water (2 ml) and dried in vacuo to afford Intermediate 59 as a white solid (0.73 g). LCMS showed MH⁺=291; T_{RET}=2.19 min.

### Intermediate 60: 4-[(1-Acetyl-4-piperidinyl)amino]-1-ethyl-1H-pyrazolo[3,4-b]pyridine-5-carboxylic acid

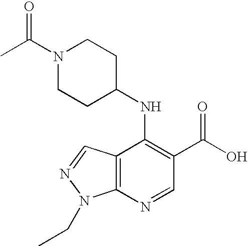

Aqueous sodium hydroxide solution (8.55 ml, 2M) was added to a solution of Example 207 (1.55 g) in EtOH (13 ml). The mixture was heated at 50° C. for 18 h then neutralised using aqueous hydrochloric acid and evaporated in vacuo to afford a mixture of 1-ethyl-4-(4-piperidinylamino)-1H-pyrazolo[3,4-b]pyridine-5-carboxylic acid and 4-[(1-acetyl-4-piperidinyl)amino]-1-ethyl-1H-pyrazolo[3,4-b]pyridine-5-carboxylic acid

Acetic acid (0.36 ml) was added to a stirred mixture of HATU (2.41 g) and N,N-diisopropylethylamine (2.21 ml) in N,N-dimethylformamide (65 ml). After stirring for 15 min the mixture was added to the mixture of 1-ethyl-4-(4-piperidinylamino)-1H-pyrazolo[3,4-b]pyridine-5-carboxylic acid and 4-[(1-acetyl-4-piperidinyl)amino]-1-ethyl-1H-pyrazolo[3,4-b]pyridine-5-carboxylic acid and the reaction stirred for 15 h. The reaction mixture was evaporated in vacuo and the residue purified by chromatography using Biotage (silica 90 g) eluting with DCM:MeOH (0%-5% MeOH) to afford Intermediate 60 (1.36 g) as a white solid. LCMS showed MH⁺=334; T_{RET}=2.06 min.

### Intermediate 61: 4-(Cyclohexylamino)-1-ethyl-1H-pyrazolo[3,4-b]pyridine-5-carboxylic acid

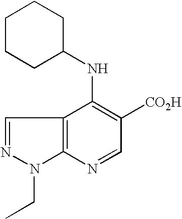

A solution of Example 2 (5.37 g, 17 mmol) in ethanol (30 ml) was treated with a solution of sodium hydroxide (2.72 g, 68 mmol) in water (20 ml), and the resulting mixture was stirred at 50° C. for 3 h. The reaction mixture was concentrated in vacuo, dissolved in water (250 ml) and the cooled solution was acidified to pH 1 with 5M-hydrochloric acid. The resultant solid was collected by filtration and dried in vacuo to afford Intermediate 61 as a white solid (4.7 g). LCMS showed MH⁺=289; T_{RET}=2.83 min.

### Intermediate 62: 1,1-Dimethylethyl (4,4-difluorocyclohexyl)carbamate

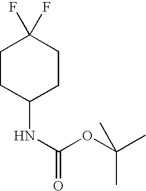

(Diethylamino)sulphur trifluoride (DAST), (0.06 ml, 0.47 mmol), was added to a stirred solution of 1,1-dimethylethyl(4-oxocyclohexyl)carbamate, (250 mg, 1.17 mmol, commercially available from AstaTech Inc., Philadelphia, USA) in anhydrous dichloromethane (5 ml) and the mixture was stirred under nitrogen at 20° C. After 22 h, the reaction mixture was cooled to 0° C., treated with saturated sodium hydrogen carbonate solution (4 ml), and then allowed to warm to ambient temperature. The phases were separated by passage through a hydrophobic frit and the aqueous phase was further extracted with DCM (5 ml). The combined organic phases were concentrated in vacuo to give an orange solid (369 mg) which was further purified by chromatography using a SPE cartridge (silica, 10 g), eluting with DCM to afford Intermediate 62 (140 mg) containing 20% of 1,1-dimethylethyl (4-fluoro-3-cyclohexen-1-yl)carbamate. ¹H NMR (400 MHz in CDCl₃, 27° C., δppm)

Minor component: 65.11 (dm, 16 Hz, 1H), 4.56 (br, 1H), 3.80 (br, 1H) 2.45-1.45 (m's, 6H excess), 1.43 (s, 9H). Major component: 64.43 (br, 1H), 3.58 (br, 1H), 2.45-1.45 (m's, 8H excess), 1.45 (s, 9H).

### Intermediate 63: (4,4-Difluorocyclohexyl)amine hydrochloride

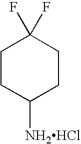

A solution of hydrogen chloride in dioxane (4M, 1.6 ml) was added at 20° C. to a stirred solution of Intermediate 62 (140 mg, 0.6 mmol), in dioxane (1.6 ml). After 3 h, the reaction mixture was concentrated in vacuo to afford intermediate 63 (96.5 mg) containing 4-fluoro-3-cyclohexen-1-amine. ¹H NMR (400 MHz in d₆-DMSO, 27° C., δppm) Minor component: 68.22 (br, 3H excess), 5.18 (dm, 16 Hz, 1H), 3.28-3.13 (m, 1H excess), 2.41-1.53 (m's, 6H excess). Major component: 68.22 (br, 3H excess), 3.28-3.13 (m, 1H excess), 2.41-1.53 (m's, 8H excess). Impurities are also present.

### Intermediate 64: 4-Chloro-1-ethyl-N-methyl-1H-pyrazolo[3,4-b]pyridine-5-carboxamide

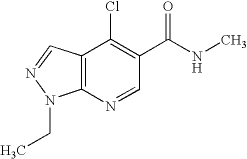

Intermediate 15 (0.06 g, 0.266 mmol) was treated with thionyl chloride (0.48 ml). The mixture was stirred and heated at 75° C. for 2 h. Excess thionyl chloride was removed in vacuo and the residual oil azeotroped with dichloromethane (DCM) to afford Intermediate 16, presumed to be the acid chloride derivative of Intermediate 15, as a white solid. Intermediate 16 was dissolved in anhydrous tetrahydrofuran (THF) (2 ml) and treated with N,N-diisopropylethylamine (DIPEA) (0.069 ml), then with methylamine (2M in tetrahydrofuran, 0.15 ml) and the mixture stirred under nitrogen for 16 h. A further 0.05 ml of methylamine (2M in THF) was added and the solution stirred for 2 h. The mixture was concentrated in vacuo, then partitioned between dichloromethane (2 ml) and aqueous sodium hydroxide solution (2M, 2 ml), then the organic layer washed with water (2 ml). The layers were separated and the organics concentrated in vacuo to afford Intermediate 64 (0.052 g). LCMS showed MH⁺=239; T_{RET}=2.17 min.

### Intermediate 65: Ethyl 4-[(1-{[(1,1-dimethylethyl)oxy]carbonyl}-4-piperidinyl)amino]-1-ethyl-1H-pyrazolo[3,4-b]pyridine-5-carboxylate

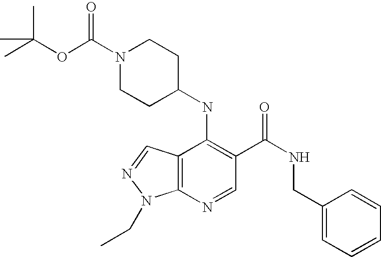

A mixture of Intermediate 17 (2.0 g, 6.37 mmol), 1,1-dimethylethyl 4-amino-1-piperidinecarboxylate (2.04 g, 10.2 mmol) and N,N,-diisopropylethylamine (5.54 ml, 31.9 mmol) in MeCN (40 ml) was heated at 85° C. for 42 h. The reaction was evaporated and the residues partitioned between DCM and water. The organic phase was dried (MgSO₄) then evaporated in vacuo. The residue was chromatographed on silica (Biotage, 90 g) eluting with cyclohexane:EtOAc (1:1) to give Intermediate 65 as a white solid (2.70 g). LCMS showed MH⁺=479; T_{RET}=3.37 min.

### Intermediate 67: 3-Amino-N-cyclohexyl-N-methylbenzamide

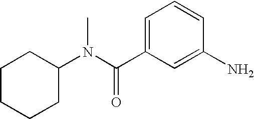

A solution of 3-nitrobenzoyl chloride (2.0 g, 10.78 mmol) in DCM (20 ml) was added dropwise to a stirred mixture of N-methylcyclohexylamine (1.83 ml, 14.01 mmol), N,N,-diisopropylethylamine (3.76 ml, 21.56 mmol) and N,N-dimethylaminopyridine (0.01 g) in DCM at 20° C. The reaction mixture was stirred for 56 h then evaporated in vacuo. The residue was partitioned between ethyl acetate and water. The organic phase was washed with aqueous HCl then dried (MgSO₄) and evaporated in vacuo. The residue was purified by chromatography on silica eluting with cyclohexane:EtOAc (9:1 followed by 2:1) to afford N-cyclohexyl-N-methyl-3-nitrobenzamide (1.40 g). MS showed MH⁺=263.

A mixture of N-Cyclohexyl-N-methyl-3-nitrobenzamide (1.40 g, 5.35 mmol) and palladium on carbon (5%, 0.140 g) in ethanol (10 ml) was stirred under an atmosphere of hydrogen for 1 hour. The reaction mixture was filtered through Celite and the filtrate evaporated to afford Intermediate 67 as a brown solid (0.107 g). LCMS showed MH⁺=233; T_{RET}=2.56 min.

### Intermediate 68: N-Ethyl-4-oxo-1-piperidinecarboxamide

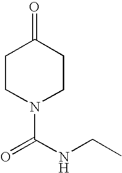

A solution of ethyl isocyanate (2.31 g, 32.5 mmol) in DCM (40 ml) was added, dropwise over 15 min, to a vigorously stirred solution of 4-piperidone monohydrate hydrochloride (5.0 g, 32.5 mmol, commercially available from Aldrich) and sodium hydrogen carbonate (8.2 g, 97.5 mmol) in water (60 ml) at 0° C. The reaction mixture was stirred at room temperature for 20 h. Sodium chloride (7.0 g) was added to the reaction mixture and the organic phase was separated. The aqueous phase was extracted with further DCM (3×75 ml). The combined organic extracts were dried (Na₂SO₄) and evaporated in vacuo to give a white solid (4.0 g). Recrystallisation from ethyl acetate:cyclohexane (10:1) afforded Intermediate 68 as a white solid (2.3 g).

TLC (silica) gave R_{f}=0.24 (ethyl acetate). Anal. Found: C, 56.7; H, 8.3; N, 16.35. C₈H₁₄N₂O₂ requires C, 56.5; H, 8.3; N, 16.5.

### Intermediate 69: 4-Amino-N-ethyl-1-piperidinecarboxamide

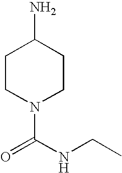

A solution of Intermediate 68 (1.5 g, 8.8 mmol) and benzylamine (1.04 g, 9.7 mmol) in absolute ethanol (60 ml) was hydrogenated over pre-reduced 10% palladium on charcoal catalyst (0.6 g) in ethanol (20 ml) until the uptake of hydrogen had ceased (22 h). The reaction mixture was filtered through filter agent (Celite), and then through silica gel (100 ml) eluting with ethanol:0.88-ammonia (100:1) to give a black oil. The oil was dissolved in ethanol (30 ml) and treated with a solution of hydrogen chloride in ethanol (3M) until the solution was acidic. The solvent was evaporated and the residue was triturated with ethanol to afford Intermediate 69 as a white solid (1.09 g).

TLC (silica) gave R_{f}=0.73 (ethyl acetate:methanol, 10:1). Anal. Found: C, 45.9; H, 8.4; N, 19.8. C₈H₁₈ClN₃O requires C, 46.3; H, 8.7; N, 20.2.

### Intermediate 70:1,1-Dimethylethyl ({4-[(cyclopropylamino)carbonyl]phenyl}methyl)carbamate

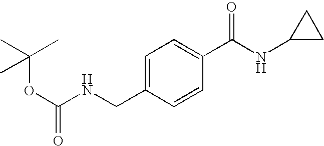

Cyclopropylamine (0.136 g, 2.39 mmol) and diisopropylethylamine (0.68 ml, 3.9 mmol) were added to a stirred solution of 4-[({[(1,1-dimethylethyl)oxy]carbonyl}amino)-methyl]benzoic acid (0.501 g, 2.0 mmol), EDC (0.612 g, 3.2 mmol) and HOBT (0.35 g, 2.6 mmol) in DMF (2 ml). The resulting mixture was stirred at room temperature overnight. Solvents were removed in vacuo, and the residue was dissolved in ethyl acetate (20 ml) and washed with 0.5M-hydrochloric acid (3×20 ml). The organic phase was dried (Na₂SO₄) and evaporated in vacuo to give the crude product which was purified by Biotage chromatography (silica) eluting with ethyl acetate:cyclohexane (1.3:1) to afford Intermediate 70 as a white solid (0.512 g). LCMS showed MH⁺=291; T_{RET}=2.75 min.

### Intermediate 71: 4-(Aminomethyl)-N-cyclopropylbenzamide hydrochloride

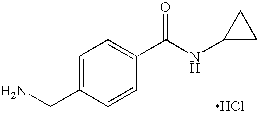

Intermediate 70 (0.506 g, 1.74 mmol) was dissolved in a solution of hydrogen chloride in dioxan (20 ml, 4M) under nitrogen. After 1 h, methanol (3 ml) was added to the mixture and stirring was continued at room temperature overnight. Solvents were removed in vacuo to afford Intermediate 71 as a white solid (0.416 g). LCMS showed MH⁺=191; T_{RET}=0.82 min.

### Intermediate 72

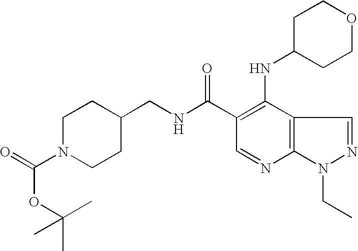

Intermediate 33 (1.36 g, 4.7 mmol), EDC (1.26 g, 6.57 mmol) and HOBT (0.76 g, 5.62 mmol) were suspended in DMF (50 ml) and stirred vigorously at room temperature for 0.5 h, before adding 1,1-dimethylethyl 4-(aminomethyl)-1-piperidinecarboxylate (1.3 g, 6.07 mmol, commercially available from Maybridge Chemical Co. Ltd.,). After stirring at room temperature overnight, a further quantity of 1,1-dimethylethyl 4-(aminomethyl)-1-piperidinecarboxylate (1.01 g, 4.7 mmol) was added to the reaction mixture which was then heated at 50° C. After 6 h, diisopropylethylamine (0.25 ml, 1.44 mmol) was added, and the mixture was maintained at 50° C. for a further 6 h. Solvents were removed in vacuo and the residue was partitioned between DCM (100 ml) and water (100 ml). The phases were separated by passage through a hydrophobic frit, and the organic phase was evaporated in vacuo to give the crude product. Further purification using SPE cartridges (aminopropyl followed by silica) afford Intermediate 72 as a cream solid (1.24 g). LCMS showed MH⁺=487; T_{RET}=2.97 min.

### Intermediate 73

Intermediate 73 is used in situ in the general procedure for Examples 360-414.

### Intermediate 74: 1,1-Dimethylethyl ({3-[(acetylamino)methyl]phenyl}methyl)carbamate

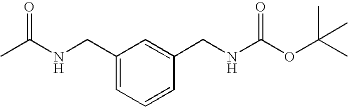

Acetic anhydride (0.52 ml, 5.5 mmol) was added to a mixture of tert-butyl N-[3-aminomethyl)benzyl]carbamate (1.1 g, 4.65 mmol commercially available from Astatech) and triethylamine (0.7 ml, 5 mmol) in THF (20 ml). The reaction mixture was stirred at 20° C. from 16 h then concentrated in vacuo. The residue was partitioned between EtOAc and water. The organic phase was dried (MgSO₄) and evaporated in vacuo. The residue was chromatographed over silica eluting with hexanes:EtOAc (1:1) followed by EtOAc to afford Intermediate 74 (1.2 g) as a colourless oil. Anal. Found: C, 64.79; H, 7.93; N, 10.10. C₁₅H₂₂N₂O₃ requires C, 64.73; H, 7.97; N, 10.06. MS (M+Na)⁺ 301.

### Intermediate 75: N-{[3-(Aminomethyl)phenyl]methyl}acetamide hydrochloride

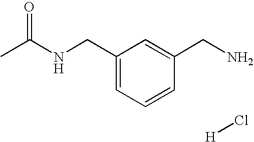

Hydrogen chloride in dioxane (4 ml, 4M) was added to a solution of Intermediate 74 (1.0 g, 3.6 mmol) in dioxane (10 ml) and the resultant mixture stirred for 6 hours at 20° C. The reaction was diluted with Et₂O (20 ml) and filtered to afford Intermediate 75 (0.7 g) as a white solid. MS MH⁺=179. ¹H NMR (300 MHz in d6-DMSO, 27° C., δppm) δ 8.6-8.4 (br m, 3H), 7.38-7.26 (m, 3H), 7.22 (bm, 1H), 4.24 (d, J=5.7 Hz, 2H), 3.95 (dd, J=11.6, 5.7 Hz, 2H), 1.87 (s, 3H).

### Intermediate 76 1-Ethyl-4-{[(1SR,3RS)-3-hydroxycyclohexyl]amino}-1H-pyrazolo[3,4-b]pyridine-5-carboxylic acid

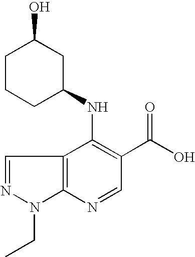

(cis-3-hydroxycyclohex-1-ylamino group, racemic)

A solution of Example 665 (0.681 g, 2.05 mmol) in ethanol (7 ml) was treated with a solution of sodium hydroxide (0.362 g, 9.05 mmol) in water (2.9 ml). The resulting mixture was stirred at 50° C. After 3 h, the reaction mixture was concentrated in vacuo to give a residual oil which was dissolved in water (3 ml), then cooled and acidified to pH 3 with 2M-hydrochloric acid. After stirring at 0° C. for 1 h, the resulting precipitate was collected by filtration, washed with cooled water (0.5 ml) and dried in vacuo to afford Intermediate 76 as a white solid (0.491 g). LCMS showed MH⁺=305; T_{RET}=2.14 min.

Table of Examples

| Example | |
|---|---|
| Number | Name |
| 1 | Ethyl 4-(cyclopentylamino)-1-ethyl-1H-pyrazolo[3,4-b]pyridine-5-carboxylate |
| 2 | Ethyl 4-(cyclohexylamino)-1-ethyl-1H-pyrazolo[3,4-b]pyridine-5-carboxylate |
| 3 | Ethyl 1-ethyl-4-(tetrahydro-2H-pyran-4-ylamino)-1H-pyrazolo[3,4-b]pyridine-5- |
| | carboxylate |
| 5 | Ethyl 4-[(1-acetylpiperidin-4-yl)amino]-1-ethyl-1H-pyrazolo[3,4-b]pyridine-5- |
| | carboxylate |
| 6 | Ethyl 4-(cyclopentylamino)-1-methyl-1H-pyrazolo[3,4-b]pyridine-5-carboxylate |
| 7 | Ethyl 1-methyl-4-(tetrahydro-2H-pyran-4-ylamino)-1H-pyrazolo[3,4-b]pyridine-5- |
| | carboxylate |
| 8 | Ethyl 1-ethyl-4-[(3S)-tetrahydrofuran-3-ylamino]-1H-pyrazolo[3,4-b]pyridine-5- |
| | carboxylate |
| 9 | Ethyl 1-ethyl-4-[(3R)-tetrahydrofuran-3-ylamino]-1H-pyrazolo[3,4-b]pyridine-5- |
| | carboxylate |
| 10 | Ethyl 1-ethyl-4-(tetrahydro-2H-thiopyran-4-ylamino)-1H-pyrazolo[3,4-b]pyridine- |
| | 5-carboxylate |
| 11 | Ethyl 1-ethyl-4-(tetrahydrothien-3-ylamino)-1H-pyrazolo[3,4-b]pyridine-5- |
| | carboxylate |
| 12 | Ethyl 4-(cyclopropylamino)-1-ethyl-1H-pyrazolo[3,4-b]pyridine-5-carboxylate |
| 13 | Ethyl 4-[(1,1-dioxidotetrahydrothien-3-yl)amino]-1-ethyl-1H-pyrazolo[3,4- |
| | b]pyridine-5-carboxylate |
| 14 | Ethyl 4-[(1,1-dioxidotetrahydro-2H-thiopyran-4-yl)amino]-1-ethyl-1H- |
| | pyrazolo[3,4-b]pyridine-5-carboxylate |
| 21 | N-Benzyl-1-ethyl-4-(tetrahydro-2H-pyran-4-ylamino)-1H-pyrazolo[3,4- |
| | b]pyridine-5-carboxamide |
| 22 | 1-Ethyl-N-(4-fluorophenyl)-4-(tetrahydro-2H-pyran-4-ylamino)-1H-pyrazolo[3,4- |
| | b]pyridine-5-carboxamide |
| 23 | N-Cyclopentyl-4-(cyclopentylamino)-1-ethyl-1H-pyrazolo[3,4-b]pyridine-5- |
| | carboxamide |
| 24 | 4-(Cyclohexylamino)-N-cyclopentyl-1-ethyl-1H-pyrazolo[3,4-b]pyridine-5- |
| | carboxamide |
| 25 | N-Cyclopentyl-1-ethyl-4-(tetrahydro-2H-pyran-4-ylamino)-1H-pyrazolo[3,4- |
| | b]pyridine-5-carboxamide |
| 27 | 4-[(1-Acetylpiperidin-4-yl)amino]-N-cyclopentyl-1-ethyl-1H-pyrazolo[3,4- |
| | b]pyridine-5-carboxamide |
| 28 | N-Cyclopentyl-1-ethyl-5-(pyrrolidin-1-ylcarbonyl)-1H-pyrazolo[3,4-b]pyridin-4- |
| | amine |
| 29 | N-Cyclohexyl-1-ethyl-5-(pyrrolidin-1-ylcarbonyl)-1H-pyrazolo[3,4-b]pyridin-4- |
| | amine |
| 30 | 1-Ethyl-5-(pyrrolidin-1-ylcarbonyl)-N-tetrahydro-2H-pyran-4-yl-1H-pyrazolo[3,4- |
| | b]pyridin-4-amine |
| 31 | 4-(Cyclopentylamino)-1-ethyl-N-(pyridin-4-ylmethyl)-1H-pyrazolo[3,4- |
| | b]pyridine-5-carboxamide |
| 32 | 4-(Cyclohexylamino)-1-ethyl-N-(pyridin-4-ylmethyl)-1H-pyrazolo[3,4-b]pyridine- |
| | 5-carboxamide |
| 33 | 1-Ethyl-N-(pyridin-4-ylmethyl)-4-(tetrahydro-2H-pyran-4-ylamino)-1H- |
| | pyrazolo[3,4-b]pyridine-5-carboxamide |
| 34 | 4-(Cyclopentylamino)-1-ethyl-1H-pyrazolo[3,4-b]pyridine-5-carboxamide |
| 35 | 4-(Cyclohexylamino)-1-ethyl-1H-pyrazolo[3,4-b]pyridine-5-carboxamide |
| 36 | 1-Ethyl-4-(tetrahydro-2H-pyran-4-ylamino)-1H-pyrazolo[3,4-b]pyridine-5- |
| | carboxamide |
| 39 | N-Benzyl-4-(cyclopentylamino)-1-ethyl-1H-pyrazolo[3,4-b]pyridine-5- |
| | carboxamide |
| 40 | N-Benzyl-4-(cyclohexylamino)-1-ethyl-1H-pyrazolo[3,4-b]pyridine-5- |
| | carboxamide |
| 41 | 4-[(1-Acetylpiperidin-4-yl)amino]-N-benzyl-1-ethyl-1H-pyrazolo[3,4-b]pyridine- |
| | 5-carboxamide |
| 42 | 4-(Cyclopentylamino)-1-ethyl-N-(2-ethylbutyl)-1H-pyrazolo[3,4-b]pyridine-5- |
| | carboxamide |
| 43 | 4-(Cyclohexylamino)-1-ethyl-N-(2-ethylbutyl)-1H-pyrazolo[3,4-b]pyridine-5- |
| | carboxamide |
| 44 | 1-Ethyl-N-(2-ethylbutyl)-4-(tetrahydro-2H-pyran-4-ylamino)-1H-pyrazolo[3,4- |
| | b]pyridine-5-carboxamide |
| 45 | 1-Ethyl-N-(2-ethylbutyl)-4-[(1-methylpiperidin-4-yl)amino]-1H-pyrazolo[3,4- |
| | b]pyridine-5-carboxamide |
| 46 | 4-[(1-Acetylpiperidin-4-yl)amino]-1-ethyl-N-(2-ethylbutyl)-1H-pyrazolo[3,4- |
| | b]pyridine-5-carboxamide |
| 47 | 4-(Cyclopentylamino)-1-ethyl-N-(4-fluorophenyl)-1H-pyrazolo[3,4-b]pyridine-5- |
| | carboxamide |
| 48 | 4-(Cyclohexylamino)-1-ethyl-N-(4-fluorophenyl)-1H-pyrazolo[3,4-b]pyridine-5- |
| | carboxamide |
| 49 | 1-Ethyl-N-(4-fluorophenyl)-4-[(1-methylpiperidin-4-yl)amino]-1H-pyrazolo[3,4- |
| | b]pyridine-5-carboxamide |
| 50 | 4-[(1-Acetylpiperidin-4-yl)amino]-1-ethyl-N-(4-fluorophenyl)-1H-pyrazolo[3,4- |
| | b]pyridine-5-carboxamide |
| 51 | 4-(Cyclopentylamino)-1-ethyl-N-n-propyl-1H-pyrazolo[3,4-b]pyridine-5- |
| | carboxamide |
| 52 | 4-(Cyclohexylamino)-1-ethyl-N-n-propyl-1H-pyrazolo[3,4-b]pyridine-5- |
| | carboxamide |
| 53 | 1-Ethyl-N-n-propyl-4-(tetrahydro-2H-pyran-4-ylamino)-1H-pyrazolo[3,4- |
| | b]pyridine-5-carboxamide |
| 55 | 4-[(1-Acetylpiperidin-4-yl)amino]-1-ethyl-N-n-propyl-1H-pyrazolo[3,4- |
| | b]pyridine-5-carboxamide |
| 57 | 4-[(1-Acetylpiperidin-4-yl)amino]-1-ethyl-N-(pyridin-4-ylmethyl)-1H- |
| | pyrazolo[3,4-b]pyridine-5-carboxamide |
| 61 | N-Benzyl-4-(cyclopentylamino)-1-methyl-1H-pyrazolo[3,4-b]pyridine-5- |
| | carboxamide |
| 62 | N-Benzyl-4-(cyclohexylamino)-1-methyl-1H-pyrazolo[3,4-b]pyridine-5- |
| | carboxamide |
| 63 | N-Benzyl-1-methyl-4-(tetrahydro-2H-pyran-4-ylamino)-1H-pyrazolo[3,4- |
| | b]pyridine-5-carboxamide |
| 64 | 4-(Cyclopentylamino)-N-(2-ethylbutyl)-1-methyl-1H-pyrazolo[3,4-b]pyridine-5- |
| | carboxamide |
| 65 | 4-(Cyclohexylamino)-N-(2-ethylbutyl)-1-methyl-1H-pyrazolo[3,4-b]pyridine-5- |
| | carboxamide |
| 66 | N-(2-Ethylbutyl)-1-methyl-4-(tetrahydro-2H-pyran-4-ylamino)-1H-pyrazolo[3,4- |
| | b]pyridine-5-carboxamide |
| 67 | 4-(Cyclopentylamino)-N-(4-fluorophenyl)-1-methyl-1H-pyrazolo[3,4-b]pyridine- |
| | 5-carboxamide |
| 68 | 4-(Cyclohexylamino)-N-(4-fluorophenyl)-1-methyl-1H-pyrazolo[3,4-b]pyridine-5- |
| | carboxamide |
| 69 | N-(4-Fluorophenyl)-1-methyl-4-(tetrahydro-2H-pyran-4-ylamino)-1H- |
| | pyrazolo[3,4-b]pyridine-5-carboxamide |
| 70 | 4-(Cyclopentylamino)-1-methyl-1H-pyrazolo[3,4-b]pyridine-5-carboxamide |
| 71 | 4-(Cyclohexylamino)-1-methyl-1H-pyrazolo[3,4-b]pyridine-5-carboxamide |
| 74 | 4-[(1-Acetylpiperidin-4-yl)amino]-N-benzyl-1-methyl-1H-pyrazolo[3,4- |
| | b]pyridine-5-carboxamide |
| 81 | 1-Ethyl-N-methyl-4-(tetrahydro-2H-pyran-4-ylamino)-1H-pyrazolo[3,4- |
| | b]pyridine-5-carboxamide |
| 82 | 1-Ethyl-N,N-dimethyl-4-(tetrahydro-2H-pyran-4-ylamino)-1H-pyrazolo[3,4- |
| | b]pyridine-5-carboxamide |
| 83 | 1-Ethyl-N-ethyl-4-(tetrahydro-2H-pyran-4-ylamino)-1H-pyrazolo[3,4-b]pyridine- |
| | 5-carboxamide |
| 84 | 1-Ethyl-N-isopropyl-4-(tetrahydro-2H-pyran-4-ylamino)-1H-pyrazolo[3,4- |
| | b]pyridine-5-carboxamide |
| 85 | N-Benzyl-1-ethyl-4-[(3S)-tetrahydrofuran-3-ylamino]-1H-pyrazolo[3,4- |
| | b]pyridine-5-carboxamide |
| 86 | N-Benzyl-1-ethyl-4-[(3R)-tetrahydrofuran-3-ylamino]-1H-pyrazolo[3,4- |
| | b]pyridine-5-carboxamide |
| 87 | N-Benzyl-1-ethyl-4-(tetrahydrothien-3-ylamino)-1H-pyrazolo[3,4-b]pyridine-5- |
| | carboxamide |
| 88 | N-Benzyl-4-(cyclopropylamino)-1-ethyl-1H-pyrazolo[3,4-b]pyridine-5- |
| | carboxamide |
| 89 | N-Benzyl-4-[(1,1-dioxidotetrahydrothien-3-yl)amino]-1-ethyl-1H-pyrazolo[3,4- |
| | b]pyridine-5-carboxamide |
| 90 | N-Benzyl-4-[(1,1-dioxidotetrahydro-2H-thiopyran-4-yl)amino]-1-ethyl-1H- |
| | pyrazolo[3,4-b]pyridine-5-carboxamide |
| 91 | N-Benzyl-1-ethyl-4-(tetrahydro-2H-thiopyran-4-ylamino)-1H-pyrazolo[3,4- |
| | b]pyridine-5-carboxamide |
| 92 | 1-Ethyl-N-(4-fluorophenyl)-4-[(3S)-tetrahydrofuran-3-ylamino]-1H-pyrazolo[3,4- |
| | b]pyridine-5-carboxamide |
| 93 | 1-Ethyl-N-(4-fluorophenyl)-4-[(3R)-tetrahydrofuran-3-ylamino]-1H-pyrazolo[3,4- |
| | b]pyridine-5-carboxamide |
| 94 | 1-Ethyl-N-(4-fluorophenyl)-4-(tetrahydro-2H-thiopyran-4-ylamino)-1H- |
| | pyrazolo[3,4-b]pyridine-5-carboxamide |
| 95 | 1-Ethyl-N-(4-fluorophenyl)-4-(tetrahydrothien-3-ylamino)-1H-pyrazolo[3,4- |
| | b]pyridine-5-carboxamide |
| 96 | 4-(Cyclopropylamino)-1-ethyl-N-(4-fluorophenyl)-1H-pyrazolo[3,4-b]pyridine-5- |
| | carboxamide |
| 97 | 4-[(1,1-Dioxidotetrahydrothien-3-yl)amino]-1-ethyl-N-(4-fluorophenyl)-1H- |
| | pyrazolo[3,4-b]pyridine-5-carboxamide |
| 98 | 4-[(1,1-Dioxidotetrahydro-2H-thiopyran-4-yl)amino]-1-ethyl-N-(4-fluorophenyl)- |
| | 1H-pyrazolo[3,4-b]pyridine-5-carboxamide |
| 100 | 1-Ethyl-N-[4-(methylsulfonyl)benzyl]-4-(tetrahydro-2H-pyran-4- |
| | ylamino)-1H-pyrazolo[3,4-b]pyridine-5-carboxamide |
| 102 | 1-Ethyl-N-[3-(methylsulfonyl)benzyl]-4-(tetrahydro-2H-pyran-4- |
| | ylamino)-1H-pyrazolo[3,4-b]pyridine-5-carboxamide |
| 103 | 1-Ethyl-5-{[5-methoxy-6-(trifluoromethyl)-2,3-dihydro-1H-indol-1- |
| | yl]carbonyl}-N-tetrahydro-2H-pyran-4-yl-1H-pyrazolo[3,4-b]pyridin-4- |
| | amine |
| 104 | N-[(5-Chloropyridin-2-yl)methyl]-1-ethyl-4-(tetrahydro-2H-pyran-4- |
| | ylamino)-1H-pyrazolo[3,4-b]pyridine-5-carboxamide |
| 105 | N-(4-Chlorobenzyl)-1-ethyl-N-isopropyl-4-(tetrahydro-2H-pyran-4- |
| | ylamino)-1H-pyrazolo[3,4-b]pyridine-5-carboxamide |
| 106 | N-(3-Chlorobenzyl)-1-ethyl-N-(2-hydroxyethyl)-4-(tetrahydro-2H- |
| | pyran-4-ylamino)-1H-pyrazolo[3,4-b]pyridine-5-carboxamide |
| 107 | 1-Ethyl-N-[(5-methyl-3-phenylisoxazol-4-yl)methyl]-4-(tetrahydro-2H- |
| | pyran-4-ylamino)-1H-pyrazolo[3,4-b]pyridine-5-carboxamide |
| 108 | N-(2-tert-Butoxyethyl)-1-ethyl-4-(tetrahydro-2H-pyran-4-ylamino)-1H- |
| | pyrazolo[3,4-b]pyridine-5-carboxamide |
| 109 | 1-Ethyl-4-(tetrahydro-2H-pyran-4-ylamino)-N-(1,3-thiazol-2-ylmethyl)- |
| | 1H-pyrazolo[3,4-b]pyridine-5-carboxamide |
| 110 | 1-Ethyl-N-(pyrimidin-4-ylmethyl)-4-(tetrahydro-2H-pyran-4-ylamino)- |
| | 1H-pyrazolo[3,4-b]pyridine-5-carboxamide |
| 111 | 1-Ethyl-N-[(2-methyl-1,3-thiazol-4-yl)methyl]-4-(tetrahydro-2H-pyran- |
| | 4-ylamino)-1H-pyrazolo[3,4-b]pyridine-5-carboxamide |
| 112 | N-[3-(tert-Butoxymethyl)benzyl]-1-ethyl-4-(tetrahydro-2H-pyran-4- |
| | ylamino)-1H-pyrazolo[3,4-b]pyridine-5-carboxamide |
| 113 | 1-Ethyl-N-{2-[methyl(methylsulfonyl)amino]ethyl}-4-(tetrahydro-2H- |
| | pyran-4-ylamino)-1H-pyrazolo[3,4-b]pyridine-5-carboxamide |
| 114 | 1-Ethyl-N-(pyrazin-2-ylmethyl)-4-(tetrahydro-2H-pyran-4-ylamino)-1H- |
| | pyrazolo[3,4-b]pyridine-5-carboxamide |
| 115 | 1-Ethyl-5-{[4-(pyridin-2-ylcarbonyl)piperazin-1-yl]carbonyl}-N- |
| | tetrahydro-2H-pyran-4-yl-1H-pyrazolo[3,4-b]pyridin-4-amine |
| 116 | N-(2-Chloro-6-fluorobenzyl)-1-ethyl-4-(tetrahydro-2H-pyran-4- |
| | ylamino)-1H-pyrazolo[3,4-b]pyridine-5-carboxamide |
| 117 | 1-Ethyl-N-[(6-oxo-1,6-dihydropyridin-3-yl)methyl]-4-(tetrahydro-2H- |
| | pyran-4-ylamino)-1H-pyrazolo[3,4-b]pyridine-5-carboxamide |
| 118 | N-[3-(Aminocarbonyl)benzyl]-1-ethyl-4-(tetrahydro-2H-pyran-4- |
| | ylamino)-1H-pyrazolo[3,4-b]pyridine-5-carboxamide |
| 119 | 1-Ethyl-N-{4-[(methylamino)carbonyl]phenyl}-4-(tetrahydro-2H-pyran- |
| | 4-ylamino)-1H-pyrazolo[3,4-b]pyridine-5-carboxamide |
| 120 | 1-Ethyl-N-[2-(1-methyl-1H-imidazol-4-yl)ethyl]-4-(tetrahydro-2H- |
| | pyran-4-ylamino)-1H-pyrazolo[3,4-b]pyridine-5-carboxamide |
| 121 | N-{2-[(Anilinocarbonyl)amino]ethyl}-1-ethyl-4-(tetrahydro-2H-pyran-4- |
| | ylamino)-1H-pyrazolo[3,4-b]pyridine-5-carboxamide |
| 122 | 1-Ethyl-N-(1H-tetraazol-5-ylmethyl)-4-(tetrahydro-2H-pyran-4- |
| | ylamino)-1H-pyrazolo[3,4-b]pyridine-5-carboxamide hydrochloride |
| 123 | 1-Ethyl-4-(tetrahydro-2H-pyran-4-ylamino)-N-[2-(1H-1,2,4-triazol-1- |
| | yl)ethyl]-1H-pyrazolo[3,4-b]pyridine-5-carboxamide |
| 125 | 1-Ethyl-4-(tetrahydro-2H-pyran-4-ylamino)-N-[4- |
| | (trifluoromethyl)phenyl]-1H-pyrazolo[3,4-b]pyridine-5-carboxamide |
| 126 | tert-Butyl 4-({[1-ethyl-4-(tetrahydro-2H-pyran-4-ylamino)-1H- |
| | pyrazolo[3,4-b]pyridin-5-yl]carbonyl}amino)piperidine-1-carboxylate |
| 127 | 1-Ethyl-N-{3-[(methylsulfonyl)amino]propyl}-4-(tetrahydro-2H-pyran- |
| | 4-ylamino)-1H-pyrazolo[3,4-b]pyridine-5-carboxamide |
| 128 | N-[2-(Dimethylamino)benzyl]-1-ethyl-4-(tetrahydro-2H-pyran-4- |
| | ylamino)-1H-pyrazolo[3,4-b]pyridine-5-carboxamide |
| 129 | 1-Ethyl-N-[(1-ethylpyrrolidin-2-yl)methyl]-4-(tetrahydro-2H-pyran-4- |
| | ylamino)-1H-pyrazolo[3,4-b]pyridine-5-carboxamide |
| 130 | 1-Ethyl-N-(tetrahydrofuran-2-ylmethyl)-4-(tetrahydro-2H-pyran-4- |
| | ylamino)-1H-pyrazolo[3,4-b]pyridine-5-carboxamide |
| 131 | 1-ethyl-N-tetrahydro-2H-pyran-4-yl-4-(tetrahydro-2H-pyran-4-ylamino)- |
| | 1H-pyrazolo[3,4-b]pyridine-5-carboxamide |
| 132 | N-{4-[(Dimethylamino)sulfonyl]benzyl}-1-ethyl-4-(tetrahydro-2H- |
| | pyran-4-ylamino)-1H-pyrazolo[3,4-b]pyridine-5-carboxamide |
| 133 | 1-Ethyl-N-{3-[(methylsulfonyl)amino]benzyl}-4-(tetrahydro-2H-pyran- |
| | 4-ylamino)-1H-pyrazolo[3,4-b]pyridine-5-carboxamide |
| 135 | 1-Ethyl-N-(4-methoxyphenyl)-4-(tetrahydro-2H-pyran-4-ylamino)-1H- |
| | pyrazolo[3,4-b]pyridine-5-carboxamide |
| 136 | 1-Ethyl-N-[3-(2-oxopyrrolidin-1-yl)propyl]-4-(tetrahydro-2H-pyran-4- |
| | ylamino)-1H-pyrazolo[3,4-b]pyridine-5-carboxamide |
| 137 | 1-Ethyl-N-[2-(1-methylpyrrolidin-2-yl)ethyl]-4-(tetrahydro-2H-pyran-4- |
| | ylamino)-1H-pyrazolo[3,4-b]pyridine-5-carboxamide |
| 138 | 1-Ethyl-N-(pyridin-3-ylmethyl)-4-(tetrahydro-2H-pyran-4-ylamino)-1H- |
| | pyrazolo[3,4-b]pyridine-5-carboxamide |
| 139 | 1-Ethyl-N-(1-methylpiperidin-4-yl)-4-(tetrahydro-2H-pyran-4-ylamino)- |
| | 1H-pyrazolo[3,4-b]pyridine-5-carboxamide |
| 140 | 1-Ethyl-N-(1-ethylpropyl)-4-(tetrahydro-2H-pyran-4-ylamino)-1H- |
| | pyrazolo[3,4-b]pyridine-5-carboxamide |
| 141 | 1-Ethyl-N-(2-piperidin-1-ylethyl)-4-(tetrahydro-2H-pyran-4-ylamino)- |
| | 1H-pyrazolo[3,4-b]pyridine-5-carboxamide |
| 142 | 1-Ethyl-N-(3-morpholin-4-ylpropyl)-4-(tetrahydro-2H-pyran-4- |
| | ylamino)-1H-pyrazolo[3,4-b]pyridine-5-carboxamide |
| 143 | N-(3-Ethoxypropyl)-1-ethyl-4-(tetrahydro-2H-pyran-4-ylamino)-1H- |
| | pyrazolo[3,4-b]pyridine-5-carboxamide |
| 144 | N-(Cyclohexylmethyl)-1-ethyl-4-(tetrahydro-2H-pyran-4-ylamino)-1H- |
| | pyrazolo[3,4-b]pyridine-5-carboxamide |
| 145 | N-[3-(Dimethylamino)propyl]-1-ethyl-4-(tetrahydro-2H-pyran-4- |
| | ylamino)-1H-pyrazolo[3,4-b]pyridine-5-carboxamide |
| 146 | 1-Ethyl-N-neopentyl-4-(tetrahydro-2H-pyran-4-ylamino)-1H- |
| | pyrazolo[3,4-b]pyridine-5-carboxamide |
| 147 | 1-ethyl-N-(4-methoxybenzyl)-4-(tetrahydro-2H-pyran-4-ylamino)-1H- |
| | pyrazolo[3,4-b]pyridine-5-carboxamide |
| 148 | 1-Ethyl-N-{2-[(phenylsulfonyl)amino]ethyl}-4-(tetrahydro-2H-pyran-4- |
| | ylamino)-1H-pyrazolo[3,4-b]pyridine-5-carboxamide |
| 149 | N-[2-(Acetylamino)ethyl]-1-ethyl-4-(tetrahydro-2H-pyran-4-ylamino)- |
| | 1H-pyrazolo[3,4-b]pyridine-5-carboxamide |
| 150 | 1-Ethyl-N-{2-[(methylsulfonyl)amino]ethyl}-4-(tetrahydro-2H-pyran-4- |
| | ylamino)-1H-pyrazolo[3,4-b]pyridine-5-carboxamide |
| 152 | 1-Ethyl-N-{2-[(2-methoxyphenyl)(methyl)amino]ethyl}-4-(tetrahydro- |
| | 2H-pyran-4-ylamino)-1H-pyrazolo[3,4-b]pyridine-5-carboxamide |
| 153 | 1-Ethyl-N-(2-oxo-2-phenylethyl)-4-(tetrahydro-2H-pyran-4-ylamino)- |
| | 1H-pyrazolo[3,4-b]pyridine-5-carboxamide |
| 154 | N-(2,5-Difluorobenzyl)-1-ethyl-4-(tetrahydro-2H-pyran-4-ylamino)-1H- |
| | pyrazolo[3,4-b]pyridine-5-carboxamide |
| 155 | 1-Ethyl-4-(tetrahydro-2H-pyran-4-ylamino)-N-[4- |
| | (trifluoromethyl)benzyl]-1H-pyrazolo[3,4-b]pyridine-5-carboxamide |
| 156 | N,1-Diethyl-N-propyl-4-(tetrahydro-2H-pyran-4-ylamino)-1H- |
| | pyrazolo[3,4-b]pyridine-5-carboxamide |
| 157 | N-Cyclopropyl-1-ethyl-4-(tetrahydro-2H-pyran-4-ylamino)-1H- |
| | pyrazolo[3,4-b]pyridine-5-carboxamide |
| 158 | N-(2-amino-2-oxoethyl)-1-ethyl-4-(tetrahydro-2H-pyran-4-ylamino)- |
| | 1H-pyrazolo[3,4-b]pyridine-5-carboxamide |
| 159 | 1-Ethyl-N-(3-methoxyphenyl)-4-(tetrahydro-2H-pyran-4-ylamino)-1H- |
| | pyrazolo[3,4-b]pyridine-5-carboxamide |
| 160 | N-(3,4-Difluorobenzyl)-1-ethyl-4-(tetrahydro-2H-pyran-4-ylamino)-1H- |
| | pyrazolo[3,4-b]pyridine-5-carboxamide |
| 161 | Ethyl 3-({[1-ethyl-4-(tetrahydro-2H-pyran-4-ylamino)-1H-pyrazolo[3,4- |
| | b]pyridin-5-yl]carbonyl}amino)propanoate |
| 162 | N-(1-Benzylpiperidin-4-yl)-1-ethyl-4-(tetrahydro-2H-pyran-4-ylamino)- |
| | 1H-pyrazolo[3,4-b]pyridine-5-carboxamide |
| 163 | N-Butyl-4-{[1-ethyl-4-(tetrahydro-2H-pyran-4-ylamino)-1H- |
| | pyrazolo[3,4-b]pyridin-5-yl]carbonyl}piperazine-1-carboxamide |
| 164 | 1-Ethyl-4-(tetrahydro-2H-pyran-4-ylamino)-N-(1,3,4-thiadiazol-2-yl)- |
| | 1H-pyrazolo[3,4-b]pyridine-5-carboxamide |
| 165 | N-(2,3-Dihydro-1H-inden-2-yl)-1-ethyl-4-(tetrahydro-2H-pyran-4- |
| | ylamino)-1H-pyrazolo[3,4-b]pyridine-5-carboxamide |
| 166 | 1-Ethyl-N-[2-(2-oxoimidazolidin-1-yl)ethyl]-4-(tetrahydro-2H-pyran-4- |
| | ylamino)-1H-pyrazolo[3,4-b]pyridine-5-carboxamide |
| 167 | N-(3,4-Dimethoxybenzyl)-1-ethyl-4-(tetrahydro-2H-pyran-4-ylamino)- |
| | 1H-pyrazolo[3,4-b]pyridine-5-carboxamide |
| 168 | N-(3-Chlorobenzyl)-1-ethyl-4-(tetrahydro-2H-pyran-4-ylamino)-1H- |
| | pyrazolo[3,4-b]pyridine-5-carboxamide |
| 169 | 1-Ethyl-5-[(4-methylpiperazin-1-yl)carbonyl]-N-tetrahydro-2H-pyran-4- |
| | yl-1H-pyrazolo[3,4-b]pyridin-4-amine |
| 170 | 1-Ethyl-N-(2-hydroxyethyl)-4-(tetrahydro-2H-pyran-4-ylamino)-1H- |
| | pyrazolo[3,4-b]pyridine-5-carboxamide |
| 171 | 1-Ethyl-5-{[4-(4-methoxyphenyl)piperazin-1-yl]carbonyl}-N-tetrahydro- |
| | 2H-pyran-4-yl-1H-pyrazolo[3,4-b]pyridin-4-amine |
| 172 | 1-Ethyl-N-{4-[(methylsulfonyl)methyl]phenyl}-4-(tetrahydro-2H-pyran- |
| | 4-ylamino)-1H-pyrazolo[3,4-b]pyridine-5-carboxamide |
| 173 | N-[3-(dimethylamino)-3-oxopropyl]-1-ethyl-4-(tetrahydro-2H-pyran-4- |
| | ylamino)-1H-pyrazolo[3,4-b]pyridine-5-carboxamide |
| 174 | 1-Ethyl-N-[(1-methyl-1H-imidazol-5-yl)methyl]-4-(tetrahydro-2H- |
| | pyran-4-ylamino)-1H-pyrazolo[3,4-b]pyridine-5-carboxamide |
| 175 | 1-Ethyl-N-{4-[(methylamino)sulfonyl]phenyl}-4-(tetrahydro-2H-pyran- |
| | 4-ylamino)-1H-pyrazolo[3,4-b]pyridine-5-carboxamide |
| 176 | N-(2-Cyanoethyl)-1-ethyl-4-(tetrahydro-2H-pyran-4-ylamino)-1H- |
| | pyrazolo[3,4-b]pyridine-5-carboxamide |
| 178 | 1-Ethyl-N-[(1-methyl-1H-pyrazol-4-yl)methyl]-4-(tetrahydro-2H-pyran- |
| | 4-ylamino)-1H-pyrazolo[3,4-b]pyridine-5-carboxamide |
| 179 | 1-Ethyl-N-methyl-N-[(1-methyl-1H-imidazol-2-yl)methyl]-4- |
| | (tetrahydro-2H-pyran-4-ylamino)-1H-pyrazolo[3,4-b]pyridine-5- |
| | carboxamide |
| 180 | 1-Ethyl-4-(tetrahydro-2H-pyran-4-ylamino)-N-(2-thien-2-ylethyl)-1H- |
| | pyrazolo[3,4-b]pyridine-5-carboxamide |
| 181 | N-[2-(4-Chlorophenyl)ethyl]-1-ethyl-4-(tetrahydro-2H-pyran-4- |
| | ylamino)-1H-pyrazolo[3,4-b]pyridine-5-carboxamide |
| 182 | 1-Ethyl-N-[2-(2-methoxyphenyl)ethyl]-4-(tetrahydro-2H-pyran-4- |
| | ylamino)-1H-pyrazolo[3,4-b]pyridine-5-carboxamide |
| 183 | Ethyl 4-(cyclohexylamino)-1-(3-ethoxy-3-oxopropyl)-1H- |
| | pyrazolo[3,4-b]pyridine-5-carboxylate |
| 185 | Ethyl 1-n-propyl-4-(tetrahydro-2H-pyran-4-ylamino)-1H-pyrazolo[3,4- |
| | b]pyridine-5-carboxylate |
| 186 | Ethyl 1-(2-hydroxyethyl)-4-(tetrahydro-2H-pyran-4-ylamino)-1H- |
| | pyrazolo[3,4-b]pyridine-5-carboxylate |
| 187 | N-[4-(Methylsulfonyl)benzyl]-1-n-propyl-4-(tetrahydro-2H-pyran-4- |
| | ylamino)-1H-pyrazolo[3,4-b]pyridine-5-carboxamide |
| 188 | N-(4-Fluorophenyl)-1-n-propyl-4-(tetrahydro-2H-pyran-4-ylamino)-1H- |
| | pyrazolo[3,4-b]pyridine-5-carboxamide |
| 189 | Ethyl 1-ethyl-6-methyl-4-(tetrahydro-2H-pyran-4-ylamino)-1H- |
| | pyrazolo[3,4-b]pyridine-5-carboxylate |
| 190 | Ethyl 4-(cyclohexylamino)-1-ethyl-6-methyl-1H-pyrazolo[3,4- |
| | b]pyridine-5-carboxylate |
| 191 | 4-(Cyclohexylamino)-1-ethyl-6-methyl-N-[4-(methylsulfonyl)benzyl]- |
| | 1H-pyrazolo[3,4-b]pyridine-5-carboxamide |
| 192 | N-Benzyl-4-(cyclohexylamino)-1-ethyl-6-methyl-1H-pyrazolo[3,4- |
| | b]pyridine-5-carboxamide |
| 193 | 4-(Cyclohexylamino)-1-ethyl-N-(4-fluorophenyl)-6-methyl-1H- |
| | pyrazolo[3,4-b]pyridine-5-carboxamide |
| 194 | 4-(Cyclohexylamino)-1-ethyl-6-methyl-N-[4-(trifluoromethyl)benzyl]- |
| | 1H-pyrazolo[3,4-b]pyridine-5-carboxamide |
| 195 | 4-(Cyclohexylamino)-N-(2,3-dihydro-1H-inden-2-yl)-1-ethyl-6-methyl- |
| | 1H-pyrazolo[3,4-b]pyridine-5-carboxamide |
| 196 | N-Benzyl-1-ethyl-6-methyl-4-(tetrahydro-2H-pyran-4-ylamino)-1H- |
| | pyrazolo[3,4-b]pyridine-5-carboxamide |
| 197 | N-Benzyl-1-ethyl-4-[(2-oxoazepan-3-yl)amino]-1H-pyrazolo[3,4- |
| | b]pyridine-5-carboxamide |
| 198 | N-Benzyl-1-ethyl-4-[(3-hydroxycyclohexyl)amino]-1H-pyrazolo[3,4- |
| | b]pyridine-5-carboxamide; also called |
| | N-benzyl-1-ethyl-4-[(3-hydroxycyclohexan-1-yl)amino]-1H- |
| | pyrazolo[3,4-b]pyridine-5-carboxamide |
| 199 | N-Benzyl-1-ethyl-4-[(4-hydroxycyclohexyl)amino]-1H-pyrazolo[3,4- |
| | b]pyridine-5-carboxamide; also called |
| | N-benzyl-1-ethyl-4-[(4-hydroxycyclohexan-1-yl)amino]-1H- |
| | pyrazolo[3,4-b]pyridine-5-carboxamide |
| 200 | N-Benzyl-1-ethyl-4-[(3-hydroxycyclopentyl)amino]-1H-pyrazolo[3,4- |
| | b]pyridine-5-carboxamide; also called |
| | N-benzyl-1-ethyl-4-[(3-hydroxycyclopentan-1-yl)amino]-1H- |
| | pyrazolo[3,4-b]pyridine-5-carboxamide |
| 201 | N-Benzyl-1-ethyl-4-[(4-oxocyclohexyl)amino]-1H-pyrazolo[3,4- |
| | b]pyridine-5-carboxamide; also called |
| | N-Benzyl-1-ethyl-4-[(4-oxocyclohexan-1-yl)amino]-1H-pyrazolo[3,4- |
| | b]pyridine-5-carboxamide |
| 202 | 1-Ethyl-N-(2-hydroxy-1-methylethyl)-4-(tetrahydro-2H-pyran-4- |
| | ylamino)-1H-pyrazolo[3,4-b]pyridine-5-carboxamide |
| 203 | Methyl (2S)-2-({[1-ethyl-4-(tetrahydro-2H-pyran-4-ylamino)-1H- |
| | pyrazolo[3,4-b]pyridin-5-yl]carbonyl}amino)-3-hydroxypropanoate |

Example no. Name
204 Ethyl 1-ethyl-4-[(4-hydroxycyclohexyl)amino]-1H-pyrazolo[3,4-b]pyridine-5-carboxylate
205 Ethyl 1-ethyl-4-[(4-oxocyclohexyl)amino]-1H-pyrazolo[3,4-b]pyridine-5-carboxylate
207 Ethyl 4-[(1-acetyl-4-piperidinyl)amino]-1-ethyl-1H-pyrazolo[3,4-b]pyridine-5-carboxylate
209 Ethyl 4-[(4-aminocyclohexyl)amino]-1-ethyl-1H-pyrazolo[3,4-b]pyridine-5-carboxylate
210 Ethyl-N-[(1-oxido-3-pyridinyl)methyl]-4-(tetrahydro-2H-pyran-4-ylamino)-1H-pyrazolo[3,4-b]pyridine-5-carboxamide
211 1-Ethyl-N-[(1-oxido-2-pyridinyl)methyl]-4-(tetrahydro-2H-pyran-4-ylamino)-1H-pyrazolo[3,4-b]pyridine-5-carboxamide
212 1-Ethyl-N-[(1-oxido-4-pyridinyl)methyl]-4-(tetrahydro-2H-pyran-4-ylamino)-1H-pyrazolo[3,4-b]pyridine-5-carboxamide
214 4-[(cis-4-Aminocyclohexyl)amino]-1-ethyl-N-(phenylmethyl)-1H-pyrazolo[3,4-b]pyridine-5-carboxamide
221 4-(Cyclobutylamino)-1-ethyl-N-(phenylmethyl)-1H-pyrazolo[3,4-b]pyridine-5-carboxamide
222 4-(Cycloheptylamino)-1-ethyl-N-(phenylmethyl)-1H-pyrazolo[3,4-b]pyridine-5-carboxamide
223 1-Ethyl-4-[(4-methylcyclohexyl)amino]-N-(phenylmethyl)-1H-pyrazolo[3,4-b]pyridine-5-carboxamide
224 1-Ethyl-4-[(3-methylcyclohexyl)amino]-N-(phenylmethyl)-1H-pyrazolo[3,4-b]pyridine-5-carboxamide
225 1-Ethyl-4-[(1-methylcyclohexyl)amino]-N-(phenylmethyl)-1H-pyrazolo[3,4-b]pyridine-5-carboxamide
226 4-[(1R,2R,4S)-Bicyclo[2.2.1]hept-2-ylamino]-1-ethyl-N-(phenylmethyl)-1H-pyrazolo[3,4-b]pyridine-5-carboxamide
227 4-[(1R,2S,4S)-Bicyclo[2.2.1]hept-2-ylamino]-1-ethyl-N-(phenylmethyl)-1H-pyrazolo[3,4-b]pyridine-5-carboxamide
228 1-Ethyl-4-{[(3S)-2-oxo-3-pyrrolidinyl]amino}-N-(phenylmethyl)-1H-pyrazolo[3,4-b]pyridine-5-carboxamide
229 4-[(2,5-Dioxo-3-pyrrolidinyl)amino]-1-ethyl-N-(phenylmethyl)-1H-pyrazolo[3,4-b]pyridine-5-carboxamide
230 4-(1-Azabicyclo[2.2.2]oct-3-ylamino)-1-ethyl-N-(phenylmethyl)-1H-pyrazolo[3,4-b]pyridine-5-carboxamide
231 1-Ethyl-4-[(1-methylcyclohexyl)amino]-N-{[4-(methyloxy)phenyl]methyl}-1H-pyrazolo[3,4-b]pyridine-5-carboxamide
233 4-(Cyclobutylamino)-1-ethyl-N-{[4-(methyloxy)phenyl]methyl}-1H-pyrazolo[3,4-b]pyridine-5-carboxamide
234 4-(Cycloheptylamino)-1-ethyl-N-{[4-(methyloxy)phenyl]methyl}-1H-pyrazolo[3,4-b]pyridine-5-carboxamide
235 4-[(1R,2R,4S)-Bicyclo[2.2.1]hept-2-ylamino]-1-ethyl-N-{[4-(methyloxy)phenyl]methyl}-1H-pyrazolo[3,4-b]pyridine-5-carboxamide
236 1-Ethyl-4-[(4-methylcyclohexyl)amino]-N-{[4-(methyloxy)phenyl]methyl}-1H-pyrazolo[3,4-b]pyridine-5-carboxamide
237 1-Ethyl-4-[(3-methylcyclohexyl)amino]-N-{[4-(methyloxy)phenyl]methyl}-1H-pyrazolo[3,4-b]pyridine-5-carboxamide
238 4-[(1R,2S,4S)-Bicyclo[2.2.1]hept-2-ylamino]-1-ethyl-N-{[4-(methyloxy)phenyl]methyl}-1H-pyrazolo[3,4-b]pyridine-5-carboxamide
239 4-[(cis-4-Aminocyclohexyl)amino]-1-ethyl-N-{[4-(methyloxy)phenyl]methyl}-1H-pyrazolo[3,4-b]pyridine-5-carboxamide
240 4-(Cycloheptylamino)-1-ethyl-N-({4-[(methylsulfonyl)amino]phenyl}methyl)-1H-pyrazolo[3,4-b]pyridine-5-carboxamide
241 4-(Cyclobutylamino)-1-ethyl-N-({4-[(methylsulfonyl)amino]phenyl}methyl)-1H-pyrazolo[3,4-b]pyridine-5-carboxamide
242 4-[(1R,2R,4S)-Bicyclo[2.2.1]hept-2-ylamino]-1-ethyl-N-({4-[(methylsulfonyl)amino]phenyl}methyl)-1H-pyrazolo[3,4-b]pyridine-5-carboxamide
243 4-[(1R,2S,4S)-Bicyclo[2.2.1]hept-2-ylamino]-1-ethyl-N-({4-[(methylsulfonyl)amino]phenyl}methyl)-1H-pyrazolo[3,4-b]pyridine-5-carboxamide
244 1-Ethyl-4-[(4-methylcyclohexyl)amino]-N-({4-[(methylsulfonyl)amino]phenyl}methyl)-1H-pyrazolo[3,4-b]pyridine-5-carboxamide
245 1-Ethyl-4-[(3-methylcyclohexyl)amino]-N-({4-[(methylsulfonyl)amino]phenyl}methyl)-1H-pyrazolo[3,4-b]pyridine-5-carboxamide
247 1-Ethyl-4-[(1-methylcyclohexyl)amino]-N-({4-[(methylsulfonyl)amino]phenyl}methyl)-1H-pyrazolo[3,4-b]pyridine-5-carboxamide
248 4-[(cis-4-Aminocyclohexyl)amino]-1-ethyl-N-({4-[(methylsulfonyl)amino]phenyl}methyl)-1H-pyrazolo[3,4-b]pyridine-5-carboxamide
249 4-(Cyclohexylamino)-1-ethyl-N-({4-[(methylsulfonyl)amino]phenyl}methyl)-1H-pyrazolo[3,4-b]pyridine-5-carboxamide
250 4-(Cycloheptylamino)-N-(2,3-dihydro-1H-inden-2-yl)-1-ethyl-1H-pyrazolo[3,4-b]pyridine-5-carboxamide
251 4-(Cyclobutylamino)-N-(2,3-dihydro-1H-inden-2-yl)-1-ethyl-1H-pyrazolo[3,4-b]pyridine-5-carboxamide
253 N-(2,3-Dihydro-1H-inden-2-yl)-1-ethyl-4-[(3-methylcyclohexyl)amino]-1H-pyrazolo[3,4-b]pyridine-5-carboxamide
254 N-(2,3-Dihydro-1H-inden-2-yl)-1-ethyl-4-[(4-methylcyclohexyl)amino]-1H-pyrazolo[3,4-b]pyridine-5-carboxamide
255 4-[(1R,2R,4S)-Bicyclo[2.2.1]hept-2-ylamino]-N-(2,3-dihydro-1H-inden-2-yl)-1-ethyl-1H-pyrazolo[3,4-b]pyridine-5-carboxamide
256 4-[(1R,2S,4S)-Bicyclo[2.2.1]hept-2-ylamino]-N-(2,3-dihydro-1H-inden-2-yl)-1-ethyl-1H-pyrazolo[3,4-b]pyridine-5-carboxamide
257 N-(2,3-Dihydro-1H-inden-2-yl)-1-ethyl-4-[(1-methylcyclohexyl)amino]-1H-pyrazolo[3,4-b]pyridine-5-carboxamide
258 4-[(cis-4-Aminocyclohexyl)amino]-N-(2,3-dihydro-1H-inden-2-yl)-1-ethyl-1H-pyrazolo[3,4-b]pyridine-5-carboxamide
259 1-Ethyl-N-{4-[(methylsulfonyl)methyl]phenyl}-4-[(4-oxocyclohexyl)amino]-1H-pyrazolo[3,4-b]pyridine-5-carboxamide
260 N-[(2,4-Dimethylphenyl)methyl]-1-ethyl-4-[(4-oxocyclohexyl)amino]-1H-pyrazolo[3,4-b]pyridine-5-carboxamide
261 N-[(3,4-Dimethylphenyl)methyl]-1-ethyl-4-[(4-oxocyclohexyl)amino]-1H-pyrazolo[3,4-b]pyridine-5-carboxamide
262 N-[(3,4-Dichlorophenyl)methyl]-1-ethyl-4-[(4-oxocyclohexyl)amino]-1H-pyrazolo[3,4-b]pyridine-5-carboxamide
263 1-Ethyl-N-{[4-(methyloxy)phenyl]methyl}-4-[(4-oxocyclohexyl)amino]-1H-pyrazolo[3,4-b]pyridine-5-carboxamide
264 1-Ethyl-N-({4-[(methylsulfonyl)amino]phenyl}methyl)-4-[(4-oxocyclohexyl)amino]-1H-pyrazolo[3,4-b]pyridine-5-carboxamide
265 N-{[4-(Dimethylamino)phenyl]methyl}-1-ethyl-4-[(4-oxocyclohexyl)amino]-1H-pyrazolo[3,4-b]pyridine-5-carboxamide
266 N-({4-[(Difluoromethyl)oxy]phenyl}methyl)-1-ethyl-4-[(4-oxocyclohexyl)amino]-1H-pyrazolo[3,4-b]pyridine-5-carboxamide
267 1-Ethyl-4-[(4-oxocyclohexyl)amino]-N-{[4-(trifluoromethyl)phenyl]methyl}-1H-pyrazolo[3,4-b]pyridine-5-carboxamide
268 1-Ethyl-N-{[4-(methylsulfonyl)phenyl]methyl}-4-[(4-oxocyclohexyl)amino]-1H-pyrazolo[3,4-b]pyridine-5-carboxamide
269 1-Ethyl-N-(4-fluorophenyl)-4-[(4-oxocyclohexyl)amino]-1H-pyrazolo[3,4-b]pyridine-5-carboxamide
270 1-Ethyl-4-[(4-oxocyclohexyl)amino]-N-(2-pyridinylmethyl)-1H-pyrazolo[3,4-b]pyridine-5-carboxamide trifluoroacetate
271 N-(2,3-Dihydro-1H-inden-2-yl)-1-ethyl-4-[(4-oxocyclohexyl)amino]-1H-pyrazolo[3,4-b]pyridine-5-carboxamide
272 N-(1-Acetyl-4-piperidinyl)-1-ethyl-4-[(4-oxocyclohexyl)amino]-1H-pyrazolo[3,4-b]pyridine-5-carboxamide
273 1-Ethyl-N-[(1-methyl-1H-pyrazol-4-yl)methyl]-4-[(4-oxocyclohexyl)amino]-1H-pyrazolo[3,4-b]pyridine-5-carboxamide
274 N,1-Diethyl-4-[(4-oxocyclohexyl)amino]-1H-pyrazolo[3,4-b]pyridine-5-carboxamide
275 1-Ethyl-4-[(4-oxocyclohexyl)amino]-N-(1,3-thiazol-2-ylmethyl)-1H-pyrazolo[3,4-b]pyridine-5-carboxamide
276 1-Ethyl-N-(phenylmethyl)-4-(tetrahydro-2H-pyran-3-ylamino)-1H-pyrazolo[3,4-b]pyridine-5-carboxamide
277 N-({4-[(Difluoromethyl)oxy]phenyl}methyl)-1-ethyl-4-(tetrahydro-2H-pyran-3-ylamino)-1H-pyrazolo[3,4-b]pyridine-5-carboxamide
278 1-Ethyl-4-(tetrahydro-2H-pyran-3-ylamino)-N-{[4-(trifluoromethyl)phenyl]methyl}-1H-pyrazolo[3,4-b]pyridine-5-carboxamide
279 1-Ethyl-N-{[4-(methylsulfonyl)phenyl]methyl}-4-(tetrahydro-2H-pyran-3-ylamino)-1H-pyrazolo[3,4-b]pyridine-5-carboxamide
280 1-Ethyl-N-{4-[(methylsulfonyl)methyl]phenyl}-4-(tetrahydro-2H-pyran-3-ylamino)-1H-pyrazolo[3,4-b]pyridine-5-carboxamide
281 1-Ethyl-N-(4-fluorophenyl)-4-(tetrahydro-2H-pyran-3-ylamino)-1H-pyrazolo[3,4-b]pyridine-5-carboxamide
282 1-Ethyl-N-(2-pyridinylmethyl)-4-(tetrahydro-2H-pyran-3-ylamino)-1H-pyrazolo[3,4-b]pyridine-5-carboxamide trifluoroacetate
283 N-(2,3-Dihydro-1H-inden-2-yl)-1-ethyl-4-(tetrahydro-2H-pyran-3-ylamino)-1H-pyrazolo[3,4-b]pyridine-5-carboxamide
284 N-(1-Acetyl-4-piperidinyl)-1-ethyl-4-(tetrahydro-2H-pyran-3-ylamino)-1H-pyrazolo[3,4-b]pyridine-5-carboxamide
285 1-Ethyl-N-[(1-methyl-1H-pyrazol-4-yl)methyl]-4-(tetrahydro-2H-pyran-3-ylamino)-1H-pyrazolo[3,4-b]pyridine-5-carboxamide
286 N,1-Diethyl-4-(tetrahydro-2H-pyran-3-ylamino)-1H-pyrazolo[3,4-b]pyridine-5-carboxamide
287 1-Ethyl-4-(tetrahydro-2H-pyran-3-ylamino)-N-(1,3-thiazol-2-ylmethyl)-1H-pyrazolo[3,4-b]pyridine-5-carboxamide
288 4-[(4,4-Difluorocyclohexyl)amino]-1-ethyl-N-(phenylmethyl)-1H-pyrazolo[3,4-b]pyridine-5-carboxamide
289 1-Ethyl-4-[(4-fluoro-3-cyclohexen-1-yl)amino]-N-(phenylmethyl)-1H-pyrazolo[3,4-b]pyridine-5-carboxamide.
290 4-[(1-Acetyl-4-piperidinyl)amino]-N-(2,3-dihydro-1H-inden-2-yl)-1-ethyl-1H-pyrazolo[3,4-b]pyridine-5-carboxamide
291 4-[(1-Acetyl-4-piperidinyl)amino]-N-[(3,4-dichlorophenyl)methyl]-1-ethyl-1H-pyrazolo[3,4-b]pyridine-5-carboxamide
292 4-[(1-Acetyl-4-piperidinyl)amino]-1-ethyl-N-[(3-fluorophenyl)methyl]-1H-pyrazolo[3,4-b]pyridine-5-carboxamide
293 4-[(1-Acetyl-4-piperidinyl)amino]-N-[(3,4-difluorophenyl)methyl]-1-ethyl-1H-pyrazolo[3,4-b]pyridine-5-carboxamide
294 4-[(1-Acetyl-4-piperidinyl)amino]-N-[(2,5-difluorophenyl)methyl]-1-ethyl-1H-pyrazolo[3,4-b]pyridine-5-carboxamide
295 4-[(1-Acetyl-4-piperidinyl)amino]-1-ethyl-N-{[3-(trifluoromethyl)phenyl]methyl}-1H-pyrazolo[3,4-b]pyridine-5-carboxamide
296 4-[(1-Acetyl-4-piperidinyl)amino]-1-ethyl-N-{[4-(trifluoromethyl)phenyl]methyl}-1H-pyrazolo[3,4-b]pyridine-5-carboxamide
297 4-[(1-Acetyl-4-piperidinyl)amino]-N-[(2,6-difluorophenyl)methyl]-1-ethyl-1H-pyrazolo[3,4-b]pyridine-5-carboxamide
298 4-[(1-Acetyl-4-piperidinyl)amino]-N-[(3-chlorophenyl)methyl]-1-ethyl-1H-pyrazolo[3,4-b]pyridine-5-carboxamide
299 4-[(1-Acetyl-4-piperidinyl)amino]-1-ethyl-N-{[4-(methyloxy)phenyl]methyl}-1H-pyrazolo[3,4-b]pyridine-5-carboxamide
300 4-[(1-Acetyl-4-piperidinyl)amino]-1-ethyl-N-[4-(methyloxy)phenyl]-1H-pyrazolo[3,4-b]pyridine-5-carboxamide
301 4-[(1-Acetyl-4-piperidinyl)amino]-N-({4-[(dimethylamino)sulfonyl]phenyl}methyl)-1-ethyl-1H-pyrazolo[3,4-b]pyridine-5-carboxamide
302 4-[(1-Acetyl-4-piperidinyl)amino]-1-ethyl-N-(1,2,3,4-tetrahydro-1-naphthalenyl)-1H-pyrazolo[3,4-b]pyridine-5-carboxamide
303 4-[(1-Acetyl-4-piperidinyl)amino]-N-{[2-(dimethylamino)phenyl]methyl}-1-ethyl-1H-pyrazolo[3,4-b]pyridine-5-carboxamide
304 4-[(1-Acetyl-4-piperidinyl)amino]-N-[(2,4-dichlorophenyl)methyl]-1-ethyl-1H-pyrazolo[3,4-b]pyridine-5-carboxamide
305 4-[(1-Acetyl-4-piperidinyl)amino]-1-ethyl-N-[(2-fluorophenyl)methyl]-1H-pyrazolo[3,4-b]pyridine-5-carboxamide
306 4-[(1-Acetyl-4-piperidinyl)amino]-N-[(2-chloro-6-fluorophenyl)methyl]-1-ethyl-1H-pyrazolo[3,4-b]pyridine-5-carboxamide
307 4-[(1-Acetyl-4-piperidinyl)amino]-N-({4-[(difluoromethyl)oxy]phenyl}methyl)-1-ethyl-1H-pyrazolo[3,4-b]pyridine-5-carboxamide
308 4-[(1-Acetyl-4-piperidinyl)amino]-N-{[3-chloro-4-(methyloxy)phenyl]methyl}-1-ethyl-1H-pyrazolo[3,4-b]pyridine-5-carboxamide
309 4-[(1-Acetyl-4-piperidinyl)amino]-N-[(5-chloro-2-pyridinyl)methyl]-1-ethyl-1H-pyrazolo[3,4-b]pyridine-5-carboxamide
310 4-[(1-Acetyl-4-piperidinyl)amino]-N-(5-chloro-2,3-dihydro-1H-inden-2-yl)-1-ethyl-1H-pyrazolo[3,4-b]pyridine-5-carboxamide
311 4-[(1-Acetyl-4-piperidinyl)amino]-1-ethyl-N-(1,3-thiazol-2-ylmethyl)-1H-pyrazolo[3,4-b]pyridine-5-carboxamide
312 4-[(1-Acetyl-4-piperidinyl)amino]-1-ethyl-N-{[4-(methylsulfonyl)phenyl]methyl}-1H-pyrazolo[3,4-b]pyridine-5-carboxamide
313 4-[(1-Acetyl-4-piperidinyl)amino]-N-(2,2-diphenylethyl)-1-ethyl-1H-pyrazolo[3,4-b]pyridine-5-carboxamide
314 4-[(1-Acetyl-4-piperidinyl)amino]-1-ethyl-N-({4-[(methylsulfonyl)amino]phenyl}methyl)-1H-pyrazolo[3,4-b]pyridine-5-carboxamide
315 4-[(1-Acetyl-4-piperidinyl)amino]-1-ethyl-N-({4-[(methylamino)carbonyl]phenyl}methyl)-1H-pyrazolo[3,4-b]pyridine-5-carboxamide
316 4-[(1-Acetyl-4-piperidinyl)amino]-N-{[4-(aminosulfonyl)phenyl]methyl}-1-ethyl-1H-pyrazolo[3,4-b]pyridine-5-carboxamide
317 4-[(1-Acetyl-4-piperidinyl)amino]-1-ethyl-N-({3-[(methylamino)carbonyl]phenyl}methyl)-1H-pyrazolo[3,4-b]pyridine-5-carboxamide
318 4-[(1-Acetyl-4-piperidinyl)amino]-N-{[4-(aminocarbonyl)phenyl]methyl}-1-ethyl-1H-pyrazolo[3,4-b]pyridine-5-carboxamide
319 4-[(1-Acetyl-4-piperidinyl)amino]-1-ethyl-N-{[6-(methyloxy)-3-pyridinyl]methyl}-1H-pyrazolo[3,4-b]pyridine-5-carboxamide
320 1-Ethyl-N-4-piperidinyl-4-(tetrahydro-2H-pyran-4-ylamino)-1H-pyrazolo[3,4-b]pyridine-5-carboxamide
321 1-Ethyl-N-(4-piperidinylmethyl)-4-(tetrahydro-2H-pyran-4-ylamino)-1H-pyrazolo[3,4-b]pyridine-5-carboxamide
322 1-Ethyl-N-[1-(ethylsulfonyl)-4-piperidinyl]-4-(tetrahydro-2H-pyran-4-ylamino)-1H-pyrazolo[3,4-b]pyridine-5-carboxamide
323 1-Ethyl-N-{1-[(1-methylethyl)sulfonyl]-4-piperidinyl}-4-(tetrahydro-2H-pyran-4-ylamino)-1H-pyrazolo[3,4-b]pyridine-5-carboxamide
324 N-[1-(Cyclopentylsulfonyl)-4-piperidinyl]-1-ethyl-4-(tetrahydro-2H-pyran-4-ylamino)-1H-pyrazolo[3,4-b]pyridine-5-carboxamide
325 1-Ethyl-N-[1-(methylsulfonyl)-4-piperidinyl]-4-(tetrahydro-2H-pyran-4-ylamino)-1H-pyrazolo[3,4-b]pyridine-5-carboxamide
326 1-Ethyl-N-{1-[(phenylmethyl)sulfonyl]-4-piperidinyl}-4-(tetrahydro-2H-pyran-4-ylamino)-1H-pyrazolo[3,4-b]pyridine-5-carboxamide
327 1-Ethyl-N-[1-(phenylsulfonyl)-4-piperidinyl]-4-(tetrahydro-2H-pyran-4-ylamino)-1H-pyrazolo[3,4-b]pyridine-5-carboxamide
328 1-Ethyl-N-[1-(propylsulfonyl)-4-piperidinyl]-4-(tetrahydro-2H-pyran-4-ylamino)-1H-pyrazolo[3,4-b]pyridine-5-carboxamide
329 N-[1-(Cyclopropylcarbonyl)-4-piperidinyl]-1-ethyl-4-(tetrahydro-2H-pyran-4-ylamino)-1H-pyrazolo[3,4-b]pyridine-5-carboxamide
330 1-Ethyl-N-[1-(3-furanylcarbonyl)-4-piperidinyl]-4-(tetrahydro-2H-pyran-4-ylamino)-1H-pyrazolo[3,4-b]pyridine-5-carboxamide
331 N-[1-(3,3-Dimethylbutanoyl)-4-piperidinyl]-1-ethyl-4-(tetrahydro-2H-pyran-4-ylamino)-1H-pyrazolo[3,4-b]pyridine-5-carboxamide
332 1-Ethyl-N-[1-(2-ethylbutanoyl)-4-piperidinyl]-4-(tetrahydro-2H-pyran-4-ylamino)-1H-pyrazolo[3,4-b]pyridine-5-carboxamide
333 N-[1-(Cyclopentylacetyl)-4-piperidinyl]-1-ethyl-4-(tetrahydro-2H-pyran-4-ylamino)-1H-pyrazolo[3,4-b]pyridine-5-carboxamide
334 1-Ethyl-N-[1-(2-methylpropanoyl)-4-piperidinyl]-4-(tetrahydro-2H-pyran-4-ylamino)-1H-pyrazolo[3,4-b]pyridine-5-carboxamide
335 1-Ethyl-4-(tetrahydro-2H-pyran-4-ylamino)-N-[1-(tetrahydro-2H-pyran-4-ylcarbonyl)-4-piperidinyl]-1H-pyrazolo[3,4-b]pyridine-5-carboxamide
336 1-Ethyl-N-(1-propanoyl-4-piperidinyl)-4-(tetrahydro-2H-pyran-4-ylamino)-1H-pyrazolo[3,4-b]pyridine-5-carboxamide
337 N-[1-(N-Acetylglycyl)-4-piperidinyl]-1-ethyl-4-(tetrahydro-2H-pyran-4-ylamino)-1H-pyrazolo[3,4-b]pyridine-5-carboxamide
338 1-Ethyl-N-[1-(4-morpholinylacetyl)-4-piperidinyl]-4-(tetrahydro-2H-pyran-4-ylamino)-1H-pyrazolo[3,4-b]pyridine-5-carboxamide
339 1-Ethyl-N-{1-[(4-oxocyclohexyl)carbonyl]-4-piperidinyl}-4-(tetrahydro-2H-pyran-4-ylamino)-1H-pyrazolo[3,4-b]pyridine-5-carboxamide
340 1-Ethyl-N-[1-(1-piperidinylacetyl)-4-piperidinyl]-4-(tetrahydro-2H-pyran-4-ylamino)-1H-pyrazolo[3,4-b]pyridine-5-carboxamide
341 1-Ethyl-N-{1-[(1-methyl-5-oxo-3-pyrrolidinyl)carbonyl]-4-piperidinyl}-4-(tetrahydro-2H-pyran-4-ylamino)-1H-pyrazolo[3,4-b]pyridine-5-carboxamide
342 1-Ethyl-N-{1-[(3-methyl-3-oxetanyl)carbonyl]-4-piperidinyl}-4-(tetrahydro-2H-pyran-4-ylamino)-1H-pyrazolo[3,4-b]pyridine-5-carboxamide
343 1-Ethyl-N-{1-[(4-fluorophenyl)acetyl]-4-piperidinyl}-4-(tetrahydro-2H-pyran-4-ylamino)-1H-pyrazolo[3,4-b]pyridine-5-carboxamide
344 N-{[1-(3,3-Dimethylbutanoyl)-4-piperidinyl]methyl}-1-ethyl-4-(tetrahydro-2H-pyran-4-ylamino)-1H-pyrazolo[3,4-b]pyridine-5-carboxamide
345 N-{[1-(Cyclopentylacetyl)-4-piperidinyl]methyl}-1-ethyl-4-(tetrahydro-2H-pyran-4-ylamino)-1H-pyrazolo[3,4-b]pyridine-5-carboxamide
346 N-{[1-(Cyclopropylcarbonyl)-4-piperidinyl]methyl}-1-ethyl-4-(tetrahydro-2H-pyran-4-ylamino)-1H-pyrazolo[3,4-b]pyridine-5-carboxamide
347 1-Ethyl-N-({1-[(4-oxocyclohexyl)carbonyl]-4-piperidinyl}methyl)-4-(tetrahydro-2H-pyran-4-ylamino)-1H-pyrazolo[3,4-b]pyridine-5-carboxamide
348 1-Ethyl-N-({1-[(4-fluorophenyl)acetyl]-4-piperidinyl}methyl)-4-(tetrahydro-2H-pyran-4-ylamino)-1H-pyrazolo[3,4-b]pyridine-5-carboxamide
349 1-Ethyl-N-({1-[(1-methyl-5-oxo-3-pyrrolidinyl)carbonyl]-4-piperidinyl}methyl)-4-(tetrahydro-2H-pyran-4-ylamino)-1H-pyrazolo[3,4-b]pyridine-5-carboxamide
350 Methyl 3-[(1-ethyl-5-{[(phenylmethyl)amino]carbonyl}-1H-pyrazolo[3,4-b]pyridin-4-yl)amino]cyclohexanecarboxylate
351 3-[(1-Ethyl-5-{[(phenylmethyl)amino]carbonyl}-1H-pyrazolo[3,4-b]pyridin-4-yl)amino]cyclohexanecarboxylic acid
352 1-Ethyl-N-(phenylmethyl)-4-(4-piperidinylamino)-1H-pyrazolo[3,4-b]pyridine-5-carboxamide
353 Ethyl 1-ethyl-4-({1-[(methyloxy)acetyl]-4-piperidinyl}amino)-1H-pyrazolo[3,4-b]pyridine-5-carboxylate
354 Ethyl 1-(1-methylethyl)-4-(tetrahydro-2H-pyran-4-ylamino)-1H-pyrazolo[3,4-b]pyridine-5-carboxylate
355 4-(Cyclohexylamino)-1-ethyl-N-methyl-1H-pyrazolo[3,4-b]pyridine-5-carboxamide
356 1-Ethyl-N-(4-fluorophenyl)-6-methyl-4-(tetrahydro-2H-pyran-4-ylamino)-1H-pyrazolo[3,4-b]pyridine-5-carboxamide
357 1-Ethyl-6-methyl-N-{[4-(methylsulfonyl)phenyl]methyl}-4-(tetrahydro-2H-pyran-4-ylamino)-1H-pyrazolo[3,4-b]pyridine-5-carboxamide
358 N-(2,3-Dihydro-1H-inden-2-yl)-1-ethyl-6-methyl-4-(tetrahydro-2H-pyran-4-ylamino)-1H-pyrazolo[3,4-b]pyridine-5-carboxamide
360 1-Ethyl-N-[3-(1-piperidinylcarbonyl)phenyl]-4-(tetrahydro-2H-pyran-4-ylamino)-1H-pyrazolo[3,4-b]pyridine-5-carboxamide
361 1-Ethyl-N-[4-(1-methylethyl)phenyl]-4-(tetrahydro-2H-pyran-4-ylamino)-1H-pyrazolo[3,4-b]pyridine-5-carboxamide
362 1-Ethyl-N-(2-fluorophenyl)-4-(tetrahydro-2H-pyran-4-ylamino)-1H-pyrazolo[3,4-b]pyridine-5-carboxamide
363 N-{3-[(Dimethylamino)carbonyl]phenyl}-1-ethyl-4-(tetrahydro-2H-pyran-4-ylamino)-1H-pyrazolo[3,4-b]pyridine-5-carboxamide
364 N-{4-[(Difluoromethyl)oxy]phenyl}-1-ethyl-4-(tetrahydro-2H-pyran-4-ylamino)-1H-pyrazolo[3,4-b]pyridine-5-carboxamide
365 N-{4-[Acetyl(methyl)amino]phenyl}-1-ethyl-4-(tetrahydro-2H-pyran-4-ylamino)-1H-pyrazolo[3,4-b]pyridine-5-carboxamide
366 1-Ethyl-N-(4-hydroxyphenyl)-4-(tetrahydro-2H-pyran-4-ylamino)-1H-pyrazolo[3,4-b]pyridine-5-carboxamide
367 1-Ethyl-N-[4-(4-morpholinyl)-2-(trifluoromethyl)phenyl]-4-(tetrahydro-2H-pyran-4-ylamino)-1H-pyrazolo[3,4-b]pyridine-5-carboxamide
368 1-Ethyl-N-4-pyridinyl-4-(tetrahydro-2H-pyran-4-ylamino)-1H-pyrazolo[3,4-b]pyridine-5-carboxamide
369 1-Ethyl-N-{4-[(4-methyl-1-piperazinyl)carbonyl]phenyl}-4-(tetrahydro-2H-pyran-4-ylamino)-1H-pyrazolo[3,4-b]pyridine-5-carboxamide
370 1-Ethyl-N-[2-(2-oxo-1-pyrrolidinyl)phenyl]-4-(tetrahydro-2H-pyran-4-ylamino)-1H-pyrazolo[3,4-b]pyridine-5-carboxamide
371 1-Ethyl-N-[3-(methylsulfonyl)phenyl]-4-(tetrahydro-2H-pyran-4-ylamino)-1H-pyrazolo[3,4-b]pyridine-5-carboxamide
372 N-{3-[Acetyl(methyl)amino]phenyl}-1-ethyl-4-(tetrahydro-2H-pyran-4-ylamino)-1H-pyrazolo[3,4-b]pyridine-5-carboxamide
373 1-Ethyl-N-{3-[(methylsulfonyl)amino]phenyl}-4-(tetrahydro-2H-pyran-4-ylamino)-1H-pyrazolo[3,4-b]pyridine-5-carboxamide
374 1-Ethyl-N-(4-fluoro-2-hydroxyphenyl)-4-(tetrahydro-2H-pyran-4-ylamino)-1H-pyrazolo[3,4-b]pyridine-5-carboxamide
375 N-(4-Chlorophenyl)-1-ethyl-4-(tetrahydro-2H-pyran-4-ylamino)-1H-pyrazolo[3,4-b]pyridine-5-carboxamide
376 N-(3-Chloro-2-cyanophenyl)-1-ethyl-4-(tetrahydro-2H-pyran-4-ylamino)-1H-pyrazolo[3,4-b]pyridine-5-carboxamide
377 1-Ethyl-N-[3-(1-piperidinylsulfonyl)phenyl]-4-(tetrahydro-2H-pyran-4-ylamino)-1H-pyrazolo[3,4-b]pyridine-5-carboxamide
379 1-Ethyl-N-[2-(methylsulfonyl)phenyl]-4-(tetrahydro-2H-pyran-4-ylamino)-1H-pyrazolo[3,4-b]pyridine-5-carboxamide
380 N-{2-[Acetyl(methyl)amino]phenyl}-1-ethyl-4-(tetrahydro-2H-pyran-4-ylamino)-1H-pyrazolo[3,4-b]pyridine-5-carboxamide
371 1-Ethyl-N-[3-(4-morpholinylcarbonyl)phenyl]-4-(tetrahydro-2H-pyran-4-ylamino)-1H-pyrazolo[3,4-b]pyridine-5-carboxamide
382 N-(4-Chloro-3-cyanophenyl)-1-ethyl-4-(tetrahydro-2H-pyran-4-ylamino)-1H-pyrazolo[3,4-b]pyridine-5-carboxamide
383 1-Ethyl-N-(3-hydroxyphenyl)-4-(tetrahydro-2H-pyran-4-ylamino)-1H-pyrazolo[3,4-b]pyridine-5-carboxamide
384 N-(3-Chlorophenyl)-1-ethyl-4-(tetrahydro-2H-pyran-4-ylamino)-1H-pyrazolo[3,4-b]pyridine-5-carboxamide
386 N-[3-[(Acetylamino)methyl]-4-(methyloxy)phenyl]-1-ethyl-4-(tetrahydro-2H-pyran-4-ylamino)-1H-pyrazolo[3,4-b]pyridine-5-carboxamide
387 1-Ethyl-N-[4-(1-piperidinylsulfonyl)phenyl]-4-(tetrahydro-2H-pyran-4-ylamino)-1H-pyrazolo[3,4-b]pyridine-5-carboxamide
388 N-(3-{[Cyclohexyl(methyl)amino]carbonyl}phenyl)-1-ethyl-4-(tetrahydro-2H-pyran-4-ylamino)-1H-pyrazolo[3,4-b]pyridine-5-carboxamide
389 1-Ethyl-N-[2-(4-morpholinyl)phenyl]-4-(tetrahydro-2H-pyran-4-ylamino)-1H-pyrazolo[3,4-b]pyridine-5-carboxamide
390 N-{3-[(Acetylamino)sulfonyl]phenyl}-1-ethyl-4-(tetrahydro-2H-pyran-4-ylamino)-1H-pyrazolo[3,4-b]pyridine-5-carboxamide
391 N-(3-Chloro-4-hydroxyphenyl)-1-ethyl-4-(tetrahydro-2H-pyran-4-ylamino)-1H-pyrazolo[3,4-b]pyridine-5-carboxamide
392 1-Ethyl-N-{4-[(methylsulfonyl)amino]phenyl}-4-(tetrahydro-2H-pyran-4-ylamino)-1H-pyrazolo[3,4-b]pyridine-5-carboxamide
393 1-Ethyl-N-{3-[(methylamino)carbonyl]phenyl}-4-(tetrahydro-2H-pyran-4-ylamino)-1H-pyrazolo[3,4-b]pyridine-5-carboxamide
394 1-Ethyl-4-(tetrahydro-2H-pyran-4-ylamino)-N-[3-(trifluoromethyl)phenyl]-1H-pyrazolo[3,4-b]pyridine-5-carboxamide
395 1-Ethyl-N-3-pyridinyl-4-(tetrahydro-2H-pyran-4-ylamino)-1H-pyrazolo[3,4-b]pyridine-5-carboxamide
396 N-(3,4-Dichlorophenyl)-1-ethyl-4-(tetrahydro-2H-pyran-4-ylamino)-1H-pyrazolo[3,4-b]pyridine-5-carboxamide
397 N-[3-(Aminosulfonyl)-4-chlorophenyl]-1-ethyl-4-(tetrahydro-2H-pyran-4-ylamino)-1H-pyrazolo[3,4-b]pyridine-5-carboxamide
398 1-Ethyl-N-[3-(4-morpholinyl)phenyl]-4-(tetrahydro-2H-pyran-4-ylamino)-1H-pyrazolo[3,4-b]pyridine-5-carboxamide
399 1-Ethyl-N-[4-(4-morpholinylsulfonyl)phenyl]-4-(tetrahydro-2H-pyran-4-ylamino)-1H-pyrazolo[3,4-b]pyridine-5-carboxamide
400 1-Ethyl-N-{2-[(4-methyl-1-piperazinyl)carbonyl]phenyl}-4-(tetrahydro-2H-pyran-4-ylamino)-1H-pyrazolo[3,4-b]pyridine-5-carboxamide
401 N-{2-[(Dimethylamino)carbonyl]phenyl}-1-ethyl-4-(tetrahydro-2H-pyran-4-ylamino)-1H-pyrazolo[3,4-b]pyridine-5-carboxamide
402 N-[2-Chloro-4-(trifluoromethyl)phenyl]-1-ethyl-4-(tetrahydro-2H-pyran-4-ylamino)-1H-pyrazolo[3,4-b]pyridine-5-carboxamide
403 N-{2-[(Acetylamino)methyl]phenyl}-1-ethyl-4-(tetrahydro-2H-pyran-4-ylamino)-1H-pyrazolo[3,4-b]pyridine-5-carboxamide
404 N-(2-Chlorophenyl)-1-ethyl-4-(tetrahydro-2H-pyran-4-ylamino)-1H-pyrazolo[3,4-b]pyridine-5-carboxamide
405 N-(3-Chloro-2-fluorophenyl)-1-ethyl-4-(tetrahydro-2H-pyran-4-ylamino)-1H-pyrazolo[3,4-b]pyridine-5-carboxamide
406 1-Ethyl-N-(3-fluorophenyl)-4-(tetrahydro-2H-pyran-4-ylamino)-1H-pyrazolo[3,4-b]pyridine-5-carboxamide
407 N-(2-Cyano-3-fluorophenyl)-1-ethyl-4-(tetrahydro-2H-pyran-4-ylamino)-1H-pyrazolo[3,4-b]pyridine-5-carboxamide
408 1-Ethyl-N-[4-(propylsulfonyl)phenyl]-4-(tetrahydro-2H-pyran-4-ylamino)-1H-pyrazolo[3,4-b]pyridine-5-carboxamide
409 N-{4-[(Dimethylamino)carbonyl]phenyl}-1-ethyl-4-(tetrahydro-2H-pyran-4-ylamino)-1H-pyrazolo[3,4-b]pyridine-5-carboxamide
411 1-Ethyl-N-[4-(methylsulfonyl)phenyl]-4-(tetrahydro-2H-pyran-4-ylamino)-1H-pyrazolo[3,4-b]pyridine-5-carboxamide
413 N-{4-[(Acetylamino)methyl]phenyl}-1-ethyl-4-(tetrahydro-2H-pyran-4-ylamino)-1H-pyrazolo[3,4-b]pyridine-5-carboxamide
414 1-Ethyl-4-(tetrahydro-2H-pyran-3-ylamino)-1H-pyrazolo[3,4-b]pyridine-5-carboxamide
415 N-[2-(Aminosulfonyl)ethyl]-4-(cyclohexylamino)-1-ethyl-1H-pyrazolo[3,4-b]pyridine-5-carboxamide
416 N-(2-Amino-2-oxoethyl)-4-(cyclohexylamino)-1-ethyl-1H-pyrazolo[3,4-b]pyridine-5-carboxamide (non-preferred name)
417 4-(Cyclohexylamino)-1-ethyl-N-{2-[(methylsulfonyl)amino]ethyl}-1H-pyrazolo[3,4-b]pyridine-5-carboxamide
418 4-(Cyclohexylamino)-1-ethyl-N-(tetrahydro-2H-pyran-4-yl)-1H-pyrazolo[3,4-b]pyridine-5-carboxamide
419 4-(Cyclohexylamino)-1-ethyl-N-[(1-methyl-1H-pyrazol-4-yl)methyl]-1H-pyrazolo[3,4-b]pyridine-5-carboxamide
420 4-(Cyclohexylamino)-1-ethyl-N-{[3-(methylsulfonyl)phenyl]methyl}-1H-pyrazolo[3,4-b]pyridine-5-carboxamide
421 N-{[3-(Aminocarbonyl)phenyl]methyl}-4-(cyclohexylamino)-1-ethyl-1H-pyrazolo[3,4-b]pyridine-5-carboxamide
422 4-(Cyclohexylamino)-1-ethyl-N-(tetrahydro-2-furanylmethyl)-1H-pyrazolo[3,4-b]pyridine-5-carboxamide
423 4-(Cyclohexylamino)-N-({4-[(dimethylamino)sulfonyl]phenyl}methyl)-1-ethyl-1H-pyrazolo[3,4-b]pyridine-5-carboxamide
424 N-[(5-Chloro-2-pyridinyl)methyl]-4-(cyclohexylamino)-1-ethyl-1H-pyrazolo[3,4-b]pyridine-5-carboxamide
425 4-(Cyclohexylamino)-1-ethyl-N-{[4-(methylsulfonyl)phenyl]methyl}-1H-pyrazolo[3,4-b]pyridine-5-carboxamide
426 4-(Cyclohexylamino)-1-ethyl-N-{[6-(methyloxy)-3-pyridinyl]methyl}-1H-pyrazolo[3,4-b]pyridine-5-carboxamide
427 4-(Cyclohexylamino)-1-ethyl-N-{4-[(methylamino)carbonyl]phenyl}-1H-pyrazolo[3,4-b]pyridine-5-carboxamide
428 4-(Cyclohexylamino)-1-ethyl-N-({3-[(methylamino)carbonyl]phenyl}methyl)-1H-pyrazolo[3,4-b]pyridine-5-carboxamide
429 N-{[4-(Aminocarbonyl)phenyl]methyl}-4-(cyclohexylamino)-1-ethyl-1H-pyrazolo[3,4-b]pyridine-5-carboxamide
430 4-(Cyclohexylamino)-1-ethyl-N-[(4-hydroxyphenyl)methyl]-1H-pyrazolo[3,4-b]pyridine-5-carboxamide
431 4-(Cyclohexylamino)-1-ethyl-N-{[4-(methyloxy)phenyl]methyl}-1H-pyrazolo[3,4-b]pyridine-5-carboxamide
432 4-(Cyclohexylamino)-N-[(3,4-difluorophenyl)methyl]-1-ethyl-1H-pyrazolo[3,4-b]pyridine-5-carboxamide
433 4-(Cyclohexylamino)-1-ethyl-N-{[4-(trifluoromethyl)phenyl]methyl}-1H-pyrazolo[3,4-b]pyridine-5-carboxamide
434 4-(Cyclohexylamino)-1-ethyl-N-({3-[(methylsulfonyl)amino]phenyl}methyl)-1H-pyrazolo[3,4-b]pyridine-5-carboxamide
435 4-(Cyclohexylamino)-N-[(2,5-difluorophenyl)methyl]-1-ethyl-1H-pyrazolo[3,4-b]pyridine-5-carboxamide
436 4-(Cyclohexylamino)-1-ethyl-N-[(4-methylphenyl)methyl]-1H-pyrazolo[3,4-b]pyridine-5-carboxamide
438 4-(Cyclohexylamino)-1-ethyl-N-(2-{4-[(methylsulfonyl)amino]phenyl}ethyl)-1H-pyrazolo[3,4-b]pyridine-5-carboxamide
439 4-(Cyclohexylamino)-1-ethyl-N-[(2-hydroxyphenyl)methyl]-1H-pyrazolo[3,4-b]pyridine-5-carboxamide
440 4-(Cyclohexylamino)-N-[(3,4-dichlorophenyl)methyl]-1-ethyl-1H-pyrazolo[3,4-b]pyridine-5-carboxamide
441 4-(Cyclohexylamino)-N-[(3,5-dichlorophenyl)methyl]-1-ethyl-1H-pyrazolo[3,4-b]pyridine-5-carboxamide
442 4-(Cyclohexylamino)-1-ethyl-N-(2-phenylethyl)-1H-pyrazolo[3,4-b]pyridine-5-carboxamide
443 4-(Cyclohexylamino)-1-ethyl-N-(1,2,3,4-tetrahydro-1-naphthalenyl)-1H-pyrazolo[3,4-b]pyridine-5-carboxamide
444 4-(Cyclohexylamino)-1-ethyl-N-{[2-(methylsulfinyl)phenyl]methyl}-1H-pyrazolo[3,4-b]pyridine-5-carboxamide
445 4-(Cyclohexylamino)-1-ethyl-N-[2-(4-hydroxyphenyl)ethyl]-1H-pyrazolo[3,4-b]pyridine-5-carboxamide
446 N-{2-[4-(Aminosulfonyl)phenyl]ethyl}-4-(cyclohexylamino)-1-ethyl-1H-pyrazolo[3,4-b]pyridine-5-carboxamide
447 4-(Cyclohexylamino)-1-ethyl-N-({2-[(methylamino)carbonyl]phenyl}methyl)-1H-pyrazolo[3,4-b]pyridine-5-carboxamide
448 4-(Cyclohexylamino)-1-ethyl-N-{[2-(methylsulfonyl)phenyl]methyl}-1H-pyrazolo[3,4-b]pyridine-5-carboxamide
449 Methyl 2-[({[4-(cyclohexylamino)-1-ethyl-1H-pyrazolo[3,4-b]pyridin-5-yl]carbonyl}amino)methyl]benzoate
450 4-(Cyclohexylamino)-1-ethyl-N-{2-[4-(methylsulfonyl)phenyl]ethyl}-1H-pyrazolo[3,4-b]pyridine-5-carboxamide
451 N-[4,5-Bis(methyloxy)-2,3-dihydro-1H-inden-2-yl]-4-(cyclohexylamino)-1-ethyl-1H-pyrazolo[3,4-b]pyridine-5-carboxamide
452 4-(Cyclohexylamino)-1-ethyl-N-{[2-fluoro-3-(trifluoromethyl)phenyl]methyl}-1H-pyrazolo[3,4-b]pyridine-5-carboxamide
453 4-(Cyclohexylamino)-N-[(3,4-dimethylphenyl)methyl]-1-ethyl-1H-pyrazolo[3,4-b]pyridine-5-carboxamide
454 4-(Cyclohexylamino)-1-ethyl-N-[2-(4-fluorophenyl)ethyl]-1H-pyrazolo[3,4-b]pyridine-5-carboxamide
455 4-(Cyclohexylamino)-1-ethyl-N-[2-(4-methylphenyl)ethyl]-1H-pyrazolo[3,4-b]pyridine-5-carboxamide
456 4-(Cyclohexylamino)-1-ethyl-N-{2-[4-(methyloxy)phenyl]ethyl}-1H-pyrazolo[3,4-b]pyridine-5-carboxamide
457 4-(Cyclohexylamino)-1-ethyl-N-(2-pyridinylmethyl)-1H-pyrazolo[3,4-b]pyridine-5-carboxamide trifluoroacetate
458 4-(Cyclohexylamino)-N-[(3,5-difluorophenyl)methyl]-1-ethyl-1H-pyrazolo[3,4-b]pyridine-5-carboxamide
459 4-(Cyclohexylamino)-N-(2,3-dihydro-1H-inden-1-yl)-1-ethyl-1H-pyrazolo[3,4-b]pyridine-5-carboxamide
460 4-(Cyclohexylamino)-N-{[4-(dimethylamino)phenyl]methyl}-1-ethyl-1H-pyrazolo[3,4-b]pyridine-5-carboxamide trifluoroacetate
461 4-(Cyclohexylamino)-1-ethyl-N-[(2-fluorophenyl)methyl]-1H-pyrazolo[3,4-b]pyridine-5-carboxamide
462 N-{[2,4-Bis(methyloxy)phenyl]methyl}-4-(cyclohexylamino)-1-ethyl-1H-pyrazolo[3,4-b]pyridine-5-carboxamide
463 N-[(6-Chloro-2-pyridinyl)methyl]-4-(cyclohexylamino)-1-ethyl-1H-pyrazolo[3,4-b]pyridine-5-carboxamide trifluoroacetate
464 N-({2-[Acetyl(methyl)amino]phenyl}methyl)-4-(cyclohexylamino)-1-ethyl-1H-pyrazolo[3,4-b]pyridine-5-carboxamide trifluoroacetate
465 4-(Cyclohexylamino)-1-ethyl-N-{[4-fluoro-3-(trifluoromethyl)phenyl]methyl}-1H-pyrazolo[3,4-b]pyridine-5-carboxamide
466 4-(Cyclohexylamino)-N-[(1R)-2,3-dihydro-1H-inden-1-yl]-1-ethyl-1H-pyrazolo[3,4-b]pyridine-5-carboxamide
467 4-(Cyclohexylamino)-N-[(2,6-dichlorophenyl)methyl]-1-ethyl-1H-pyrazolo[3,4-b]pyridine-5-carboxamide
468 Methyl 3-[({[4-(cyclohexylamino)-1-ethyl-1H-pyrazolo[3,4-b]pyridin-5-yl]carbonyl}amino)methyl]benzoate
469 4-(Cyclohexylamino)-N-(2,3-dihydro-1H-inden-2-yl)-1-ethyl-1H-pyrazolo[3,4-b]pyridine-5-carboxamide
470 Methyl 4-[({[4-(cyclohexylamino)-1-ethyl-1H-pyrazolo[3,4-b]pyridin-5-yl]carbonyl}amino)methyl]benzoate
471 4-(Cyclohexylamino)-1-ethyl-N-(1H-tetrazol-5-ylmethyl)-1H-pyrazolo[3,4-b]pyridine-5-carboxamide
472 4-(Cyclohexylamino)-N-({4-[(difluoromethyl)oxy]phenyl}methyl)-1-ethyl-1H-pyrazolo[3,4-b]pyridine-5-carboxamide
473 4-(Cyclohexylamino)-1-ethyl-N-[(2-methyl-1,3-thiazol-4-yl)methyl]-1H-pyrazolo[3,4-b]pyridine-5-carboxamide
474 N-[(2-Chloro-6-fluorophenyl)methyl]-4-(cyclohexylamino)-1-ethyl-1H-pyrazolo[3,4-b]pyridine-5-carboxamide
475 N-{[2-(Aminocarbonyl)phenyl]methyl}-4-(cyclohexylamino)-1-ethyl-1H-pyrazolo[3,4-b]pyridine-5-carboxamide
477 4-(Cyclohexylamino)-N-{[2-(dimethylamino)phenyl]methyl}-1-ethyl-1H-pyrazolo[3,4-b]pyridine-5-carboxamide
478 4-(Cyclohexylamino)-1-ethyl-N-[(4-fluorophenyl)methyl]-1H-pyrazolo[3,4-b]pyridine-5-carboxamide
479 4-(Cyclohexylamino)-1-ethyl-N-{[3-(trifluoromethyl)phenyl]methyl}-1H-pyrazolo[3,4-b]pyridine-5-carboxamide
480 4-(Cyclohexylamino)-N-[(2,6-difluorophenyl)methyl]-1-ethyl-1H-pyrazolo[3,4-b]pyridine-5-carboxamide
481 4-(Cyclohexylamino)-1-ethyl-N-[(3-fluorophenyl)methyl]-1H-pyrazolo[3,4-b]pyridine-5-carboxamide
482 4-(Cyclohexylamino)-1-ethyl-N-{[2-(trifluoromethyl)phenyl]methyl}-1H-pyrazolo[3,4-b]pyridine-5-carboxamide
483 N-(5-Chloro-2,3-dihydro-1H-inden-2-yl)-4-(cyclohexylamino)-1-ethyl-1H-pyrazolo[3,4-b]pyridine-5-carboxamide
484 4-(Cyclohexylamino)-1-ethyl-N-({4-[(methylamino)carbonyl]phenyl}methyl)-1H-pyrazolo[3,4-b]pyridine-5-carboxamide
485 4-(Cyclohexylamino)-1-ethyl-N-[4-(methyloxy)phenyl]-1H-pyrazolo[3,4-b]pyridine-5-carboxamide
486 4-(cyclohexylamino)-1-ethyl-N-[(6-oxo-1,6-dihydro-3-pyridinyl)methyl]-1H-pyrazolo[3,4-b]pyridine-5-carboxamide
487 4-(Cyclohexylamino)-1-ethyl-N-(3-pyridinylmethyl)-1H-pyrazolo[3,4-b]pyridine-5-carboxamide
488 4-[({[4-(Cyclohexylamino)-1-ethyl-1H-pyrazolo[3,4-b]pyridin-5-yl]carbonyl}amino)methyl]benzoic acid
489 3-[({[4-(Cyclohexylamino)-1-ethyl-1H-pyrazolo[3,4-b]pyridin-5-yl]carbonyl}amino)methyl]benzoic acid
490 4-(Cyclohexylamino)-N-(2,3-dihydro-1H-inden-2-yl)-1-ethyl-1H-pyrazolo[3,4-b]pyridine-5-carboxamide hydrochloride
491 4-(Cyclohexylamino)-N-(2,3-dihydro-1H-inden-2-yl)-1-ethyl-1H-pyrazolo[3,4-b]pyridine-5-carboxamide methanesulphonate
492 N-({2-[(1,1-Dimethylethyl)oxy]-3-pyridinyl}methyl)-1-ethyl-4-(tetrahydro-2H-pyran-4-ylamino)-1H-pyrazolo[3,4-b]pyridine-5-carboxamide trifluoroacetate
493 N-[(3-Chloro-4-methylphenyl)methyl]-1-ethyl-4-(tetrahydro-2H-pyran-4-ylamino)-1H-pyrazolo[3,4-b]pyridine-5-carboxamide
494 N-[(4-Chloro-2-methylphenyl)methyl]-1-ethyl-4-(tetrahydro-2H-pyran-4-ylamino)-1H-pyrazolo[3,4-b]pyridine-5-carboxamide
495 N-({2-[(Difluoromethyl)oxy]phenyl}methyl)-1-ethyl-4-(tetrahydro-2H-pyran-4-ylamino)-1H-pyrazolo[3,4-b]pyridine-5-carboxamide
496 1-Ethyl-N-({2-[(1-methylethyl)oxy]phenyl}methyl)-4-(tetrahydro-2H-pyran-4-ylamino)-1H-pyrazolo[3,4-b]pyridine-5-carboxamide
497 1-Ethyl-N-({3-[(1-methylethyl)oxy]phenyl}methyl)-4-(tetrahydro-2H-pyran-4-ylamino)-1H-pyrazolo[3,4-b]pyridine-5-carboxamide
498 N-({3-[(Difluoromethyl)oxy]phenyl}methyl)-1-ethyl-4-(tetrahydro-2H-pyran-4-ylamino)-1H-pyrazolo[3,4-b]pyridine-5-carboxamide
499 1-Ethyl-N-{[4-hydroxy-3-(methyloxy)phenyl]methyl}-4-(tetrahydro-2H-pyran-4-ylamino)-1H-pyrazolo[3,4-b]pyridine-5-carboxamide
500 N-[(5-Acetyl-2-hydroxyphenyl)methyl]-1-ethyl-4-(tetrahydro-2H-pyran-4-ylamino)-1H-pyrazolo[3,4-b]pyridine-5-carboxamide
501 1-Ethyl-4-(tetrahydro-2H-pyran-4-ylamino)-N-{2-[3-(trifluoromethyl)phenyl]ethyl}-1H-pyrazolo[3,4-b]pyridine-5-carboxamide
502 N-{[4-(Acetylamino)phenyl]methyl}-1-ethyl-4-(tetrahydro-2H-pyran-4-ylamino)-1H-pyrazolo[3,4-b]pyridine-5-carboxamide
503 1-Ethyl-N-[2-(3-hydroxyphenyl)ethyl]-4-(tetrahydro-2H-pyran-4-ylamino)-1H-pyrazolo[3,4-b]pyridine-5-carboxamide
504 N-[2-(3-Chlorophenyl)ethyl]-1-ethyl-4-(tetrahydro-2H-pyran-4-ylamino)-1H-pyrazolo[3,4-b]pyridine-5-carboxamide
505 1-Ethyl-4-(tetrahydro-2H-pyran-4-ylamino)-N-(2-{4-[(trifluoromethyl)oxy]phenyl}ethyl)-1H-pyrazolo[3,4-b]pyridine-5-carboxamide
506 1-Ethyl-N-{2-[3-(methyloxy)phenyl]ethyl}-4-(tetrahydro-2H-pyran-4-ylamino)-1H-pyrazolo[3,4-b]pyridine-5-carboxamide
507 N-[2-(4-Acetylphenyl)ethyl]-1-ethyl-4-(tetrahydro-2H-pyran-4-ylamino)-1H-pyrazolo[3,4-b]pyridine-5-carboxamide
508 N-[2-(3,4-Dichlorophenyl)ethyl]-1-ethyl-4-(tetrahydro-2H-pyran-4-ylamino)-1H-pyrazolo[3,4-b]pyridine-5-carboxamide
509 N-{2-[3-(Aminosulfonyl)phenyl]ethyl}-1-ethyl-4-(tetrahydro-2H-pyran-4-ylamino)-1H-pyrazolo[3,4-b]pyridine-5-carboxamide
510 N-{2-[3,4-Bis(methyloxy)phenyl]ethyl}-1-ethyl-4-(tetrahydro-2H-pyran-4-ylamino)-1H-pyrazolo[3,4-b]pyridine-5-carboxamide
512 N-[2-(2,3-Dichlorophenyl)ethyl]-1-ethyl-4-(tetrahydro-2H-pyran-4-ylamino)-1H-pyrazolo[3,4-b]pyridine-5-carboxamide
513 N-{2-[3,5-Bis(methyloxy)phenyl]ethyl}-1-ethyl-4-(tetrahydro-2H-pyran-4-ylamino)-1H-pyrazolo[3,4-b]pyridine-5-carboxamide
514 1-Ethyl-N-{2-[3-methyl-4-(methyloxy)phenyl]ethyl}-4-(tetrahydro-2H-pyran-4-ylamino)-1H-pyrazolo[3,4-b]pyridine-5-carboxamide
515 N-[2-(2,6-Difluorophenyl)ethyl]-1-ethyl-4-(tetrahydro-2H-pyran-4-ylamino)-1H-pyrazolo[3,4-b]pyridine-5-carboxamide
516 N-{2-[2,6-Bis(methyloxy)phenyl]ethyl}-1-ethyl-4-(tetrahydro-2H-pyran-4-ylamino)-1H-pyrazolo[3,4-b]pyridine-5-carboxamide
517 1-Ethyl-N-[2-(2-methylphenyl)ethyl]-4-(tetrahydro-2H-pyran-4-ylamino)-1H-pyrazolo[3,4-b]pyridine-5-carboxamide
518 N-[(3,4-Dimethylphenyl)methyl]-1-ethyl-4-(tetrahydro-2H-pyran-4-ylamino)-1H-pyrazolo[3,4-b]pyridine-5-carboxamide
519 N-[4,5-Bis(methyloxy)-2,3-dihydro-1H-inden-2-yl]-1-ethyl-4-(tetrahydro-2H-pyran-4-ylamino)-1H-pyrazolo[3,4-b]pyridine-5-carboxamide
521 N-{2-[4-(Aminosulfonyl)phenyl]ethyl}-1-ethyl-4-(tetrahydro-2H-pyran-4-ylamino)-1H-pyrazolo[3,4-b]pyridine-5-carboxamide
522 1-Ethyl-N-{[2-(methylsulfinyl)phenyl]methyl}-4-(tetrahydro-2H-pyran-4-ylamino)-1H-pyrazolo[3,4-b]pyridine-5-carboxamide
523 1-Ethyl-N-(2-phenylethyl)-4-(tetrahydro-2H-pyran-4-ylamino)-1H-pyrazolo[3,4-b]pyridine-5-carboxamide
524 N-{[4-(Dimethylamino)phenyl]methyl}-1-ethyl-4-(tetrahydro-2H-pyran-4-ylamino)-1H-pyrazolo[3,4-b]pyridine-5-carboxamide
525 1-Ethyl-N-[2-(4-fluorophenyl)ethyl]-4-(tetrahydro-2H-pyran-4-ylamino)-1H-pyrazolo[3,4-b]pyridine-5-carboxamide
526 1-Ethyl-N-[2-(4-methylphenyl)ethyl]-4-(tetrahydro-2H-pyran-4-ylamino)-1H-pyrazolo[3,4-b]pyridine-5-carboxamide
527 N-{[3-(Amino sulfonyl)phenyl]methyl}-1-ethyl-4-(tetrahydro-2H-pyran-4-ylamino)-1H-pyrazolo[3,4-b]pyridine-5-carboxamide
528 1-Ethyl-N-[(4-methylphenyl)methyl]-4-(tetrahydro-2H-pyran-4-ylamino)-1H-pyrazolo[3,4-b]pyridine-5-carboxamide
530 1-Ethyl-N-{[4-fluoro-3-(trifluoromethyl)phenyl]methyl}-4-(tetrahydro-2H-pyran-4-ylamino)-1H-pyrazolo[3,4-b]pyridine-5-carboxamide
531 Methyl 2-[({[1-ethyl-4-(tetrahydro-2H-pyran-4-ylamino)-1H-pyrazolo[3,4-b]pyridin-5-yl]carbonyl}amino)methyl]benzoate
532 N-[(6-Chloro-2-pyridinyl)methyl]-1-ethyl-4-(tetrahydro-2H-pyran-4-ylamino)-1H-pyrazolo[3,4-b]pyridine-5-carboxamide trifluoroacetate
533 N-(2,3-Dihydro-1H-inden-1-yl)-1-ethyl-4-(tetrahydro-2H-pyran-4-ylamino)-1H-pyrazolo[3,4-b]pyridine-5-carboxamide
534 N-({2-[Acetyl(methyl)amino]phenyl}methyl)-1-ethyl-4-(tetrahydro-2H-pyran-4-ylamino)-1H-pyrazolo[3,4-b]pyridine-5-carboxamide
535 N-[(1S)-2,3-Dihydro-1H-inden-1-yl]-1-ethyl-4-(tetrahydro-2H-pyran-4-ylamino)-1H-pyrazolo[3,4-b]pyridine-5-carboxamide
536 N-[(1R)-2,3-Dihydro-1H-inden-1-yl]-1-ethyl-4-(tetrahydro-2H-pyran-4-ylamino)-1H-pyrazolo[3,4-b]pyridine-5-carboxamide
537 1-Ethyl-N-({3-[(methylsulfonyl)amino]phenyl}methyl)-4-(tetrahydro-2H-pyran-4-ylamino)-1H-pyrazolo[3,4-b]pyridine-5-carboxamide
538 1-Ethyl-N-(phenylmethyl)-N-propyl-4-(tetrahydro-2H-pyran-4-ylamino)-1H-pyrazolo[3,4-b]pyridine-5-carboxamide
540 N-[2-(Dimethylamino)ethyl]-1-ethyl-N-(phenylmethyl)-4-(tetrahydro-2H-pyran-4-ylamino)-1H-pyrazolo[3,4-b]pyridine-5-carboxamide
541 N-Butyl-1-ethyl-N-(phenylmethyl)-4-(tetrahydro-2H-pyran-4-ylamino)-1H-pyrazolo[3,4-b]pyridine-5-carboxamide
542 N,1-Diethyl-N-(phenylmethyl)-4-(tetrahydro-2H-pyran-4-ylamino)-1H-pyrazolo[3,4-b]pyridine-5-carboxamide
544 1-Ethyl-N-(1-phenyl-4-piperidinyl)-4-(tetrahydro-2H-pyran-4-ylamino)-1H-pyrazolo[3,4-b]pyridine-5-carboxamide
545 1-ethyl-N-{1-[(ethylamino)carbonyl]-4-piperidinyl}-4-(tetrahydro-2H-pyran-4-ylamino)-1H-pyrazolo[3,4-b]pyridine-5-carboxamide
546 Formic acid-1-ethyl-N-[1-methyl-2-(4-methyl-1-piperazinyl)ethyl]-4-(tetrahydro-2H-pyran-4-ylamino)-1H-pyrazolo[3,4-b]pyridine-5-carboxamide (1:1)
547 Methyl [4-({[1-ethyl-4-(tetrahydro-2H-pyran-4-ylamino)-1H-pyrazolo[3,4-b]pyridin-5-yl]carbonyl}amino)-1-piperidinyl]acetate
548 1-Ethyl-N-{[4-(4-morpholinylmethyl)phenyl]methyl}-4-(tetrahydro-2H-pyran-4-ylamino)-1H-pyrazolo[3,4-b]pyridine-5-carboxamide trifluoroacetate
549 1-Ethyl-N-({3-[(4-methyl-1-piperazinyl)methyl]phenyl}methyl)-4-(tetrahydro-2H-pyran-4-ylamino)-1H-pyrazolo[3,4-b]pyridine-5-carboxamide trifluoroacetate
550 N-{[5-(Aminocarbonyl)-3-pyridinyl]methyl}-1-ethyl-4-(tetrahydro-2H-pyran-4-ylamino)-1H-pyrazolo[3,4-b]pyridine-5-carboxamide trifluoroacetate
551 1-Ethyl-N-{[4-(1-methylethyl)phenyl]methyl}-4-(tetrahydro-2H-pyran-4-ylamino)-1H-pyrazolo[3,4-b]pyridine-5-carboxamide
552 N-{[3-(Cyclopentyloxy)-4-(methyloxy)phenyl]methyl}-1-ethyl-4-(tetrahydro-2H-pyran-4-ylamino)-1H-pyrazolo[3,4-b]pyridine-5-carboxamide
553 1-Ethyl-N-({4-[(4-methyl-1-piperazinyl)methyl]phenyl}methyl)-4-(tetrahydro-2H-pyran-4-ylamino)-1H-pyrazolo[3,4-b]pyridine-5-carboxamide trifluoroacetate
554 N-[(2,4-Dichlorophenyl)methyl]-1-ethyl-4-(tetrahydro-2H-pyran-4-ylamino)-1H-pyrazolo[3,4-b]pyridine-5-carboxamide
555 N-[(2,4-Difluorophenyl)methyl]-1-ethyl-4-(tetrahydro-2H-pyran-4-ylamino)-1H-pyrazolo[3,4-b]pyridine-5-carboxamide
556 N-[(2-Chloro-4-fluorophenyl)methyl]-1-ethyl-4-(tetrahydro-2H-pyran-4-ylamino)-1H-pyrazolo[3,4-b]pyridine-5-carboxamide
557 N-{2-[2-Chloro-3-(methyloxy)phenyl]ethyl}-1-ethyl-4-(tetrahydro-2H-pyran-4-ylamino)-1H-pyrazolo[3,4-b]pyridine-5-carboxamide
558 Methyl 3-[({[1-ethyl-4-(tetrahydro-2H-pyran-4-ylamino)-1H-pyrazolo[3,4-b]pyridin-5-yl]carbonyl}amino)methyl]benzoate
559 1-Ethyl-N-{[3-(1-pyrrolidinylmethyl)phenyl]methyl}-4-(tetrahydro-2H-pyran-4-ylamino)-1H-pyrazolo[3,4-b]pyridine-5-carboxamide trifluoroacetate
560 1-Ethyl-N-(2-{4-[(methylsulfonyl)amino]phenyl}ethyl)-4-(tetrahydro-2H-pyran-4-ylamino)-1H-pyrazolo[3,4-b]pyridine-5-carboxamide
561 N-{[2,5-Bis(methyloxy)phenyl]methyl}-1-ethyl-4-(tetrahydro-2H-pyran-4-ylamino)-1H-pyrazolo[3,4-b]pyridine-5-carboxamide
562 N-{[2,6-Bis(methyloxy)phenyl]methyl}-1-ethyl-4-(tetrahydro-2H-pyran-4-ylamino)-1H-pyrazolo[3,4-b]pyridine-5-carboxamide
563 1-Ethyl-N-[(2-fluorophenyl)methyl]-4-(tetrahydro-2H-pyran-4-ylamino)-1H-pyrazolo[3,4-b]pyridine-5-carboxamide
564 N-[(3,5-Difluorophenyl)methyl]-1-ethyl-4-(tetrahydro-2H-pyran-4-ylamino)-1H-pyrazolo[3,4-b]pyridine-5-carboxamide
565 N-[(4-Chlorophenyl)methyl]-1-ethyl-4-(tetrahydro-2H-pyran-4-ylamino)-1H-pyrazolo[3,4-b]pyridine-5-carboxamide
567 N-Cyclohexyl-1-ethyl-4-(tetrahydro-2H-pyran-4-ylamino)-1H-pyrazolo[3,4-b]pyridine-5-carboxamide
568 1-Ethyl-N-{2-[4-(methylsulfonyl)phenyl]ethyl}-4-(tetrahydro-2H-pyran-4-ylamino)-1H-pyrazolo[3,4-b]pyridine-5-carboxamide
569 1-Ethyl-N-{[2-fluoro-3-(trifluoromethyl)phenyl]methyl}-4-(tetrahydro-2H-pyran-4-ylamino)-1H-pyrazolo[3,4-b]pyridine-5-carboxamide
570 N-({4-[(Cyclopropylamino)carbonyl]phenyl}methyl)-1-ethyl-4-(tetrahydro-2H-pyran-4-ylamino)-1H-pyrazolo[3,4-b]pyridine-5-carboxamide
571 1-Ethyl-N-{[4-(4-methyl-1-piperazinyl)phenyl]methyl}-4-(tetrahydro-2H-pyran-4-ylamino)-1H-pyrazolo[3,4-b]pyridine-5-carboxamide
572 1-Ethyl-N-{[4-(1-pyrrolidinylmethyl)phenyl]methyl}-4-(tetrahydro-2H-pyran-4-ylamino)-1H-pyrazolo[3,4-b]pyridine-5-carboxamide
573 1-Ethyl-N-[6-(methyloxy)-1-oxo-2,3-dihydro-1H-inden-2-yl]-4-(tetrahydro-2H-pyran-4-ylamino)-1H-pyrazolo[3,4-b]pyridine-5-carboxamide
574 N-[(2,5-Dichlorophenyl)methyl]-1-ethyl-4-(tetrahydro-2H-pyran-4-ylamino)-1H-pyrazolo[3,4-b]pyridine-5-carboxamide
575 N-[(3,5-Diethylphenyl)methyl]-1-ethyl-4-(tetrahydro-2H-pyran-4-ylamino)-1H-pyrazolo[3,4-b]pyridine-5-carboxamide
576 N-[(2,3-Difluorophenyl)methyl]-1-ethyl-4-(tetrahydro-2H-pyran-4-ylamino)-1H-pyrazolo[3,4-b]pyridine-5-carboxamide
577 1-Ethyl-N-{[2-(methylsulfonyl)phenyl]methyl}-4-(tetrahydro-2H-pyran-4-ylamino)-1H-pyrazolo[3,4-b]pyridine-5-carboxamide
578 1-Ethyl-N-[(3-hydroxyphenyl)methyl]-4-(tetrahydro-2H-pyran-4-ylamino)-1H-pyrazolo[3,4-b]pyridine-5-carboxamide
579 N-{[3,5-Bis(methyloxy)phenyl]methyl}-1-ethyl-4-(tetrahydro-2H-pyran-4-ylamino)-1H-pyrazolo[3,4-b]pyridine-5-carboxamide
580 1-Ethyl-N-[2-(4-hydroxyphenyl)ethyl]-4-(tetrahydro-2H-pyran-4-ylamino)-1H-pyrazolo[3,4-b]pyridine-5-carboxamide
581 N-[(3,5-Dichlorophenyl)methyl]-1-ethyl-4-(tetrahydro-2H-pyran-4-ylamino)-1H-pyrazolo[3,4-b]pyridine-5-carboxamide
582 N-{[2,4-Bis(methyloxy)phenyl]methyl}-1-ethyl-4-(tetrahydro-2H-pyran-4-ylamino)-1H-pyrazolo[3,4-b]pyridine-5-carboxamide
583 1-Ethyl-N-{[2-(methyloxy)phenyl]methyl}-4-(tetrahydro-2H-pyran-4-ylamino)-1H-pyrazolo[3,4-b]pyridine-5-carboxamide
584 N-[(2,4-Dimethylphenyl)methyl]-1-ethyl-4-(tetrahydro-2H-pyran-4-ylamino)-1H-pyrazolo[3,4-b]pyridine-5-carboxamide
585 1-Ethyl-N-({2-[(methylamino)carbonyl]phenyl}methyl)-4-(tetrahydro-2H-pyran-4-ylamino)-1H-pyrazolo[3,4-b]pyridine-5-carboxamide
586 1-Ethyl-N-{2-[4-(methyloxy)phenyl]ethyl}-4-(tetrahydro-2H-pyran-4-ylamino)-1H-pyrazolo[3,4-b]pyridine-5-carboxamide
587 N-[(2-Chlorophenyl)methyl]-1-ethyl-4-(tetrahydro-2H-pyran-4-ylamino)-1H-pyrazolo[3,4-b]pyridine-5-carboxamide
588 1-Ethyl-N-[(2-hydroxyphenyl)methyl]-4-(tetrahydro-2H-pyran-4-ylamino)-1H-pyrazolo[3,4-b]pyridine-5-carboxamide
589 N-(1,3-Benzodioxol-5-ylmethyl)-1-ethyl-4-(tetrahydro-2H-pyran-4-ylamino)-1H-pyrazolo[3,4-b]pyridine-5-carboxamide
590 1-Ethyl-N-[3-(methyloxy)phenyl]-4-(tetrahydro-2H-pyran-4-ylamino)-1H-pyrazolo[3,4-b]pyridine-5-carboxamide
591 N-(Cyclohexylmethyl)-1-ethyl-4-(tetrahydro-2H-pyran-4-ylamino)-1H-pyrazolo[3,4-b]pyridine-5-carboxamide
592 1-Ethyl-N-(1,2,3,4-tetrahydro-1-naphthalenyl)-4-(tetrahydro-2H-pyran-4-ylamino)-1H-pyrazolo[3,4-b]pyridine-5-carboxamide
593 Methyl 4-[({[1-ethyl-4-(tetrahydro-2H-pyran-4-ylamino)-1H-pyrazolo[3,4-b]pyridin-5-yl]carbonyl}amino)methyl]benzoate
594 N-[(3,4-Dichlorophenyl)methyl]-1-ethyl-4-(tetrahydro-2H-pyran-4-ylamino)-1H-pyrazolo[3,4-b]pyridine-5-carboxamide
595 N-{[4-(Aminocarbonyl)phenyl]methyl}-1-ethyl-4-(tetrahydro-2H-pyran-4-ylamino)-1H-pyrazolo[3,4-b]pyridine-5-carboxamide
596 N-[(2,6-Difluorophenyl)methyl]-1-ethyl-4-(tetrahydro-2H-pyran-4-ylamino)-1H-pyrazolo[3,4-b]pyridine-5-carboxamide
597 N-{[3-(Aminocarbonyl)phenyl]methyl}-1-ethyl-4-(tetrahydro-2H-pyran-4-ylamino)-1H-pyrazolo[3,4-b]pyridine-5-carboxamide
598 1-Ethyl-N-[(4-hydroxyphenyl)methyl]-4-(tetrahydro-2H-pyran-4-ylamino)-1H-pyrazolo[3,4-b]pyridine-5-carboxamide
599 1-Ethyl-N-{[6-(methyloxy)-3-pyridinyl]methyl}-4-(tetrahydro-2H-pyran-4-ylamino)-1H-pyrazolo[3,4-b]pyridine-5-carboxamide
600 1-Ethyl-N-(2-pyridinylmethyl)-4-(tetrahydro-2H-pyran-4-ylamino)-1H-pyrazolo[3,4-b]pyridine-5-carboxamide
601 1-Ethyl-4-(tetrahydro-2H-pyran-4-ylamino)-N-{[3-(trifluoromethyl)phenyl]methyl}-1H-pyrazolo[3,4-b]pyridine-5-carboxamide
602 N-[4-(2-Amino-2-oxoethyl)phenyl]-1-ethyl-4-(tetrahydro-2H-pyran-4-ylamino)-1H-pyrazolo[3,4-b]pyridine-5-carboxamide
603 1-Ethyl-N-({4-[(methylamino)carbonyl]phenyl}methyl)-4-(tetrahydro-2H-pyran-4-ylamino)-1H-pyrazolo[3,4-b]pyridine-5-carboxamide
604 1-Ethyl-N-{4-[2-(methylamino)-2-oxoethyl]phenyl}-4-(tetrahydro-2H-pyran-4-ylamino)-1H-pyrazolo[3,4-b]pyridine-5-carboxamide
605 1-Ethyl-N-[(3-fluorophenyl)methyl]-4-(tetrahydro-2H-pyran-4-ylamino)-1H-pyrazolo[3,4-b]pyridine-5-carboxamide
606 1-Ethyl-N-({4-[(methylsulfonyl)amino]phenyl}methyl)-4-(tetrahydro-2H-pyran-4-ylamino)-1H-pyrazolo[3,4-b]pyridine-5-carboxamide
607 N-{[4-(Aminosulfonyl)phenyl]methyl}-1-ethyl-4-(tetrahydro-2H-pyran-4-ylamino)-1H-pyrazolo[3,4-b]pyridine-5-carboxamide
608 N-{[2-(Aminocarbonyl)phenyl]methyl}-1-ethyl-4-(tetrahydro-2H-pyran-4-ylamino)-1H-pyrazolo[3,4-b]pyridine-5-carboxamide
609 N-({4-[(Difluoromethyl)oxy]phenyl}methyl)-1-ethyl-4-(tetrahydro-2H-pyran-4-ylamino)-1H-pyrazolo[3,4-b]pyridine-5-carboxamide
610 N-({3-[(Dimethylamino)methyl]phenyl}methyl)-1-ethyl-4-(tetrahydro-2H-pyran-4-ylamino)-1H-pyrazolo[3,4-b]pyridine-5-carboxamide
611 N-{[3-Chloro-4-(methyloxy)phenyl]methyl}-1-ethyl-4-(tetrahydro-2H-pyran-4-ylamino)-1H-pyrazolo[3,4-b]pyridine-5-carboxamide
612 N-(1-Acetyl-4-piperidinyl)-1-ethyl-4-(tetrahydro-2H-pyran-4-ylamino)-1H-pyrazolo[3,4-b]pyridine-5-carboxamide
613 1-Ethyl-4-(tetrahydro-2H-pyran-4-ylamino)-N-{[2-(trifluoromethyl)phenyl]methyl}-1H-pyrazolo[3,4-b]pyridine-5-carboxamide
615 N-(5-Chloro-2,3-dihydro-1H-inden-2-yl)-1-ethyl-4-(tetrahydro-2H-pyran-4-ylamino)-1H-pyrazolo[3,4-b]pyridine-5-carboxamide
616 N-({3-[(Acetylamino)methyl]phenyl}methyl)-1-ethyl-4-(tetrahydro-2H-pyran-4-ylamino)-1H-pyrazolo[3,4-b]pyridine-5-carboxamide
617 1-Ethyl-N-[(4-fluorophenyl)methyl]-4-(tetrahydro-2H-pyran-4-ylamino)-1H-pyrazolo[3,4-b]pyridine-5-carboxamide
618 1-Ethyl-N-{[4-fluoro-2-(trifluoromethyl)phenyl]methyl}-4-(tetrahydro-2H-pyran-4-ylamino)-1H-pyrazolo[3,4-b]pyridine-5-carboxamide
619 1-Ethyl-N-[(2-ethylphenyl)methyl]-4-(tetrahydro-2H-pyran-4-ylamino)-1H-pyrazolo[3,4-b]pyridine-5-carboxamide
620 1-Ethyl-N-{[2-fluoro-5-(trifluoromethyl)phenyl]methyl}-4-(tetrahydro-2H-pyran-4-ylamino)-1H-pyrazolo[3,4-b]pyridine-5-carboxamide
621 1-Ethyl-4-(tetrahydro-2H-pyran-4-ylamino)-N-[(2,3,4-trifluorophenyl)methyl]-1H-pyrazolo[3,4-b]pyridine-5-carboxamide
622 N-[(4-Chloro-2-fluorophenyl)methyl]-1-ethyl-4-(tetrahydro-2H-pyran-4-ylamino)-1H-pyrazolo[3,4-b]pyridine-5-carboxamide
623 N-[(4-Bromo-2-fluorophenyl)methyl]-1-ethyl-4-(tetrahydro-2H-pyran-4-ylamino)-1H-pyrazolo[3,4-b]pyridine-5-carboxamide
624 N-[(3,5-Dimethylphenyl)methyl]-1-ethyl-4-(tetrahydro-2H-pyran-4-ylamino)-1H-pyrazolo[3,4-b]pyridine-5-carboxamide
625 N-[(2,3-Dimethylphenyl)methyl]-1-ethyl-4-(tetrahydro-2H-pyran-4-ylamino)-1H-pyrazolo[3,4-b]pyridine-5-carboxamide
626 N-[(2,3-Dichlorophenyl)methyl]-1-ethyl-4-(tetrahydro-2H-pyran-4-ylamino)-1H-pyrazolo[3,4-b]pyridine-5-carboxamide
627 N-[(4-Cyanophenyl)methyl]-1-ethyl-4-(tetrahydro-2H-pyran-4-ylamino)-1H-pyrazolo[3,4-b]pyridine-5-carboxamide
628 N-[(4-Bromophenyl)methyl]-1-ethyl-4-(tetrahydro-2H-pyran-4-ylamino)-1H-pyrazolo[3,4-b]pyridine-5-carboxamide
629 1-Ethyl-N-{[5-fluoro-2-(trifluoromethyl)phenyl]methyl}-4-(tetrahydro-2H-pyran-4-ylamino)-1H-pyrazolo[3,4-b]pyridine-5-carboxamide
630 1-Ethyl-N-[(4-iodophenyl)methyl]-4-(tetrahydro-2H-pyran-4-ylamino)-1H-pyrazolo[3,4-b]pyridine-5-carboxamide
631 N-{[4-(1,1-Dimethylethyl)phenyl]methyl}-1-ethyl-4-(tetrahydro-2H-pyran-4-ylamino)-1H-pyrazolo[3,4-b]pyridine-5-carboxamide
632 N-[(3-Cyanophenyl)methyl]-1-ethyl-4-(tetrahydro-2H-pyran-4-ylamino)-1H-pyrazolo[3,4-b]pyridine-5-carboxamide
633 N-[(2,6-Dichlorophenyl)methyl]-1-ethyl-4-(tetrahydro-2H-pyran-4-ylamino)-1H-pyrazolo[3,4-b]pyridine-5-carboxamide
634 N-[(5-Chloro-2-methylphenyl)methyl]-1-ethyl-4-(tetrahydro-2H-pyran-4-ylamino)-1H-pyrazolo[3,4-b]pyridine-5-carboxamide
635 N-[(3,5-Dibromophenyl)methyl]-1-ethyl-4-(tetrahydro-2H-pyran-4-ylamino)-1H-pyrazolo[3,4-b]pyridine-5-carboxamide
636 1-Ethyl-N-[(4-ethylphenyl)methyl]-4-(tetrahydro-2H-pyran-4-ylamino)-1H-pyrazolo[3,4-b]pyridine-5-carboxamide
637 1-Ethyl-N-{[3-fluoro-4-(trifluoromethyl)phenyl]methyl}-4-(tetrahydro-2H-pyran-4-ylamino)-1H-pyrazolo[3,4-b]pyridine-5-carboxamide
638 1-Ethyl-N-[(2-iodophenyl)methyl]-4-(tetrahydro-2H-pyran-4-ylamino)-1H-pyrazolo[3,4-b]pyridine-5-carboxamide
639 N-[(2-Bromophenyl)methyl]-1-ethyl-4-(tetrahydro-2H-pyran-4-ylamino)-1H-pyrazolo[3,4-b]pyridine-5-carboxamide
640 1-Ethyl-N-{[4-(hydroxymethyl)phenyl]methyl}-4-(tetrahydro-2H-pyran-4-ylamino)-1H-pyrazolo[3,4-b]pyridine-5-carboxamide
641 1-Ethyl-N-{[3-(hydroxymethyl)phenyl]methyl}-4-(tetrahydro-2H-pyran-4-ylamino)-1H-pyrazolo[3,4-b]pyridine-5-carboxamide
642 1-Ethyl-N-{[3-(hydroxymethyl)-2-methylphenyl]methyl}-4-(tetrahydro-2H-pyran-4-ylamino)-1H-pyrazolo[3,4-b]pyridine-5-carboxamide
643 N-{[2,3-Dichloro-6-(hydroxymethyl)phenyl]methyl}-1-ethyl-4-(tetrahydro-2H-pyran-4-ylamino)-1H-pyrazolo[3,4-b]pyridine-5-carboxamide
644 N-[(2,4-Dichloro-6-methylphenyl)methyl]-1-ethyl-4-(tetrahydro-2H-pyran-4-ylamino)-1H-pyrazolo[3,4-b]pyridine-5-carboxamide
645 1-Ethyl-N-{[4-(2-methylpropyl)phenyl]methyl}-4-(tetrahydro-2H-pyran-4-ylamino)-1H-pyrazolo[3,4-b]pyridine-5-carboxamide
646 N-[(2,5-dimethylphenyl)methyl]-1-ethyl-4-(tetrahydro-2H-pyran-4-ylamino)-1H-pyrazolo[3,4-b]pyridine-5-carboxamide
647 1-Ethyl-4-(tetrahydro-2H-pyran-4-ylamino)-N-[(2,4,5-trifluorophenyl)methyl]-1H-pyrazolo[3,4-b]pyridine-5-carboxamide
648 1-Ethyl-N-{[2-fluoro-4-(trifluoromethyl)phenyl]methyl}-4-(tetrahydro-2H-pyran-4-ylamino)-1H-pyrazolo[3,4-b]pyridine-5-carboxamide
649 N-[(2-Chloro-6-methylphenyl)methyl]-1-ethyl-4-(tetrahydro-2H-pyran-4-ylamino)-1H-pyrazolo[3,4-b]pyridine-5-carboxamide
650 4-[({[1-Ethyl-4-(tetrahydro-2H-pyran-4-ylamino)-1H-pyrazolo[3,4-b]pyridin-5-yl]carbonyl}amino)methyl]benzoic acid sodium salt
651 3-[({[1-Ethyl-4-(tetrahydro-2H-pyran-4-ylamino)-1H-pyrazolo[3,4-b]pyridin-5-yl]carbonyl}amino)methyl]benzoic acid
652 Ethyl 1-ethyl-4-{[4-(hydroxyimino)cyclohexyl]amino}-1H-pyrazolo[3,4-b]pyridine-5-carboxylate
653 1-Ethyl-4-{[4-(hydroxyimino)cyclohexyl]amino}-N-{[4-(methyloxy)phenyl]methyl}-1H-pyrazolo[3,4-b]pyridine-5-carboxamide
654 N-{[4-(Dimethylamino)phenyl]methyl}-1-ethyl-4-{[4-(hydroxyimino)cyclohexyl]amino}-1H-pyrazolo[3,4-b]pyridine-5-carboxamide
655 1-Ethyl-4-({4-[(ethyloxy)imino]cyclohexyl}amino)-N-{[4-(methyloxy)phenyl]methyl}-1H-pyrazolo[3,4-b]pyridine-5-carboxamide
656 1-Ethyl-4-({4-[(methyloxy)imino]cyclohexyl}amino)-N-{[4-(methyloxy)phenyl]methyl}-1H-pyrazolo[3,4-b]pyridine-5-carboxamide
657 4-[(4-{[(1,1-Dimethylethyl)oxy]imino}cyclohexyl)amino]-1-ethyl-N-{[4-(methyloxy)phenyl]methyl}-1H-pyrazolo[3,4-b]pyridine-5-carboxamide
658 1-Ethyl-N-{[4-(methyloxy)phenyl]methyl}-4-[(7-oxohexahydro-1H-azepin-4-yl)amino]-1H-pyrazolo[3,4-b]pyridine-5-carboxamide
659 Ethyl 1-ethyl-4-[(7-oxohexahydro-1H-azepin-4-yl)amino]-1H-pyrazolo[3,4-b]pyridine-5-carboxylate
660 4-{[cis-4-(Butylamino)cyclohexyl]amino}-N-(2,3-dihydro-1H-inden-2-yl)-1-ethyl-1H-pyrazolo[3,4-b]pyridine-5-carboxamide
661 4-[(trans-4-Aminocyclohexyl)amino]-1-ethyl-N-(phenylmethyl)-1H-pyrazolo[3,4-b]pyridine-5-carboxamide
662 4-[(trans-2-Aminocyclohexyl)amino]-1-ethyl-N-(phenylmethyl)-1H-pyrazolo[3,4-b]pyridine-5-carboxamide
663 4-[(cis-2-Aminocyclohexyl)amino]-1-ethyl-N-(phenylmethyl)-1H-pyrazolo[3,4-b]pyridine-5-carboxamide
664 4-[(3-Aminocyclohexyl)amino]-1-ethyl-N-(phenylmethyl)-1H-pyrazolo[3,4-b]pyridine-5-carboxamide

### Example Name

### No.

665 Ethyl 1-ethyl-4-{[(1SR,3RS)-3-hydroxycyclohexyl]amino}-1H-pyrazolo[3,4-b]pyridine-5-carboxylate
666 N,1-Diethyl-4-{[(1SR,3RS)-3-hydroxycyclohexyl]amino}-1H-pyrazolo[3,4-b]pyridine-5-carboxamide
667 1-Ethyl-N-(4-fluorophenyl)-4-{[(1SR,3RS)-3-hydroxycyclohexyl]amino}-1H-pyrazolo[3,4-b]pyridine-5-carboxamide
668 1-Ethyl-4-{[(1SR,3RS)-3-hydroxycyclohexyl]amino}-N-(1,3-thiazol-2-ylmethyl)-1H-pyrazolo[3,4-b]pyridine-5-carboxamide
669 1-ethyl-N-[(4-fluorophenyl)methyl]-4-{[(1SR,3RS)-3-hydroxycyclohexyl]amino}-1H-pyrazolo[3,4-b]pyridine-5-carboxamide
670 1-ethyl-4-{[(1SR,3RS)-3-hydroxycyclohexyl]amino}-N-{[4-(methylsulfonyl)phenyl]methyl}-1H-pyrazolo[3,4-b]pyridine-5-carboxamide
671 N-{[3,4-bis(methyloxy)phenyl]methyl}-1-ethyl-4-{[(1SR,3RS)-3-hydroxycyclohexyl]amino}-1H-pyrazolo[3,4-b]pyridine-5-carboxamide
672 1-ethyl-4-{[(1SR,3RS)-3-hydroxycyclohexyl]amino}-N-(2-pyridinylmethyl)-1H-pyrazolo[3,4-b]pyridine-5-carboxamide
673 1-ethyl-4-{[(1SR,3RS)-3-hydroxycyclohexyl]amino}-N-[(1-methyl-1H-pyrazol-4-yl)methyl]-1H-pyrazolo[3,4-b]pyridine-5-carboxamide
674 N-[(3,4-dimethylphenyl)methyl]-1-ethyl-4-{[(1SR,3RS)-3-hydroxycyclohexyl]amino}-1H-pyrazolo[3,4-b]pyridine-5-carboxamide
675 1-ethyl-4-{[(1SR,3RS)-3-hydroxycyclohexyl]amino}-N-{[4-(methyloxy)phenyl]methyl}-1H-pyrazolo[3,4-b]pyridine-5-carboxamide
676 N-[(2,4-dimethylphenyl)methyl]-1-ethyl-4-{[(1SR,3RS)-3-hydroxycyclohexyl]amino}-1H-pyrazolo[3,4-b]pyridine-5-carboxamide
667 N-[(2,3-Dichlorophenyl)methyl]-1-ethyl-4-[(4-oxocyclohexyl)amino]-1H-pyrazolo[3,4-b]pyridine-5-carboxamide
678 N-[(3-Chloro-4-methylphenyl)methyl]-1-ethyl-4-[(4-oxocyclohexyl)amino]-1H-pyrazolo[3,4-b]pyridine-5-carboxamide
679 N-[(4-Chloro-2-methylphenyl)methyl]-1-ethyl-4-[(4-oxocyclohexyl)amino]-1H-pyrazolo[3,4-b]pyridine-5-carboxamide
680 N-[(2,4-Dimethylphenyl)methyl]-1-ethyl-4-{[4-(hydroxyimino)cyclohexyl]amino}-1H-pyrazolo[3,4-b]pyridine-5-carboxamide
681 N-[(3,4-Dimethylphenyl)methyl]-1-ethyl-4-{[4-(hydroxyimino)cyclohexyl]amino}-1H-pyrazolo[3,4-b]pyridine-5-carboxamide
682 N-[(2,3-Dichlorophenyl)methyl]-1-ethyl-4-{[4-(hydroxyimino)cyclohexyl]amino}-1H-pyrazolo[3,4-b]pyridine-5-carboxamide
683 N-[(3-Chloro-4-methylphenyl)methyl]-1-ethyl-4-{[4-(hydroxyimino)cyclohexyl]amino}-1H-pyrazolo[3,4-b]pyridine-5-carboxamide
684 N-[(4-Chloro-2-methylphenyl)methyl]-1-ethyl-4-{[4-(hydroxyimino)cyclohexyl]amino}-1H-pyrazolo[3,4-b]pyridine-5-carboxamide
685 N-({4-[(Difluoromethyl)oxy]phenyl}methyl)-1-ethyl-4-{[4-(hydroxyimino)cyclohexyl]amino}-1H-pyrazolo[3,4-b]pyridine-5-carboxamide
686 1-Ethyl-4-{[4-(hydroxyimino)cyclohexyl]amino}-N-{[4-(trifluoromethyl)phenyl]methyl}-1H-pyrazolo[3,4-b]pyridine-5-carboxamide

### Example 1

### Ethyl 4-(cyclopentylamino)-1-ethyl-1H-pyrazolo[3,4-b]pyridine-5-carboxylate

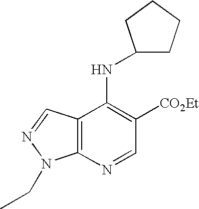 [1186] That is, Example 1 is

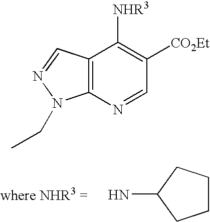

Intermediate 1 (0.051 g) and cyclopentyl amine (0.019 g) were suspended in ethanol (2 ml) and triethylamine (0.14 ml) was added. The mixture was stirred under nitrogen and heated at 80° C. for 16 h. After cooling to room temperature, ethanol was removed by evaporation under a stream of nitrogen and the residue partitioned between dichloromethane (DCM) and water. The layers were separated and the organic layer was loaded directly onto an solid phase extraction (SPE) cartridge (silica, 5 g) which was eluted sequentially with; (i) DCM, (ii) DCM:Et₂O (2:1), (iii) DCM:Et₂O (1:1), (iv) Et₂O, (v) EtOAc, (vi) MeOH. Fractions containing desired material were combined and concentrated in vacuo to afford Example 1 (0.074 g). LCMS showed MH⁺=303; T_{RET}=3.45 min.

Similarly prepared were the following:

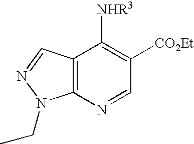

| | | | MH⁺ | T_{RET} |
|---|---|---|---|---|
| | NHR³ | Amine reagent | ion | (min) |
| Example 2 | 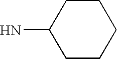 | Cyclohexyl amine | 317 | 3.65 |
| Example 3(= Intermediate 32) | 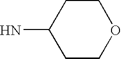 | 4-Aminotetrahydropyran | 319 | 2.93 |
| Example 5(= Intermediate 207*) | 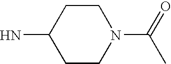 | Intermediate 6 | 360 | 3.20 |

*For alternative synthesis of Example 5, see Example 207 hereinafter

### Example 3

### (=Intermediate 32): Ethyl 1-ethyl-4-(tetrahydro-2H-pyran-4-ylamino)-1H-pyrazolo[3,4-b]pyridine-5-carboxylate

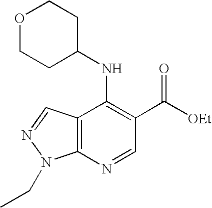

Instead of the method shown above for Examples 1-5 (called Method A), the compound of Example 3 can also be made: either using the minor variation of Method A described in detail under “Intermediate 32” hereinabove, or using the following Method B:

### Example 3, Method B

Intermediate 1 (2.5 g) was dissolved in acetonitrile (15 ml). 4-Aminotetrahydropyran hydrochloride (1.1 g) and N,N-diisopropylethylamine (9.4 ml) were added and the mixture stirred under nitrogen at 85° C. for 16 h. A trace of starting material remained, so an additional portion of 4-aminotetrahydropyran hydrochloride (0.11 g) was added and stirring continued at 85° C. for a further 16 h. The mixture was then concentrated in vacuo. The residue was partitioned between DCM and water. The layers were separated and the organic layer was washed with further water (2×20 ml) then dried (Na₂SO₄) and concentrated in vacuo. The residue was further purified by chromatography using Biotage (silica, 90 g), eluting with cyclohexane:ethyl acetate to afford Example 3 (2.45 g). LCMS showed MH⁺=319; T_{RET}=2.90 min.

### Example 6

### Ethyl 4-(cyclopentylamino)-1-methyl-1H-pyrazolo[3,4-b]pyridine-5-carboxylate

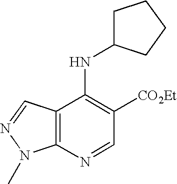

Intermediate 3 (0.045 g) was placed in a Reactivial™ and treated with cyclopentyl amine (0.07 ml). The mixture was heated at 90° C. for 2 h, then allowed to cool to room temperature and partitioned between chloroform (2 ml) and water (1 ml). The layers were separated and the organic phase was evaporated to a brown solid, which was purified by mass directed autoprep HPLC, to afford Example 6 as a white solid (0.008 g). LCMS showed MH⁺=289; T_{RET}=3.22 min.

### Example 7

### Ethyl 1-methyl-4-(tetrahydro-2H-pyran-4-ylamino)-1H-pyrazolo[3,4-b]pyridine-5-carboxylate

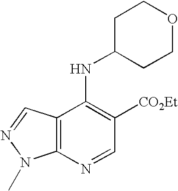

Intermediate 3 (0.035 g) was placed in a Reactivial™ and treated with 4-amino tetrahydropyran (0.06 ml). The mixture was heated at 90° C. for 2 h, then allowed to cool to room temperature and partitioned between chloroform (2 ml) and water (1 ml). The layers were separated and the organic phase was concentrated, then applied to a preparative TLC plate (silica, 20 cm×20 cm×1 mm) which was eluted with ethyl acetate. The required band was removed from the plate and the silica washed with ethyl acetate (2×15 ml). Concentration of the ethyl acetate solution in vacuo afforded Example 7 as a white solid (0.008 g). LCMS showed MH⁺=305; T_{RET}=2.67 min.

### Example 8

### Ethyl 1-ethyl-4-[(3S)-tetrahydrofuran-3-ylamino]-1H-pyrazolo[3,4-b]pyridine-5-carboxylate

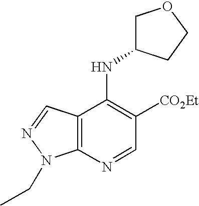 [1197] that is:

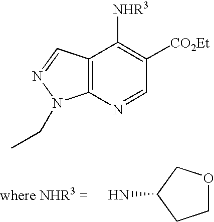

Intermediate 1 (0.05 g) and (S)-(−)-3-aminotetrahydrofuran 4-toluene sulphonate (0.052 g) were suspended in ethanol (1 ml) and triethylamine (0.14 ml) was added. The mixture was stirred under nitrogen and heated at 80° C. for 24 h. After cooling to room temperature, ethanol was removed by evaporation under a stream of nitrogen and the residue partitioned between DCM (2 ml) and water (1.5 ml). The layers were separated and the organic layer concentrated to dryness. Purification was carried out using an SPE cartridge (silica, 5 g), eluting with a gradient of EtOAc:cyclohexane; (1:16 then, 1:8, 1:4, 1:2, 1:1 and 1:0). Fractions containing desired material were combined and concentrated in vacuo to afford Example 8 (0.052 g). LCMS showed MH⁺=305; T_{RET}=2.70 min.

Similarly prepared were the following:

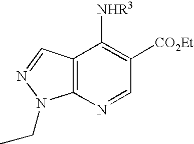

| | | | MH⁺ | T_{RET} |
|---|---|---|---|---|
| | NHR³ | Amine Reagent | ion | (min) |
| Example 9 | 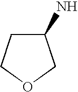 | (R)-(+)-3-Aminotetrahydrofuran4-toluene sulphonate | 305 | 2.73 |
| Example 10 | 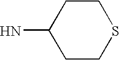 | Intermediate 11 | 335 | 3.21 |
| Example 11(mixture ofenantiomers) | 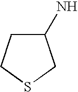 | Intermediate 12 | 321 | 3.10 |
| Example 12 | 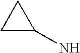 | Cyclopropyl amine | 275 | 2.98 |

### Example 13

### Ethyl 4-[(1,1-dioxidotetrahydrothien-3-yl)amino]-1-ethyl-1H-pyrazolo[3,4-b]pyridine-5-carboxylate

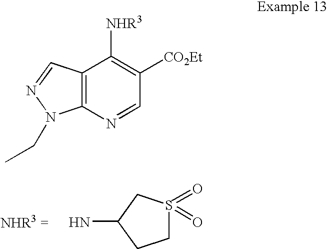

Intermediate 1 (0.05 g) and Intermediate 13 (0.027 g) were suspended in ethanol (1 ml) and triethylamine (0.14 ml) was added. The mixture was stirred under nitrogen and heated at 80° C. for 24 h. After cooling to room temperature, ethanol was removed by evaporation under a stream of nitrogen and the residue partitioned between DCM (2 ml) and water (1.5 ml). The layers were separated and the organic layer concentrated to dryness. Purification was carried out using an SPE cartridge (silica, 5 g), eluting with a gradient of EtOAc:cyclohexane; (1:8 then 1:4, 1:2, 1:1 and 1:0). Fractions containing desired material were combined and concentrated in vacuo to afford Example 13 (0.045 g) as a mixture of enantiomers. LCMS showed MH⁺=353; T_{RET}=2.60 min.

Similarly prepared was the following:

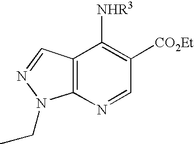

| | | | MH⁺ | T_{RET} |
|---|---|---|---|---|
| | NHR³ | Amine Reagent | ion | (min) |
| Example 14 | 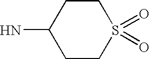 | Intermediate 14 | 367 | 2.64 |

### Example 19

### (reference example, as an intermediate): Ethyl 4-(cyclopentylamino)-1H-pyrazolo[3,4-b]pyridine-5-carboxylate

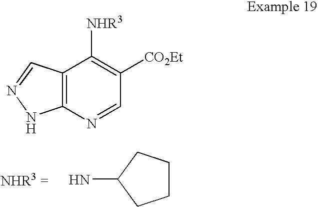

Intermediate 2 (0.035 g) was placed in a Reactivial™ and treated with cyclopentyl amine (0.05 ml). The mixture was heated at 90° C. for 1.5 h, then allowed to cool to room temperature and partitioned between chloroform (2 ml) and water (1 ml). The layers were separated and the organic phase was concentrated. The residual solid was triturated with Et₂O and the insoluble off-white solid collected and air-dried to afford Example 19 (0.016 g). LCMS showed MH⁺=275; T_{RET}=2.58 min.

### Example 20

### (reference example, as an intermediate): Ethyl 4-(tetrahydro-2H-pyran-4-ylamino)-1H-pyrazolo[3,4-b]pyridine-5-carboxylate

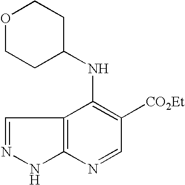 [1206] that is:

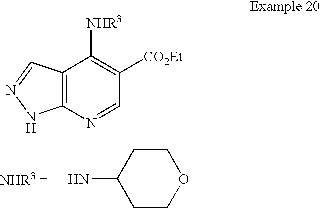

Intermediate 2 (0.035 g) was placed in a Reactivial™ and treated with 4-aminotetrahydropyran (0.05 ml). The mixture was heated at 90° C. for 1.5 h, then allowed to cool to room temperature and partitioned between chloroform (2 ml) and water (1 ml). The layers were separated and the organic phase was concentrated. The crude product was purified by mass directed autoprep HPLC to afford Example 20 as an off-white solid (0.011 g). LCMS showed MH⁺=291; T_{RET}=2.08 min.

### Alternative Synthetic Method for Example 20

Intermediate 2 (2 g) was suspended in 4-aminotetrahydropyran (2 g), and the mixture was heated at 90° C. for 6 h. The residual mixture was allowed to cool to room temperature and partitioned between chloroform (50 ml) and water (50 ml). The phases were separated and the organic phase was evaporated to dryness. The residue was triturated with Et₂O (30 ml) and the insoluble solid was collected and dried to afford Example 20 as a cream solid (2.24 g). LCMS showed MH⁺=291; T_{RET}=2.19 min.

### Example 21

### N-benzyl-1-ethyl-4-(tetrahydro-2H-pyran-4-ylamino)-1H-pyrazolo[3,4-b]pyridine-5-carboxamide

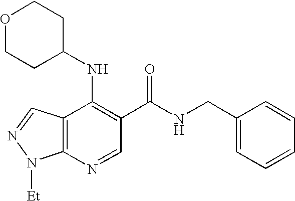 [1210] that is, Example 21 is:

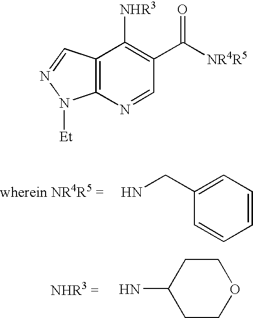

Three alternative methods, A, B and C, have been used to make Example 21, as follows:

### Example 21, Method A

A solution of the 4-chloro Intermediate 17 (0.031 g, 0.1 mmol) in ethanol (1.9 ml) was treated with triethylamine (0.07 ml, 0.5 mmol), followed by a 0.1M ethanolic solution of 4-aminotetrahydropyran (Intermediate 8, 1.1 ml of the 0.1M ethanolic solution=0.11 mmol). The mixture was heated at reflux (80° C.) for 18 h. A further portion of 4-amino-tetrahydropyran (0.01 ml of undiluted amine, not a solution thereof) was then added and heating continued for a further 24 h. Volatiles were removed in vacuo and the residue dissolved in dichloromethane (DCM), then applied to an solid phase extraction (SPE) cartridge (aminopropyl, 1 g) which was eluted first with DCM, then with methanol. Fractions containing desired material were concentrated in vacuo to afford Example 21 (0.004 g). LCMS showed MH⁺=380; T_{RET}=2.92 min.

### Example 21, Method B

Intermediate 17 (0.031 g, 0.1 mmol) was dissolved in acetonitrile (1 ml). 4-Aminotetrahydropyran hydrochloride (Intermediate 8A, 0.015 g, 0.11 mmol) and N,N-diisopropylethylamine (0.08 ml, 0.5 mmol) were added and the mixture stirred under nitrogen at 85° C. for 16 h, then concentrated in vacuo. The residue was partitioned between dichloromethane (DCM) and water. The layers were separated and the organic layer was concentrated in vacuo to afford Example 21 (0.027 g). LCMS showed MH⁺=380; T_{RET}=2.92 min.

### Example 21, Method C

This alternative route C to Example 21 involves formation of the ester of Example 3=Intermediate 32

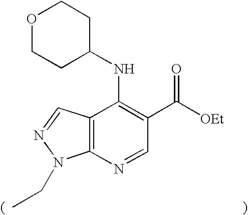

using one of the methods described above, conversion of the ester of Example 3/Intermediate 32 into the carboxylic acid (Intermediate 33) using the method given above for Intermediate 33, and then amide bond formation to form Example 21 using the method of Examples 81-84 below.

The following compounds can be similarly prepared using one or more of Methods A, B or C above, preferably Method A or B:

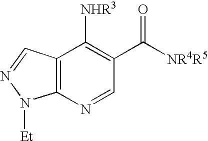

| | | | Starting | | | |
|---|---|---|---|---|---|---|
| | | | Material (for | | MH⁺ | T_{RET} |
| | NR⁴R⁵ | NHR³ | Method A or B) | Amine Reagent | ion | (min) |
| Example 22 | 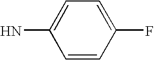 | 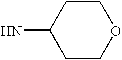 | Intermediate 19 | 4-aminotetrahydropyran | 384 | 3.09 |
| Example 23 | 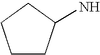 | 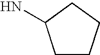 | Intermediate 20 | Cyclopentylamine | 342 | 3.29 |
| Example 24 | 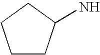 | 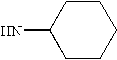 | Intermediate 20 | Cyclohexylamine | 356 | 3.47 |
| Example 25 | 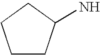 | 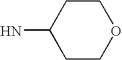 | Intermediate 20 | 4-aminotetrahydropyran | 358 | 2.79 |
| Example 27 | 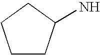 | 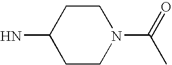 | Intermediate 20 | Intermediate 6 | 400 | 2.64 |
| Example 28 | 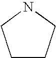 | 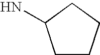 | Intermediate 21 | Cyclopentylamine | 328 | 2.69 |
| Example 29 | 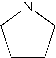 | 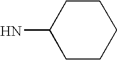 | Intermediate 21 | Cyclohexylamine | 342 | 2.87 |
| Example 30 | 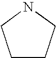 | 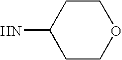 | Intermediate 21 | 4-aminotetrahydropyran | 344 | 2.33 |
| Example 31 | 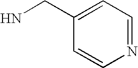 | 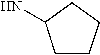 | Intermediate 22 | Cyclopentylamine | 365 | 2.38 |
| Example 32 | 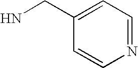 | 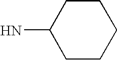 | Intermediate 22 | Cyclohexylamine | 379 | 2.54 |
| Example 33 | 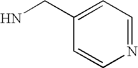 | 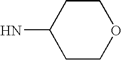 | Intermediate 22 | 4-aminotetrahydropyran | 381 | 2.09 |
| Example 34 | 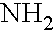 | 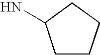 | Intermediate 24 | Cyclopentylamine | 274 | 2.59 |
| Example 35 | 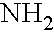 | 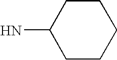 | Intermediate 24 | Cyclohexylamine | 288 | 2.79 |
| Example 36 | 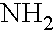 | 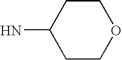 | Intermediate 24 | 4-aminotetrahydropyran | 290 | 2.22 |

### Example 39

### N-Benzyl-4-(cyclopentylamino)-1-ethyl-1H-pyrazolo[3,4-b]pyridine-5-carboxamide

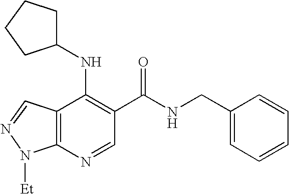 [1217] that is, Example 39 is:

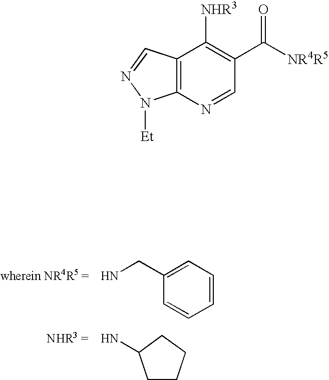

A solution of Intermediate 17 (0.031 g, 0.1 mmol) in ethanol (1 ml) was treated with triethylamine (0.07 ml, 0.5 mmol), followed by a 0.1M ethanolic solution of cyclopentyl amine (1.1 ml of the 0.1M ethanolic solution=0.11 mmol). The mixture was heated at reflux (80° C.) for 18 h. A further portion of cyclopentyl amine (0.009 ml of undiluted amine, not a solution thereof) was then added and heating continued for a further 24 h. Volatiles were removed in vacuo and the residue dissolved in DCM, then applied to an SPE cartridge (aminopropyl, 1 g) which was eluted first with DCM, then with methanol. The DCM fraction was concentrated in vacuo, then applied to an SPE cartridge (silica, 0.5 g) which was eluted sequentially with (i) DCM, (ii) Et₂O, (iii) EtOAc and (iv) MeOH. Fractions containing desired material were combined to afford Example 39 (0.007 g). LCMS showed MH⁺=364; T_{RET}=3.38 min.

Similarly prepared were the following:

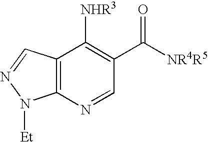

| | | | Starting | | MH⁺ | T_{RET} |
|---|---|---|---|---|---|---|
| | NR⁴R⁵ | NHR³ | Material | Amine reagent | ion | (min) |
| Example 40 | 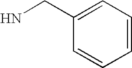 | 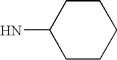 | Intermediate 17 | Cyclohexylamine | 378 | 3.43 |
| Example 41 | 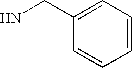 | 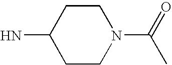 | Intermediate 17 | Intermediate 6 | 421 | 2.75 |
| Example 42 | 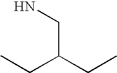 | 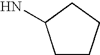 | Intermediate 18 | Cyclopentylamine | 358 | 3.63 |
| Example 43 | 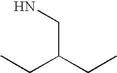 | 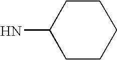 | Intermediate 18 | Cyclohexylamine | 372 | 3.79 |
| Example 44 | 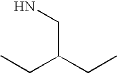 | 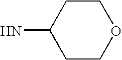 | Intermediate 18 | 4-aminotetrahydro-pyran | 374 | 3.13 |
| Example 45 | 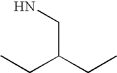 | 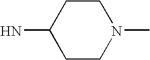 | Intermediate 18 | Intermediate 7 | 387 | 2.37 |
| Example 46 | 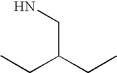 | 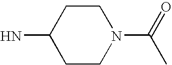 | Intermediate 18 | Intermediate 6 | 415 | 2.92 |
| Example 47 | 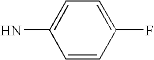 | 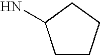 | Intermediate 19 | Cyclopentylamine | 368 | 3.61 |
| Example 48 | 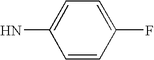 | 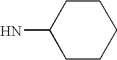 | Intermediate 19 | Cyclohexylamine | 382 | 3.76 |
| Example 49 | 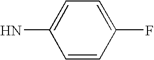 | 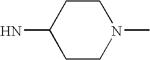 | Intermediate 19 | Intermediate 7 | 397 | 2.29 |
| Example 50 | 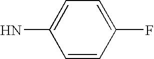 | 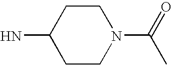 | Intermediate 19 | Intermediate 6 | 425 | 2.88 |
| Example 51 | 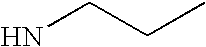 | 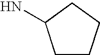 | Intermediate 23 | Cyclopentylamine | 316 | 3.05 |
| Example 52 | 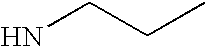 | 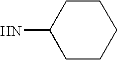 | Intermediate 23 | Cyclohexylamine | 330 | 3.26 |
| Example 53 | 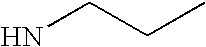 | 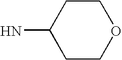 | Intermediate 23 | 4-aminotetrahydro-pyran | 332 | 2.58 |
| Example 55 | 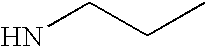 | 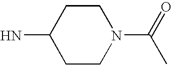 | Intermediate 23 | Intermediate 6 | 373 | 2.46 |

### Example 57

### 4-[(1-Acetylpiperidin-4-yl)amino]-1-ethyl-N-(pyridin-4-ylmethyl)-1H-pyrazolo[3,4-b]pyridine-5-carboxamide

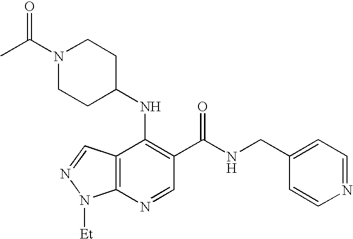 [1221] that is, Example 57 is:

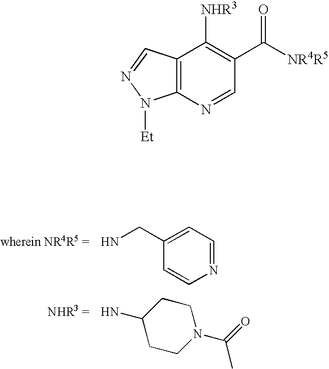

A solution of Intermediate 22 (0.03 g, ca. 0.1 mmol) in ethanol (1 ml) was treated with triethylamine (0.07 ml, 0.5 mmol), followed by a 0.1M ethanolic solution of Intermediate 6 (1.1 ml of the solution=0.11 mmol). The mixture was heated at reflux (80° C.) for 18 h. A further portion of Intermediate 6 (0.01 ml, undiluted) was then added and heating continued for a further 24 h. Volatiles were removed in vacuo and the residue dissolved in DCM, then applied to an SPE cartridge (aminopropyl, 1 g) which was eluted first with DCM, then with methanol.

The DCM fraction was concentrated in vacuo, then applied to an SPE cartridge (silica, 0.5 g) eluting with (I) DCM, (ii) EtOAc and (iii) a stepwise gradient of chloroform:methanol (from 99:1 up to 4:1). Fractions containing desired material were combined to afford Example 57 (0.003 g). LCMS showed MH⁺=422; T_{RET}=2.1 min.

### Example 61

### N-Benzyl-4-(cyclopentylamino)-1-methyl-1H-pyrazolo[3,4-b]pyridine-5-carboxamide

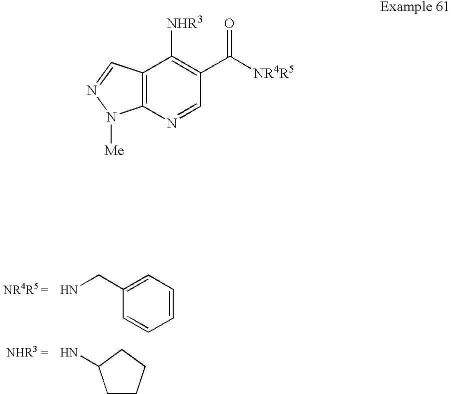

A solution of Intermediate 28 (0.03 g, 0.1 mmol) in ethanol (1 ml) was treated with a 0.1M ethanolic solution of cyclopentyl amine (1.1 ml of solution=0.11 mmol). Triethylamine (0.07 ml, 0.5 mmol) was then added and the mixture heated at reflux (85° C.), under nitrogen for 12 h. A further portion of cyclopentyl amine (0.009 ml, undiluted) was then added and heating continued for a further 36 h. The mixtures were concentrated in vacuo and the residue treated with chloroform. A small amount of insoluble material was collected by filtration, then the filtrate applied to an SPE cartridge (aminopropyl, 1 g) which was eluted first with DCM, then with methanol. Fractions containing desired material were combined to afford Example 61 (0.039 g). LCMS showed MH⁺=350; T_{RET}=2.88 min.

Similarly prepared were the following:

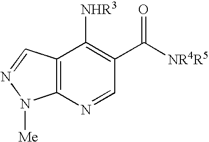

| | | | Starting | | MH⁺ | T_{RET} |
|---|---|---|---|---|---|---|
| | NR⁴R⁵ | NHR³ | Material | Amine Reagent | ion | (min) |
| Example 62 | 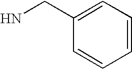 | 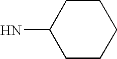 | Intermediate 28 | Cyclohexylamine | 364 | 3.05 |
| Example 63 | 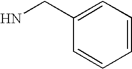 | 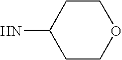 | Intermediate 28 | 4-aminotetrahydropyran | 366 | 2.52 |
| Example 64 | 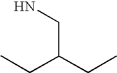 | 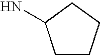 | Intermediate 30 | Cyclopentylamine | 344 | 3.06 |
| Example 65 | 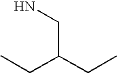 | 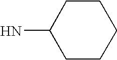 | Intermediate 30 | Cyclohexylamine | 358 | 3.23 |
| Example 66 | 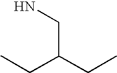 | 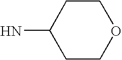 | Intermediate 30 | 4-aminotetrahydropyran | 360 | 2.69 |
| Example 67 | 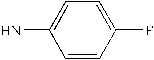 | 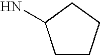 | Intermediate 29 | Cyclopentylamine | 354 | 3.17 |
| Example 68 | 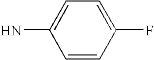 | 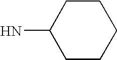 | Intermediate 29 | Cyclohexylamine | 368 | 3.33 |
| Example 69 | 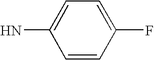 | 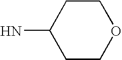 | Intermediate 29 | 4-aminotetrahydropyran | 370 | 2.72 |
| Example 70 | 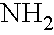 | 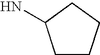 | Intermediate 31 | Cyclopentylamine | 260 | 2.10 |
| Example 71 | 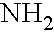 | 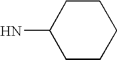 | Intermediate 31 | Cyclohexylamine | 274 | 2.29 |

### Example 74

### 4-[(1-Acetylpiperidin-4-yl)amino]-N-benzyl-1-methyl-1H-pyrazolo[3,4-b]pyridine-5-carboxamide

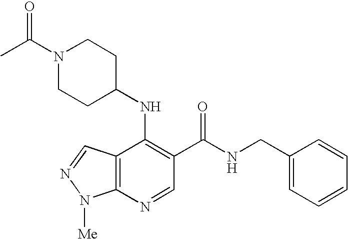 [1228] that is, Example 74 is:

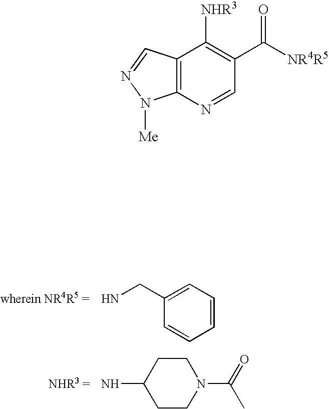

A solution of Intermediate 28 (0.03 g, 0.1 mmol) in ethanol (1 ml) was treated with a 0.1M ethanolic solution of Intermediate 6 (1.1 ml of solution=0.11 mmol). Triethylamine (0.07 ml, 0.5 mmol) was then added and the mixture heated at reflux (85° C.), under nitrogen for 12 h. A further portion of Intermediate 6 (0.1 mmol) was then added and heating continued for a further 36 h. The mixtures were concentrated in vacuo and the residue treated with chloroform. A small amount of insoluble material was collected by filtration, then the filtrate applied to an SPE cartridge (aminopropyl, 1 g) which was eluted first with DCM, then with methanol. Fractions containing desired material were combined and concentrated in vacuo. The residue was further purified by SPE (silica, 0.5 g) eluting with (i) DCM, (ii) chloroform, (iii) EtOAc and (iv) a stepwise gradient of chloroform:methanol (from 99:1 up to 4:1). Fractions containing desired material were combined to afford Example 74 (0.029 g). LCMS showed MH⁺=407; T_{RET}=2.57 min.

### Example 81

### 1-Ethyl-N-methyl-4-(tetrahydro-2H-pyran-4-ylamino)-1H-pyrazolo[3,4-b]pyridine-5-carboxamide

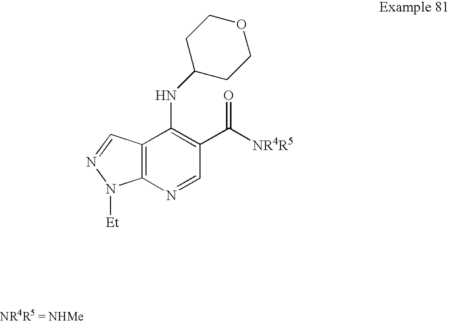

To a stirred suspension of Intermediate 33 (0.025 g, ca. 0.08 to 0.09 mmol) in chloroform (2 ml) was added thionyl chloride (0.025 ml) and the mixture stirred at room temperature for 1 h. The mixture was cooled to 0° C. and methylamine added (2M solution in THF, 0.69 ml=1.38 mmol). After returning to room temperature the mixture was stirred for a further 1 h, then quenched by addition of water (4 ml) and the layers separated. The organic layer was concentrated then applied to an SPE cartridge (silica, 1 g) which was eluted with (i) DCM, (ii) Et₂O (2:1), (iii) EtOAc, (iv) MeOH:EtOAc (1:9). Fractions containing desired material were combined to afford Example 81 (0.019 g). LCMS showed MH⁺=304; T_{RET}=2.19 min.

Similarly prepared:

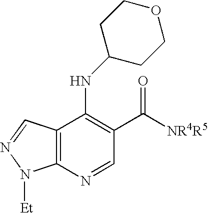

| | | | MH⁺ | T_{RET} |
|---|---|---|---|---|
| | NR⁴R⁵ | Amine reagent | ion | (min) |
| Example 82 | NMe₂ | Dimethylamine (2 M in THF) | 318 | 2.06 |
| Example 83 | NHEt | Ethylamine (2 M in THF) | 318 | 2.31 |
| Example 84 | NHⁱPr | Isopropylamine (2 M in THF) | 332 | 2.44 |

### Example 83

### N,1-Diethyl-4-(tetrahydro-2H-pyran-4-ylamino)-1H-pyrazolo[3,4-b]pyridine-5-carboxamide; also named 1-ethyl-N-ethyl-4-(tetrahydro-2H-pyran-4-ylamino)-1H-pyrazolo[3,4-b]pyridine-5-carboxamide

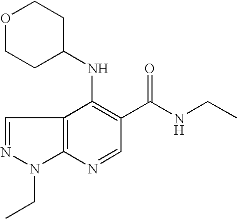

In an alternative embodiment to the process described for Examples 81-84 above, Example 83 can be made according to the following method:

A mixture of Intermediate 33 (3.0 g, 10.33 mmol), EDC (2.25 g, 11.7 mmol), and HOBT (1.68 g, 12.4 mmol) was stirred at room temperature for 1 hour. Ethylamine (6.2 ml, 12.4 mmol, 2M-solution in THF) was added, and stirring was continued at room temperature for 22 hours. The solvents were removed in vacuo, and the residual solid was dissolved in chloroform (250 ml) and washed successively with water (70 ml) and 5%-sodium hydrogen carbonate solution (70 ml). After drying over anhydrous sodium sulphate, the organic solution was evaporated in vacuo to give a pale orange solid (4.15 g). This solid was dissolved in a mixture of dichloromethane (15 ml) and chloroform (5 ml) and purified by column chromatography (Biotage, silica, 100 g), eluting initially with EtOAc-cyclohexane (2:1) and finally with neat EtOAc. The product containing fractions were combined and evaporated to give Example 83 as a pale yellow solid (3.05 g). LCMS showed MH⁺=318; T_{RET}=2.33 min. ¹H NMR (400 MHz in d₆-DMSO, 27° C., δppm) 9.76 (d, 1H) 8.35 (s, 1H) 7.94 (s, 1H) 5.99 (br m, 1H) 4.47 (q, 2H) 4.16-4.01 (m's, 3H) 3.62 (m, 2H) 3.48 (m, 2H) 2.13 (m, 2H) 1.77 (m, 2H) 1.49 (t, 3H) 1.28 (t, 3H).

### Example 85

### N-Benzyl-1-ethyl-4-[(3S)-tetrahydrofuran-3-ylamino]-1H-pyrazolo[3,4-b]pyridine-5-carboxamide

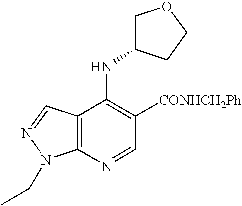 [1237] That is, Example 85 is:

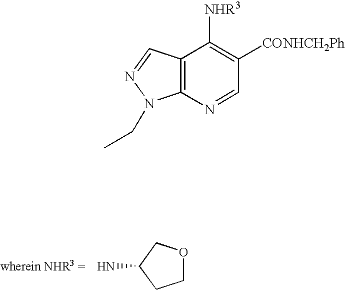

Intermediate 41 (0.017 g, 0.062 mmol) was dissolved in DMF (2 ml), then treated with HATU (0.023 g) followed by diisopropylethyl amine (0.021 ml) and the mixture stirred for 10 min. Benzylamine (0.007 ml) was then added and stirring continued for a further 64 h. The mixture was concentrated in vacuo and the residue dissolved in DCM (1.5 ml) then treated with saturated aqueous sodium bicarbonate solution (1.5 ml). This mixture was stirred for 30 min, then the layers were separated and the organic layer was applied to an SPE cartridge (silica, 1 g) which was eluted sequentially with a gradient of ethyl acetate:cyclohexane (1:4, then 1:2, 1:1, 2:1 and 1:0). Fractions containing desired material were concentrated in vacuo to afford Example 85 (0.017 g). LCMS showed MH⁺=366; T_{RET}=2.80 min.

Similarly prepared were the following:

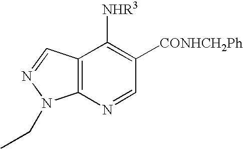

| | | Starting | MH⁺ | T_{RET} |
|---|---|---|---|---|
| | NHR³ | material | ion | (min) |
| Example 86 | 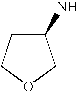 | Intermediate 42 | 366 | 2.80 |
| Example 87 | 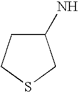 | Intermediate 44 | 382 | 3.11 |
| Example 88 | 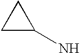 | Intermediate 45 | 336 | 3.00 |
| Example 89 | 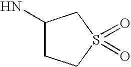 | Intermediate 46 | 414 | 2.69 |
| Example 90 | 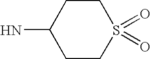 | Intermediate 47 | 428 | 2.75 |

### Example 91

### N-Benzyl-1-ethyl-4-(tetrahydro-2H-thiopyran-4-ylamino)-1H-pyrazolo[3,4-b]pyridine-5-carboxamide

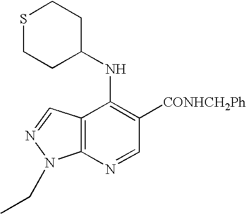

Intermediate 43 (0.019 g) was dissolved in DMF (2 ml), then treated with HATU (0.024 g) followed by diisopropylethyl amine (0.022 ml) and the mixture stirred for 10 min. Benzylamine (0.007 ml) was then added and stirring continued for a further 64 h. The mixture was concentrated in vacuo and the residue dissolved in DCM (1.5 ml) then treated with saturated aqueous sodium bicarbonate solution (1.5 ml). This mixture was stirred for 30 min, then the layers were separated and the organic layer applied to an SPE cartridge (silica, 1 g) which was eluted sequentially with a gradient of ethyl acetate:cyclohexane (1:4, then 1:2, 1:1 and 1:0). Fractions containing desired material were concentrated in vacuo to afford Example 91 (0.023 g). LCMS showed MH⁺=396; T_{RET}=3.26 min.

### Example 92

### 1-Ethyl-N-(4-fluorophenyl)-4-[(3S)-tetrahydrofuran-3-ylamino]-1H-pyrazolo[3,4-b]pyridine-5-carboxamide

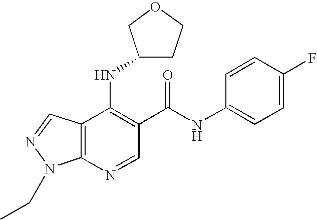 [1243] that is, Example 92 is:

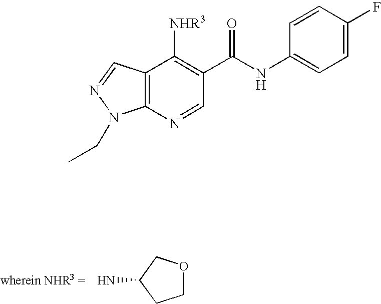

Intermediate 41 (0.017 g) was dissolved in DMF (2 ml), then treated with HATU (0.023 g) followed by diisopropylethyl amine (0.021 ml) and the mixture stirred for 10 min. 4-Fluoroaniline (0.006 ml) was then added and stirring continued for a further 64 h. The mixture was concentrated in vacuo and the residue dissolved in DCM (1.5 ml) then treated with saturated aqueous sodium bicarbonate solution (1.5 ml). This mixture was stirred for 30 min, then the layers were separated and the organic layer concentrated in vacuo. The crude mixture was purified by mass directed autoprep HPLC to afford Example 92 (0.013 g). LCMS showed MH⁺=370; T_{RET}=2.91 min.

Similarly prepared were the following:

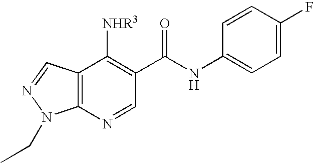

| | | Starting | MH⁺ | T_{RET} |
|---|---|---|---|---|
| | NHR³ | material | ion | (min) |
| Example 93 | 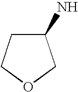 | Intermediate 42 | 370 | 2.91 |
| Example 94 | 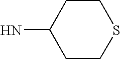 | Intermediate 43 | 400 | 3.37 |
| Example 95 | 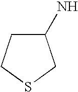 | Intermediate 44 | 386 | 3.27 |
| Example 96 | 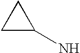 | Intermediate 45 | 340 | 3.21 |
| Example 97 | 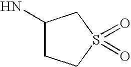 | Intermediate 46 | 418 | 2.80 |
| Example 98 | 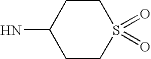 | Intermediate 47 | 432 | 2.84 |

### Example 99

In all of Examples 22 to 98, where a 4-amino 5-carboxamide Example of the following Formula I has been synthesised from the 4-chloro derivative, then an alternative final-step synthesis is as follows:

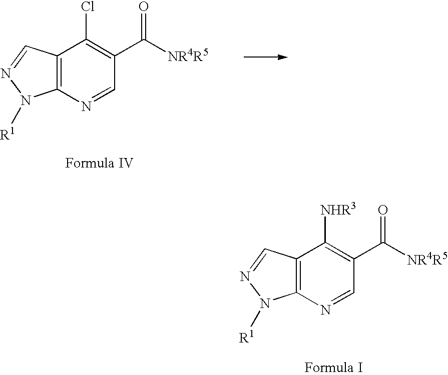

An intermediate of Formula IV above (0.1 mmol) was dissolved in acetonitrile (1 ml). An amine of formula R³NH₂ (0.11 mmol, 1.1 mole equivalents) and N,N-diisopropylethylamine (0.5 mmol, 5 mole equivalents) were added and the mixture stirred under nitrogen at 85° C. for 16 h. After concentration in vacuo, the residue was partitioned between dichloromethane (DCM) and water. The layers were separated and the organic layer was concentrated in vacuo to afford an Example of Formula I.

### Example 100

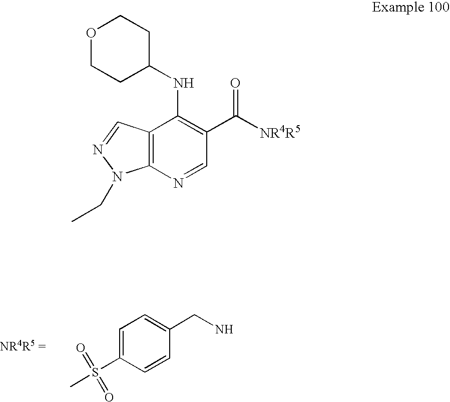

Intermediate 33 (0.048 mmol) was dissolved in DMF (0.5 ml), then treated with HATU (0.048 mmol) followed by diisopropylethyl amine (0.096 mmol) and the mixture stirred for 10 min. 4-Methylsulfonylbenzylamine (0.052 mmol, available from Acros Organics) was then added and stirring continued for a further 16 hours. The mixture was concentrated in vacuo. The crude mixture was purified by mass directed autoprep HPLC to afford Example 100 (0.013 g). LCMS showed MH⁺=458; T_{RET}=2.22 min.

Similarly prepared, but replacing the 4-methylsulfonylbenzylamine with the same or similar number of moles of another amine R⁴R⁵NH, were the following compounds (Examples 102 to 182):

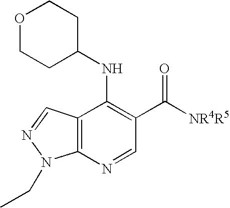

| | NR⁴R⁵ (the N | | | | |
|---|---|---|---|---|---|
| | atom linking R⁴ | | | | |
| | and R⁵ to the | | | | |
| | —CO-pyrazolo- | | | | |
| | pyridine moiety | Source of | Starting | MH⁺ | T_{RET} |
| | is underlined) | R⁴R⁵NH | Material | ion | (min) |
| Example 102 | 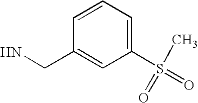 | J. Chem. Soc., 1945,633 | Intermediate 33 | 458 | 2.2 |
| Example 103 | 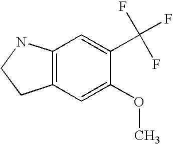 | WO 98/52943 | Intermediate 33 | 490 | 2.66 |
| Example 104 | 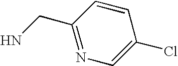 | J. Org. Chem., 1979,44(3), 396 | Intermediate 33 | 415 | 2.28 |
| Example 105 | 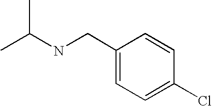 | SeriyaKhimicheskaya, 1989,(7), 1694 | Intermediate 33 | 456 | 2.65 |
| Example 106 | 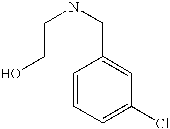 | SALOR (Aldrich) | Intermediate 33 | 458 | 2.32 |
| Example 107 | 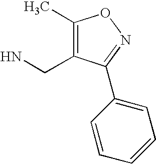 | Maybridge ChemicalCompany Ltd.TrevillettTintagelCornwall PL34 0HWUnited Kingdom | Intermediate 33 | 461 | 2.5 |
| Example 108 | 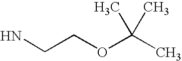 | MicroChemistry-RadaPharmaShosse Entusiastov 56Moscow, 111123Russia | Intermediate 33 | 390 | 2.28 |
| Example 109 | 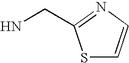 | MicroChemistry-RadaPharmaShosse Entusiastov 56Moscow, 111123Russia. Alternatively,available from:Matrix Scientific(USA), or Synthesis1998, 641, orTetrahedron 1995,51, 12731 | Intermediate 33 | 387 | 2.13 |
| Example 110 | 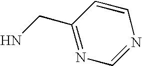 | Bulletin des SocietesChimiques Belges,(1982), 91(2), 153 | Intermediate 33 | 382 | 1.98 |
| Example 111 | 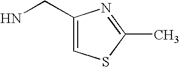 | MicroChemistry-RadaPharmaShosse Entusiastov 56Moscow, 111123Russia | Intermediate 33 | 401 | 2.14 |
| Example 112 | 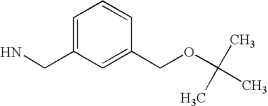 | | Intermediate 33 | 466 | 2.67 |
| Example 113 | 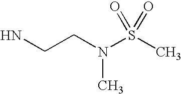 | Ultrafine (UFC Ltd.),see above for address | Intermediate 33 | 425 | 2 |
| Example 114 | 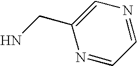 | Austin ChemicalCompany, Inc.1565 Barclay Blvd.Buffalo Grove,IL, 60089USA | Intermediate 33 | 382 | 2 |
| Example 115 | 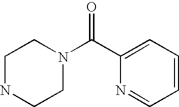 | WO 02/83624 | Intermediate 33 | 464 | 1.97 |
| Example 116 | 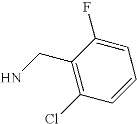 | Fluka Chemie AG | Intermediate 33 | 432 | 2.52 |
| Example 117 | 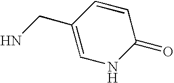 | MicroChemistry-RadaPharmaShosse Entusiastov 56Moscow, 111123Russia | Intermediate 33 | 397 | 1.96 |
| Example 118 | 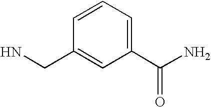 | WO 02/85860 | Intermediate 33 | 423 | 2.09 |
| Example 119 | 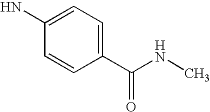 | Butt Park Ltd.Braysdown WorksPeasedown St. JohnBath, BA2 8LL,United Kingdom | Intermediate 33 | 423 | 2.19 |
| Example 120 | 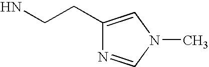 | Sigma | Intermediate 33 | 398 | 1.77 |
| Example 121 | 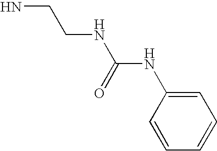 | US 4562184 | Intermediate 33 | 452 | 2.21 |
| Example 122 | 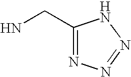 | Dynamit Nobel GmbH,Germany; or SavilleWhittle Ltd (UK agentsof Dynamit Nobel),Vickers Street,Manchester M40 8EF,United Kingdom | Intermediate 33 | 372 | 1.93 |
| Example 123 | 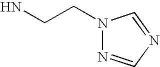 | WO 02/66470 | Intermediate 33 | 385 | 1.93 |
| Example 125 | 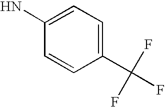 | Aldrich | Intermediate 33 | 434 | 2.84 |
| Example 126 | 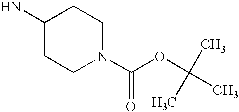 | AstaTech, Inc.8301 Torresdale Ave.19C, Philadelphia,PA, 19136, USA | Intermediate 33 | 473 | 2.5 |
| Example 127 | 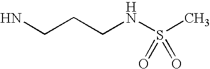 | | Intermediate 33 | 425 | 1.99 |
| Example 128 | 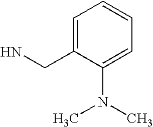 | J. Org. Chem., 2001,66(6), 1999 | Intermediate 33 | 423 | 1.97 |
| Example 129 | 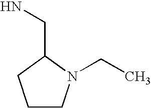 | Acros Organics | Intermediate 33 | 401 | 1.82 |
| Example 130 | 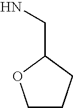 | Aldrich | Intermediate 33 | 374 | 2.08 |
| Example 131 | 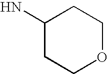 | Combi-Blocks Inc.,7949 Silverton Av.,Suite 915, San Diego,CA 92126, USA (seealso Intermediate 8A) | Intermediate 33 | 374 | 2.04 |
| Example 132 | 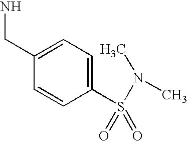 | J. Org. Chem., 1955,20, 1657 | Intermediate 33 | 487 | 2.39 |
| Example 133 | 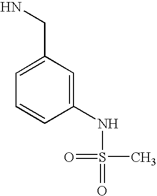 | J. Med. Chem., 1999,42(14), 2504;or variation of: Lis etal., J. Med. Chem.,1990, 33(10), 2883,see Scheme III andref. 24 | Intermediate 33 | 473 | 2.24 |
| Example 135 | 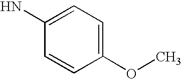 | Aldrich | Intermediate 33 | 396 | 2.42 |
| Example 136 | 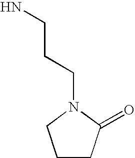 | Aldrich | Intermediate 33 | 415 | 2.03 |
| Example 137 | 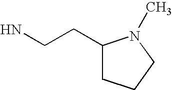 | Aldrich | Intermediate 33 | 401 | 1.78 |
| Example 138 | 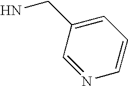 | Aldrich | Intermediate 33 | 381 | 1.81 |
| Example 139 | 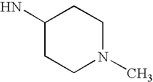 | MicroChemistry-RadaPharmaShosse Entusiastov 56Moscow, 111123Russia | Intermediate 33 | 387 | 1.74 |
| Example 140 | 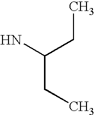 | Aldrich | Intermediate 33 | 360 | 2.16 |
| Example 141 | 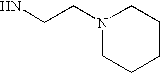 | Aldrich | Intermediate 33 | 401 | 1.81 |
| Example 142 | 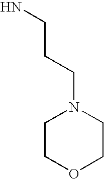 | Aldrich | Intermediate 33 | 417 | 1.75 |
| Example 143 | 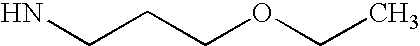 | Aldrich | Intermediate 33 | 376 | 2.16 |
| Example 144 | 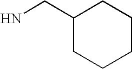 | Aldrich; or Baruah etal., Synlett, 1999, 4,409 | Intermediate 33 | 386 | 2.59 |
| Example 145 | 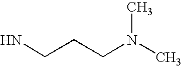 | Aldrich | Intermediate 33 | 375 | 1.73 |
| Example 146 | 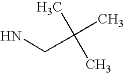 | Fluorochem Ltd.Wesley StreetOld GlossopDerbyshireSK13 7RYUnited Kingdom | Intermediate 33 | 360 | 2.16 |
| Example 147 | 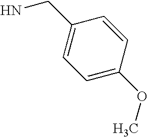 | Aldrich; or Acros; orJung et al.,tetrahedron Lett.,2002, 43(48), 8735; orMeindl et al., J. Med.Chem., 1984, 27(9),1111; or Organic lett.,2002, 4(12), 2055 | Intermediate 33 | 410 | 2.4 |
| Example 148 | 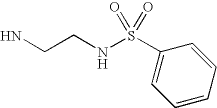 | Berk Univar plcBerk HouseP.O. Box 56Basing ViewBasingstokeHants RG21 2E6,United Kingdom | Intermediate 33 | 473 | 2.26 |
| Example 149 | 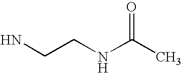 | Aldrich | Intermediate 33 | 375 | 1.9 |
| Example 150 | 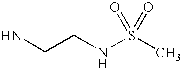 | MicroChemistry-RadaPharmaShosse Entusiastov 56Moscow, 111123Russia | Intermediate 33 | 411 | 1.95 |
| Example 152 | 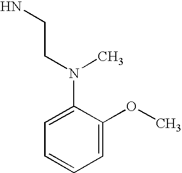 | Nippon KagakuZasshi., 1960, 81p. 962. | Intermediate 33 | 453 | 1.96 |
| Example 153 | 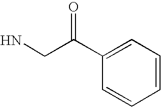 | Aldrich | Intermediate 33 | 408 | 2.35 |
| Example 154 | 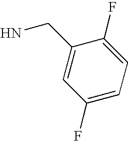 | Aldrich | Intermediate 33 | 416 | 2.5 |
| Example 155 | 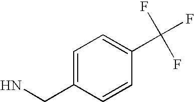 | Aldrich; or Meindl etal., J. Med. Chem.,1984, 27(9), 1111; orOrganic Letters, 2002,4(12), 2055 | Intermediate 33 | 448 | 2.68 |
| Example 156 | 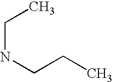 | Alfa Aesar,A Johnson MattheyCompany30 Bond StreetWard Hill, MA01835-8099USA | Intermediate 33 | 360 | 2.16 |
| Example 157 | 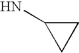 | Aldrich | Intermediate 33 | 330 | 2.04 |
| Example 158 | 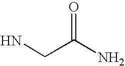 | Aldrich | Intermediate 33 | 347 | 1.83 |
| Example 159 | 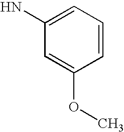 | Aldrich | Intermediate 33 | 396 | 2.49 |
| Example 160 | 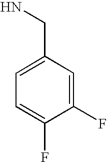 | Aldrich | Intermediate 33 | 416 | 2.53 |
| Example 161 | 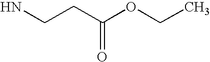 | Aldrich | Intermediate 33 | 390 | 2.18 |
| Example 162 | 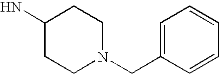 | Aldrich | Intermediate 33 | 463 | 1.96 |
| Example 163 | 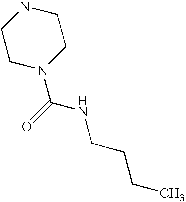 | US 4987132 | Intermediate 33 | 458 | 2.13 |
| Example 164 | 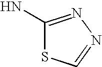 | Aldrich | Intermediate 33 | 374 | 2.22 |
| Example 165 | 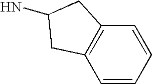 | Aldrich; or TCI-America; orMaybridge-Int. | Intermediate 33 | 406 | 2.53 |
| Example 166 | 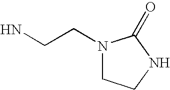 | Maybridge ChemicalCompany Ltd.TrevillettTintagelCornwall PL34 0HWUnited Kingdom | Intermediate 33 | 402 | 1.93 |
| Example 167 | 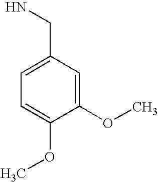 | Aldrich; or Baruah etal., Synlett, 1999, 4,409 | Intermediate 33 | 440 | 2.3 |
| Example 168 | 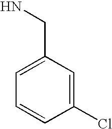 | Aldrich; or Meindl etal., J. Med. Chem.,1984, 27(9), 1111,; orOrganic Letters,2002, 4(12), 2055 | Intermediate 33 | 414 | 2.58 |
| Example 169 | 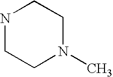 | Aldrich | Intermediate 33 | 373 | 1.64 |
| Example 170 | 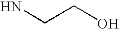 | Aldrich | Intermediate 33 | 334 | 1.85 |
| Example 171 | 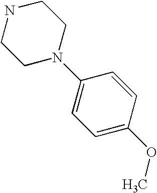 | Aldrich | Intermediate 33 | 465 | 2.29 |
| Example 172 | 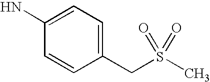 | EP 666258 | Intermediate 33 | 458 | 2.25 |
| Example 173 | 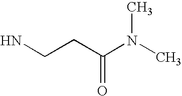 | J. Chem. Soc., 1954,1171 | Intermediate 33 | 389 | 1.98 |
| Example 174 | 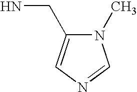 | Peakdale MolecularLtd., Peakdale SciencePark, Sheffield Road,Chapel-en-le-Frith,High Peak SK23 0PG,United Kingdom | Intermediate 33 | 384 | 1.76 |
| Example 175 | 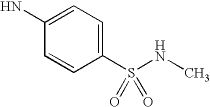 | Fluorochem Ltd.Wesley StreetOld GlossopDerbyshire SK13 7RYUnited Kingdom | Intermediate 33 | 459 | 2.36 |
| Example 176 | 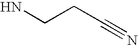 | Lancaster SynthesisLtd, Newgate, WhiteLund, Morecambe,Lancashire LA3 3DY,United Kingdom | Intermediate 33 | 343 | 2.01 |
| Example 178 | 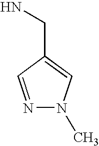 | TimTec, Inc.P O Box 8941Newark, DE, 19714-8941USA | Intermediate 33 | 384 | 2.03 |
| Example 179 | 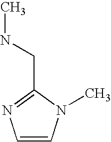 | ChemBridge Europe,4 Clark's Hill Rise,Hampton Wood,Evesham,Worcestershire WR116FW, UnitedKingdom | Intermediate 33 | 398 | 1.70 |
| Example 180 | 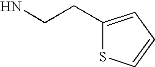 | Aldrich | Intermediate 33 | 400 | 2.41 |
| Example 181 | 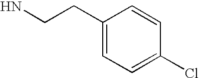 | Aldrich | Intermediate 33 | 428 | 2.61 |
| Example 182 | 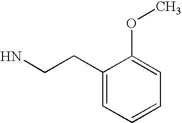 | Aldrich | Intermediate 33 | 424 | 2.49 |

### Example 109

### 1-Ethyl-4-(tetrahydro-2H-pyran-4-ylamino)-N-(1,3-thiazol-2-ylmethyl)-1H-pyrazolo[3,4-b]pyridine-5-carboxamide

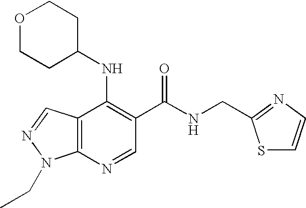

An alternative process for preparing Example 109 is given below:

1-Hydroxybenzotriazole (0.215 g, 1.59 mmol) and 1-[3-(dimethylamino)propyl]-3-ethyl-carbodiimide hydrochloride (0.357 g, 1.86 mmol) were added to a suspension of Intermediate 33 (0.384 g, 1.32 mmol) in DMF (10 ml). After stirring at room temperature for 30 minutes, (1,3-thiazol-2-ylmethyl)amine (0.182 g, 1.59 mmol) (commercially available from MicroChemistry Building Blocks (Russia) or Matrix Scientific (USA), or preparable as disclosed in Synthesis 1998, 641, or Tetrahedron 1995, 51, 12731) was added. The reaction was stirred for 18 hours and then partitioned between ether and water. The organic phase was washed with brine, dried (MgSO₄) and evaporated in vacuo. The residue was purified by chromatography (Biotage, silica 90 g) eluting with cyclohexane:EtOAc followed by EtOAc. The material was triturated with cyclohexane and filtered to afford Example 109 (0.244 g) as a pale yellow solid. LCMS showed MH⁺ 387; T_{RET}=2.49 min. ¹H NMR (400 MHz in CDCl₃, δppm) δ 9.74 (d, 1H) 8.50 (s, 1H) 7.94 (s, 1H) 7.74 (d, 1H), 7.33 (d, 1H), 7.17 (m, 1H), 4.94 (d, 2H) 4.45 (q, 2H) 4.15-4.00 (m, 3H), 3.63 (m, 2H). 2.15 (m, 2H) 1.85-1.73 (m, 3H) 1.48 (t, 3H).

### Example 167

### N-{[3,4-Bis(methyloxy)phenyl]methyl}-1-ethyl-4-(tetrahydro-2H-pyran-4-ylamino)-1H-pyrazolo[3,4-b]pyridine-5-carboxamide

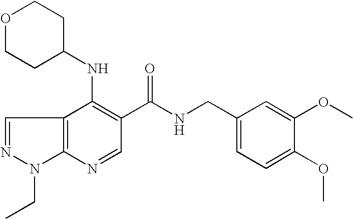

In an alternative embodiment to the process described above for Examples 100-182, Example 167 can be made according to the following method:

A mixture of Intermediate 33 (0.498 g, 1.72 mmol), EDC (0.46 g, 2.41 mmol), and HOBT (0.278 g, 1.68 mmol) was stirred at room temperature for 0.25 hours. Veratrylamine (3,4-dimethoxybenzylamine, 0.31 ml, 2.05 mmol, obtainable from Aldrich or Synlett, 1999, 4, 409) was added, and stirring was continued at room temperature for 22 hours. The reaction mixture was partitioned between Et₂O and water. The aqueous phase was extracted with Et₂O and the combined organic phases washed with brine, dried (MgSO₄) and evaporated in vacuo. The residue was purified by chromatography (Biotage, silica 40 g) eluting with EtOAc:cyclohexane (2:1). The material was further purified by SPE (SCX-2, 10 g) eluting with methanol then ammonia in methanol (0.5M). The ammonia methanol fractions were combined and evaporated in vacuo to afford Example 167 as a white foam (0.633 g). LCMS showed MH⁺=440; T_{RET}=2.65 min. ¹H NMR (400 MHz in CDCl₃, 27° C., δppm) 9.78 (d, 1H) 8.37 (s, 1H) 7.94 (s, 1H) 6.94-6.82 (m, 3H) 6.29 (br m, 1H) 4.56 (d, 2H) 4.46 (q, 2H) 4.15-4.01 (m's, 3H) 3.89 (s, 6H) 3.63 (m, 2H) 2.15 (m, 2H) 1.78 (m, 2H) 1.49 (t, 3H).

### Example 178

### 1-Ethyl-N-[(1-methyl-1H-pyrazol-4-yl)methyl]-4-(tetrahydro-2H-pyran-4-ylamino)-1H-pyrazolo[3,4-b]pyridine-5-carboxamide

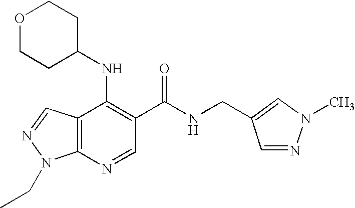

The ¹H NMR data for Example 178 (as prepared by the process described in Examples 100-182 above) was as follows:

¹H NMR (400 MHz in CDCl₃, δppm) δ 9.90 (m, 1H) 8.37 (s, 1H) 7.94 (s, 1H) 7.49 (s, 1H), 7.40 (s, 1H) 6.39 (m, 1H) 4.50-4.42 (m, 4H) 4.15-4.00 (m, 3H) 3.89 (s, 3H), 3.63 (m, 2H) 2.52 (m, 2H) 2.20-2.10 (m, 2H) 1.85-1.73 (m, 3H) 1.48 (t, 3H).

### Example 183

### Ethyl 4-(cyclohexylamino)-1-(3-ethoxy-3-oxopropyl)-1H-pyrazolo[3,4-b]pyridine-5-carboxylate

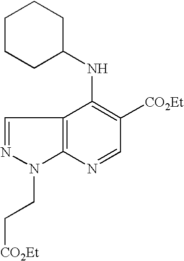

A vigorously stirred mixture of Intermediate 48 (40 mg), anhydrous potassium carbonate (57 mg) and ethyl 3-bromopropanoate (0.027 ml) in anhydrous DMF (1 ml) was heated at 65° C. overnight. The reaction mixture was concentrated, and the residue was partitioned between dichloromethane (5 ml) and water (5 ml). The phases were separated and the organic phase was evaporated to a residual oil which was purified by mass directed autoprep HPLC to afford Example 183 (5 mg). LCMS showed MH⁺=389; T_{RET}=3.65 min.

### Example 185

### Ethyl 1-n-propyl-4-(tetrahydro-2H-pyran-4-ylamino)-1H-pyrazolo[3,4-b]pyridine-5-carboxylate

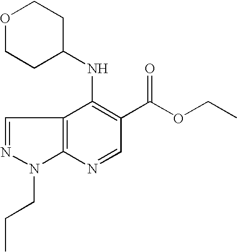

Sodium hydride (0.067 g, 60% dispersion in oil) was added to a stirred solution of Example 20 (0.47 g) in DMF (19 ml), followed by n-propyl iodide (0.17 ml). The mixture was stirred at 23° C. for 16 hours, then concentrated, diluted with chloroform (30 ml) and washed with 1:1 water:brine solution (30 ml), separated and the organic layer concentrated. The residue was purified on a SPE catridge (silica, 10 g) eluting with 10 ml volumes of dichloromethane, 1:1 diethyl ether:cyclohexane, and diethyl ether. The combined 1:1 diethyl ether: cyclohexane, and diethyl ether, fractions were concentrated to give Example 185 as a clear gum (0.23 g). LCMS showed MH⁺=333; T_{RET}=3.14 min.

### Example 186

### Ethyl 1-(2-hydroxyethyl)-4-(tetrahydro-2H-pyran-4-ylamino)-1H-pyrazolo[3,4-b]pyridine-5-carboxylate

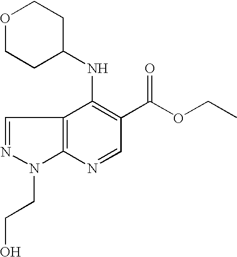

2-Bromoethanol (0.008 ml) was added to a solution of Example 20 (0.03 g) in anhydrous DMF (1.5 ml), with 2-tert-butylimino-2-diethylamino-1,3-dimethyl-perhydro-1,3,2-diazaphosphorine (polymer bound, 2.3 mmol/g loading, 0.045 g). The mixture was shaken at 23° C. for 16 hours, then the solution drained from the resin, and the resin was washed with DMF. The combined organics were concentrated, and the residue purified on a SPE cartridge (silica, 1 g) eluting with 70-100% ethyl acetate in cyclohexane. The combined fractions were concentrated to give Example 186 as a white solid (0.011 g). LCMS showed MH⁺=335; T_{RET}=2.47 min.

### Example 187

### N-[4-(Methylsulfonyl)benzyl]-1-n-propyl-4-(tetrahydro-2H-pyran-4-ylamino)-1H-pyrazolo[3,4-b]pyridine-5-carboxamide

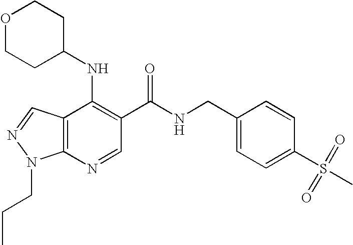

Intermediate 50 (0.03 g) was stirred in DMF (1 ml) with DIPEA (0.035 ml) and HATU (0.038 g) for 20 min. 4-(Methylsulfonyl)benzylamine hydrochloride (0.024 g) was added to the mixture and the solution was stirred for 8 hours at 23° C. The solution was concentrated and the residue dissolved in dichloromethane (6 ml) then washed with saturated sodium bicarbonate solution (6 ml) and 1:1 brine:water (6 ml), separated by hydrophobic frit. The organic layer was concentrated to give Example 187 as a white solid (0.039 g). LCMS showed MH⁺=472; T_{RET}=2.67 min.

### Example 188

### N-(4-Fluorophenyl)-1-n-propyl-4-(tetrahydro-2H-pyran-4-ylamino)-1H-pyrazolo[3,4-b]pyridine-5-carboxamide

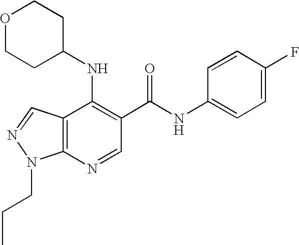

The synthetic method is as described in Example 187, except that in place of 4-(methylsulfonyl)benzylamine hydrochloride, 4-fluoroaniline (0.01 ml) was added to the mixture. The resultant product required further purification, which was performed by mass directed autoprep HPLC, giving Example 188 as a clear gum (0.03 g). LCMS showed MH⁺=398; T_{RET}=3.13 min.

### Example 189

### Ethyl 1-ethyl-6-methyl-4-(tetrahydro-2H-pyran-4-ylamino)-1H-pyrazolo[3,4-b]pyridine-5-carboxylate

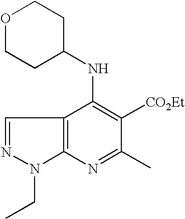

4-Aminotetrahydropyran hydrochloride (Intermediate 8A, 0.413 g, 3.0 mmol) was added to a mixture of Intermediate 51 (0.268 g, 1.0 mmol) and N,N-diisopropylethylamine (0.87 ml, 5.0 mmol) in acetonitrile (3 ml). The resulting mixture was heated at 85° C. for 24 hours. Volatiles were removed in vacuo and the residue was dissolved in chloroform (1.5 ml) and applied to a SPE cartridge (silica, 5 g). The cartridge was eluted successively with Et₂O, EtOAc and EtOAc-MeOH (9/1). Fractions containing the desired product were combined and concentrated in vacuo to give the desired product contaminated with starting material (Intermediate 51). Further purification using a SPE cartridge (silica, 5 g) eluting with ethyl acetate-cyclohexane (1/3) afforded Example 189 (0.248 g). LCMS showed MH⁺=333; T_{RET}=2.75 min.

### Example 190

### Ethyl 4-(cyclohexylamino)-1-ethyl-6-methyl-1H-pyrazolo[3,4-b]pyridine-5-carboxylate

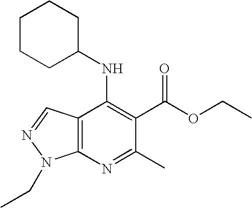

Cyclohexylamine (0.149 g, 1.5 mmol) was added to a mixture of Intermediate 51 (0.201 g, 0.75 mmol) and N,N-diisopropylethylamine (0.65 ml, 3.73 mmol) in acetonitrile (3 ml). The resulting mixture was heated at 85° C. for 40 hours. Volatiles were removed in vacuo and the residue was dissolved in chloroform (1.5 ml) and applied to a SPE cartridge (silica, 5 g). The cartridge was eluted successively with Et₂O, EtOAc and MeOH. Fractions containing the desired product were combined and concentrated in vacuo to afford Example 190 (0.128 g). LCMS showed MH⁺=331; T_{RET}=3.64 min.

### Example 191

### 4-(Cyclohexylamino)-1-ethyl-6-methyl-N-[4-(methylsulfonyl)benzyl]-1H-pyrazolo[3,4-b]pyridine-5-carboxamide

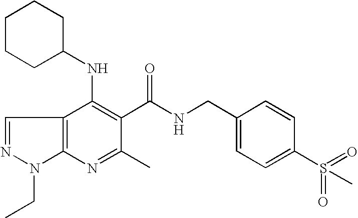

A mixture of Intermediate 52 (0.014 g, 0.046 mmol), HATU (0.018 g, 0.048 mmol) and DIPEA (0.022 ml, 0.125 mmol) in DMF (1 ml) was shaken at room temperature for 10 min. 1-[4-(Methylsulfonyl)phenyl]methanamine (0.009 g, 0.046 mmol) was then added, and the mixture was shaken for several minutes to give a solution. This solution was stored at room temperature for 16 hours. The solution was concentrated in vacuo, and the residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc-MeOH (9:1, 1.5 ml). Fractions containing the desired product were concentrated in vacuo to afford Example 191 (0.005 g). LCMS showed MH⁺=470; T_{RET}=2.54 min.

### Example 192

### N-Benzyl-4-(cyclohexylamino)-1-ethyl-6-methyl-1H-pyrazolo[3,4-b]pyridine-5-carboxamide

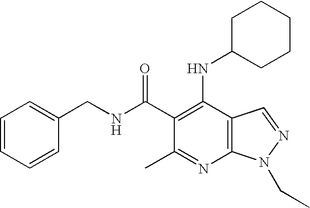

Example 192 was prepared from Intermediate 52 using a method analagous to Example 191. LCMS showed MH⁺=392: T_{RET}=2.43.

### Example 193

### 4-(Cyclohexylamino)-1-ethyl-N-(4-fluorophenyl)-6-methyl-1H-pyrazolo[3,4-b]pyridine-5-carboxamide

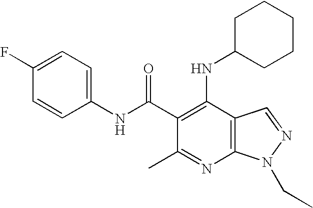

Example 193 was prepared from Intermediate 52 using an analagous method to Example 191. LCMS showed MH⁺=396; T_{RET}=2.6 min.

### Example 194

### 4-(Cyclohexylamino)-1-ethyl-6-methyl-N-[4-(trifluoromethyl)benzyl]-1H-pyrazolo[3,4-b]pyridine-5-carboxamide

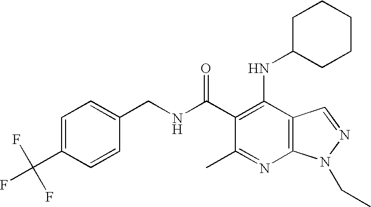

Example 194 was prepared from Intermediate 52 using an analagous method to Example 191. LCMS showed MH⁺=460; T_{RET}=2.74 min.

### Example 195

### 4-(Cyclohexylamino)-N-(2,3-dihydro-1H-inden-2-yl)-1-ethyl-6-methyl-1H-pyrazolo[3,4-b]pyridine-5-carboxamide

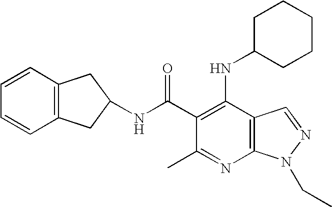

Example 195 was prepared from Intermediate 52 using an analagous method to Example 191. LCMS showed MH⁺=418; T_{RET}=2.55 min.

### Example 196

### N-Benzyl-1-ethyl-6-methyl-4-(tetrahydro-2H-pyran-4-ylamino)-1H-pyrazolo[3,4-b]pyridine-5-carboxamide

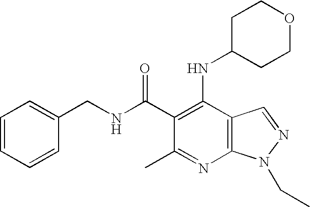

Example 196 was prepared from Intermediate 53 using an analagous method to Example 191. LCMS showed MH⁺=394; T_{RET}=2.02 min.

### Example 197

### N-Benzyl-1-ethyl-4-[(2-oxoazepan-3-yl)amino]-1H-pyrazolo[3,4-b]pyridine-5-carboxamide

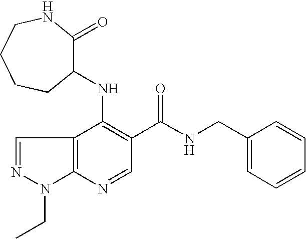

3-Aminoazepan-2-one (0.043 g, 0.335 mmol, commercially available from Sigma-Aldrich Company Ltd) was added to a mixture of Intermediate 17 (0.021 g, 0.067 mmol) and DIPEA (0.058 ml, 0.335 mmol) in acetonitrile (0.5 ml). The resulting mixture was heated at 85° C. for 48 hours. Volatiles were removed in vacuo, and the residue was dissolved in chloroform (0.5 ml) and applied to a SPE cartridge (silica, 0.5 g) which was eluted successively with diethyl ether (1.5 ml), ethyl acetate (1.5 ml) and ethyl acetate-methanol (9/1, 1.5 ml). Fractions containing the desired material were concentrated in vacuo to afford Example 197 (0.009 g). LCMS showed MH⁺=407; T_{RET}=2.81 min.

Similarly prepared, but replacing the 3-aminoazepan-2-one with the same or similar number of moles of another amine R³NH₂ were the following compounds:

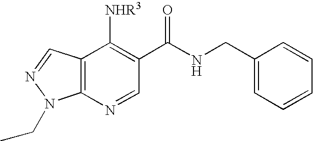

| Example | | Source of | Starting | | T_{RET} |
|---|---|---|---|---|---|
| Number | NHR³ | R³NH₂ | Material | MH⁺ ion | (min) |
| Example 198 | 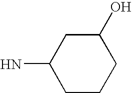 | J. Chem. Soc.,Perkin Trans. 1,1994, 537 | Intermediate 17 | 394 | 2.75 |
| Example 199 | 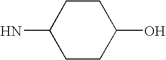 | Aldrich; orTCI-America | Intermediate 17 | 394 | 2.82 |
| Example 200 | 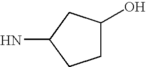 | US 4219660 | Intermediate 17 | 380 | 2.70 |

### Example 201

### N-Benzyl-1-ethyl-4-[(4-oxocyclohexyl)amino]-1H-pyrazolo[3,4-b]pyridine-5-carboxamide

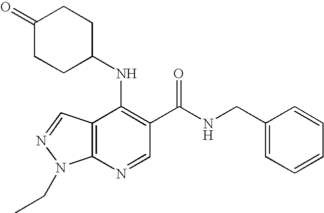

Intermediate 54 (0.048 g, 0.32 mmol) was added to a mixture of Intermediate 17 (0.050 g, 0.16 mmol) and DIPEA (0.17 ml, 0.98 mmol) in acetonitrile (3 ml). The resulting mixture was heated under reflux. After 12 hours, further quantities of Intermediate 54 (0.044 g, 0.29 mmol), DIPEA (0.17 ml, 0.98 mmol) and acetonitrile (1 ml) were added to reaction mixture which was maintained under reflux. After 36 hours, the reaction mixture was concentrated in vacuo, and the residual oil was dissolved in dichloromethane (8 ml) and washed with 5% sodium bicarbonate solution (2 ml). Evaporation of the organic solution gave a viscous oil which was dissolved in dichloromethane (2 ml) and applied to a SPE cartridge (silica, 5 g). The cartridge was eluted successively with a gradient of ethyl acetate-cyclohexane (1:16, then 1:8, 1:4, 1:2, 1:1 and 1:0). Fractions containing the desired material were concentrated in vacuo to afford Example 201 (0.018 g). LCMS showed MH⁺=392; T_{RET}=2.95 min.

### Example 202

### 1-Ethyl-N-(2-hydroxy-1-methylethyl)-4-(tetrahydro-2H-pyran-4-ylamino)-1H-pyrazolo[3,4-b]pyridine-5-carboxamide

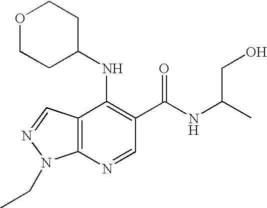

Intermediate 33 (0.1 g, 0.34 mmol), EDC (0.066 g, 0.34 mmol) and HOBT (0.05 g, 0.37 mmol) were suspended in DMF (2 ml) and stirred at room temperature under nitrogen for 15 min. 2-aminopropan-1-ol (0.026 g, 0.34 mmol) and triethylamine (0.036 g, 0.36 mmol) were added and the mixture was stirred at room temperature under nitrogen for 6 hours. Solvents were removed in vacuo and the residue partitioned between DCM and water. The organic layer was concentrated and applied to an SPE cartridge (aminopropyl, 5 g), which was eluted with methanol. Concentration in vacuo afforded Example 202 (0.095 g). LCMS showed MH⁺=348, T_{RET}=2.15 min.

### Example 203

### Methyl (2S)-2-({[1-ethyl-4-(tetrahydro-2H-pyran-4-ylamino)-1H-pyrazolo[3,4-b]pyridin-5-yl]carbonyl}amino)-3-hydroxypropanoate

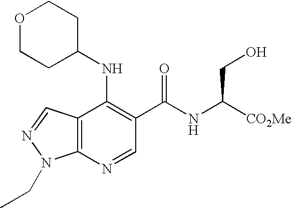

### Reaction Scheme:

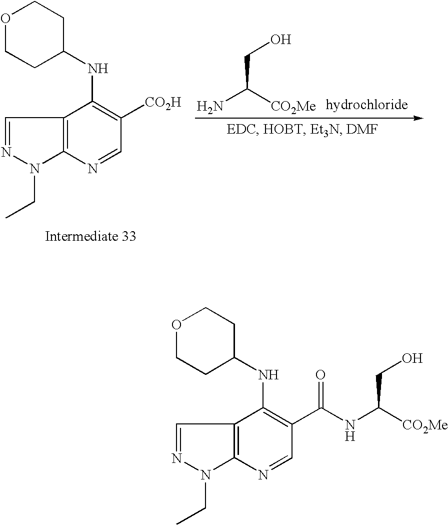

Intermediate 33 (0.1 g, 0.34 mmol), EDC (0.066 g, 0.34 mmol) and HOBT (0.05 g, 0.37 mmol) were suspended in DMF (2 ml) and stirred at room temperature under nitrogen for 15 mins. L-Serine methyl ester hydrochloride (0.054 g, 0.34 mmol) and triethylamine (0.036 g, 0.36 mmol) were added and the mixture stirred at room temperature under nitrogen for 18 hours. Solvents were removed in vacuo and the residue was partitioned between DCM and water. The organic layer was concentrated in vacuo and applied to an SPE cartridge (aminopropyl, 5 g), which was eluted with methanol. Concentration in vacuo afforded an impure residue which was further purified by SPE cartridge (silica, 5 g), eluting with ethyl acetate followed by 5% methanol/ethyl acetate. The desired fractions were concentrated in vacuo to afford Example 203 (0.055 g). LCMS showed MH⁺=393; T_{RET}=2.22 min.

### Example 204

### Ethyl 1-ethyl-4-[(4-hydroxycyclohexyl)amino]-1H-pyrazolo[3,4-b]pyridine-5-carboxylate

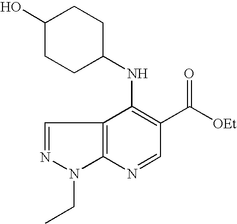

Intermediate 1 (1.5 g, 5.9 mmol) was dissolved in acetonitrile (80 ml). Trans-4-aminocyclohexanol (0.817 g, 7.1 mmol, commercially available from TCI-America; alternatively (e.g. as the HCl salt) from Aldrich) and diisopropylethylamine (6.18 ml, 35.5 mmol) were added and the mixture was stirred at 85° C. for 16 h. The mixture was concentrated in vacuo, and the residue was partitioned between DCM (120 ml) and water (30 ml). The phases were separated and the organic phase was dried (Na₂SO₄) and evaporated to give a pale yellow solid. The solid was dissolved in a mixture of DCM (10 ml) and chloroform (3 ml), and applied in equal portions to two SPE cartridges (silica, 20 g) which were eluted sequentially with a gradient of EtOAc:cyclohexane (1:16, then 1:8, 1:4, 1:2, 1:1 and 1:0). Fractions containing the desired material were combined and evaporated in vacuo to give Example 204 (1.893 g) as a white solid. LCMS showed MH⁺=333; T_{RET}=2.79 min.

### Example 205

### Ethyl 1-ethyl-4-[(4-oxocyclohexyl)amino]-1H-pyrazolo[3,4-b]pyridine-5-carboxylate

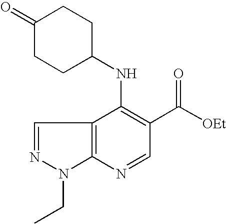

Example 204 (1.893 g, 5.7 mmol) was suspended in acetone (12 ml) and the stirred suspension was treated at 0° C. with Jones reagent (1.81 ml). After 30 min, a further quantity of Jones reagent (1.81 ml) was added to the reaction mixture which was maintained at 0° C. After a further 2 h, a final portion of Jones reagent (1.44 ml) was added to the reaction mixture, and stirring at 0° C. was continued for 1 h. Isopropanol (3.8 ml) was added to the reaction mixture, followed by water (15 ml). The resulting mixture was extracted with ethyl acetate (2×40 ml). The combined organic extracts were washed with water (8 ml), dried (Na₂SO₄) and evaporated to a grey solid. The solid was dissolved in DCM (10 ml) and applied in equal portions to two SPE cartridges (silica, 20 g) which were eluted sequentially with a gradient of ethyl acetate:cyclohexane (1:16, then 1:8, 1:4, 1:2, and 1:1). Fractions containing the desired material were combined and evaporated in vacuo to give Example 205 (1.893 g) as a white solid. LCMS showed MH⁺=331; T_{RET}=2.84 min.

### Example 207

### (=Example 5): Ethyl 4-[(1-acetyl-4-piperidinyl)amino]-1-ethyl-1H-pyrazolo[3,4-b]pyridine-5-carboxylate

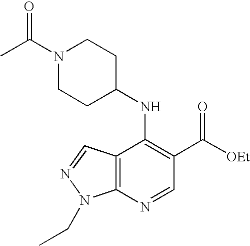

Intermediate 1 (2.58 g), Intermediate 6 (2.0 g) and N,N-diisopropylethylamine (8.9 ml) were dissolved in acetonitrile (98 ml). The reaction mixture was heated at 85° C. for 24 h then an additional portion of Intermediate 6 (0.18 g) was added and heating continued for a further 10 h. The reaction was concentrated in vacuo and the residues partitioned between DCM and water. The phases were separated and the organic phase evaporated in vacuo. The residue was purified by chromatography using Biotage (silica 90 g) eluting with DCM: MeOH (5%) to afford Example 207 (1.55 g) as a white solid. LCMS showed MH⁺ 360; T_{RET}=2.71 min.

### Example 209

### Ethyl 4-[(4-aminocyclohexyl)amino]-1-ethyl-1H-pyrazolo[3,4-b]pyridine-5-carboxylate

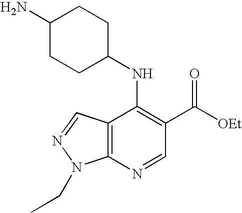

Example 209 was prepared from Intermediate 1 and (4-aminocyclohexyl)amine using an analogous method to that used for the preparation of Example 207. LCMS showed MH⁺=332; T_{RET}=2.18 min

### Example 210

### 1-Ethyl-N-[(1-oxido-3-pyridinyl)methyl]-4-(tetrahydro-2H-pyran-4-ylamino)-1H-pyrazolo[3,4-b]pyridine-5-carboxamide

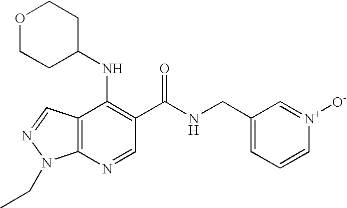

A solution of meta-chloroperoxybenzoic acid (45 mg, 0.26 mmol) in chloroform (1 ml) was added dropwise at 0° C. to a stirred solution of Example 138 (0.1 g, 0.26 mmol) in chloroform (1.5 ml). After 1.5 h at 0° C., a further quantity of meta-chloroperoxybenzoic acid (45 mg, 0.26 mmol) in chloroform (1 ml) was added, and stirring was continued at 0° C. for 1.5 h. A trace of starting material remained, so an additional quantity of meta-chloroperoxybenzoic acid (22 mg, 0.13 mmol) in chloroform (0.6 ml) was added. After 3.5 h at 0° C., 2M sodium carbonate solution (1 ml), was added to the reaction mixture. The phases were separated by passage through a hydrophobic frit and the aqueous phase was extracted with more chloroform (2 ml). The combined organic extracts were evaporated to a residual foam which was purified by mass directed autoprep HPLC to afford Example 210 (44 mg). LCMS showed MH⁺=397; T_{RET}=2.13 min.

### Example 211

### 1-Ethyl-N-[(1-oxido-2-pyridinyl)methyl]-4-(tetrahydro-2H-pyran-4-ylamino)-1H-pyrazolo[3,4-b]pyridine-5-carboxamide

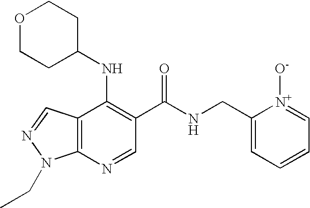

Example 211 was prepared from Example 600 using an analogous method to that used for the preparation of Example 210. LCMS showed MH⁺=397; T_{RET}=2.20 min

### Example 212

### 1-Ethyl-N-[(1-oxido-4-pyridinyl)methyl]-4-(tetrahydro-2H-pyran-4-ylamino)-1H-pyrazolo[3,4-b]pyridine-5-carboxamide

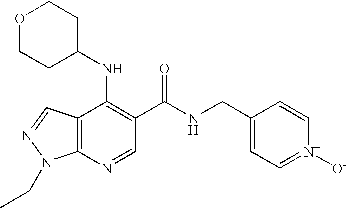

Example 212 was prepared from Example 33 using an analogous method to that used for the preparation of Example 210. LCMS showed MH⁺=397; T_{RET}=2.13 min Examples 214 to 230

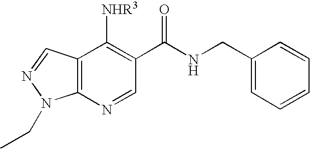

### General Procedure

Intermediate 17 (0.15 mmol) was treated with an aliquot of the amine (0.95 ml, equivalent to 0.19 mmol) from a stock solution in acetonitrile (0.2M) and N,N-diisopropylethylamine (0.24 mmol). The mixture was heated at reflux for 20 h then concentrated in vacuo. The residue was purified by SPE (silica) to give the desired product.

| | | | | | LC-MC |
|---|---|---|---|---|---|
| | | Source of | Starting | MH + | Retention |
| Example no. | NHR³ | R³NH₂ | Material | ion | time |
| 214 | 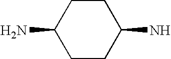 | J. Med. Chem.,1994, 37(17),2360 | Intermediate 17 | 393 | 2.16 |
| 221 | 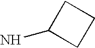 | Aldrich | Intermediate 17 | 350 | 3.18 |
| 222 | 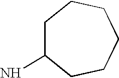 | Aldrich | Intermediate 17 | 392 | 3.62 |
| 223 | 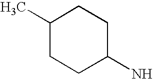 | Aldrich | Intermediate 17 | 392 | 3.63,3.68 |
| 224 | 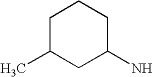 | Pfaltz-Bauer | Intermediate 17 | 392 | 3.61,3.66 |
| 225 | 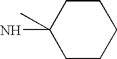 | J. Org. Chem.,1985, 50(11),1859 | Intermediate 17 | 392 | 3.54 |
| 226 | 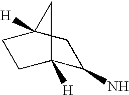 | Aldrich | Intermediate 17 | 390 | 3.56 |
| 227 | 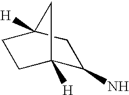 | Aldrich | Intermediate 17 | 390 | 3.52 |
| 228 | 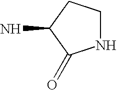 | WO 99/12933 | Intermediate 17 | 379 | 2.66 |
| 229 | 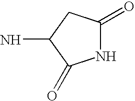 | EP 1188744 | Intermediate 17 | 393 | 2.58 |
| 230 | 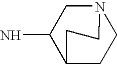 | Aldrich | Intermediate 17 | 405 | 2.19 |

### Example 225

### 1-ethyl-4-[(1-methylcyclohexyl)amino]-N-(phenylmethyl)-1H-pyrazolo[3,4-b]pyridine-5-carboxamide

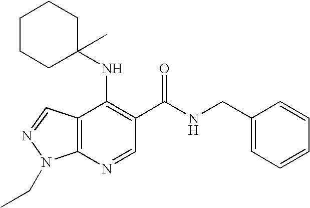

A preferred method for the preparation of Example 225 involving 1-methylcyclohexylamine and a longer reaction time is as follows:

A solution of Intermediate 17 (46 mg), 1-methylcyclohexylamine (26 mg) and diisopropylethylamine (94 mg) in acetonitrile (1 ml) was stirred and heated at reflux for 77 h. More 1-methylcyclohexylamine (102 mg), diisopropylethylamine (93 mg) and acetonitrile (1 ml) were added and the reaction mixture was heated at reflux for a further 68 h. The solution was cooled and concentrated in vacuo. The residue was triturated in ethyl acetate and filtered. The filtrate was purified by mass directed autoprep. HPLC to give Example 225 (19 mg). LCMS showed MH⁺=392; T_{RET}=3.46 min.

Examples 231, 247 and 257, shown below and also involving 1-methylcyclohexylamine, can also preferably be prepared in a similar manner.

### Examples 231-239

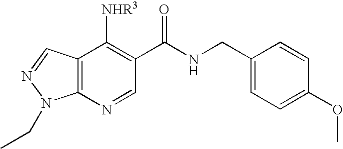

### General Procedure

Intermediate 55 (0.15 mmol) was treated with an aliquot of the amine (0.95 ml, equivalent to 0.19 mmol) from a stock solution in acetonitrile (0.2M) and N,N-diisopropylethylamine (0.24 mmol). The mixture was heated at reflux for 20 h then concentrated in vacuo. The residue was purified by SPE (silica) to give the desire product.

| | | | | | LC-MC |
|---|---|---|---|---|---|
| | | Source of | Starting | MH + | Retention |
| Example no. | NHR³ | R³NH₂ | Material | ion | time |
| 231 | 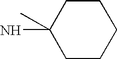 | J. Org. Chem.,1985, 50(11),1859 | Intermediate 55 | 422 | 3.43 |
| 233 | 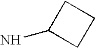 | Aldrich | Intermediate 55 | 380 | 3.20 |
| 234 | 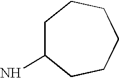 | Aldrich | Intermediate 55 | 422 | 3.58 |
| 235 | 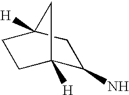 | Aldrich | Intermediate 55 | 420 | 3.52 |
| 236 | 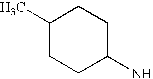 | Aldrich | Intermediate 55 | 422 | 3.57,3.64 |
| 237 | 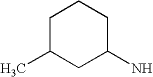 | Pfaltz-Bauer | Intermediate 55 | 422 | 3.56,3.62 |
| 238 | 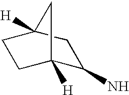 | Aldrich | Intermediate 55 | 420 | 3.48 |
| 239 | 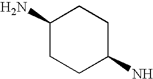 | J. Med. Chem.,1994, 37(17),2360 | Intermediate 55 | 423 | 2.16 |

### Examples 240-249

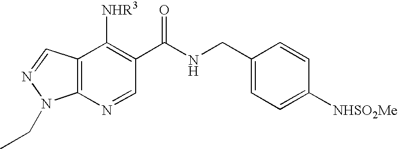

### General Procedure

Intermediate 56 (0.15 mmol) was treated with an aliquot of the amine (0.95 ml, equivalent to 0.19 mmol) from a stock solution in acetonitrile (0.2M) and N,N-diisopropylethylamine (0.24 mmol). The mixture was heated at reflux for 20 h then concentrated in vacuo. The residue was purified by SPE (silica) to give the desire product.

| | | | | | LC-MC |
|---|---|---|---|---|---|
| | | Source of | Starting | MH + | Retention |
| Example no. | NHR³ | R³NH₂ | Material | ion | time |
| 240 | 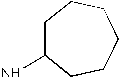 | Aldrich | Intermediate 56 | 485 | 3.26 |
| 241 | 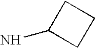 | Aldrich | Intermediate 56 | 443 | 2.94 |
| 242 | 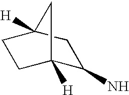 | Aldrich | Intermediate 56 | 483 | 3.20 |
| 243 | 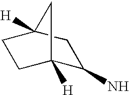 | Aldrich | Intermediate 56 | 483 | 3.14 |
| 244 | 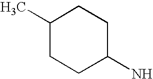 | Aldrich | Intermediate 56 | 485 | 3.25,3.33 |
| 245 | 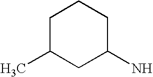 | Pfatlz-Bauer | Intermediate 56 | 485 | 3.24,3.31 |
| 247 | 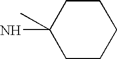 | J. Org. Chem.,1985, 50(11),1859 | Intermediate 56 | 483 | 3.10 |
| 248 | 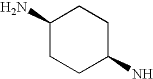 | J. Med. Chem.,1994, 37(17),360 | Intermediate 56 | 486 | 2.05 |
| 249 | 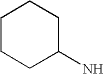 | Aldrich | Intermediate 56 | 471 | 3.21 |

### Examples 250-258

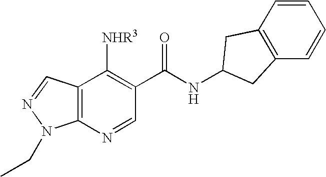

### General Procedure

Intermediate 57 (0.15 mmol) was treated with an aliquot of the amine (0.95 ml, equivalent to 0.19 mmol) from a stock solution in acetonitrile (0.2M) and N,N-diisopropylethylamine (0.24 mmol). The mixture was heated at reflux for 20 h then concentrated in vacuo. The residue was purified by SPE (silica) to give the desire product.

| | | | | | LC-MC |
|---|---|---|---|---|---|
| | | Source of | Starting | MH + | Retention |
| Example no. | NHR³ | NH₂R³ | Material | ion | time |
| 250 | 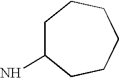 | Aldrich | Intermediate 57 | 418 | 3.78 |
| 251 | 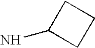 | Aldrich | Intermediate 57 | 376 | 3.42 |
| 253 | 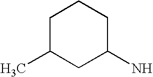 | Pfaltz-Bauer | Intermediate 57 | 418 | 3.78,3.84 |
| 254 | 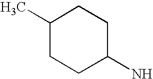 | Aldrich | Intermediate 57 | 418 | 3.82,3.86 |
| 255 | 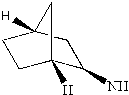 | Aldrich | Intermediate 57 | 416 | 3.66 |
| 256 | 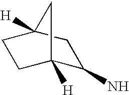 | Aldrich | Intermediate 57 | 416 | 3.77 |
| 257 | 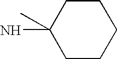 | J. Org. Chem.,1985, 50(11),1859 | Intermediate 57 | 418 | 3.74 |
| 258 | 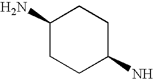 | J. Med. Chem.,1994, 37(17),2360 | Intermediate 57 | 419 | 2.38 |

### Examples 259-275

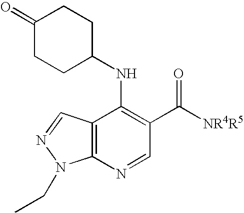

### General Procedure

A mixture of Intermediate 58 (0.1 mmol), HATU (0.1 mmol) and DIPEA (0.4 mmol) in DMF (0.4 ml) was shaken at room temperature for 10 min. A solution of the amine (0.1 mmol) in DMF (0.2 ml) was then added and the mixture agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

| | | | | | LC-MC |
|---|---|---|---|---|---|
| | | Source of | Starting | MH + | Retention |
| Example no. | NR⁴R⁵ | HNR⁴R₅ | Material | ion | time |
| 201 | 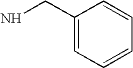 | Aldrich | Intermediate 58 | 392 | 2.60 |
| 259 | 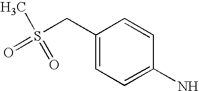 | EP 666258 | Intermediate 58 | 470 | 2.44 |
| 260 | 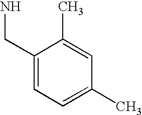 | Salor; or 1CNBiomedicals,Inc.; orSynthesis, 1982,12, 1036 | Intermediate 58 | 420 | 3.09 |
| 261 | 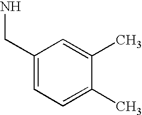 | CHMSRV-AS;or MatrixScientific; orChem. Ber.,1969, 102,2770 | Intermediate 58 | 420 | 3.09 |
| 262 | 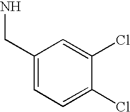 | Aldrich; orMeindl et al.,J. Med. Chem.,1984, 27(9),1111. | Intermediate 58 | 454 | 3.20 |
| 263 | 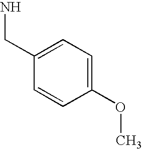 | Acros; orAldrich;TetrahedronLett., 2002,43(48), 8735; orJ. Med. Chem.,1984, 27(9),1111; or Org.Lett., 2002,4(12), 2055 | Intermediate 58 | 422 | 2.86 |
| 264 | 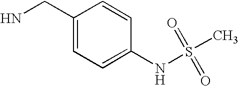 | Lis et al., J.Med. Chem.,1990, 33(10),2883; seeScheme III andref. 24 | Intermediate 58 | 485 | 2.64 |
| 265 | 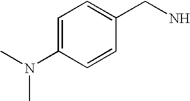 | Aldrich | Intermediate 58 | 435 | 2.54 |
| 266 | 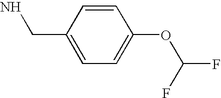 | Fluorochem; orWO 98/45268 | Intermediate 58 | 458 | 2.81 |
| 267 | 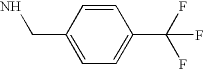 | Aldrich; orMeindl et al., J.Med. Chem.,1984, 27(9),1111; or Org.Lett., 2002,4(12), 2055 | Intermediate 58 | 460 | 2.96 |
| 268 | 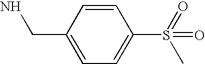 | Peakdale | Intermediate 58 | 470 | 2.39 |
| 269 | 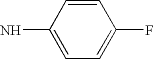 | Aldrich | Intermediate 58 | 396 | 2.80 |
| 270 (asCF₃CO₂Hsalt) | 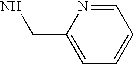 | Aldrich | Intermediate 58 | 393 | 1.89 |
| 271 | 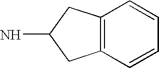 | TCI-America;or Aldrich; orMaybridge-Int | Intermediate 58 | 418 | 2.77 |
| 272 | 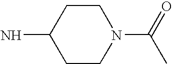 | WO 99/38877 | Intermediate 58 | 427 | 2.13 |
| 273 | 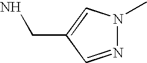 | N. D. ZelinskyInstitute | Intermediate 58 | 396 | 2.15 |
| 274 | 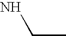 | Aldrich | Intermediate 58 | 330 | 2.10 |
| 275 | 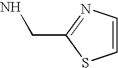 | MatrixScientific | Intermediate 58 | 399 | 2.29 |

### Example 260

### Alternative Procedure

### N-[(2,4-dimethylphenyl)methyl]-1-ethyl-4-[(4-oxocyclohexyl)amino]-1H-pyrazolo[3,4-b]pyridine-5-carboxamide

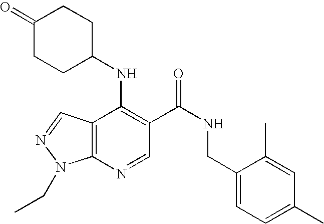

### Alternative Procedure for Preparing Example 260

A solution of Intermediate 58 (45 mg), HATU (63 mg) and DIPEA (39 mg) in acetonitrile (5 ml) was stirred for 10 min. A solution of 2,4-dimethylbenzylamine (24 mg) (available from Salor; or ICN Biomedicals, Inc.; or Synthesis, 1982, 12, 1036) in acetonitrile (1 ml) was added. The reaction mixture was stirred for 18 h. The solution was concentrated and the residue partitioned between ethyl acetate (25 ml) and 0.5M sodium bicarbonate (20 ml). The organic phase was separated, washed with water (20 ml), dried over Na₂SO₄ and concentrated to leave a gum which was applied to an SPE cartridge (5 g). The cartridge was eluted with ethyl acetate. Fractions containing the desired compound were combined and concentrated in vacuo to give Example 260 (32 mg). LC-MS showed MH⁺=420; T_{RET}=3.16 min. δ_{H} (CDCl₃): 1.49 (3H, t), 2.11 (2H, m), 2.33 (3H, s), 2.35 (3H, s), 2.40 (2H, m), 2.52 (2H, m), 2.61 (2H, m), 4.36 (1H, m), 4.47 (2H, q), 4.55 (2H, d), 6.14 (1H, t), 7.01+7.18 (2H, AA′BB′), 7.04 (1H, s), 8.01 (1H, s), 8.36 (1H, s), 9.96 (1H, d).

### Example 276-287

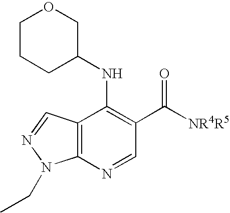

### General Procedure

A mixture of Intermediate 59 (0.1 mmol), HATU (0.1 mmol) and DIPEA (0.4 mmol) in DMF (0.4 ml) was shaken at room temperature for 10 min. A solution of the amine (0.1 mmol) in DMF (0.2 ml) was then added and the mixture agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

| | | | | MH | LC-MC |
|---|---|---|---|---|---|
| | | Source of | Starting | + | Retention |
| Example no. | NR⁴R⁵ | HNR⁴R⁵ | Material | ion | time |
| 276 | 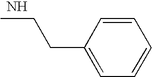 | Aldrich | Intermediate59 | 392 | 2.60 |
| 277 | 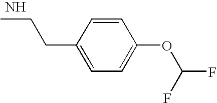 | Fluorochem; orWO 98/45268 | Intermediate59 | 446 | 2.84 |
| 278 | 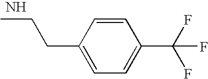 | Aldrich; orMeindl et al., J.Med. Chem.,1984, 27(9),1111; or Org.Lett., 2002,4(12), 2055 | Intermediate59 | 448 | 3.0 |
| 279 | 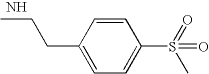 | Acros | Intermediate59 | 458 | 2.40 |
| 280 | 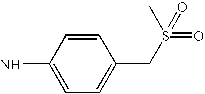 | EP 382570 | Intermediate59 | 458 | 2.47 |
| 281 | 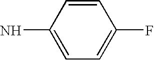 | Aldrich | Intermediate59 | 384 | 2.85 |
| 282 (asCF₃CO₂Hsalt) | 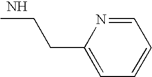 | Aldrich | Intermediate59 | 381 | 1.89 |
| 283 | 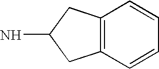 | TCI-America;or Aldrich; orMaybridge-Int | Intermediate59 | 406 | 2.80 |
| 284 | 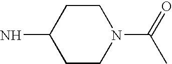 | WO 99/38877 | Intermediate59 | 415 | 2.14 |
| 285 | 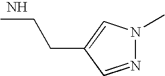 | N.D. ZelinskyInstitute | Intermediate59 | 384 | 2.16 |
| 286 | 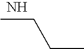 | Aldrich | Intermediate59 | 318 | 2.11 |
| 287 | 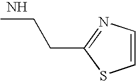 | MatrixScientific | Intermediate59 | 399 | 2.29 |

### Example 288

### 4-[(4,4-Difluorocyclohexyl)amino]-1-ethyl-N-(phenylmethyl)-1H-pyrazolo[3,4-b]pyridine-5-carboxamide and

### Example 289

### 1-Ethyl-4-[(4-fluoro-3-cyclohexen-1-yl)amino]-N-(phenylmethyl)-1H-pyrazolo[3,4-b]pyridine-5-carboxamide

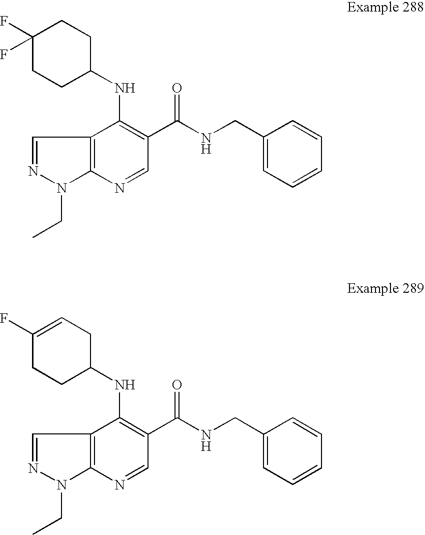

Diisopropylethylamine (0.113 ml, 0.65 mmol) was added to a stirred mixture of Intermediate 17 (40 mg, 0.13 mmol) and Intermediate 63 (45 mg, 0.26 mmol) in acetonitrile (2 ml). The mixture was stirred at 85° C. After 18 h, a further portion of Intermediate 63 (22.5 mg, 0.13 mmol) and diisopropylethylamine (0.113 ml, 0.65 mmol) was added to the reaction mixture and stirring was continued at 90° C. for 24 h. The mixture was then concentrated in vacuo and the residue was partitioned between DCM (20 ml) and water (5 ml). The phases were separated and the aqueous phase was extracted with further DCM (10 ml). The combined organic extracts were dried (Na₂SO₄) and evaporated in vacuo to give a brown oil (65 mg) which was partially purified on a SPE cartridge (silica, 10 g), eluting with ethyl acetate:petroleum ether (1:8; 1:4; 1:2; 1:1 and 1:0). The resulting two-component pale-brown oil (34 mg) was separated by mass directed auto prep HPLC to give Example 288 (19 mg) as a white foam (LCMS showed MH⁺=414; T_{RET}=3.24 min) and Example 289 (9 mg) as a white solid (LCMS showed MH⁺=394; T_{RET}=3.21 min).

### Examples 290-319

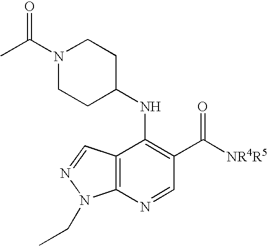

### General Procedure

A mixture of Intermediate 60 (0.1 mmol), HATU (0.1 mmol) and DIPEA (0.4 mmol) in DMF (0.4 ml) was shaken at room temperature for 10 min. A solution of the amine (0.1 mmol) in DMF (0.2 ml) was then added and the mixture agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

| | | | | | LC-MC |
|---|---|---|---|---|---|
| | | Source of | Starting | MH + | Retention |
| Example no. | NR⁴R⁵ | HNR⁴R₅ | Material | ion | time |
| 290 | 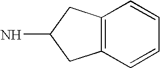 | Aldrich; orTCI-America;or Maybridge-Int | Intermediate 60 | 447 | 2.96 |
| 291 | 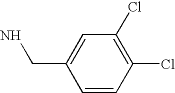 | Aldrich; orMeindl et al., J.Med. Chem.,1984, 27(9),1111. | Intermediate 60 | 488/490 | 3.16 |
| 292 | 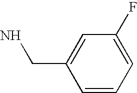 | Aldrich; orMeindl et al., J.Med. Chem.,1984, 27(9),1111; or Org.Lett., 2002,4(12), 2055 | Intermediate 60 | 439 | 2.84 |
| 293 | 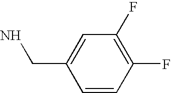 | Aldrich | Intermediate 60 | 457 | 2.92 |
| 294 | 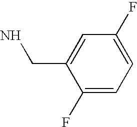 | Aldrich | Intermediate 60 | 457 | 2.87 |
| 295 | 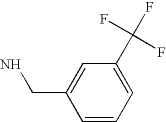 | Aldrich; orMeindl et al., J.Med. Chem.,1984, 27(9),1111. | Intermediate 60 | 489 | 3.06 |
| 296 | 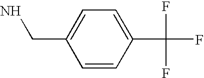 | Aldrich; orMeindl et al., J.Med. Chem.,1984, 27(9),1111; or Org.Lett., 2002,4(12), 2055 | Intermediate 60 | 489 | 3.08 |
| 297 | 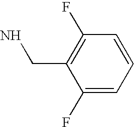 | Aldrich | Intermediate 60 | 457 | 2.82 |
| 298 | 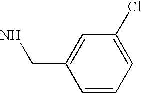 | Aldrich; orMeindl et al., J.Med. Chem.,1984, 27(9),1111; or Org.Lett., 2002,4(12), 2055 | Intermediate 60 | 455 | 2.98 |
| 299 | 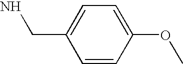 | Aldrich; orAcros, or Jung etal., TetrahedronLett., 2002,43(48), 8735; orMeindl et al., J.Med. Chem.,1984, 27(9),1111; or Org.Lett., 2002,4(12), 2055 | Intermediate 60 | 451 | 2.79 |
| 300 | 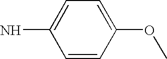 | Aldrich | Intermediate 60 | 437 | 2.82 |
| 301 | 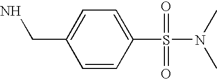 | PeakdaleMolecular Ltd | Intermediate 60 | 528 | 2.76 |
| 302 | 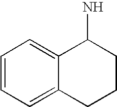 | Aldrich | Intermediate 60 | 461 | 3.00 |
| 303 | 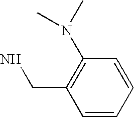 | PeakdaleMolecular Ltd | Intermediate 60 | 464 | 2.31 |
| 304 | 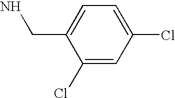 | Aldrich; orMeindl et al.,J. Med. Chem.,1984, 27(9),1111. | Intermediate 60 | 489 | 3.16 |
| 305 | 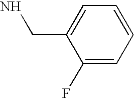 | Aldrich; or Org.Lett., 2002,4(12), 2055 | Intermediate 60 | 439 | 2.84 |
| 306 | 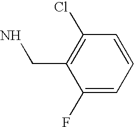 | Fluka | Intermediate 60 | 473 | 2.92 |
| 307 | 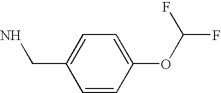 | FluorochemLtd; orWO 98/45268 | Intermediate 60 | 487 | 2.95 |
| 308 | 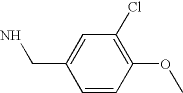 | Apin | Intermediate 60 | 485 | 2.94 |
| 309 | 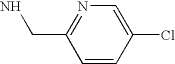 | Key OrganicsLtd | Intermediate 60 | 456 | 2.65 |
| 310 | 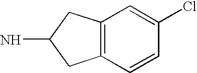 | J. Med. Chem.,2001, 44(26),4628 | Intermediate 60 | 481 | 3.16 |
| 311 | 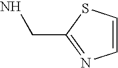 | ManchesterOrganics Ltd | Intermediate 60 | 428 | 2.28 |
| 312 | 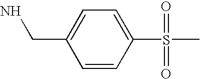 | Acros Chimica | Intermediate 60 | 499 | 2.37 |
| 313 | 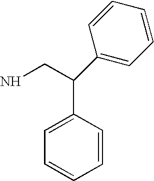 | Aldrich | Intermediate 60 | 511 | 3.18 |
| 314 | 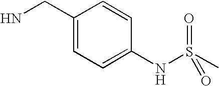 | Lis et al., J.Med. Chem.,1990, 33(10),2883, seeScheme III andref. 24 | Intermediate 60 | 514 | 2.60 |
| 315 | 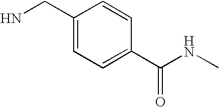 | WO 94/17035 | Intermediate 60 | 478 | 2.47 |
| 316 | 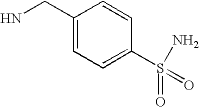 | Sigma | Intermediate 60 | 500 | 2.50 |
| 317 | 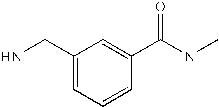 | PeakdaleMolecular Ltd | Intermediate 60 | 478 | 2.49 |
| 318 | 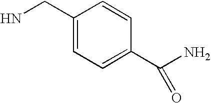 | WO 02/85860 | Intermediate 60 | 464 | 2.42 |
| 319 | 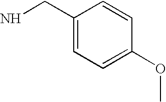 | Syngene | Intermediate 60 | 452 | 2.45 |

### Example 320

### 1-Ethyl-N-4-piperidinyl-4-(tetrahydro-2H-pyran-4-ylamino)-1H-pyrazolo[3,4-b]pyridine-5-carboxamide

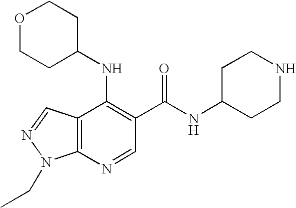

A solution of hydrogen chloride in dioxan (30 ml, 4M, 0.12 mol) was added to a suspension of Example 126 (1.3 g, 2.75 mmol), in dioxan (10 ml) and the mixture was stirred at room temperature for 6 h. The reaction mixture was left to stand for 14 h, then the solution was evaporated, azeotroping with DCM to give a white solid the hydrochloride salt. The solid was suspended in ethyl acetate (50 ml) and washed with sodium hydroxide solution (2N, 50 ml). The organic layer was dried over Na₂SO₄ and concentrated in vacuo to give Example 318 as a white solid (995 mg). LCMS showed MH⁺=373; T_{RET}=1.89 min.

### Example 321

### 1-Ethyl-N-(4-piperidinylmethyl)-4-(tetrahydro-2H-pyran-4-ylamino)-1H-pyrazolo[3,4-b]pyridine-5-carboxamide

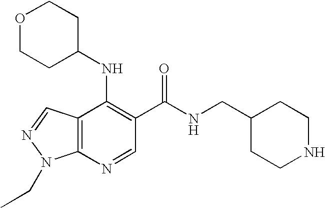

A solution of hydrogen chloride in dioxan (30 ml, 4M, 0.12 mol) was added to a suspension of Intermediate 72 (1.2 g, 2.5 mmol), in dioxan (10 ml) and the mixture was stirred at room temperature for 6 h. The reaction mixture was left to stand for 14 h, then the solution was evaporated, azeotroping with DCM to give a white solid (1.24 g). A portion of the solid (68 mg) was suspended in ethyl acetate and washed with 2M-sodium hydroxide solution. The organic layer was dried over Na₂SO₄ and concentrated in vacuo to afford Example 321 as a white solid (60 mg). LCMS showed MH⁺=387; T_{RET}=1.92 min.

### Example 322

### 1-Ethyl-N-[1-(ethylsulfonyl)-4-piperidinyl]-4-(tetrahydro-2H-pyran-4-ylamino)-1H-pyrazolo[3,4-b]pyridine-5-carboxamide

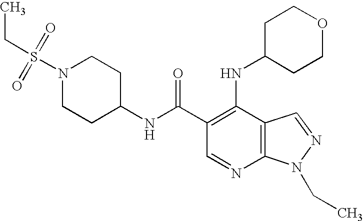

Triethylamine (0.023 ml, 0.16 mmol) was added to a solution of Example 320 (0.043 g, 0.1115 mol) in DCM (1 ml). The mixture was cooled (ice/water bath for 10 min) and ethane sulfonyl chloride (0.014 ml, 0.138 mmol) was added. The resultant solution was stirred at room temperature for 18 h, then the solvent was removed with a steam of nitrogen. The residue was dissolved in dichloromethane (1.5 ml) and stirred with water (1.5 ml). The organic layer was separated and blown down with nitrogen, and applied to a SPE cartridge (silica, 2 g) eluting with 60%-100% ethyl acetate in cyclohexane. The desired fractions were concentrated in vacuo to afford Example 322 as a white solid (32 mg). LCMS showed MH⁺=465; T_{RET}=2.52 min

Similarly prepared were the following, using the same or a similar number of moles of reagents and the same or similar volumes of solvents:

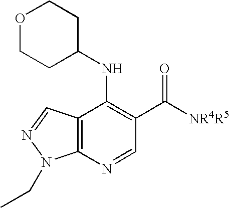

| | | | Source of | | LC-MC |
|---|---|---|---|---|---|
| | | Sulfonyl | sulfonyl | MH + | Retention |
| Example no. | NR⁴R⁵ | chloride | chloride | ion | time |
| 323 | 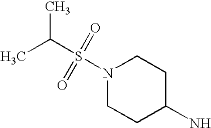 | 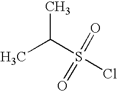 | Aldrich | 479 | 2.58 |
| 324 | 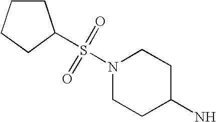 | 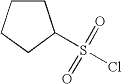 | J. Org.Chem.,1952, 17,1529 | 505 | 2.75 |
| 325 | 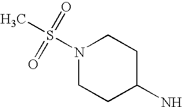 | 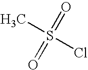 | Aldrich | 451 | 2.41 |
| 326 | 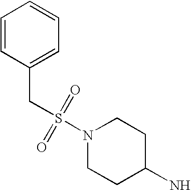 | 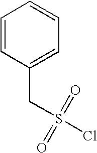 | Aldrich | 527 | 2.90 |
| 327 | 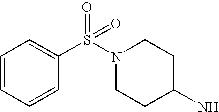 | 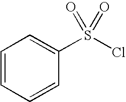 | Aldrich | 513 | 2.66 |
| 328 | 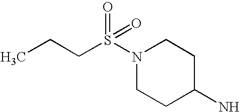 | 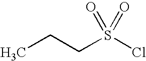 | Aldrich | 479 | 2.42 |

### Example 329

### N-[1-(Cyclopropylcarbonyl)-4-piperidinyl]-1-ethyl-4-(tetrahydro-2H-pyran-4-ylamino)-1H-pyrazolo[3,4-b]pyridine-5-carboxamide

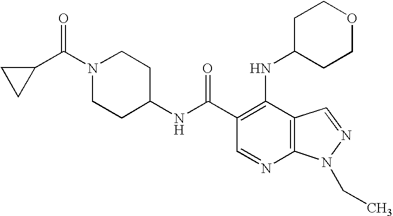

Cyclopropane carboxylic acid (0.011 ml, 0.138 mmol), EDC (0.031 g, 0.161 mmol) and HOBT (0.019 g, 0.138 mmol) were suspended in DMF (2 ml) and stirred at room temperature for 1 h. Example 320 (0.043 g, 0.115 mmol) was added and the mixture was stirred at room temperature for 16 hours. Most of the solvent was removed using a stream of nitrogen and the residue was partitioned between DCM (3 ml) and water (3 ml). The organic layer was blown down with nitrogen and applied to a SPE cartridge (aminopropyl, 1 g), which was eluted with methanol. Concentration by blowing down with nitrogen afforded an impure residue which was further purified by SPE cartridge (silica, 1 g), eluting with 50-100% EtOAc in cyclohexane followed by 5% methanol in EtOAc. The desired fractions were concentrated in vacuo to afford Example 329 as a white solid (49 mg). LCMS showed MH⁺=441; T_{RET}=2.23 min

Similarly prepared, using the same or similar numbers of moles of reagents and volumes of solvents, and using Example 320 as the starting material to make Examples 330 to 343, but using Example 321 (similar number of moles) instead of Example 320 as the starting material to make Examples 344 to 349, were the following:

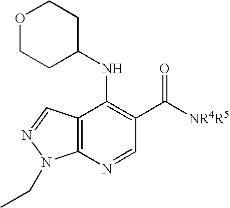

| | | | Source of | | LC-MC |
|---|---|---|---|---|---|
| | | Carboxylic | Carboxylic | MH + | Retention |
| Example no. | NR⁴R⁵ | acid | acid | ion | time |
| 330 | 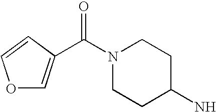 | 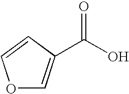 | Aldrich | 467 | 2.50 |
| 331 | 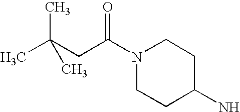 | 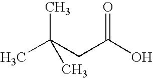 | Aldrich | 471 | 2.73 |
| 332 | 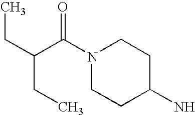 | 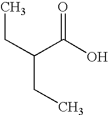 | Aldrich | 471 | 2.72 |
| 333 | 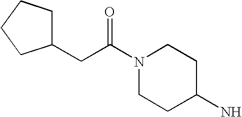 | 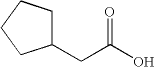 | Aldrich | 483 | 2.81 |
| 334 | 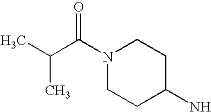 | 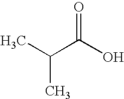 | Aldrich | 443 | 2.27 |
| 335 | 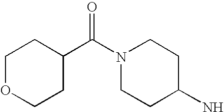 | 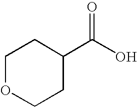 | Combi-Blocks | 485 | 2.17 |
| 336 | 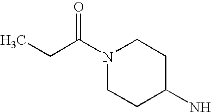 | 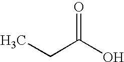 | Aldrich | 429 | 2.38 |
| 337 | 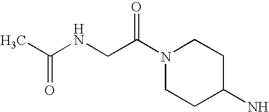 | 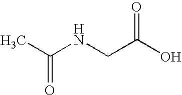 | Aldrich | 472 | 2.20 |
| 338 | 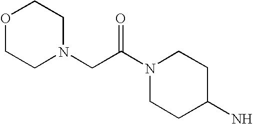 | 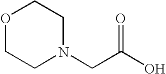 | Synchem OHG | 500 | 1.91 |
| 339 | 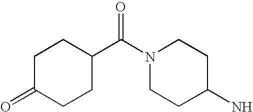 | 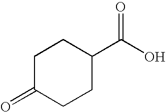 | J. Med. Chem.,1998, 41(5),760 | 497 | 2.17 |
| 340 | 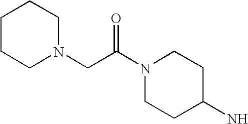 | 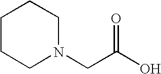 | Micro-ChemistryBuildingBlocks | 498 | 1.94 |
| 341 | 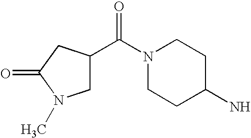 | 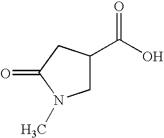 | InterchimIntermediates | 498 | 2.07 |
| 342 | 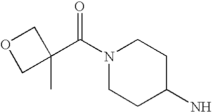 | 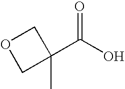 | DE 3618135 | 471 | 2.33 |
| 343 | 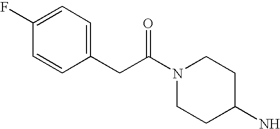 | 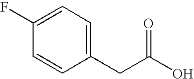 | Aldrich | 509 | 2.75 |
| 344 | 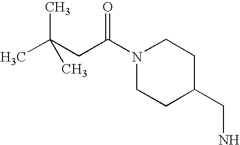 | 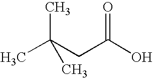 | Aldrich | 485 | 2.78 |
| 345 | 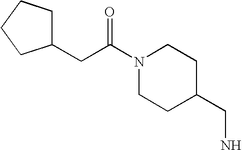 | 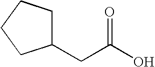 | Aldrich | 497 | 2.85 |
| 346 | 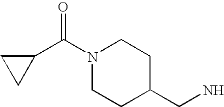 | 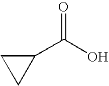 | Aldrich | 455 | 2.50 |
| 347 | 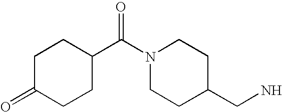 | 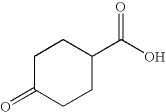 | J. Med. Chem.,1998, 41(5),760 | 511 | 2.42 |
| 348 | 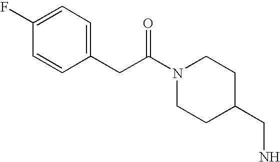 | 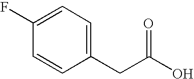 | Aldrich | 523 | 2.78 |
| 349 | 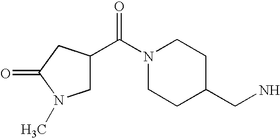 | 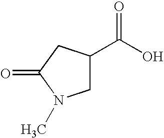 | InterchimIntermediates | 512 | 2.29 |

### Example 350

### Methyl 3-[(1-ethyl-5-{[(phenylmethyl)amino]carbonyl}-1H-pyrazolo[3,4-b]pyridin-4-yl)amino]cyclohexanecarboxylate

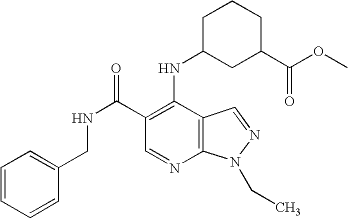

Example 350 was prepared from Intermediate 17 and using an analogous method to that used for the preparation of Example 207. LCMS showed MH⁺=436; T_{RET}=3.20.

### Example 351

### 3-[(1-Ethyl-5-{[(phenylmethyl)amino]carbonyl}-1H-pyrazolo[3,4-b]pyridin-4-yl)amino]cyclohexanecarboxylic acid

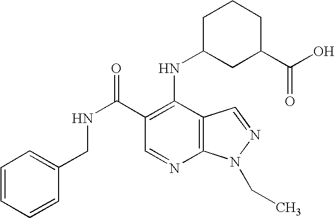

2M-Sodium hydroxide solution (0.5 ml) was added to a stirred suspension of Example 350 (0.12 g, 0.275 mmol) in methanol (3.5 ml) and water (0.8 ml). After stirring overnight at room temperature, the reaction solution was concentrated, diluted with water (3 ml) and acidified with 2M-hydrochloric acid. The resulting precipitate was collected by filtration, washed with water and dried to give Example 351, as a white solid (0.105 g). LCMS showed MH⁺=422; T_{RET}=2.95 min.

### Example 352

### 1-Ethyl-N-(phenylmethyl)-4-(4-piperidinylamino)-1H-pyrazolo[3,4-b]pyridine-5-carboxamide

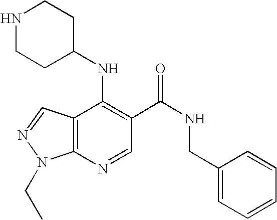

Aqueous hydrochloric acid (20 ml, 5M) was added to a solution of Intermediate 65 (2.58 g, 5.40 mmol) in tetrahydrofuran (10 ml). The reaction mixture was stirred at 20° C. for 22 h then evaporated in vacuo. The residue was partitioned between DCM and water. The aqueous phase was basified with aqueous sodium hydroxide solution (2M) and extracted with diethyl ether. The organic phases was evaporated in vacuo to give Example 352 as a white solid (2.04 g). LCMS showed MH⁺=379; T_{RET}=2.10 min.

### Example 353

### Ethyl 1-ethyl-4-({1-[(methyloxy)acetyl]-4-piperidinyl}amino)-1H-pyrazolo[3,4-b]pyridine-5-carboxylate

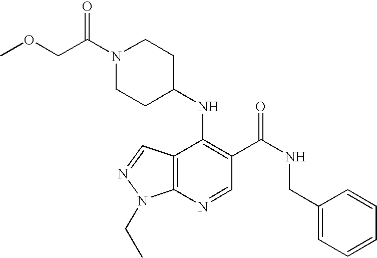

Methoxyacetyl chloride (0.016 mg, 0.144 mmol) and triethylamine (0.02 mol, 0.144 mmol) were added to a solution of Example 352 (0.046 g, 0.122 mmol) in DCM in a Reactivial. The reaction was stirred for 22 h at 20° C. then diluted with DCM and washed with aqueous sodium hydrogen carbonate solution. The organic phase was separated and applied directly to a SPE cartridge (silica 2 g). The cartridge was eluted with DCM MeOH (1% followed by 3%) to give Example 353 as a white solid (0.05 g). LCMS showed MH⁺=451; T_{RET}=2.66 min.

### Example 354

### Ethyl 1-(1-methylethyl)-4-(tetrahydro-2H-pyran-4-ylamino)-1H-pyrazolo[3,4-b]pyridine-5-carboxylate

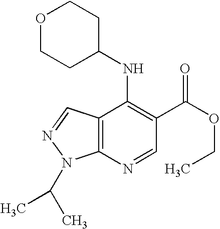

Prepared in a similar manner to example 186 using Example 20 (0.03 g, 0.1 mmol), with isopropylbromide (10 uL, 0.11 mmol), a further 0.11 mmol of alkylating agent was added after 16 hours. The final compound was formed as a clear gum (16 mg). LCMS showed MH⁺=333; T_{RET}=3.16 min.

### Example 355

### 4-(Cyclohexylamino)-1-ethyl-N-methyl-1H-pyrazolo[3,4-b]pyridine-5-carboxamide

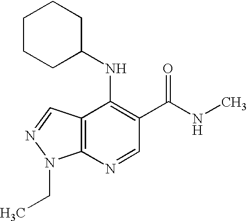

Intermediate 64 (0.02 g, 0.084 mmol) and diisopropylethylamine (0.044 ml, 0.252 mmol) were suspended in N-methylpyrrolidinone (1 ml) and cyclohexylamine (0.012 ml, 0.1 mmol) was added. The mixture was heated at 85° C. with stirring in a Reactivial™ for 8 h, then concentrated in vacuo. The residue was partitioned between DCM (2 ml) and water (2 ml). The layers were separated and the organic layer was concentrated in vacuo, then purified by mass directed autoprep HPLC to afford Example 355 (0.012 g). LCMS showed MH⁺=302; T_{RET}=2.85 min.

### Example 356

### 1-Ethyl-N-(4-fluorophenyl)-6-methyl-4-(tetrahydro-2H-pyran-4-ylamino)-1H-pyrazolo[3,4-b]pyridine-5-carboxamide

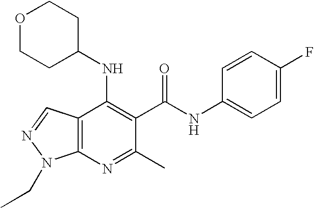

Example 356 was prepared from Intermediate 53 using an analogous method to Example 191. LCMS showed MH⁺=398; T_{RET}=2.18 min.

### Example 357

### 1-Ethyl-6-methyl-N-{[4-(methylsulfonyl)phenyl]methyl}-4-(tetrahydro-2H-pyran-4-ylamino)-1H-pyrazolo[3,4-b]pyridine-5-carboxamide

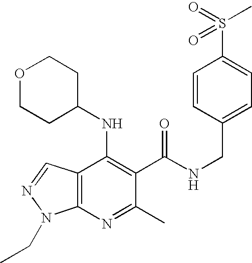

Example 357 was prepared from Intermediate 53 using an analogous method to Example 191. LCMS showed MH⁺=472; T_{RET}=2.15 min.

### Example 358

### N-(2,3-Dihydro-1H-inden-2-yl)-1-ethyl-6-methyl-4-(tetrahydro-2H-pyran-4-ylamino)-1H-pyrazolo[3,4-b]pyridine-5-carboxamide

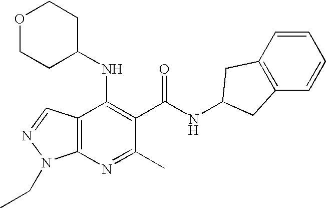

Example 358 was prepared from Intermediate 53 using an analogous method to Example 191. LCMS showed MH⁺=394; T_{RET}=2.04 min.

### Examples 360-414

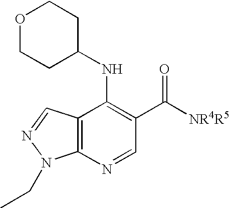

### General Procedure

Intermediate 33 (1.89 g) was treated with thionyl chloride (10 ml) and the mixture heated under reflux for 2 h. Excess thionyl chloride was removed in vacuo to afford Intermediate 73, presumed to be the acid chloride of Intermediate 33 as a cream solid. The solid was suspended in tetrahydrofuran (32.5 ml) and an aliquot of the suspension added to a mixture of the amine (0.11 mmol) and N,N-diisopropylethylamine (0.165-0.22 mmol) in THF (0.5 ml). The reaction mixture was agitated for 24 h and the solvent removed in vacuo. The residue was purified by mass directed autoprep HPLC.

| | | | | | LC-MC |
|---|---|---|---|---|---|
| Example | | Source of | Starting | MH + | Retention |
| Number | NR⁴R⁵ | HNR⁴R⁵ | Material | ion | time |
| 360 | 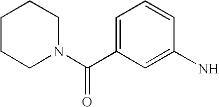 | InterchimIntermediates | Intermediate 33 | 477 | 2.98 |
| 361 | 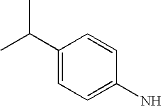 | Aldrich | Intermediate 33 | 408 | 3.45 |
| 362 | 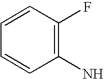 | Aldrich | Intermediate 33 | 384 | 3.09 |
| 363 | 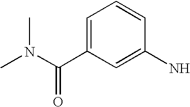 | Butt Park Ltd. | Intermediate 33 | 437 | 2.69 |
| 364 | 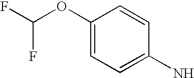 | Aldrich | Intermediate 33 | 432 | 3.21 |
| 365 | 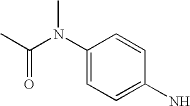 | MaybridgeChemicalCompany Ltd. | Intermediate 33 | 437 | 2.72 |
| 366 | 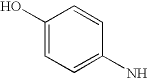 | Aldrich | Intermediate 33 | 382 | 2.67 |
| 367 | 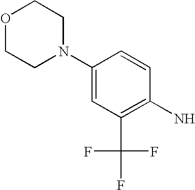 | InterchimIntermediates | Intermediate 33 | 519 | 3.01 |
| 368 | 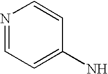 | Aldrich | Intermediate 33 | 367 | 2.19 |
| 369 | 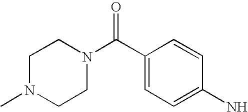 | Butt Park Ltd. | Intermediate 33 | 492 | 2.21 |
| 370 | 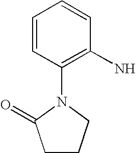 | J. Chem. Soc. C,1969, 1444 | Intermediate 33 | 449 | 2.72 |
| 371 | 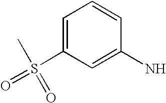 | PeakdaleTechnologiesLimitedM | Intermediate 33 | 444 | 2.81 |
| 372 | 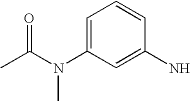 | J. Heterocycl.Chem., 1975,3312(2), 225 | Intermediate 33 | 437 | 2.74 |
| 373 | 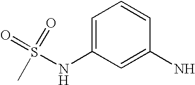 | InterchimIntermediates | Intermediate 33 | 459 | 2.79 |
| 374 | 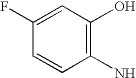 | Apollo ScientificLtd. | Intermediate 33 | 400 | 2.99 |
| 375 | 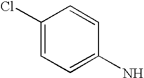 | Aldrich | Intermediate 33 | 400 | 3.35 |
| 376 | 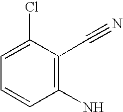 | Lancaster | Intermediate 33 | 425 | 3.07 |
| 377 | 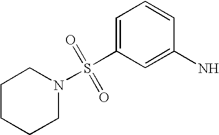 | MaybridgeCombiChem | Intermediate 33 | 513 | 3.33 |
| 379 | 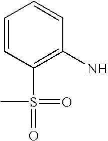 | PeakdaleTechnologiesLimited | Intermediate 33 | 444 | 2.99 |
| 380 | 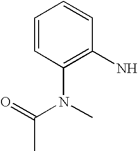 | J. Heterocycl.Chem., 1975,3312(2), 225 | Intermediate 33 | 437 | 2.64 |
| 381 | 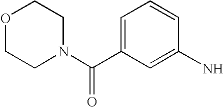 | InterchimIntermediates | Intermediate 33 | 479 | 2.68 |
| 382 | 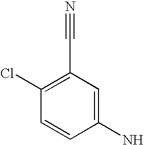 | AcetoCorporation | Intermediate 33 | 425 | 3.38 |
| 383 | 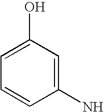 | Aldrich | Intermediate 33 | 382 | 2.78 |
| 384 | 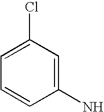 | Aldrich | Intermediate 33 | 400 | 3.38 |
| 386 | 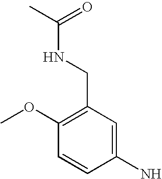 | WO 03/32986 | Intermediate 33 | 467 | 2.65 |
| 387 | 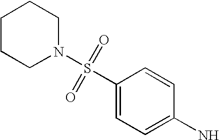 | MaybridgeChemicalCompany Ltd. | Intermediate 33 | 513 | 3.35 |
| 388 | 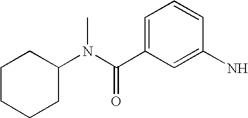 | Intermediate 67 | Intermediate 33 | 505 | 3.23 |
| 389 | 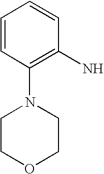 | Lancaster | Intermediate 33 | 451 | 3.17 |
| 390 | 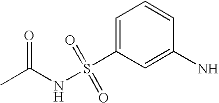 | EP 538945 | Intermediate 33 | 487 | 2.80 |
| 391 | 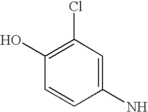 | Aldrich | Intermediate 33 | 416 | 2.99 |
| 392 | 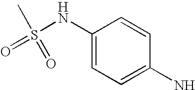 | InterchimIntermediates | Intermediate 33 | 459 | 2.74 |
| 393 | 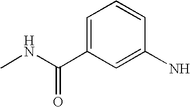 | Butt Park Ltd. | Intermediate 33 | 423 | 2.66 |
| 394 | 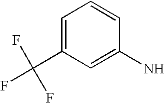 | Aldrich | Intermediate 33 | 434 | 3.43 |
| 395 | 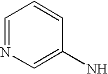 | Aldrich | Intermediate 33 | 367 | 2.40 |
| 396 | 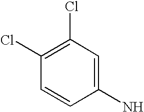 | Aldrich; orReetz, Synthesis,1999, 9, 1555 | Intermediate 33 | 434 | 3.67 |
| 397 | 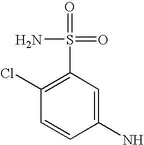 | Bayer AG | Intermediate 33 | 479 | 2.89 |
| 398 | 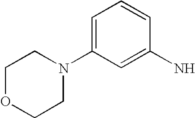 | ExploratoryLibrary | Intermediate 33 | 451 | 2.91 |
| 399 | 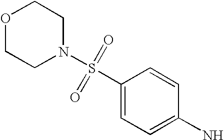 | MaybridgeChemicalCompany Ltd. | Intermediate 33 | 515 | 3.02 |
| 400 | 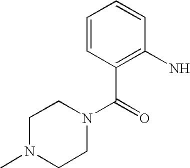 | TimTec | Intermediate 33 | 492 | 2.20 |
| 401 | 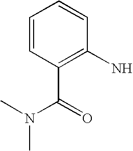 | ExploratoryLibrary | Intermediate 33 | 437 | 2.68 |
| 402 | 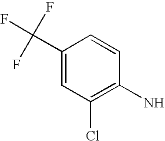 | Lancaster | Intermediate 33 | 468 | 3.53 |
| 403 | 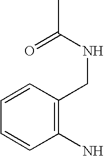 | Heterocycles,1983 20(3), 445 | Intermediate 33 | 437 | 2.70 |
| 404 | 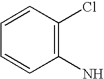 | Aldrich | Intermediate 33 | 400 | 3.09 |
| 405 | 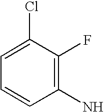 | Aldrich | Intermediate 33 | 418 | 3.21 |
| 406 | 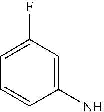 | Aldrich | Intermediate 33 | 384 | 3.19 |
| 407 | 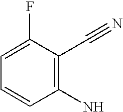 | Aldrich | Intermediate 33 | 409 | 2.95 |
| 408 | 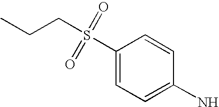 | Helv. Chim.Acta, 198366(4), 1046 | Intermediate 33 | 472 | 3.07 |
| 409 | 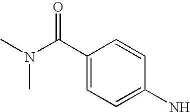 | Butt Park Ltd. | Intermediate 33 | 437 | 2.68 |
| 411 | 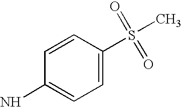 | Salor | Intermediate 33 | 444 | 2.69 |
| 413 | 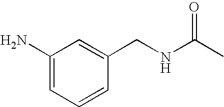 | PeakdaleMolecular Limited | Intermediate 33 | 437 | 2.35 |

### Example 414

### 1-Ethyl-4-(tetrahydro-2H-pyran-3-ylamino)-1H-pyrazolo[3,4-b]pyridine-5-carboxamide

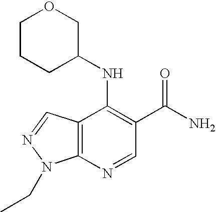

Example 414 was prepared from Intermediate 59 using the general method described for examples 360-413 method. LCMS showed MH⁺=398; T_{RET}=2.90 min.

### Examples 415-487

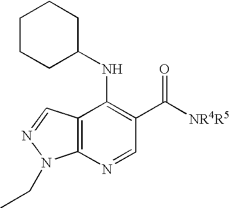

### General Procedure

A mixture of Intermediate 61 (0.1 mmol), HATU (0.1 mmol) and DIPEA (0.4 mmol) in DMF (0.4 ml) was shaken at room temperature for 10 min. A solution of the amine (0.1 mmol) in DMF (0.2 ml) was then added and the mixture agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

| | | | | | LC-MC |
|---|---|---|---|---|---|
| Example | | Source of | Starting | MH + | Retention |
| number | NR⁴R⁵ | HNR⁴R⁵ | Material | ion | time |
| 415 | 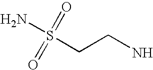 | RareChemicalsGmbH | Intermediate 61 | 395 | 2.80 |
| 416 | 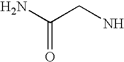 | Aldrich | Intermediate 61 | 345 | 2.64 |
| 417 | 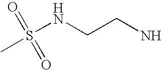 | Ultrafine(UFC Ltd) | Intermediate 61 | 409 | 2.84 |
| 418 | 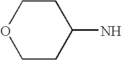 | Intermediate8A; orIntermediate 8(Combi-Blocks) | Intermediate 61 | 372 | 3.03 |
| 419 | 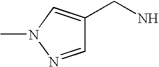 | N. D. ZelinskyInstituteOrganicChemistry | Intermediate 61 | 382 | 2.96 |
| 420 | 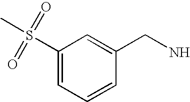 | PeakdaleMolecularLtd. | Intermediate 61 | 456 | 3.22 |
| 421 | 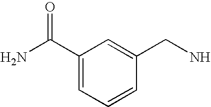 | PeakdaleMolecularLtd. | Intermediate 61 | 421 | 3.03 |
| 422 | 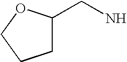 | Aldrich | Intermediate 61 | 372 | 3.09 |
| 423 | 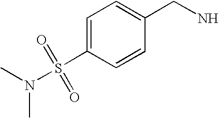 | J. Org.Chem., 1955,20, 1657 | Intermediate 61 | 485 | 3.44 |
| 424 | 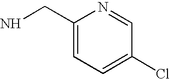 | Key OrganicsLtd | Intermediate 61 | 413 | 3.39 |
| 425 | 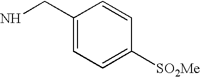 | Acros | Intermediate 61 | 456 | 3.19 |
| 426 | 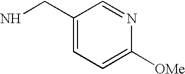 | WO 00/17163 | Intermediate 61 | 409 | 3.3 |
| 427 | 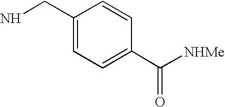 | PeakdaleMolecular Ltd | Intermediate 61 | 421 | 3.23 |
| 428 | 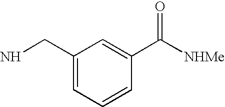 | PeakdaleMolecular Ltd | Intermediate 61 | 435 | 3.07 |
| 429 | 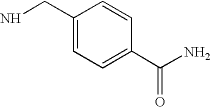 | PeakdaleMolecular Ltd | Intermediate 61 | 421 | 2.97 |
| 430 | 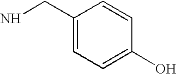 | Apin | Intermediate 61 | 394 | 3.25 |
| 431 | 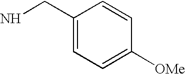 | Acros; orAldrich; orJung et al.,TetrahedronLett., 2002,43(48), 8735;or Meindl etal., J. Med.Chem., 1984,27(9), 1111; orOrg. Lett.,2002, 4, 2055 | Intermediate 61 | 408 | 3.51 |
| 432 | 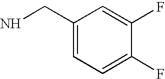 | Aldrich | Intermediate 61 | 414 | 3.68 |
| 433 | 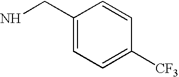 | Aldrich; orMeindl et al., J.Med. Chem.,1984, 27(9),1111. | Intermediate 61 | 446 | 3.81 |
| 434 | 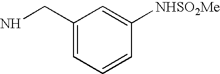 | J. Med. | Intermediate 61 | 471 | 3.23 |
| 435 | 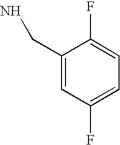 | AldrichChem., 1999,42(14), 2504 | Intermediate 61 | 414 | 3.66 |
| 436 | 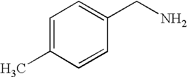 | Aldrich; orOrganicLetters, 2002,4(12), 2055 | Intermediate 61 | 392 | 3.69 |
| 438 | 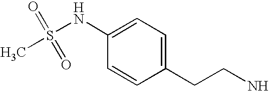 | Key OrganicsLtd | Intermediate 61 | 485 | 3.25 |
| 439 | 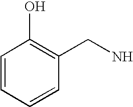 | Buttpark | Intermediate 61 | 394 | 3.52 |
| 440 | 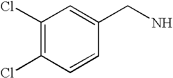 | Aldrich; orMeindl et al., J.Med. Chem.,1984, 27(9),1111. | Intermediate 61 | 446 | 4 |
| 441 | 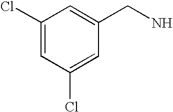 | Lancaster;or Meindlet al., J.Med.Chem.,1984,27(9),1111. | Intermediate 61 | 446 | 4.08 |
| 442 | 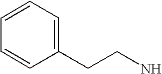 | Aldrich | Intermediate 61 | 392 | 3.62 |
| 443 | 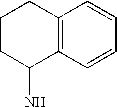 | Aldrich | Intermediate 61 | 418 | 3.83 |
| 444 | 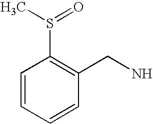 | WO 01/38323 | Intermediate 61 | 440 | 3.07 |
| 445 | 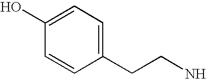 | Aldrich | Intermediate 61 | 408 | 3.31 |
| 446 | 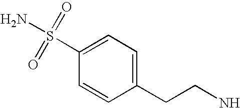 | Acros | Intermediate 61 | 471 | 3.13 |
| 447 | 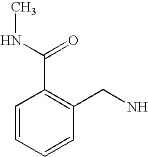 | PeakdaleMolecular Ltd | Intermediate 61 | 435 | 3.13 |
| 448 | 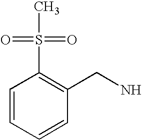 | PeakdaleMolecular Ltd | Intermediate 61 | 456 | 3.32 |
| 449 | 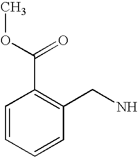 | PeakdaleMolecular Ltd | Intermediate 61 | 436 | 3.56 |
| 450 | 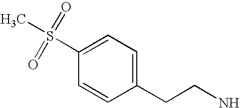 | Aldrich | Intermediate 61 | 471 | 2.79 |
| 451 | 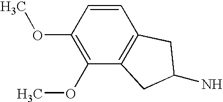 | J. Med.Chem., 1982,25(12), 1442 | Intermediate 61 | 465 | 3.11 |
| 452 | 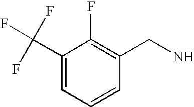 | ABCR | Intermediate 61 | 464 | 3.47 |
| 453 | 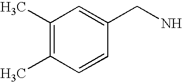 | MatrixScientific; orChem. Ber.,1969, 102,2770 | Intermediate 61 | 407 | 3.35 |
| 454 | 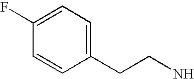 | Aldrich | Intermediate 61 | 411 | 3.18 |
| 455 | 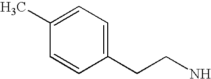 | Aldrich | Intermediate 61 | 407 | 3.3 |
| 456 | 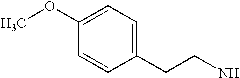 | Aldrich | Intermediate 61 | 423 | 3.09 |
| 457 (asCF₃C(O)OHsalt | 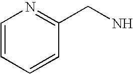 | Aldrich | Intermediate 61 | 379 | 2.92 |
| 458 | 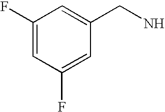 | Aldrich | Intermediate 61 | 414 | 3.68 |
| 459 | 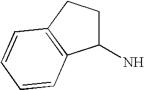 | Aldrich | Intermediate 61 | 404 | 3.72 |
| 460 (asCF₃C(O)OHsalt) | 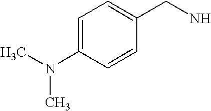 | Aldrich | Intermediate 61 | 421 | 3.29 |
| 461 | 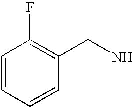 | Aldrich | Intermediate 61 | 396 | 3.58 |
| 462 | 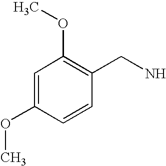 | Aldrich | Intermediate 61 | 438 | 3.53 |
| 463 (asCF₃C(O)OHsalt) | 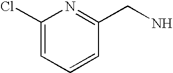 | InorganicChemistry,1997, 36(9),1967 | Intermediate 61 | 413 | 3.4 |
| 464 (asCF₃C(O)OHsalt) | 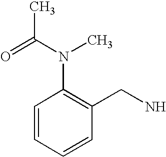 | PeakdaleMolecular Ltd | Intermediate 61 | 449 | 3.18 |
| 465 | 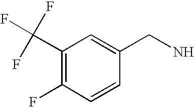 | ABCR | Intermediate 61 | 422 | 3.77 |
| 466 | 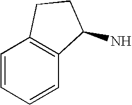 | Aldrich | Intermediate 61 | 404 | 3.72 |
| 467 | 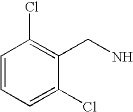 | Pfaltz-Bauer;or Meindl etal., J. Med.Chem., 1984,27(9), 1111. | Intermediate 61 | 446 | 3.85 |
| 468 | 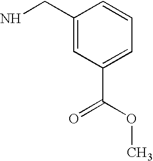 | PeakdaleMolecular Ltd | Intermediate 61 | 436 | 3.53 |
| 469 | 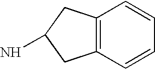 | Aldrich | Intermediate 61 | 404 | 3.66 |
| 470 | 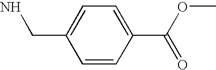 | Aldrich | Intermediate 61 | 435 | 3.52 |
| 471 | 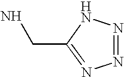 | Esprit | Intermediate 61 | 370 | 2.82 |
| 472 | 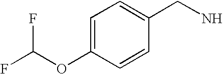 | Apollo | Intermediate 61 | 444 | 3.63 |
| 473 | 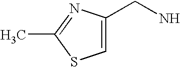 | MicroChemis-tryRadaPharma | Intermediate 61 | 399 | 3.16 |
| 474 | 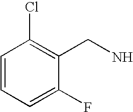 | Fluka | Intermediate 61 | 430 | 3.72 |
| 475 | 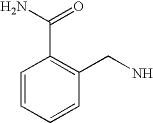 | J. Am. Chem.Soc., 1977,99, 3075 | Intermediate 61 | 421 | 3.04 |
| 477 | 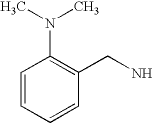 | J. Org.Chem., 2001,66(6), 1999 | Intermediate 61 | 421 | 2.89 |
| 478 | 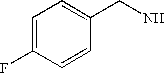 | Aldrich | Intermediate 61 | 396 | 3.59 |
| 479 | 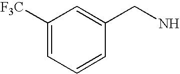 | Aldrich; orMeindl et al., J.Med. Chem.,1984, 27(9),1111. | Intermediate 61 | 446 | 3.80 |
| 480 | 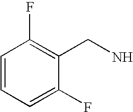 | Aldrich | Intermediate 61 | 414 | 3.57 |
| 481 | 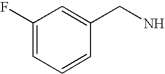 | Aldrich; orMeindl et al., J.Med. Chem.,1984, 27(9),1111. | Intermediate 61 | 396 | 3.62 |
| 482 | 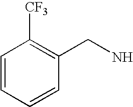 | Aldrich | Intermediate 61 | 446 | 3.82 |
| 483 | 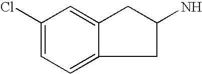 | J. Med.Chem., 2001,44(26), 4628 | Intermediate 61 | 438 | 3.95 |
| 484 | 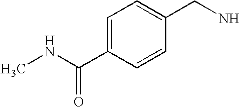 | WO 9417035 | Intermediate 61 | | |
| 485 | 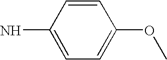 | Aldrich | Intermediate 61 | 394 | 3.61 |
| 486 | 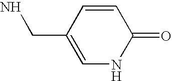 | MicroChemis-tryRadaPharma | Intermediate 61 | 395 | 2.78 |
| 487 | 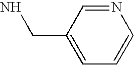 | Aldrich | Intermediate 61 | 379 | 2.71 |

### Example 488

### 4-[({[4-(Cyclohexylamino)-1-ethyl-1H-pyrazolo[3,4-b]pyridin-5-yl]carbonyl}amino)methyl]benzoic acid

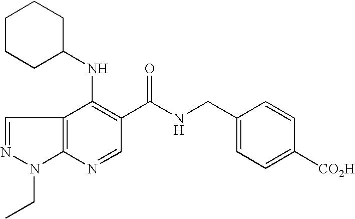

2M-Sodium hydroxide solution (29 μL, 0.058 mmol) was added to a stirred solution of Example 470 (6 mg, 0.014 mmol) in methanol (28 μL) and water (2 μL). The resulting solution was stirred at 50° C. under nitrogen. After 16 h, the mixture was diluted with water (0.5 ml) and adjusted to pH 4 with acetic acid. The precipitated solid was collected by filtration and dried in vacuo to afford Example 488 as a white solid (4.5 mg). LCMS showed MH⁺=422; T_{RET}=3.26 min.

### Example 489

### 3-[({[4-(Cyclohexylamino)-1-ethyl-1H-pyrazolo[3,4-b]pyridin-5-yl]carbonyl}amino)methyl]benzoic acid

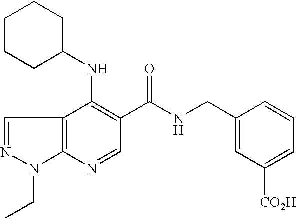

2M-Sodium hydroxide solution (83 μL, 0.166 mmol) was added to a stirred solution of Example 468 (18 mg, 0.042 mmol) in methanol (88 μL) and water (5 μL). The resulting solution was stirred at 50° C. under nitrogen. After 16 h, a further quantity of 2M-sodium hydroxide solution (29 μL, 0.058 mmol) was added to the reaction mixture. After 24 h, the reaction mixture was diluted with water (0.5 ml) and adjusted to pH 4 with acetic acid. The mixture was extracted with ethyl acetate (2×0.5 ml), and the combined extracts were dried (Na₂SO₄) and evaporated in vacuo to give a solid (21 mg). This solid was purified on an SPE cartridge (silica, 1 g) eluting with ethyl acetate:cyclohexane (1:1) followed by methanol. Fractions containing the desired product were combined and concentrated to afford Example 489 as a white solid (4.6 mg). LCMS showed MH⁺=422; T_{RET}=3.22 min.

### Example 490

### 4-(Cyclohexylamino)-N-(2,3-dihydro-1H-inden-2-yl)-1-ethyl-1H-pyrazolo[3,4-b]pyridine-5-carboxamide hydrochloride

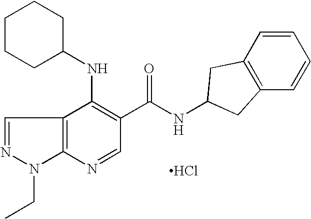

A solution of Example 469 (71 mg, 0.17 mmol) in anhydrous THF (2 ml) was treated with hydrogen chloride in dioxane (4M, 0.3 ml). After standing at ambient temperature for 16 hours the resulting solid was collected by filtration and dried under vacuum to give Example 490 as rod like crystals (36 mg). LCMS showed MH⁺=404; T_{RET}=3.60 min.

### Example 491

### 4-(Cyclohexylamino)-N-(2,3-dihydro-1H-inden-2-yl)-1-ethyl-1H-pyrazolo[3,4-b]pyridine-5-carboxamide methanesulphonate

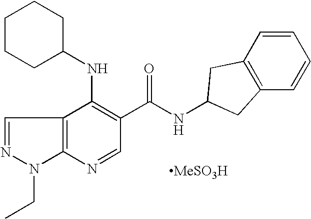

A solution of Example 469 (71 mg, 0.17 mmol) in anhydrous THF (2 ml) was treated with anhydrous methane sulphonic acid (11.4 μL, 0.17 mmol). After standing at ambient temperature for 16 hours the resulting solid was collected by filtration and dried under vacuum to give Example 491 as rod like crystals (23 mg). LCMS showed MH⁺=404; T_{RET}=3.59 min.

### Examples 492-649

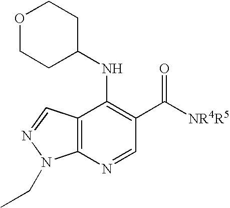

### General Procedure

A mixture of Intermediate 33 (0.1 mmol), HATU (0.1 mmol) and DIPEA (0.4 mmol) in DMF (0.4 ml) was shaken at room temperature for 10 min. A solution of the amine (0.1 mmol) in DMF (0.2 ml) was then added and the mixture agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

| | | | | | LC-MC |
|---|---|---|---|---|---|
| Example | | Source of | Starting | MH⁺ | Retention |
| number | NR⁴R⁵ | HNR⁴R⁵ | Material | ion | time |
| 492 (asCF₃C(O)OHsalt) | 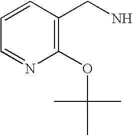 | PeakdaleMolecularLtd. | Intermediate33 | 453 | 2.90 |
| 493 | 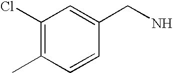 | MaybridgeChemicalCompanylLtd.; or WO 01/30745 | Intermediate33 | 428 | 2.92 |
| 494 | 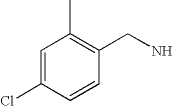 | Trans WorldChemicals,Inc.; or DE1953059 | Intermediate33 | 428 | 2.91 |
| 495 | 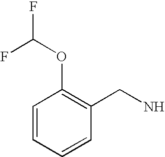 | FluorochemLtd. | Intermediate33 | 446 | 2.70 |
| 496 | 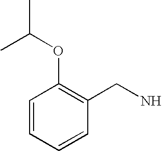 | PeakdaleMolecularLtd. | Intermediate33 | 438 | 2.83 |
| 497 | 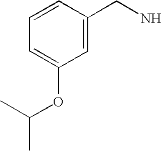 | PeakdaleMolecularLtd. | Intermediate33 | 438 | 2.79 |
| 498 | 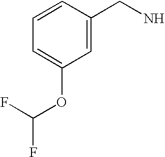 | FluorochemLtd. | Intermediate33 | 446 | 2.73 |
| 499 | 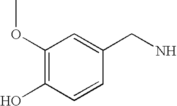 | Aldrich | Intermediate33 | 426 | 2.50 |
| 500 | 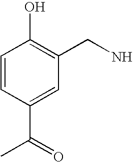 | NipponKagakuZasshi; 1952,73; 393 | Intermediate33 | 438 | 2.62 |
| 501 | 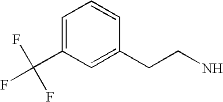 | ApolloScientific Ltd. | Intermediate33 | 462 | 2.88 |
| 502 | 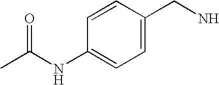 | ApinChemicalsLtd. | Intermediate33 | 437 | 2.19 |
| 503 | 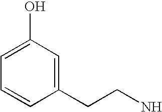 | Sigma | Intermediate33 | 410 | 2.60 |
| 504 | 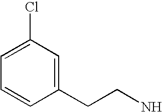 | Aldrich | Intermediate33 | 428 | 2.80 |
| 505 | 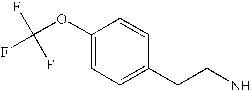 | Miteni S.p.A. | Intermediate33 | 478 | 2.97 |
| 506 | 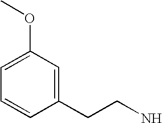 | Aldrich | Intermediate33 | 424 | 2.58 |
| 507 | 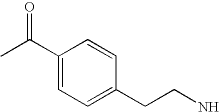 | J. Med.Chem., 1997,20(9), 1210 | Intermediate33 | 436 | 2.44 |
| 508 | 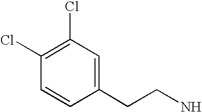 | FluorochemLtd. | Intermediate33 | 462 | 2.99 |
| 509 | 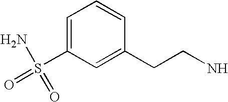 | JP 11080156 | Intermediate33 | 473 | 2.2 |
| 510 | 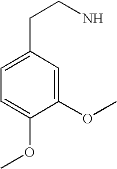 | Aldrich | Intermediate33 | 454 | 2.41 |
| 512 | 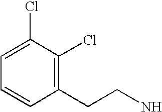 | SynchemOHG | Intermediate33 | 462 | 2.96 |
| 513 | 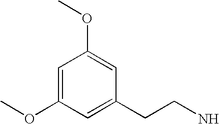 | ApinChemicalsLtd. | Intermediate33 | 454 | 2.59 |
| 514 | 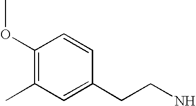 | J. Chem. Soc.Perkin Trans.1, 1977, 386 | Intermediate33 | 438 | 2.75 |
| 515 | 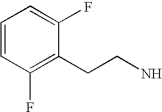 | SIGMA-RBI | Intermediate33 | 430 | 2.65 |
| 516 | 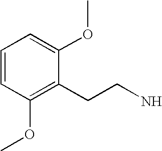 | WO 9303022 | Intermediate33 | 454 | 2.67 |
| 517 | 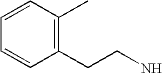 | SIGMA-RBI | Intermediate33 | 408 | 2.73 |
| 518 | 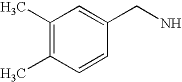 | MatrixScientific; orChem. Ber.,1969, 102,2770 | Intermediate33 | 408 | 3.2 |
| 519 | 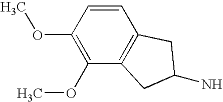 | J. Med.Chem., 198225(12), 1442 | Intermediate33 | 466 | 3 |
| 521 | 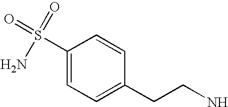 | Acros | Intermediate33 | 473 | 2.62 |
| 522 | 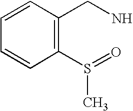 | WO 01/38323 | Intermediate33 | 445 | 2.55 |
| 523 | 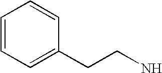 | Aldrich | Intermediate33 | 394 | 3 |
| 524 | 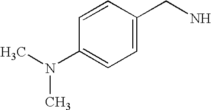 | Aldrich | Intermediate33 | 423 | 2.51 |
| 525 | 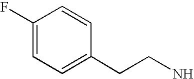 | Aldrich | Intermediate33 | 412 | 3.06 |
| 526 | 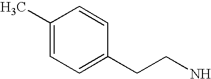 | Aldrich | Intermediate33 | 408 | 3.16 |
| 527 | 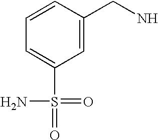 | YakugakuZasshi; 195070, 71 | Intermediate33 | 459 | 2.6 |
| 528 | 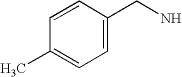 | Aldrich; orOrganicLetters, 2002,4(12), 2055 | Intermediate33 | 394 | 3.08 |
| 530 | 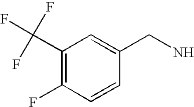 | Lancaster | Intermediate33 | 466 | 3.31 |
| 531 | 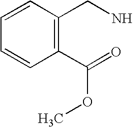 | J. AM. Chem.Soc., 1976,78(22), 6978 | Intermediate33 | 438 | 3 |
| 532 (asCF₃C(O)OHsalt) | 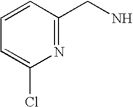 | InorganicChemistry,1997, 36(9),1967 | Intermediate33 | 415 | 2.82 |
| 533 | 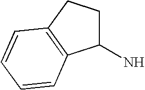 | Aldrich | Intermediate33 | 406 | 3.14 |
| 534 | 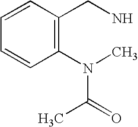 | PeakdaleMolecularLtd. | Intermediate33 | 451 | 2.71 |
| 535 | 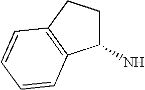 | Aldrich | Intermediate33 | 406 | 3.15 |
| 536 | 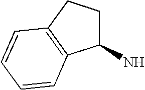 | Aldrich | Intermediate33 | 406 | 3.15 |
| 537 | 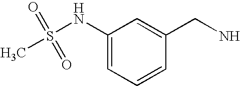 | J. Med.Chem., 1999,42(14), 2504 | Intermediate33 | 473 | 2.58 |
| 538 | 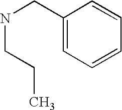 | ChemicalBuildingBlocks | Intermediate33 | 422 | 2.92 |
| 540 | 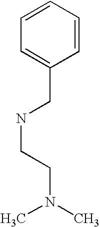 | Aldrich | Intermediate33 | 451 | 2.13 |
| 541 | 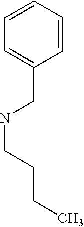 | Aldrich | Intermediate33 | 436 | 3.15 |
| 542 | 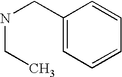 | Aldrich | Intermediate33 | 408 | 2.85 |
| 544 | 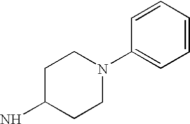 | JanssenPharma-ceuticals | Intermediate33 | 449 | 2.67 |
| 545 | 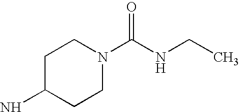 | Intermediate69 | Intermediate33 | 444 | 2.34 |
| 546 (asH—C(O)OHsalt = formicacid additionsalt) | 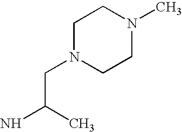 | ArzneimittelForschung,1974, 24(4a),584 | Intermediate33 | 430 | 1.95 |
| 547 | 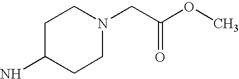 | WO 97/25323 | Intermediate33 | 445 | 1.96 |
| 548 (asCF₃C(O)OHsalt) | 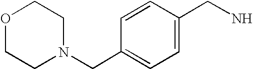 | WO 03/32980 | Intermediate33 | 479 | 2.21 |
| 549 (asCF₃C(O)OHsalt) | 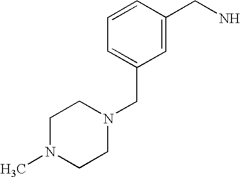 | WO 03/32980 | Intermediate33 | 492 | 2.24 |
| 550 (asCF₃C(O)OHsalt) | 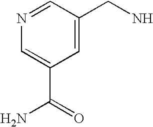 | WO 02/85860 | Intermediate33 | 424 | 2.33 |
| 551 | 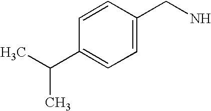 | Salor | Intermediate33 | 422 | 3.36 |
| 552 | 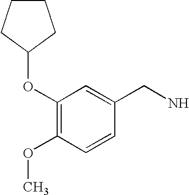 | WO 95/00516 | Intermediate33 | 494 | 3.22 |
| 553 (asCF₃C(O)OHsalt) | 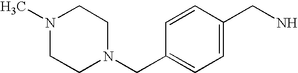 | WO 03/32980 | Intermediate33 | 492 | 2.21 |
| 554 | 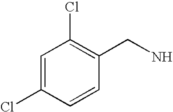 | Aldrich; orMeindl et al.,J. Med.Chem., 1984,27(9), 1111. | Intermediate33 | 448 | 3.4 |
| 555 | 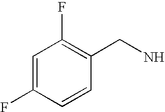 | Aldrich | Intermediate33 | 416 | 3.06 |
| 556 | 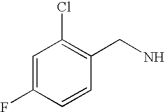 | Salor | Intermediate33 | 432 | 3.21 |
| 557 | 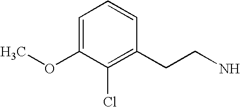 | DE 2300018 | Intermediate33 | 458 | 3.12 |
| 558 | 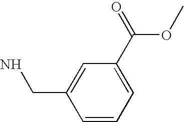 | PeakdaleMolecular Ltd. | Intermediate33 | 436 | 3.41 |
| 559 (asCF₃C(O)OHsalt) | 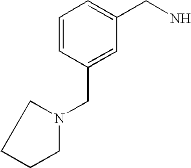 | JP 10045736 | Intermediate33 | 463 | 2.28 |
| 560 | 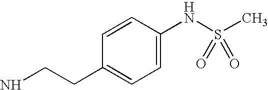 | WO 02/16318EP 338793 | Intermediate33 | 487 | 2.74 |
| 561 | 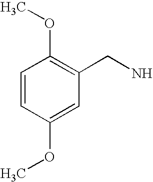 | MaybridgeChemicalCompany Ltd. | Intermediate33 | 440 | 2.99 |
| 562 | 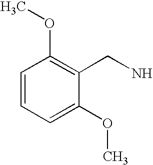 | Lancaster | Intermediate33 | 440 | 3.00 |
| 563 | 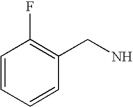 | Aldrich | Intermediate33 | 398 | 3.01 |
| 564 | 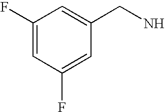 | Aldrich; orMeindl et al.,J. Med.Chem., 1984,27(9), 1111. | Intermediate33 | 416 | 3.11 |
| 565 | 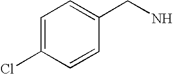 | Aldrich; orOrganicLetters, 2002,4(12), 2055 | Intermediate33 | 414 | 3.19 |
| 567 | 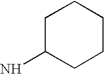 | Aldrich | Intermediate33 | 372 | 3.01 |
| 568 | 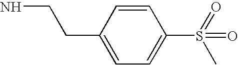 | J. Biol. Chem.,1997, 272(3),1493 | Intermediate33 | 472 | 2.69 |
| 569 | 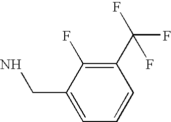 | FluorochemLtd. | Intermediate33 | 466 | 3.29 |
| 570 | 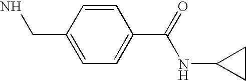 | Intermediate71 | Intermediate33 | 463 | 2.66 |
| 571 | 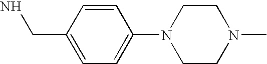 | MaybridgeReactiveintermediates | Intermediate33 | 478 | 2.25 |
| 572 | 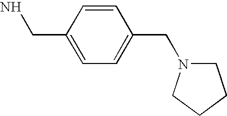 | WO 99/67204 | Intermediate33 | 463 | 2.24 |
| 573 | 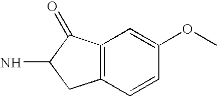 | Eur. J. Med.Chem., 1987,22(5), 417 | Intermediate33 | 450 | 2.90 |
| 574 | 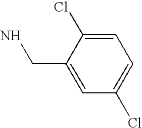 | Lancaster | Intermediate33 | 446/448/450 | 3.35 |
| 575 | 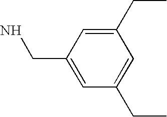 | Eur. J. Med.Chem., 1987,33(5), 363 | Intermediate33 | 436 | 3.48 |
| 576 | 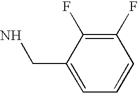 | Avocado | Intermediate33 | 416 | 3.06 |
| 577 | 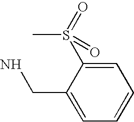 | WO 02/30930 | Intermediate33 | 458 | 2.80 |
| 578 | 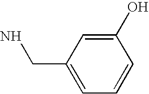 | Apin | Intermediate33 | 458 | 2.80 |
| 579 | 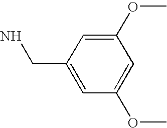 | Aldrich | Intermediate33 | 458 | 2.80 |
| 580 | 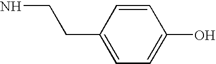 | Aldrich | Intermediate33 | | |
| 581 | 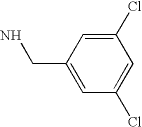 | Lancaster; orJ. Med.Chem., 1984,27(9), 1111. | Intermediate33 | 446/448/450 | 2.80 |
| 582 | 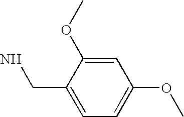 | Aldrich | Intermediate33 | 440 | 2.96 |
| 583 | 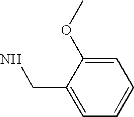 | Aldrich | Intermediate33 | 410 | 2.98 |
| 584 | 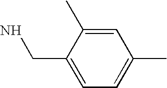 | ICNBiomedicals,Inc.; or Salor;or Synthesis,1982, 12,1036 | Intermediate33 | 408 | 3.18 |
| 585 | 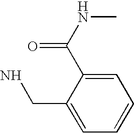 | WO 03/32986 | Intermediate33 | 437 | 2.62 |
| 586 | 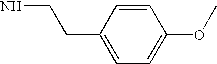 | Aldrich | Intermediate33 | 424 | 3.05 |
| 587 | 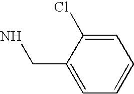 | Aldrich | Intermediate33 | 414/416 | 3.13 |
| 588 | 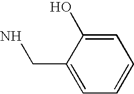 | Buttpark | Intermediate33 | 396 | 2.14 |
| 589 | 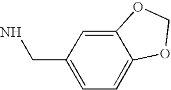 | Aldrich | Intermediate33 | 424 | 2.76 |
| 590 | 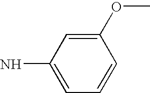 | Lancaster | Intermediate33 | 396 | 2.95 |
| 591 | 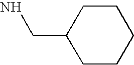 | Aldrich; orSynlett, 1999,4, 409 | Intermediate33 | 386 | 3.10 |
| 592 | 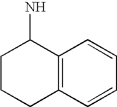 | Aldrich | Intermediate33 | | |
| 593 | 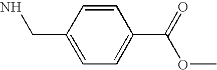 | Apin | Intermediate33 | 438 | 2.82 |
| 594 | 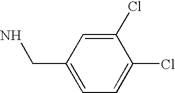 | Aldrich; orMeindl et al.,J. Med.Chem., 1984,27(9), 1111. | Intermediate33 | 448/450/452 | 3.26 |
| 595 | 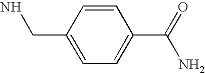 | WO 02/85860 | Intermediate33 | 423 | 2.29 |
| 596 | 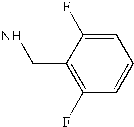 | Aldrich | Intermediate33 | 416 | 3.0 |
| 597 | 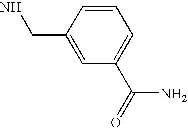 | Aldrich | Intermediate33 | 423 | 2.56 |
| 598 | 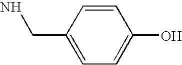 | Apin | Intermediate33 | 396 | 2.54 |
| 599 | 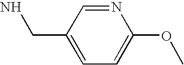 | WO 00/17163 | Intermediate33 | 411 | 2.72 |
| 600 | 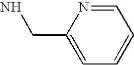 | Aldrich; orJ. Med.Chem., 2003,46(4), 461. | Intermediate33 | 381 | 1.89 |
| 601 | 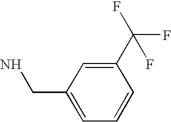 | Aldrich; orMeindl et al.,J. Med.Chem., 1984,27(9), 1111. | Intermediate33 | 448 | 2.96 |
| 602 | 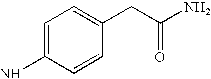 | PeakdaleMolecularLimited | Intermediate33 | 423 | 2.28 |
| 603 | 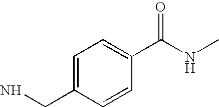 | WO 94/17035 | Intermediate33 | 437 | 2.28 |
| 604 | 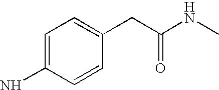 | J. Pharm Sci.,1987, 76(1),18-20 | Intermediate33 | 437 | 2.34 |
| 605 | 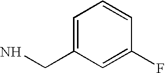 | Aldrich; orMeindl et al.,J. Med.Chem., 1984,27(9), 1111;or OrganicLetters, 2002,4(12), 2055 | Intermediate33 | 398 | 2.71 |
| 606 | 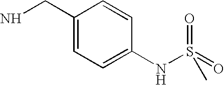 | Lis et al., J.Med. Chem.,1990, 33(10),2883, seeScheme IIIand ref. 24 | Intermediate33 | 473 | 2.40 |
| 607 | 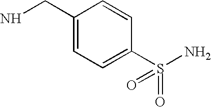 | Sigma | Intermediate33 | 459 | 2.31 |
| 608 | 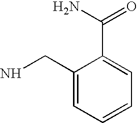 | PeakdaleMolecularLtd. | Intermediate33 | 423 | 2.55 |
| 609 | 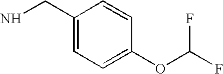 | FluorochemLtd. | Intermediate33 | 446 | 2.82 |
| 610 | 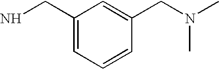 | DE19937494 | Intermediate33 | 437 | 1.86 |
| 611 | 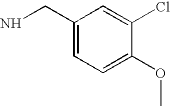 | FluorochemL | Intermediate33 | 444 | 2.80 |
| 612 | 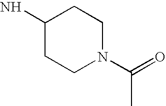 | WO00/72834 | Intermediate33 | 415 | 2.12 |
| 613 | 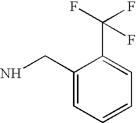 | Aldrich | Intermediate33 | 448 | 2.96 |
| 615 | 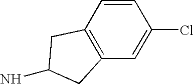 | J. Med.Chem., 2001,44(26), 4628 | Intermediate33 | 440 | 3.03 |
| 616 | 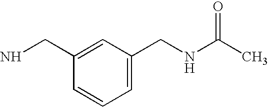 | Intermediate75 (as HClsalt) | Intermediate33 | 451 | 2.62 |
| 617 | 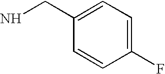 | Aldrich; orOrganicLetters, 2002,4(12), 2055 | Intermediate33 | 398 | 2.90 |
| 618 | 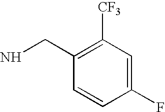 | Alfa | Intermediate33 | 466 | 2.98 |
| 619 | 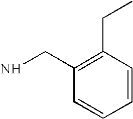 | Energy &Fuels, (1994),8(4), 990-1001 | Intermediate33 | 408 | 2.86 |
| 620 | 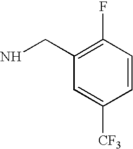 | Alfa | Intermediate33 | 466 | 2.94 |
| 621 | 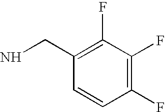 | Apollo | Intermediate33 | 434 | 2.82 |
| 622 | 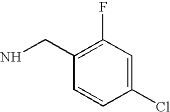 | Acros | Intermediate33 | 432 | 2.9 |
| 623 | 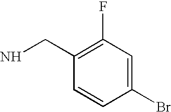 | Acros | Intermediate33 | 476 | 2.95 |
| 624 | 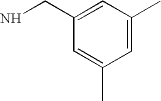 | Apollo; orEur. J. Med.Chem., 1998,33(5), 363 | Intermediate33 | 408 | 2.88 |
| 625 | 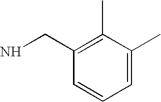 | Maybridge | Intermediate33 | 408 | 2.83 |
| 626 | 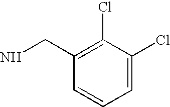 | Lancaster | Intermediate33 | 448 | 3.02 |
| 627 | 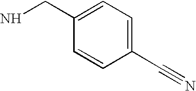 | Apin | Intermediate33 | 405 | 2.56 |
| 628 | 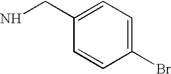 | Ubi-Chem | Intermediate33 | 458 | 2.89 |
| 629 | 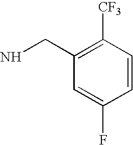 | ABCR | Intermediate33 | 466 | 2.97 |
| 630 | 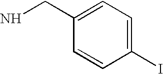 | Lancaster | Intermediate33 | 505.9 | 2.97 |
| 631 | 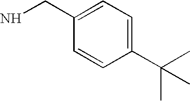 | Apollo | Intermediate33 | 436 | 3.11 |
| 632 | 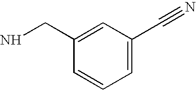 | WO98/33767; orMeindl et al.,J. Med.Chem., 1984,27(9), 1111. | Intermediate33 | 405 | 2.55 |
| 633 | 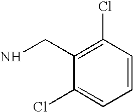 | Pfaltz-Bauer;or Meindl etal., J. Med.Chem., 1984,27(9), 1111 | Intermediate33 | 448 | 2.88 |
| 634 | 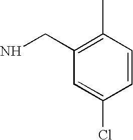 | Transworld | Intermediate33 | 428 | 3.22 |
| 635 | 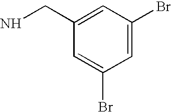 | Apin(HNR⁴R^{5used as HClsalt)} | Intermediate33 | 536 | 3.47 |
| 636 | 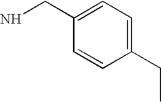 | Matrix | Intermediate33 | 408 | 3.18 |
| 637 | 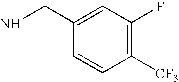 | Avocado | Intermediate33 | 466 | 3.25 |
| 638 | 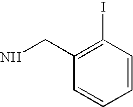 | Pfaltz-Bauer | Intermediate33 | 505.9 | 2.92 |
| 639 | 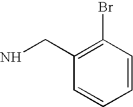 | Alfa | Intermediate33 | 458 | 3.10 |
| 640 | 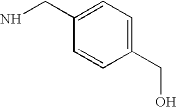 | WO 03/35621(HNR⁴R^{5used as HClsalt)} | Intermediate33 | 410 | 2.49 |
| 641 | 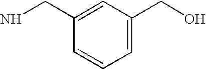 | WO 03/35621(HNR⁴R^{5used as HClsalt)} | Intermediate33 | 410 | 2.51 |
| 642 | 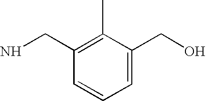 | DE 2136624(HNR⁴R^{5used as HClsalt)} | Intermediate33 | 424 | 2.55 |
| 643 | 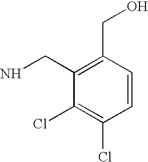 | (HNR⁴R^{5used as HClsalt)} | Intermediate33 | 478 | 2.96 |
| 644 | 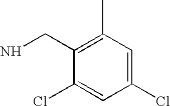 | Aldrich | Intermediate33 | 462 | 3.13 |
| 645 | 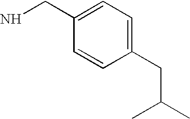 | | Intermediate33 | 436 | 3.18 |
| 646 | 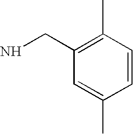 | Matrix | Intermediate33 | 408 | 2.84 |
| 647 | 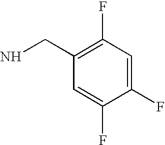 | Apollo | Intermediate33 | 434 | 2.80 |
| 648 | 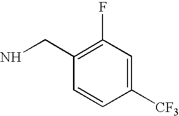 | ABCR | Intermediate33 | 466 | 2.99 |
| 649 | 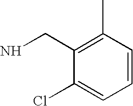 | Lancaster | Intermediate33 | 428 | 2.87 |

### Example 518

### N-[(3,4-dimethylphenyl)methyl]-1-ethyl-4-(tetrahydro-2H-pyran-4-ylamino)-1H-pyrazolo[3,4-b]pyridine-5-carboxamide; also known as: N-(3,4-dimethylbenzyl)-1-ethyl-4-(tetrahydro-2H-pyran-4-ylamino)-1H-pyrazolo[3,4-b]pyridine-5-carboxamide

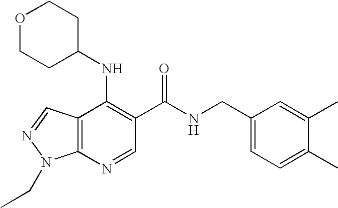

An alternative process for preparing Example 518 is given below:

To a solution of Intermediate 33 (3.5 g, 12.07 mmol) in DMF (500 ml) was added HATU (4.5 g, 12.07 mmol) and the mixture stirred at room temperature for 30 min. 3,4-Dimethylbenzylamine (1.63 g, 12.07 mmol, obtainable from Matrix Scientific, Columbia, USA or by a process described in Chem. Ber., 1969, 102, 2770) was added followed by DIPEA (4.5 ml, 26.55 mmol) and the solution stirred at room temperature for 16 hours. The solvent was removed under reduced pressure and the residue partitioned between saturated aqueous NaHCO₃ (200 ml) and ethyl acetate (250 ml), the aqueous phase re-extracted with ethyl acetate (2×200 ml), the organic extracts combined, dried (Na₂SO₄) and evaporated. The resultant viscous oil was recrystallised from hot ethyl acetate (ca. 100 ml) to give the title compound as a white crystalline solid (3.36 g, 80%). LCMS showed MH⁺=408; Tᵣₑₜ=3.06 min. δ_{H} (D₆ DMSO) 1.36 (3H, t), 1.51 (2H, m), 2.00 (2H, m), 2.18 (3H, s), 2.19 (3H, s), 2.50 (2H, m), 3.61 (2H, m), 3.83 (2H, m), 4.17 (1H, m), 4.36 (2H, q), 4.38 (2H, d), 7.02-7.09 (3H, m), 8.17 (1H, s), 8.62 (1H, s), 8.93 (1H, t), 9.96 (1H, d): δ_{C} (D₆ DMSO) 14.65, 18.91, 19.33, 32.81, 41.06, 41.86, 48.57, 64.94, 101.69, 102.18, 124.44, 128.22, 129.24, 133.28, 134.31, 135.78, 136.91, 149.26, 149.59, 151.36, 168.81

### Example 518A

### N-[(3,4-dimethylphenyl)methyl]-1-ethyl-4-(tetrahydro-2H-pyran-4-ylamino)-1H-pyrazolo[3,4-b]pyridine-5-carboxamide hydrochloride; also known as: N-(3,4-dimethylbenzyl)-1-ethyl-4-(tetrahydro-2H-pyran-4-ylamino)-1H-pyrazolo[3,4-b]pyridine-5-carboxamide hydrochloride

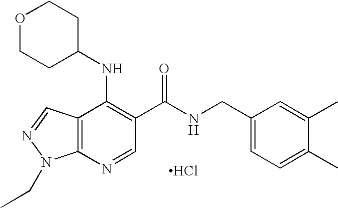

A solution of Example 518 (1.3 g, 3.19 mmol) in anhydrous tetrahydrofuran (200 ml) was treated with a solution of hydrogen chloride in dioxane (4M, 8 ml) and the mixture stirred at ambient temperature for 16 hours. The resultant white precipitate was collected by filtration and recrystallised from hot methanol (100 ml) to give the title compound Example 518A as a white crystalline solid (1.12 g, 79%).

LCMS showed MH⁺=408; Tᵣₑₜ=3.21 min. δ_{H} (D₆ DMSO) 1.39 (3H, t), 1.59 (2H, m), 2.01 (2H, m), 2.19 (3H, s), 2.20 (3H, s), 3.64 (2H, t), 3.83 (2H, m), 4.28 (1H, m), 4.40 (2H, d), 4.50 (2H, q), 7.04-7.11 (3H, m), 9.40 (1H, s (br)), 10.72 (1H, s (br)).

### Example 650

### 4-[({[1-Ethyl-4-(tetrahydro-2H-pyran-4-ylamino)-1H-pyrazolo[3,4-b]pyridin-5-yl]carbonyl}amino)methyl]benzoic acid sodium salt

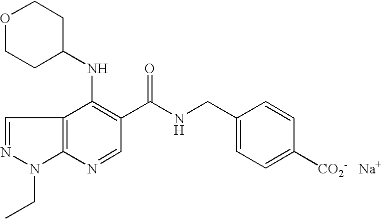

2M-Sodium hydroxide solution (98 μL, 0.196 mmol) was added to a stirred solution of Example 593 (22 mg, 0.049 mmol) in methanol (104 μL) and water (6 μL). The resulting solution was stirred at 50° C. under nitrogen. After 16 h, the reaction mixture was diluted with water (0.5 ml) and adjusted to pH 4 with acetic acid. The mixture was extracted with ethyl acetate (2×0.5 ml), and the combined extracts were dried (Na₂SO₄) and evaporated in vacuo to give a solid (15 mg). This solid was suspended in water (0.5 ml) and treated with 2M-sodium hydroxide solution (15 L). Evaporation of solvent in vacuo afforded Example 650 as a white solid (11 mg). LCMS showed MH⁺=425; T_{RET}=2.69 min.

### Example 651

### 3-[({[1-Ethyl-4-(tetrahydro-2H-pyran-4-ylamino)-1H-pyrazolo[3,4-b]pyridin-5-yl]carbonyl}amino)methyl]benzoic acid

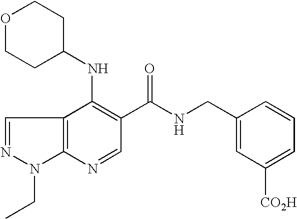

2M-Sodium hydroxide solution (98 μL, 0.196 mmol) was added to a stirred solution of Example 558 (22 mg, 0.049 mmol) in methanol (104 μL) and water (6 μL). The resulting solution was stirred at 50° C. under nitrogen. After 16 h, the reaction mixture was diluted with water (0.5 ml) and adjusted to pH 4 with acetic acid. The precipitated solid was collected by filtration and dried in vacuo to afford Example 651 as a white solid (15 mg). LCMS showed MH⁺=425; T_{RET}=2.72 min.

### Example 652

### Ethyl 1-ethyl-4-{[4-(hydroxyimino)cyclohexyl]amino}-1H-pyrazolo[3,4-b]pyridine-5-carboxylate

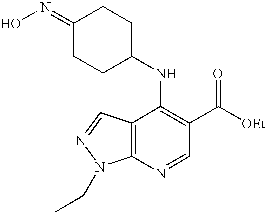

A mixture of Example 205 (200 mg), hydroxylamine hydrochloride (50 mg) and anhydrous potassium carbonate (420 mg) in acetonitrile (10 ml) was stirred and heated at reflux for 17 hours. The solution was cooled and concentrated in vacuo. The residue was partitioned between EtOAc and water. The organic phase was separated, dried over Na₂SO₄ and concentrated in vacuo to give Example 652 as a white powder (203 mg). LCMS showed MH⁺=346; T_{RET}=2.84 min.

### Example 653

### 1-Ethyl-4-{[4-(hydroxyimino)cyclohexyl]amino}-N-{[4-(methyloxy)phenyl]methyl}-1H-pyrazolo[3,4-b]pyridine-5-carboxamide

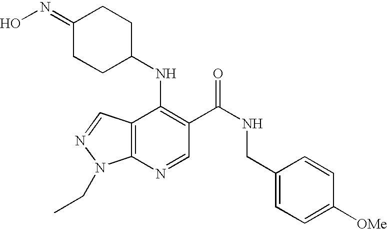

A mixture of Example 263 (217 mg), hydroxylamine hydrochloride (43 mg) and anhydrous potassium carbonate (355 mg) in acetonitrile (10 ml) was stirred and heated at reflux for 17 hours. The solution was cooled and concentrated in vacuo. The residue was partitioned between EtOAc and water. The organic phase was separated, dried over Na₂SO₄ and concentrated in vacuo to give Example 653 as a yellow solid (186 mg). LCMS showed MH⁺=437; T_{RET}=2.82 min. δ_{H} (CDCl₃) 1.49 (3H, t), 1.80 (2H, m), 2.2-2.4 (4H, m), 2.54 (1H, m), 3.13 (1H, dt), 3.81 (3H, s), 4.13 (1H, m), 4.46 (2H, q), 4.54 (2H, d), 6.28 (1H, t), 6.90+7.28 (4H, AA′BB′), 7.98 (1H, s), 8.36 (1H, s), 9.84 (1H, d). Hydroxyl proton not visible.

The following examples were prepared by a similar procedure, e.g. using the same or a similar number of moles of reagents and the same or similar volumes of solvents:

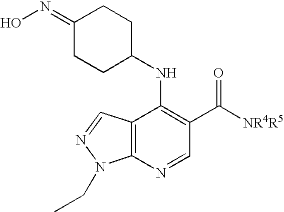

| | | | | | LC-MC |
|---|---|---|---|---|---|
| Example | | Source of | Starting | MH⁺ | Retention |
| No. | NR⁴R⁵ | HNR⁴R⁵ | Material | ion | time |
| 654 | 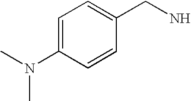 | Aldrich | Example 265 | 450 | 2.35 |
| 680 | 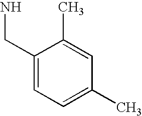 | Salor; or ICNBiomedicals,Inc.; orSynthesis,1982, 12,1036 | Example 260 | 435 | 3.10 |
| 681 | 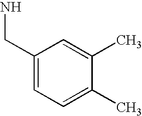 | CHMSRV-AS; orMatrixScientific; orChem. Ber.,1969, 102,2770 | Example 261 | 435 | 3.08 |
| 682 | 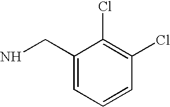 | Lancaster | Example 677 | 475 | 3.20 |
| 683 | 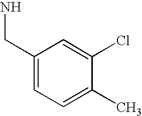 | MaybridgeChemicalCompanyLtd.; orWO 01/30745 | Example 678 | 455 | 3.17 |
| 684 | 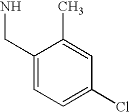 | Trans WorldChemicals,Inc.; or DE1953059 | Example 679 | 455 | 3.17 |
| 685 | 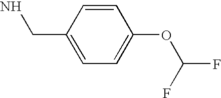 | Fluorochem;or WO 98/45268 | Example 266 | 473 | 3.00 |
| 686 | 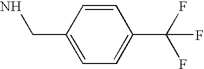 | Aldrich; orMeindl et al.,J. Med.Chem.,1984, 27(9),1111; orOrg. Lett.,2002, 4(12),2055 | Example 267 | 475 | 3.13 |

See later for alternative preparation of Example 681.

### Example 655

### 1-Ethyl-4-({4-[(ethyloxy)imino]cyclohexyl}amino)-N-{[4-(methyloxy)phenyl]methyl}-1H-pyrazolo[3,4-b]pyridine-5-carboxamide

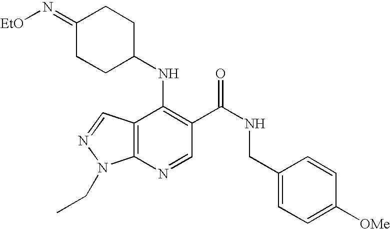

A mixture of Example 263 (25 mg), ethoxyamine hydrochloride (R²⁶ONH₂ where R²⁶=Et, 20 mg) and diisopropylethylamine (30 mg) in acetonitrile (3 ml) was stirred and heated at reflux for 3.25 hours. The solution was cooled and concentrated in vacuo. The residue was applied to an SPE cartridge (5 g). The cartridge was eluted with EtOAc. Fractions containing the desired product were concentrated in vacuo to give Example 655 as a colourless gum (20 mg). LCMS showed MH⁺=465; T_{RET}=3.28 min.

The following examples were prepared by a similar procedure, e.g. using the same or a similar number of moles of reagents and the same or similar volumes of solvents:

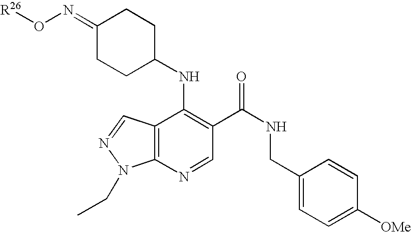

| | | | | | LC-MC |
|---|---|---|---|---|---|
| Example | | Source of | Starting | MH⁺ | Retention |
| No. | R²⁶ | R²⁶ONH₂ | Material | ion | time |
| 656 | Me | Aldrich | Example 263 | 451 | 2.52 |
| 657 | ^{t}Bu | Aldrich | Example 263 | 493 | 3.66 |

### Example 658

### 1-Ethyl-N-{[4-(methyloxy)phenyl]methyl}-4-[(7-oxohexahydro-1H-azepin-4-yl)amino]-1H-pyrazolo[3,4-b]pyridine-5-carboxamide

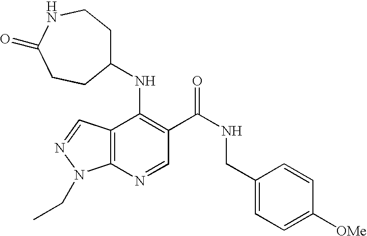

A suspension of cyanuric chloride (2,4,6-trichloro-1,3,5-triazine) (150 mg) in DMF (0.2 ml) was stirred for 30 minutes at room temperature. The suspension was diluted to 7 ml with DMF, with stirring. A 1.0 ml portion of the resultant suspension was removed and added to Example 653 (52 mg). The resultant solution was stirred for 90 hours at room temperature, then concentrated in vacuo. The residue was partitioned between EtOAc and water. The organic phase was separated and washed consecutively with saturated sodium carbonate, 10% w/v citric acid and saturated brine, dried over Na₂SO₄ and concentrated in vacuo. The residue was applied to an SPE cartridge (2 g). The cartridge was eluted successively with EtOAc:cyclohexane (1:1), EtOAc and then a (100:8:1) mixture of dichloromethane, ethanol and ammonia. Fractions containing the desired product (eluted in the ammoniacal solution) were concentrated in vacuo to give Example 658 as a colourless oil (11 mg). LCMS showed MH⁺=437; T_{RET}=2.50 min.

### Example 659

### Ethyl 1-ethyl-4-[(7-oxohexahydro-1H-azepin-4-yl)amino]-1H-pyrazolo[3,4-b]pyridine-5-carboxylate

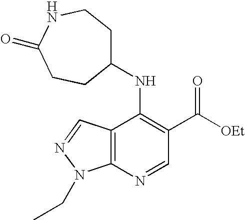

### Examples 661 to 664

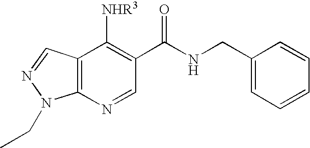

### General Procedure:

Intermediate 17 (0.16 mmol) in acetonitrile (1 ml) was treated with the R³NH₂ amine (0.8 mmol) in acetonitrile (1 ml) and N,N-diisopropylethylamine (0.8 mmol). The mixture was heated at 50° C. for 18 h then concentrated in vacuo. The residue was diluted with water (3 ml) and extracted with dichloromethane (2×5 ml). The combined organic extracts were evaporated, and the residue was purified by mass directed autoprep HPLC to give the desired product containing formic acid. This material was dissolved in chloroform-methanol (10/1, 5.5 ml) and washed with 5% sodium hydrogen carbonate solution (1 ml) to give after evaporation of solvents the pure product.

| | | | | | LC-MC |
|---|---|---|---|---|---|
| Example | | Source of | Starting | MH⁺ | Retention |
| no. | NHR³** | R³NH₂ | Material | ion | time |
| 214 | 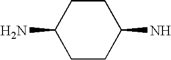 | J. Med. Chem.,1994, 37(17),2360 | Intermediate17 | 393 | 2.16 |
| 661 | 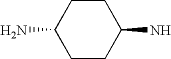 | Aldrich | Intermediate17 | 393 | 2.16 |
| 662 | 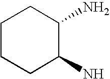 | Aldrich | Intermediate17 | 393 | 2.29 |
| 663 | 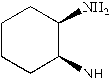 | Aldrich | Intermediate17 | 393 | 2.30 |
| 664 | 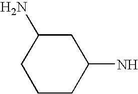 | PeakdaleMolecularLtd. | Intermediate17 | 393 | 2.21 |

^{**}For NHR³ in Examples 214 and 661-663, NHR³ is the cis or trans isomer as shown. For examples 662-664, NHR³ is the 3-amino- or 2-amino-cyclohex-1-ylamino group in a racemic form.

### Example 665

### Ethyl 1-ethyl-4-{[(1SR,3RS)-3-hydroxycyclohexyl]amino}-1H-pyrazolo[3,4-b]pyridine-5-carboxylate

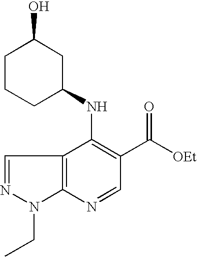

[cis-(3-hydroxycyclohex-1-yl)amino group, racemic]

3-Aminocyclohexanol (0.677 g, 5.9 mmol, as described in J. Chem. Soc., Perkin Trans 1, 1994, 537) in acetonitrile (10 ml) and ethanol (1 ml) was added at room temperature to a stirred solution of Intermediate 1 (1.24 g, 4.9 mmol) and diisopropylethylamine (4.26 ml, 24.5 mmol) in acetonitrile (25 ml). The resulting mixture was stirred at 85° C. for 17 h. The mixture was concentrated in vacuo, and the residue was partitioned between DCM (50 ml) and water (10 ml). The phases were separated and the organic phase was dried (Na₂SO₄) and evaporated to give an orange-brown oil. The oil was purified by Biotage chromatography (silica 10 g) eluting with 30-50% EtOAc in cyclohexane to give Example 665 as a white foam (0.681 g). LCMS showed MH⁺=333; T_{RET}=2.76 min.

### Examples 666-676

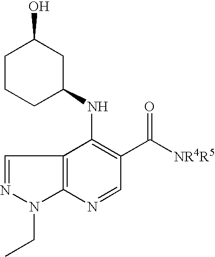

[cis-(3-hydroxycyclohex-1-yl)amino group, racemic]

### General Procedure:

A mixture of Intermediate 76 (0.1 mmol), HATU (0.1 mmol) and DIPEA (0.4 mmol) in DMF (0.5 ml) was shaken at room temperature for 10 min. A solution of the amine HNR⁴R⁵ (0.12 mmol) in DMF (0.5 ml) was then added and the mixture agitated for several minutes to give a solution. The solution was stored at room temperature for 16 h, then concentrated in vacuo. The residue was purified by mass directed autoprep HPLC.

| | | | | | LC-MC |
|---|---|---|---|---|---|
| Example | | Source of | Starting | MH⁺ | Retention |
| no. | NR⁴R⁵ | HNR⁴R⁵ | Material | ion | time |
| 666 | 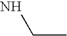 | Aldrich | Intermediate76 | 332 | 2.35 |
| 667 | 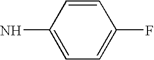 | Aldrich | Intermediate76 | 398 | 2.96 |
| 668 | 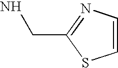 | ManchesterOrganics Ltd | Intermediate76 | 401 | 2.48 |
| 669 | 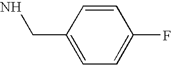 | Aldrich | Intermediate76 | 412 | 2.88 |
| 670 | 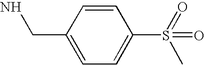 | Acros | Intermediate76 | 472 | 2.57 |
| 671 | 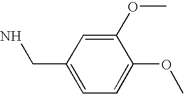 | Aldrich | Intermediate76 | 454 | 2.67 |
| 672 | 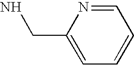 | Aldrich | Intermediate76 | 395 | 2.15 |
| 673 | 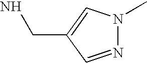 | N. D. ZelinskyInstitute | Intermediate76 | 398 | 2.35 |
| 674 | 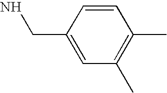 | MatrixScientific; orChem. Ber.,1969, 102,2770 | Intermediate76 | 422 | 3.08 |
| 675 | 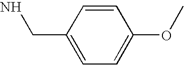 | Aldrich | Intermediate76 | 424 | 2.81 |
| 676 | 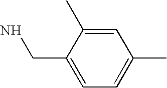 | ICNBiomedicals,Inc.; or Salor;or Synthesis,1982, 12, 1036 | Intermediate76 | 422 | 3.08 |

### Example 260

### Alternative Procedure

### N-[(2,4-dimethylphenyl)methyl]-1-ethyl-4-[(4-oxocyclohexyl)amino]-1H-pyrazolo[3,4-b]pyridine-5-carboxamide

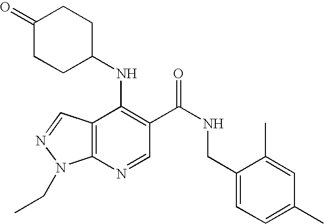

### Alternative Procedure for Preparing Example 260

A solution of Intermediate 58 (45 mg), HATU (63 mg) and DIPEA (39 mg) in acetonitrile (5 ml) was stirred for 10 min. A solution of 2,4-dimethylbenzylamine (24 mg) (available from Salor; or ICN Biomedicals, Inc.; or Synthesis, 1982, 12, 1036) in acetonitrile (1 ml) was added. The reaction mixture was stirred for 18 h. The solution was concentrated and the residue partitioned between ethyl acetate (25 ml) and 0.5M sodium bicarbonate (20 ml). The organic phase was separated, washed with water (20 ml), dried over Na₂SO₄ and concentrated to leave a gum which was applied to an SPE cartridge (5 g). The cartridge was eluted with ethyl acetate. Fractions containing the desired compound were combined and concentrated in vacuo to give Example 260 (32 mg). LC-MS showed MH⁺=420; T_{RET}=3.16 min. δ_{H} (CDCl₃): 1.49 (3H, t), 2.11 (2H, m), 2.33 (3H, s), 2.35 (3H, s), 2.40 (2H, m), 2.52 (2H, m), 2.61 (2H, m), 4.36 (1H, m), 4.47 (2H, q), 4.55 (2H, d), 6.14 (1H, t), 7.01+7.18 (2H, AA′BB′), 7.04 (1H, s), 8.01 (1H, s), 8.36 (1H, s), 9.96 (1H, d).

The following Examples 677-679 were prepared in a similar manner to Example 260 (alternative procedure above), for example using the same or a similar number of moles of reagents and the same or similar volumes of solvents:

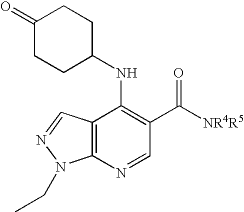

| | | | | | LC-MC |
|---|---|---|---|---|---|
| Example | | Source of | Starting | MH⁺ | Retention |
| no. | NR⁴R⁵ | HNR⁴R⁵ | Material | ion | time |
| 677 | 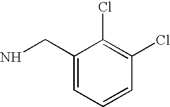 | Lancaster | Intermediate58 | 460 | 3.28 |
| 678 | 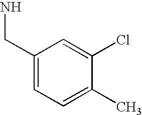 | MaybridgeChemicalCompany Ltd.;orWO 01/30745 | Intermediate58 | 440 | 3.25 |
| 679 | 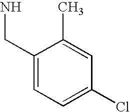 | Trans WorldChemicals, Inc.;or DE 1953059 | Intermediate58 | 440 | 3.24 |

Examples 680-686 and their preparation are shown above together with Example 653.

### Alternative Preparation of Example 681

### N-[(3,4-dimethylphenyl)methyl]-1-ethyl-4-{[4-(hydroxyimino)cyclohexyl]amino}-1H-pyrazolo[3,4-b]pyridine-5-carboxamide

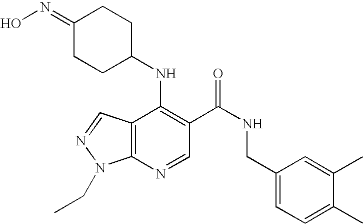

A mixture of Example 261 (35 mg), hydroxylamine hydrochloride (10 mg) and diisopropylethylamine (26 mg) in acetonitrile (4 ml) was stirred and heated at reflux for 2.5 hours. The solution was cooled and concentrated in vacuo. The residue was partitioned between EtOAc and water. The organic phase was separated, dried over Na₂SO₄ and concentrated in vacuo. The residue was applied to an SPE cartridge (10 g). The cartridge was eluted with EtOAc:cyclohexane (1:1) and then EtOAc. Fractions containing the desired compound were combined and concentrated in vacuo to give Example 681 as a white, amorphous solid (18 mg). LCMS showed MH⁺=435; T_{RET}=3.08 min. δ_{H} (CDCl₃) 1.49 (3H, t), 1.79 (2H, m), 2.24 (6H, s), 2.19-2.38 (4H, m), 2.56 (2H, dt), 4.13 (1H, m), 4.46 (2H, q), 4.53 (2H, d), 6.36 (1H, t), 7.09 (2H, t), 7.12 (1H, s), 7.98 (1H, s), 8.38 (1H, s), 9.79 (1H, d). Hydroxyl proton not visible.

1. A compound of formula (I) or a salt thereof: 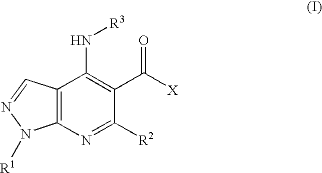
wherein:
R¹ is C₁₋₃alkyl C₁₋₂fluoroalkyl or —CH₂CH₂OH;
R² is a hydrogen atom (H), methyl or C₁fluoroalkyl;
R³ is unsubstituted C₆₋₈cycloalkyl;
or R³ is a bicyclic group of sub-formula (ee): 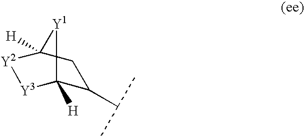
wherein Y¹, Y² and Y³ are all CH₂;
X is NR⁴R⁵ or OR^{5a}, in which:
R⁴ is a hydrogen atom (H); and
R⁵ is a hydrogen atom (H); C₁₋₈alkyl; C₁₋₈ fluoroalkyl; C₃₋₈cycloalkyl optionally substituted by a C₁₋₂alkyl group; or —(CH₂)ₙ⁴—C₃₋₈cycloalkyl optionally substituted, in the —(CH₂)ₙ⁴— moiety or in the C₃₋₈cycloalkyl moiety, by a C₁₋₂alkyl group, wherein n⁴ is 1, 2 or 3;
or R⁵ is C₂₋₆alkyl substituted by one or two independent substituents R¹¹;
wherein each substituent R¹¹, independently of any other R¹¹ substituent present, is: hydroxy (OH);
C₁₋₆alkoxy; phenyloxy; benzyloxy; —NR¹²R¹³; —NR¹⁵—C(O)R¹⁶; —NR¹⁵—C(O)—O—R¹⁶; —NR¹⁵—C(O)—NH—R¹⁵; or —NR¹⁵—SO₂R¹⁶; and wherein any R¹¹ substituent which is OH, alkoxy or —NR¹²R¹³ is not substituted at any carbon atom, of any R⁴ or R⁵ substituted alkyl, which is bonded to the nitrogen of NR⁴R⁵;
or R⁵ is —(CH₂)ₙ¹¹—C(O)R¹⁶; —(CH₂)ₙ¹²—C(O)NR¹²R¹³; —CHR¹⁹—C(O)NR¹²R¹³; —(CH₂)ₙ¹²—C(O)OR¹⁶; —(CH₂)ₙ¹²—C(O)OH; —CHR¹⁹—C(O)OR¹⁶; —CHR¹⁹—C(O)OH; —(CH₂)ₙ¹²—SO₂—NR¹²R¹³; —(CH₂)ₙ¹²—SO₂R¹⁶; or —(CH₂)ₙ¹²—CN; wherein n¹¹ is 0, 1, 2, 3 or 4 and n¹² is 1, 2, 3 or 4;
or R⁵ is —(CH₂)ₙ¹³-Het wherein n¹³ is 0, 1, 2, 3 or 4 and Het is a 4-, 5-, 6- or 7-membered saturated or partly-saturated heterocyclic ring containing one or two ring-hetero-atoms independently selected from O, S, and N; wherein any ring-hetero-atoms present are not bound to the —(CH₂)ₙ¹³— moiety when n n¹³ is 1 and are not bound to the nitrogen of NR⁴R⁵ when n¹³ is 0; wherein any ring-nitrogens which are present and which are not unsaturated are present as NR¹⁷ where R¹⁷ is as defined herein; and wherein one or two of the carbon ring-atoms independently are optionally substituted by C₁₋₂alkyl;
or R⁵ is phenyl optionally substituted with, independently, one, two or three of: a halogen atom; C₁₋₆alkyl; C₁₋₂fluoroalkyl; C₁₋₄alkoxy; C₁₋₂fluoroalkoxy; C₃₋₆cycloalkyloxy; —C(O)R^{16a}; —C(O)OR³⁰; —S(O)₂—R^{16a}; R^{16a}—S(O)₂—NR^{15a}; R⁷R⁸N—S(O)₂—; C₁₋₂alkyl-C(O)—R^{15a}N—S(O)₂—; C₁₋₄alkyl-S(O)—; Ph-S(O)—; R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-S(O)₂—CH₂—; R⁷R⁸N—S(O)₂—CH₂—; C₁₋₂alkyl-S(O)₂—NR^{15a}—CH₂—; —CH₂—OH; —CH₂CH₂—OH; —CH₂—NR⁷R⁸; —CH₂—CH₂—NR⁷R⁸; —CH₂—C(O)OR³⁰; —CH₂—C(O)—NR⁷R⁸; —CH₂—NR^{15a}—C(O)—C₁₋₃alkyl; —(CH₂)ₙ¹⁴-Het¹ where n¹⁴ is 0 or 1; cyano (CN); Ar^{5a}; or phenyl, pyridinyl or pyrimidinyl wherein the phenyl, pyridinyl or pyrimidinyl independently are optionally substituted by one or two of fluoro, chloro, C₁₋₂alkyl, C₁fluoroalkyl, C₁₋₂alkoxy or C₁fluoroalkoxy;
or where two adjacent substituents, on the R⁵ optionally substituted phenyl, taken together are —O—(CMe₂)—O— or —O—(CH₂)ₙ¹⁴—O— where n¹⁴ is 1 or 2;
wherein R⁷ and R⁸ are independently a hydrogen atom (H); C₁₋₄alkyl; C₃₋₆cycloalkyl; or phenyl optionally substituted by one or two of: fluoro, chloro, C₁₋₂alkyl, C₁fluoroalkyl, C₁₋₂alkoxy or C₁fluoroalkoxy; or R⁷ and R⁸ together are —(CH₂)ₙ⁶— or —C(O)—(CH₂)ₙ⁷— or —C(O)—(CH₂)ₙ⁷—C(O)— or —(CH₂)ₙ⁸—X⁷—(CH₂)ₙ⁹— or —C(O)—X⁷—(CH₂)ₙ¹⁰— in which: n⁶ is 3, 4, 5 or 6, n⁷ is 2, 3, 4, or 5, n⁸ and n⁹ and n¹⁰ independently are 2 or 3, and X⁷ is O or NR¹⁴ wherein R¹⁴ is H, C₁₋₂alkyl or C(O)Me;
or R⁵ has the sub-formula (x), (y), (y1) or (z): 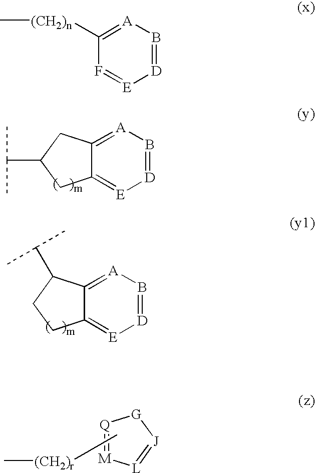
wherein in sub-formula (x), n=0, 1 or 2; in sub-formula (y) and (y1), m=1 or 2; and in sub-formula (z), r=0, 1 or 2;
wherein in sub-formula (x) and (y) and (y1), none, one or two of A, B, D, E and F are independently nitrogen or nitrogen-oxide (N⁺—O⁻) provided that no more than one of A, B, D, E and F is nitrogen-oxide; and the remaining of A, B, D, E and F are independently CH or CR⁶;
provided that when n is 0 in sub-formula (x) then one or two of A, B, D, E and F are independently nitrogen or nitrogen-oxide (N⁺—O⁻) and no more than one of A, B, D, E and F is nitrogen-oxide;
wherein, each R⁶, independently of any other R⁶ present, is: a halogen atom; C₁₋₆alkyl; C₁₋₄fluoroalkyl; C₁₋₄alkoxy; C₁₋₂fluoroalkoxy; C₃₋₆cycloalkyloxy; —C(O)R^{16a}; —C(O)OR³⁰; —S(O)₂—R^{16a}; R^{16a}—S(O)₂—NR^{15a}—; R⁷R⁸N—S(O)₂—; C₁₋₂alkyl-C(O)—R^{15a}N—S(O)₂—; C₁₋₄alkyl-S(O)—; Ph-S(O)—; R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-S(O)₂—CH₂—; R⁷R⁸N—S(O)₂—CH₂—; C₁₋₂alkyl-S(O)₂—NR^{15a}—CH₂—; —CH₂—OH; —CH₂CH₂—OH; —CH₂—NR⁷R⁸; —CH₂—CH₂—NR⁷R⁸; —CH₂—C(O)OR³⁰; —CH₂—C(O)—NR⁷R⁸; —CH₂—NR^{15a}—C(O)—C₁₋₃alkyl; —(CH₂)ₙ¹⁴-Het¹ where n¹⁴ is 0 or 1; cyano (CN); Ar^{5b}; or phenyl, pyridinyl or pyrimidinyl wherein the phenyl, pyridinyl or pyrimidinyl independently are optionally substituted by one or two of fluoro, chloro, C₁₋₂alkyl, C₁fluoroalkyl, C₁₋₂alkoxy or C₁fluoroalkoxy;
or where two adjacent R⁶ taken together are —O—(CMe₂)—O— or —O—(CH₂)ₙ¹⁴—O— where n¹⁴ is 1 or 2;
wherein R⁷ and R⁸ are as herein defined;
wherein sub-formula (y) and (y1), independently, are optionally substituted by oxo (═O) at a ring carbon adjacent the 6-membered aromatic ring;
wherein in sub-formula (z), G is O or S or NR⁹ wherein R⁹ is a hydrogen atom (H), C₁₋₄alkyl or C₁₋₄fluoroalkyl; none, one, two or three of J, L, M and Q are nitrogen; and the remaining of J, L, M and Q are independently CH or CR⁶ where R⁶, independently of any other R⁶ present, is as defined herein; and
R^{5a} is C₁₋₈alkyl; C₁₋₈ fluoroalkyl; C₃₋₈cycloalkyl; —(CH₂)ₙ^{4a}—C₃₋₆cycloalkyl wherein n^{4a} is 1 or 2; phenyl optionally substituted with one or two of: a halogen atom, C₁₋₂alkyl, trifluoromethyl, C₁₋₂alkoxy or trifluoromethoxy; or R^{5a} has the sub-formula (x), (y), (y1) or (z) as defined herein;
R¹² and R¹³ independently are H; C₁₋₅alkyl; C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
or R¹² and R¹³ together are —(CH₂)ₙ⁶— or —C(O)—(CH₂)ₙ⁷— or —C(O)—(CH₂)ₙ⁷—C(O)— or —(CH₂)ₙ⁸—X¹²—(CH₂)ₙ⁹— or —C(O)—X¹²—(CH₂)ₙ¹⁰— in which: n⁶ is 3, 4, 5 or 6, n⁷ is 2, 3, 4, or 5, n⁸ and n⁹ and n¹⁰ independently are 2 or 3 and X¹² is O or NR^{14a} wherein R^{14a} is H, C₁₋₂alkyl or C(O)Me;
R¹⁵ is a hydrogen atom (H); C₁₋₄alkyl; C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
R^{15a}, independent of other R^{15a}, is a hydrogen atom (H) or C₁₋₄alkyl;
R¹⁶ and R^{16a} independently are:
C₁₋₆alkyl;
C₃₋₆cycloalkyl optionally substituted by one oxo (═O), OH or C₁₋₂alkyl substituent;
C₃₋₆cycloalkyl-CH₂—;
pyridinyl optionally substituted on a ring carbon atom by one of: a halogen atom, C₁₋₂alkyl, C₁fluoroalkyl, C₁₋₂alkoxy or C₁fluoroalkoxy;
Ar^{5c};
phenyl optionally substituted by one or two of: a halogen atom, C₁₋₂alkyl, C₁fluoroalkyl, C₁₋₂alkoxy or C₁fluoroalkoxy;
benzyl optionally substituted at an aromatic carbon atom by one or two of: a halogen atom, C₁₋₂alkyl, C₁fluoroalkyl, C₁₋₂alkoxy or C₁fluoroalkoxy; or
a 4-, 5-, 6- or 7-membered saturated heterocyclic ring connected at a ring-carbon and containing one or two ring-hetero-atoms independently selected from O, S, and N; wherein any ring-nitrogens which are present are present as NR²⁷ where R²⁷ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen;
wherein Ar^{5a}, Ar^{5b} and Ar^{5c} independently is/are a 5-membered aromatic heterocyclic ring containing one O, S or NR^{15a} in the 5-membered ring, wherein the 5-membered ring can optionally additionally contain one or two N atoms, and wherein the heterocyclic ring is optionally substituted on a ring carbon atom by one of: a halogen atom, C₁₋₂alkyl, C₁fluoroalkyl, —CH₂OH, —CH₂—OC₁₋₂alkyl, OH (including the keto tautomer thereof) or —CH₂—NR²⁸R²⁹ wherein R²⁸ and R²⁹ independently are H or methyl;
and R¹⁷ is a hydrogen atom (H); C₁₋₄alkyl; C₁₋₂fluoroalkyl; C₃₋₆cycloalkyl; —(CH₂)ₚ⁶—C(O)R¹⁶ wherein p⁶ is 0, 1, 2 or 3; —(CH₂)ₚ⁶—C(O)NR¹²R¹³; —(CH₂)ₚ⁶—C(O)OR¹⁶; —(CH₂)ₚ⁶—C(O)OH; —SO₂R¹⁶; —C(O)—CH₂—NR¹²R¹³; —C(O)—CH₂—NR^{15a}—C(O)—C₁₋₃alkyl; —C(O)—CH₂—O—C₁₋₃alkyl; or phenyl or benzyl wherein the phenyl or benzyl is optionally substituted at an aromatic carbon atom by one or two of: a halogen atom, C₁₋₂alkyl, C₁fluoroalkyl, C₁₋₂alkoxy or C₁fluoroalkoxy;
R¹⁹ is C₁₋₄alkyl; —(CH₂)ₙ²⁰—OR²⁰ wherein n²⁰ is 1, 2, 3 or 4 and R²⁰ is a hydrogen atom (H) or C₁₋₄alkyl; —CH(Me)-OH; —CH₂—SH; —CH₂—CH₂—S-Me; benzyl; or (4-hydroxyphenyl)methyl; and
R³⁰, independent of other R³⁰, is a hydrogen atom (H), C₁₋₄alkyl or C₃₋₆cycloalkyl; and
Het¹, independent of other Het¹, is a 4-, 5-, 6- or 7-membered saturated heterocyclic ring connected at a ring-carbon and containing one or two ring-hetero-atoms independently selected from O, S, and N; wherein any ring-nitrogens which are present are present as NR³¹ where R³¹ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen.

2. (canceled)

3. A compound according to claim 1 comprising formula (IB) or a salt thereof: 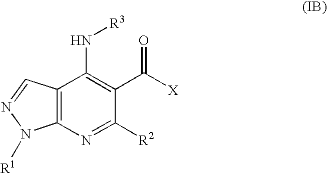
wherein:
R¹ is C₁₋₃alkyl, C₁₋₂fluoroalkyl or —CH₂CH₂OH;
R² is a hydrogen atom (H), methyl or C₁fluoroalkyl;
R³ is unsubstituted C₃₋₈cycloalkyl;
X is NR⁴R⁵ or OR^{5a}, in which:
R⁴ is a hydrogen atom (H); and
R⁵ is a hydrogen atom (H); C₁₋₈alkyl; C₁₋₈ fluoroalkyl; C₃₋₈cycloalkyl optionally substituted by a C₁₋₂alkyl group; or —(CH₂)ₙ⁴—C₃₋₈cycloalkyl optionally substituted, in the —(CH₂)ₙ⁴— moiety or in the C₃₋₈cycloalkyl moiety, by a C₁₋₂alkyl group, wherein n⁴ is 1, 2 or 3;
or R⁵ is C₂₋₆alkyl substituted by one or two independent substituents R¹¹;
wherein each substituent R¹¹, independently of any other R¹¹ substituent present, is: hydroxy (OH); C₁₋₆alkoxy; phenyloxy; benzyloxy; —NR¹²R¹³; —NR¹⁵—C(O)R¹⁶; —NR¹⁵—C(O)—O—R¹⁶; —NR¹⁵—C(O)—NH—R¹⁵; or —NR¹⁵—SO₂R¹⁶; and wherein any R¹¹ substituent which is OH, alkoxy or —NR¹²R¹³ is not substituted at any carbon atom, of any R⁴ or R⁵ substituted alkyl, which is bonded to the nitrogen of NR⁴R⁵;
or R⁵ is —(CH₂)ₙ¹¹—C(O)R¹⁶; —(CH₂)ₙ¹¹—C(O)NR¹²R¹³; —CHR¹⁹—C(O)NR¹²R¹³; —(CH₂)ₙ¹²—C(O)OR¹⁶; —CHR¹⁹—C(O)OR¹⁶; —(CH₂)ₙ¹²—SO₂—NR¹²R¹³; —(CH₂)ₙ¹²—SO₂R¹⁶; or —(CH₂)ₙ¹²—CN; wherein n¹¹ is 0, 1, 2, 3 or 4 and n¹² is 1, 2, 3 or 4;
or R⁵ is —(CH₂)ₙ¹³-Het wherein n¹³ is 0, 1, 2, 3 or 4 and Het is a 4-, 5-, 6- or 7-membered saturated or partly-saturated heterocyclic ring containing one or two ring-hetero-atoms independently selected from O, S, and N; wherein any ring-hetero-atoms present are not bound to the —(CH₂)ₙ¹³-moiety when n¹³ is 1 and are not bound to the nitrogen of NR⁴R⁵ when n¹³ is 0; wherein any ring-nitrogens which are present and which are not unsaturated are present as NR¹⁷ where R¹⁷ is as defined herein; and wherein one or two of the carbon ring-atoms independently are optionally substituted by C₁₋₂alkyl;
or R⁵ is phenyl optionally substituted with one or two of: a halogen atom; C₁₋₄alkyl; C₁₋₂fluoroalkyl; C₁₋₄alkoxy; C₁₋₂fluoroalkoxy; C₁₋₂alkylsulphonyl (C₁₋₂alkyl-SO₂—); C₁₋₂alkyl-SO₂—NH—; R⁷R⁸N—SO₂—; R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-SO₂—CH₂—; cyano (CN); or phenyl optionally substituted by one or two of fluoro, chloro, C₁₋₂alkyl, C₁fluoroalkyl, C₁₋₂alkoxy or C₁fluoroalkoxy;
wherein R⁷ and R⁸ are independently a hydrogen atom (H); C₁₋₄alkyl; C₃₋₆cycloalkyl; or phenyl optionally substituted by one or two of: fluoro, chloro, C₁₋₂alkyl, C₁fluoroalkyl, C₁₋₂alkoxy or C₁fluoroalkoxy; or R⁷ and R⁸ together are —(CH₂)ₙ⁶— or —C(O)—(CH₂)ₙ⁷— or —C(O)—(CH₂)ₙ⁷—C(O)— or —(CH₂)ₙ⁸—X⁷—(CH₂)ₙ⁹— or —C(O)—X⁷—(CH₂)ₙ¹⁰— in which: n⁶ is 3, 4, 5 or 6, n⁷ is 2, 3, 4, or 5, n⁸ and n⁹ and n¹⁰ independently are 2 or 3, and X⁷ is O or NR¹⁴ wherein R¹⁴ is H or C₁₋₂alkyl;
or R⁵ has the sub-formula (x), (y) or (z): 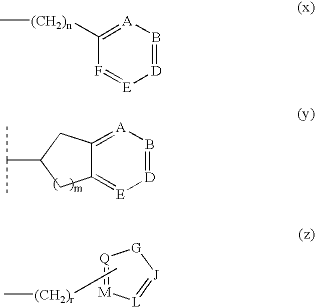
wherein in sub-formula (x), n=1 or 2; in sub-formula (y), m=1 or 2; and in sub-formula (z), r=0, 1 or 2;
wherein in sub-formula (x) and (y), none, one or two of A, B, D, E and F are nitrogen; and the remaining of A, B, D, E and F are independently CH or CR⁶;
where R⁶ is a halogen atom; C₁₋₄alkyl; C₁₋₄fluoroalkyl; C₁₋₄alkoxy; C₁₋₂fluoroalkoxy; C₁₋₂alkylsulphonyl (C₁₋₂alkyl-SO₂—); C₁₋₂alkyl-SO₂—NH—; R⁷R⁸N—SO₂—; R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-SO₂—CH₂—; cyano (CN); or phenyl optionally substituted by one or two of fluoro, chloro, C₁₋₂alkyl, C₁fluoroalkyl, C₁₋₂alkoxy or C₁fluoroalkoxy; wherein R⁷ and R⁸ are as herein defined;
wherein in sub-formula (z), G is O or S or NR⁹ wherein R⁹ is a hydrogen atom (H), C₁₋₄alkyl or C₁₋₄fluoroalkyl; none, one, two or three of J, L, M and Q are nitrogen; and the remaining of J, L, M and Q are independently CH or CR⁶ where R⁶ is as defined herein;
R¹⁷ is a hydrogen atom (H); C₁₋₄alkyl; C₁₋₂fluoroalkyl; C₃₋₆cycloalkyl; —(CH₂)ₚ⁶—C(O)R¹⁶ wherein p⁶ is 0, 1, 2 or 3; —(CH₂)ₚ⁶—C(O)NR¹²R¹³; —(CH₂)ₚ⁶—C(O)OR¹⁶; —SO₂R¹⁶; or phenyl or benzyl wherein the phenyl or benzyl is optionally substituted at an aromatic carbon atom by one or two of: a halogen atom, C₁₋₂alkyl, C₁fluoroalkyl, C₁₋₂alkoxy or C₁fluoroalkoxy;
R^{5a} is C₁₋₈alkyl; C₁₋₈ fluoroalkyl; C₃₋₈cycloalkyl; phenyl optionally substituted with one or two of: a halogen atom, C₁₋₂alkyl, trifluoromethyl, C₁₋₂alkoxy or trifluoromethoxy; or R^{5a} has the sub-formula (x), (y) or (z) as defined herein,
R¹² and R¹³ independently are H; C₁₋₅alkyl; C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
or R¹² and R¹³ together are —(CH₂)ₙ⁶— or —C(O)—(CH₂)ₙ⁷— or —C(O)—(CH₂)ₙ⁷—C(O)— or —(CH₂)ₙ⁸—X¹²—(CH₂)ₙ⁹— or —C(O)—X¹²—(CH₂)ₙ¹⁰ in which: n⁶ is 3, 4, 5 or 6, n⁷ is 2, 3, 4, or 5, n⁸ and n⁹ and n¹⁰ independently are 2 or 3 and X¹² is O or NR¹⁴ wherein R¹⁴ is H or C₁₋₂alkyl;
R¹⁵ is a hydrogen atom (H); C₁₋₄alkyl; C₃₋₆cycloalkyl; or phenyl optionally substituted by one or two of: a halogen atom, C₁₋₂alkyl, C₁fluoroalkyl, C₁₋₂alkoxy or C₁fluoroalkoxy;
R¹⁶ is C₁₋₄alkyl; C₃₋₆cycloalkyl; pyridinyl; or phenyl optionally substituted by one or two of: a halogen atom, C₁₋₂alkyl, C₁fluoroalkyl, C₁₋₂alkoxy or C₁fluoroalkoxy; and
R¹⁹ is a hydrogen atom (H); C₁₋₄alkyl; —(CH₂)ₙ²⁰—OR²⁰ wherein n²⁰ is 1, 2, 3 or 4 and R²⁰ is a hydrogen atom (H) or C₁₋₄alkyl; —CH(Me)-OH; —CH₂—SH; —CH₂—CH₂—S-Me; benzyl; or (4-hydroxyphenyl)methyl.

4. A compound or salt as claimed in claim 1 wherein R² is a hydrogen atom.

5. (canceled)

6. A compound or salt as claimed in claim 1, wherein R¹ is ethyl, n-propyl, C₂fluoroalkyl or —CH₂CH₂OH.

7. A compound or salt as claimed in claim 1, wherein R¹ is ethyl.

8-9. (canceled)

10. A compound or salt as claimed in claim 1, wherein R³ is unsubstituted C₆₋₈cycloalkyl.

11-24. (canceled)

25. A compound or salt as claimed in claim 1 wherein NHR³ is of sub-formula (c), (c 1), or (c 7):

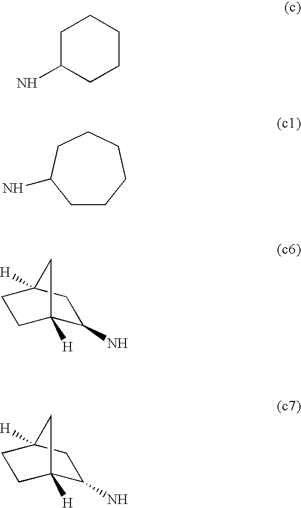

26-28. (canceled)

29. A compound or salt as claimed in claim 25 wherein NHR³ is of sub-formula (c).

30. (canceled)

31. A compound or salt as claimed in claim 1 wherein X is NR⁴R⁵.

32.-33. (canceled)

34. A compound or salt as claimed in claim 1 wherein R⁵ is:
C₁₋₈alkyl;
C₁₋₃fluoroalkyl;
C₃₋₈cycloalkyl (unsubstituted);
unsubstituted —(CH₂)ₙ⁴—C₅₋₆cycloalkyl wherein n⁴ is 1 or 2;
—(CH₂)ₙ⁵—R¹¹ wherein n⁵ is 2 or 3, and each substituent R¹¹, independently of any other R¹¹ substituent present, is C₁₋₄alkoxy, —NR¹⁵—C(O)—NH—R¹⁵, or —NR¹⁵—SO₂R¹⁶, and any R¹¹ substituent which is alkoxy is not substituted at any carbon atom, of the R⁵ substituted alkyl, which is bonded to the nitrogen of NR⁴R⁵;
or R⁵ is
—(CH₂)ₙ¹¹—C(O)R¹⁶;
—(CH₂)ₙ¹²—C(O)NR¹²R¹³;
—(CH₂)ₙ¹²—C(O)OR¹⁶;
—(CH₂)ₙ¹²—SO₂—NR¹²R¹³;
—(CH₂)ₙ¹²—SO₂R¹⁶;
or —(CH₂)ₙ¹²—CN wherein n¹¹ is 1 or 2 and n¹² is 1 or 2.

35. A compound or salt as claimed in claim 1 wherein R⁵ is —(CH₂)ₙ¹³-Het, n¹³ is 0, 1 or 2, and Het is a 5- or 6-membered saturated heterocyclic ring.

36. A compound or salt as claimed in claim 1 wherein R⁵ is phenyl optionally substituted with, independently, one or two of:
a halogen atom;
C₁₋₂alkyl;
C₁₋₂fluoroalkyl;
C₁₋₂alkoxy;
trifluoromethoxy;
C₁₋₂alkylsulphonyl (C₁₋₂alkyl-SO₂—);
C₁₋₂alkyl-SO₂—NH—;
R⁷R⁸N—SO₂—;
R⁷R⁸N—CO—;
—NR¹⁵—C(O)R¹⁶;
R⁷R⁸N; OH; C₁₋₂alkoxymethyl;
C₁₋₂alkyl-SO₂—CH₂—;
cyano (CN);
or phenyl optionally substituted by one of fluoro, C₁₋₂alkyl, C₁fluoroalkyl, C₁₋₂alkoxy or C₁fluoroalkoxy.

37. A compound or salt as claimed in claim 35, wherein R⁵ is phenyl optionally substituted with one or two of: a halogen atom, C₁₋₂alkyl, trifluoromethyl, C₁₋₂alkoxy, trifluoromethoxy, R⁷R⁸N—SO₂—, R⁷R⁸N—CO—, or C₁₋₂alkyl-SO₂—CH₂—.

38. A compound or salt as claimed in claim 1 wherein R⁵ has the sub-formula (x) or (y) or (y1) or (z).

39. A compound or salt as claimed in claim 1 wherein R⁵ has the sub-formula (x).

40. A compound or salt as claimed in claim 1 wherein n=1, m=1, and r=1.

41. A compound or salt as claimed in claim 1 wherein, in sub-formula (x), (y) and/or (y1): none, one or two of A, B, D, E and F are nitrogen; none, one, two or three of A, B, D, E and F are CR⁶; and the remaining of A, B, D, E and F are CH.

42. A compound or salt as claimed in claim 41, wherein, in sub-formula (x), (y) and/or (y1), none or one of A, B, D, E and F are nitrogen.

43. A compound or salt as claimed in claim 1 wherein in sub-formula (x), (y), (y1) and/or (z), each R⁶, independently of any other R⁶ present, is a fluorine, chlorine, bromine or iodine atom, methyl, ethyl, n-propyl, isopropyl, C₄alkyl, trifluoromethyl, —CH₂OH, methoxy, ethoxy, C₁fluoroalkoxy, OH, C₁₋₃alkylS(O)₂—, C₁₋₃alkylS(O)₂—NH—, Me₂N—S(O)₂—, H₂N—S(O)₂—, —CONH₂, —CONHMe, —CO₂H, cyano (CN), NMe₂, t-butoxymethyl, or C₁₋₃alkylS(O)₂—CH₂—.

44. A compound or salt as claimed in claim 43, wherein in sub-formula (x), (y), (y1) and/or (z), each R⁶, independently of any other R⁶ present, is a fluorine, chlorine or bromine atom, methyl, ethyl, n-propyl, isopropyl, trifluoromethyl, —CH₂OH, methoxy, difluoromethoxy, methylsulphonyl, methyl-SO₂—NH— or methyl-SO₂—CH₂—.

45. A compound or salt as claimed in claim 1 wherein R⁵ is of sub-formula (x) and is: benzyl, (monoalkyl-phenyl)methyl, [mono(fluoroalkyl)-phenyl]methyl, (monohalo-phenyl)methyl, (monoalkoxy-phenyl)methyl, [mono(fluoroalkoxy)-phenyl]methyl, [mono(N,N-dimethylamino)-phenyl]methyl, [mono(methyl-SO₂—NH—)-phenyl]methyl, [mono(methyl-SO₂—)-phenyl]methyl, (dialkyl-phenyl)methyl, (monoalkyl-monohalo-phenyl)methyl, [mono(fluoroalkyl)-monohalo-phenyl]methyl, (dihalo-phenyl)methyl, (dihalo-monoalkyl-phenyl)methyl, [dihalo-mono(hydroxymethyl)-phenyl]methyl, or (dialkoxy-phenyl)methyl.

46. A compound or salt as claimed in claim 45, wherein R⁵ is: (monoC₁₋₃alkyl-phenyl)methyl; (monoC₁fluoroalkyl-phenyl)methyl; (monoC₁₋₂alkoxy-phenyl)methyl; [mono(C₁fluoroalkoxy)-phenyl]methyl; (diC₁₋₂alkyl-phenyl)methyl; (monoC₁₋₂alkyl-monohalo-phenyl)methyl; (dihalo-phenyl)methyl; (dihalo-monoC₁₋₂alkyl-phenyl)methyl; or [dihalo-mono(hydroxymethyl)-phenyl]methyl.

47. A compound or salt as claimed in claim 46, wherein R⁵ is:
(4-C₁₋₃alkyl-phenyl)methyl; (4-C₁fluoroalkyl-phenyl)methyl; (4-C₁₋₂alkoxy-phenyl)methyl; (4-C₁fluoroalkoxy-phenyl)methyl; (3,4-dimethyl-phenyl)methyl; (2,4-dimethyl-phenyl)methyl; (3,5-dimethyl-phenyl)methyl; (2,3-dimethyl-phenyl)methyl; (2,5-dimethyl-phenyl)methyl; (4-methyl-3-chloro-phenyl)methyl; (3-methyl-4-chloro-phenyl)methyl; (2-methyl-4-chloro-phenyl)methyl; (2-chloro-4-fluorophenyl)methyl; (2,4-difluoro-phenyl)methyl, (4-bromo-2-fluorophenyl)methyl; (4-chloro-2-fluorophenyl)methyl; (3,4-dichloro-phenyl)methyl; (2,4-dichloro-phenyl)methyl; (2,6-dichloro-phenyl)methyl; (2,3-dichloro-phenyl)methyl; (2,4-dichloro-6-methyl-phenyl)methyl; or [2,3-dichloro-6-(hydroxymethyl)-phenyl]methyl.

48. A compound or salt as claimed in claim 1 wherein R⁵ has the sub-formula (z), r is 1, none or one of J, L, M or Q is CR⁶, and if one of J, L, M or Q is CR⁶ then R⁶ is methyl or C₁fluoroalkyl, and R⁹ is a hydrogen atom (H) or methyl.

49. A compound or salt as claimed in claim 1, which is:
ethyl 4-(cyclohexylamino)-1-ethyl-1H-pyrazolo[3,4-b]pyridine-5-carboxylate,
4-(cyclohexylamino)-N-cyclopentyl-1-ethyl-1H-pyrazolo[3,4-b]pyridine-5-carboxamide,
N-cyclohexyl-1-ethyl-5-(pyrrolidin-1-ylcarbonyl)-1H-pyrazolo[3,4-b]pyridin-4-amine,
N-benzyl-4-(cyclohexylamino)-1-ethyl-1H-pyrazolo[3,4-b]pyridine-5-carboxamide,
4-(cyclohexylamino)-1-ethyl-N-(2-ethylbutyl)-1H-pyrazolo[3,4-b]pyridine-5-carboxamide,
4-(cyclohexylamino)-1-ethyl-N-(4-fluorophenyl)-1H-pyrazolo[3,4-b]pyridine-5-carboxamide,
4-(cyclohexylamino)-1-ethyl-N-n-propyl-1H-pyrazolo[3,4-b]pyridine-5-carboxamide,
N-benzyl-4-(cyclohexylamino)-1-methyl-1H-pyrazolo[3,4-b]pyridine-5-carboxamide,
4-(cyclohexylamino)-N-(2-ethylbutyl)-1-methyl-1H-pyrazolo[3,4-b]pyridine-5-carboxamide,
4-(cyclohexylamino)-N-(4-fluorophenyl)-1-methyl-1H-pyrazolo[3,4-b]pyridine-5-carboxamide,
ethyl 4-(cyclohexylamino)-1-ethyl-6-methyl-1H-pyrazolo[3,4-b]pyridine-5-carboxylate,
4-(cyclohexylamino)-1-ethyl-6-methyl-N-[4-(methylsulfonyl)benzyl]-1H-pyrazolo[3,4-b]pyridine-5-carboxamide,
N-benzyl 4-(cyclohexylamino)-1-ethyl-6-methyl-1H-pyrazolo[3,4-b]pyridine-5-carboxamide,
4-(cyclohexylamino)-1-ethyl-N-(4-fluorophenyl)-6-methyl-1H-pyrazolo[3,4-b]pyridine-5-carboxamide,
4-(cyclohexylamino)-1-ethyl-6-methyl-N-[4-(trifluoromethyl)benzyl]-H-pyrazolo[3,4-b]pyridine-5-carboxamide,
4-(cyclohexylamino)-N-(2,3-dihydro-(H-inden-2-yl)-1-ethyl-6-methyl-1H-pyrazolo[3,4-b]pyridine-5-carboxamide,
4-(cyclohexylamino)-1-ethyl-N-(phenylmethyl)-1H-pyrazolo[3,4-b]pyridine-5-carboxamide,
4-(cyclohexylamino)-1-ethyl-N-(phenylmethyl)-1H-pyrazolo[3,4-b]pyridine-5-carboxamide,
4-[(1R,2R,4S)-bicyclo[2.2.1]hept-2-ylamino]-1-ethyl-N-(phenylmethyl)-1H-pyrazolo[3,4-b]pyridine-5-carboxamide,
4-[(1R,2S,4S)-bicyclo[2.2.1]hept-2-ylamino]-1-ethyl-N-(phenylmethyl)-1H-pyrazolo[3,4-b]pyridine-5-carboxamide,
4-(cycloheptylamino)-1-ethyl-N-{[4-(methyloxy)phenyl]methyl}-1H-pyrazolo[3,4-b]pyridine-5-carboxamide,
4-[(1R,2R,4S)-bicyclo[2.2.1]hept-2-ylamino]-1-ethyl-N-{[4-(methyloxy)phenyl]methyl}-1H-pyrazolo[3,4-b]pyridine-5-carboxamide,
4-[(1R,2S,4S)-bicyclo[2.2.1]hept-2-ylamino]-1-ethyl-N-{[4-(methyloxy)phenyl]methyl}-1H-pyrazolo[3,4-b]pyridine-5-carboxamide,
4-(cycloheptylamino)-1-ethyl-N-({4-[(methylsulfonyl)amino]phenyl}methyl)-1H-pyrazolo[3,4-b]pyridine-5-carboxamide,
4-[(1R,2R,4S)-bicyclo[2.2.1]hept-2-ylamino]-1-ethyl-N-({4-[(methylsulfonyl)amino]phenyl}methyl)-1H-pyrazolo[3,4-b]pyridine-5-carboxamide,
4-[(1R,2S,4S)-bicyclo[2.2.1]hept-2-ylamino]-1-ethyl-N-({4-[(methylsulfonyl)amino]phenyl}methyl)-1H-pyrazolo[3,4-b]pyridine-5-carboxamide,
4-(cyclohexylamino)-1-ethyl-N-({4-[(methylsulfonyl)amino]phenyl}methyl)-1H-pyrazolo[3,4-b]pyridine-5-carboxamide,
4-(cycloheptylamino)-N-(2,3-dihydro-1H-inden-2-yl)-1-ethyl-1H-pyrazolo[3,4-b]pyridine-5-carboxamide,
4-[(1R,2R,4S)-bicyclo[22.1]hept-2-ylamino]-N-(2,3-dihydro-1H-inden-2-yl)-1-ethyl-1H-pyrazolo[3,4-b]pyridine-5-carboxamide,
4-[(1R,2S,4S)-bicyclo[22.1]hept-2-ylamino]-N-(2,3-dihydro-1H-inden-2-yl)-1-ethyl-1H-pyrazolo[3,4-b]pyridine-5-carboxamide,
N-[2-(aminosulfonyl)ethyl]-4-(cyclohexylamino)-1-ethyl-1H-pyrazolo[3,4-b]pyridine-5-carboxamide,
N-(2-amino-2-oxoethyl)-4-(cyclohexylamino)-1-ethyl-1H-pyrazolo[3,4-b]pyridine-5-carboxamide,
4-(cyclohexylamino)-1-ethyl-N-{2-[(methylsulfonyl)amino]ethyl}-1H-pyrazolo[3,4-b]pyridine-5-carboxamide,
4-(cyclohexylamino)-1-ethyl-N-(tetrahydro-2H-pyran-4-yl)-1H-pyrazolo[3,4-b]pyridine-5-carboxamide,
4-(cyclohexylamino)-1-ethyl-N-[(1-methyl-1H-pyrazol-4-yl)methyl]-1H-pyrazolo[3,4-b]pyridine-5-carboxamide,
4-(cyclohexylamino)-1-ethyl-N-{[3-(methylsulfonyl)phenyl]methyl}-1H-pyrazolo[3,4-b]pyridine-5-carboxamide,
N-{[3-(aminocarbonyl)phenyl]methyl}-4-(cyclohexylamino)-1-ethyl-1H-pyrazolo[3,4-b]pyridine-5-carboxamide,
4-(cyclohexylamino)-1-ethyl-N-(tetrahydro-2-furanylmethyl)-1H-pyrazolo[3,4-b]pyridine-5-carboxamide,
4-(cyclohexylamino)-N-({4-[(dimethylamino)sulfonyl]phenyl}methyl)-1-ethyl-1H-pyrazolo[3,4-b]pyridine-5-carboxamide,
N-[(5-chloro-2-pyridinyl)methyl]-4-(cyclohexylamino)-1-ethyl-1H-pyrazolo[3,4-b]pyridine-5-carboxamide,
4-(cyclohexylamino)-1-ethyl-N-{[4-(methylsulfonyl)phenyl]methyl}-1H-pyrazolo[3,4-b]pyridine-5-carboxamide,
4-(cyclohexylamino)-1-ethyl-N-{[6-(methyloxy)-3-pyridinyl]methyl}-1H-pyrazolo[3,4-b]pyridine-5-carboxamide,
4-(cyclohexylamino)-1-ethyl-N-{4-[(methylamino)carbonyl]phenyl}-1H-pyrazolo[3,4-b]pyridine-5-carboxamide,
4-(cyclohexylamino)-1-ethyl-N-({3-[(methylamino)carbonyl]phenyl}methyl)-1H-pyrazolo[3,4-b]pyridine-5-carboxamide,
N-{[4-(aminocarbonyl)phenyl]methyl}-4-(cyclohexylamino)-1-ethyl-1H-pyrazolo[3,4-b]pyridine-5-carboxamide,
4-(cyclohexylamino)-1-ethyl-N-[(4-hydroxyphenyl)methyl]-1H-pyrazolo[3,4-b]pyridine-5-carboxamide,
4-(cyclohexylamino)-1-ethyl-N-{[4-(methyloxy)phenyl]methyl}-1H-pyrazolo[3,4-b]pyridine-5-carboxamide,
4-(cyclohexylamino)-N-[(3,4-difluorophenyl)methyl]-1-ethyl-1H-pyrazolo[3,4-b]pyridine-5-carboxamide,
4-(cyclohexylamino)-1-ethyl-N-{[4-(trifluoromethyl)phenyl]methyl}-1H-pyrazolo[3,4-b]pyridine-5-carboxamide,
4-(cyclohexylamino)-1-ethyl-N-({3-[(methylsulfonyl)amino]phenyl}methyl)-1H-pyrazolo[3,4-b]pyridine-5-carboxamide,
4-(cyclohexylamino)-N-[(2,5-difluorophenyl)methyl]-1-ethyl-1H-pyrazolo[3,4-b]pyridine-5-carboxamide,
4-(cyclohexylamino)-1-ethyl-N-[(4-methylphenyl)methyl]-1H-pyrazolo[3,4-b]pyridine-5-carboxamide,
4-(cyclohexylamino)-1-ethyl-N-(2-{4-[(methylsulfonyl)amino]phenyl}ethyl)-1H-pyrazolo[3,4-b]pyridine-5-carboxamide,
4-(cyclohexylamino)-1-ethyl-N-[(2-hydroxyphenyl)methyl]-1H-pyrazolo[3,4-b]pyridine-5-carboxamide,
4-(cyclohexylamino)-N-[(3,4-dichlorophenyl)methyl]-1-ethyl-1H-pyrazolo[3,4-b]pyridine-5-carboxamide,
4-(cyclohexylamino)-N-[(3,5-dichlorophenyl)methyl]-1-ethyl-1H-pyrazolo[3,4-b]pyridine-5-carboxamide,
4-(cyclohexylamino)-1-ethyl-N-(2-phenylethyl)-1H-pyrazolo[3,4-b]pyridine-5-carboxamide,
4-(cyclohexylamino)-1-ethyl-N-(1,2,3,4-tetrahydro-1-naphthalenyl)-1H-pyrazolo[3,4-b]pyridine-5-carboxamide,
4-(cyclohexylamino)-1-ethyl-N-{[2-(methylsulfinyl)phenyl]methyl}-1H-pyrazolo[3,4-b]pyridine-5-carboxamide,
4-(cyclohexylamino)-1-ethyl-N-[2-(4-hydroxyphenyl)ethyl]-1H-pyrazolo[3,4-b]pyridine-5-carboxamide,
N-{2-[4-(aminosulfonyl)phenyl]ethyl}-4-(cyclohexylamino)-1-ethyl-1H-pyrazolo[3,4-b]pyridine-5-carboxamide,
4-(cyclohexylamino)-1-ethyl-N-({2-[(methylamino)carbonyl]phenyl}methyl)-1H-pyrazolo[3,4-b]pyridine-5-carboxamide,
4-(cyclohexylamino)-1-ethyl-N-{[2-(methylsulfonyl)phenyl]methyl}-1H-pyrazolo[3,4-b]pyridine-5-carboxamide,
methyl 2-[({[4-(cyclohexylamino)-1-ethyl-1H-pyrazolo[3,4-b]pyridin-5-yl]carbonyl}amino)methyl]benzoate,
4-(cyclohexylamino)-1-ethyl-N-{2-[4-(methylsulfonyl)phenyl]ethyl}-1H-pyrazolo[3,4-b]pyridine-5-carboxamide,
N-[4,5-bis(methyloxy)-2,3-dihydro-1H-inden-2-yl]-4-(cyclohexylamino)-1-ethyl-1H-pyrazolo[3,4-b]pyridine-5-carboxamide,
4-(cyclohexylamino)-1-ethyl-N-{[2-fluoro-3-(trifluoromethyl)phenyl]methyl}-1H-pyrazolo[3,4-b]pyridine-5-carboxamide,
4-(cyclohexylamino)-N-[(3,4-dimethylphenyl)methyl]-1-ethyl-1H-pyrazolo[3,4-b]pyridine-5-carboxamide,
4-(cyclohexylamino)-1-ethyl-N-[2-(4-fluorophenyl)ethyl]-1H-pyrazolo[3,4-b]pyridine-5-carboxamide,
4-(cyclohexylamino)-1-ethyl-N-[2-(4-methylphenyl)ethyl]-1H-pyrazolo[3,4-b]pyridine-5-carboxamide,
4-(cyclohexylamino)-1-ethyl-N-{2-[4-(methyloxy)phenyl]ethyl}-1H-pyrazolo[3,4-b]pyridine-5-carboxamide,
4-(cyclohexylamino)-1-ethyl-N-(2-pyridinylmethyl)-1H-pyrazolo[3,4-b]pyridine-5-carboxamide trifluoroacetate,
4-(cyclohexylamino)-N-[(3,5-difluorophenyl)methyl]-1-ethyl-1H-pyrazolo[3,4-b]pyridine-5-carboxamide,
4-(cyclohexylamino)-N-(2,3-dihydro-1H-inden-1-yl)-1-ethyl-1H-pyrazolo[3,4-b]pyridine-5-carboxamide,
4-(cyclohexylamino)-N-{[4-(dimethylamino)phenyl]methyl}-1-ethyl-1H-pyrazolo[3,4-b]pyridine-5-carboxamide trifluoroacetate,
4-(cyclohexylamino)-1-ethyl-N-[(2-fluorophenyl)methyl]-1H-pyrazolo[3,4-b]pyridine-5-carboxamide,
N-{[2,4-bis(methyloxy)phenyl]methyl}-4-(cyclohexylamino)-1-ethyl-1H-pyrazolo[3,4-b]pyridine-5-carboxamide,
N-[(6-chloro-2-pyridinyl)methyl]-4-(cyclohexylamino)-1-ethyl-1H-pyrazolo[3,4-b]pyridine-5-carboxamide trifluoroacetate,
N-({2-[acetyl(methyl)amino]phenyl}methyl)-4-(cyclohexylamino)-1-ethyl-1H-pyrazolo[3,4-b]pyridine-5-carboxamide trifluoroacetate,
4-(cyclohexylamino)-1-ethyl-N-{[4-fluoro-3-(trifluoromethyl)phenyl]methyl}-1H-pyrazolo[3,4-b]pyridine-5-carboxamide,
4-(cyclohexylamino)-N-[(1R)-2,3-dihydro-1H-inden-1-yl]-1-ethyl-1H-pyrazolo[3,4-b]pyridine-5-carboxamide,
4-(cyclohexylamino)-N-[(2,6-dichlorophenyl)methyl]-1-ethyl-1H-pyrazolo[3,4-b]pyridine-5-carboxamide,
methyl 3-[({[4-(cyclohexylamino)-1-ethyl-1H-pyrazolo[3,4-b]pyridin-5-yl]carbonyl}amino)methyl]benzoate,
4-(cyclohexylamino)-N-(2,3-dihydro-1H-inden-2-yl)-1-ethyl-1H-pyrazolo[3,4-b]pyridine-5-carboxamide,
methyl 4-[({[4-(cyclohexylamino)-1-ethyl-1H-pyrazolo[3,4-b]pyridin-5-yl]carbonyl}amino)methyl]benzoate,
4-(cyclohexylamino)-1-ethyl-N-(1H-tetrazol-5-ylmethyl)-1H-pyrazolo[3,4-b]pyridine-5-carboxamide,
4-(cyclohexylamino)-N-({4-[(difluoromethyl)oxy]phenyl}methyl)-1-ethyl-1H-pyrazolo[3,4-b]pyridine-5-carboxamide,
4-(cyclohexylamino)-1-ethyl-N-[(2-methyl-1,3-thiazol-4-yl)methyl]-1H-pyrazolo[3,4-b]pyridine-5-carboxamide,
N-[(2-chloro-6-fluorophenyl)methyl]-4-(cyclohexylamino)-1-ethyl-1H-pyrazolo[3,4-b]pyridine-5-carboxamide,
N-{[2-(aminocarbonyl)phenyl]methyl}-4-(cyclohexylamino)-1-ethyl-1H-pyrazolo[3,4-b]pyridine-5-carboxamide,
4-(cyclohexylamino)-N-{[2-(dimethylamino)phenyl]methyl}-1-ethyl-1H-pyrazolo[3,4-b]pyridine-5-carboxamide,
4-(cyclohexylamino)-1-ethyl-N-[(4-fluorophenyl)methyl]-1H-pyrazolo[3,4-b]pyridine-5-carboxamide,
4-(cyclohexylamino)-1-ethyl-N-{[3-(trifluoromethyl)phenyl]methyl}-1H-pyrazolo[3,4-b]pyridine-5-carboxamide,
4-(cyclohexylamino)-N-[(2,6-difluorophenyl)methyl]-1-ethyl-1H-pyrazolo[3,4-b]pyridine-5-carboxamide,
4-(cyclohexylamino)-1-ethyl-N-[(3-fluorophenyl)methyl]-1H-pyrazolo[3,4-b]pyridine-5-carboxamide,
4-(cyclohexylamino)-1-ethyl-N-{[2-(trifluoromethyl)phenyl]methyl}-1H-pyrazolo[3,4-b]pyridine-5-carboxamide,
N-(5-chloro-2,3-dihydro-1H-inden-2-yl)-4-(cyclohexylamino)-1-ethyl-1H-pyrazolo[3,4-b]pyridine-5-carboxamide,
4-(cyclohexylamino)-1-ethyl-N-({4-[(methylamino)carbonyl]phenyl}methyl)-1H-pyrazolo[3,4-b]pyridine-5-carboxamide,
4-(cyclohexylamino)-1-ethyl-N-[4-(methyloxy)phenyl]-1H-pyrazolo[3,4-b]pyridine-5-carboxamide,
4-(cyclohexylamino)-1-ethyl-N-[(6-oxo-1,6-dihydro-3-pyridinyl)methyl]-1H-pyrazolo[3,4-b]pyridine-5-carboxamide,
4-(cyclohexylamino)-1-ethyl-N-(3-pyridinylmethyl)-1H-pyrazolo[3,4-b]pyridine-5-carboxamide,
4-[({[4-(cyclohexylamino)-1-ethyl-1H-pyrazolo[3,4-b]pyridin-5-yl]carbonyl}amino)methyl]benzoic acid,
3-[({[4-(cyclohexylamino)-1-ethyl-1H-pyrazolo[3,4-b]pyridin-5-yl]carbonyl}amino)methyl]benzoic acid,
4-(cyclohexylamino)-N-(2,3-dihydro-1H-inden-2-yl)-1-ethyl-1H-pyrazolo[3,4-b]pyridine-5-carboxamide hydrochloride, or
4-(cyclohexylamino)-N-(2,3-dihydro-1H-inden-2-yl)-1-ethyl-1H-pyrazolo[3,4-b]pyridine-5-carboxamide methanesulphonate;
or a salt thereof.

50-55. (canceled)

56. A pharmaceutical composition comprising a compound of formula (I) as defined in claim 1 or a pharmaceutically acceptable salt thereof, and one or more pharmaceutically acceptable carriers and/or excipients.

57. A pharmaceutical composition as claimed in claim 56 which is suitable for and/or adapted for inhaled administration.

58. A pharmaceutical composition as claimed in claim 57, in which the compound or salt is in a particle-size-reduced form, wherein the particle size (D50 value) of the size-reduced compound or salt is about 0.5 to about 10 microns.

59-61. (canceled)

62. A method of treatment and/or prophylaxis of an inflammatory and/or allergic disease or cognitive impairment in a mammal such as a human in need thereof, which method comprises administering to the mammal a therapeutically effective amount of a compound of formula (I), as defined in claim 1, or a pharmaceutically acceptable salt thereof.

63. A method as claimed in claim 62, wherein the method is for the treatment and/or prophylaxis of chronic obstructive pulmonary disease (COPD), asthma, rheumatoid arthritis or allergic rhinitis in a mammal such a human.

64-74. (canceled)
